(19) United States
(12) Patent Application Publication
(10) Pub. No.: US 2026/0103761 A1
(43) Pub. Date: Apr. 16, 2026

(54) STOMACH CANCER DETECTION KIT OR DEVICE, AND DETECTION METHOD

(71) Applicants: TORAY INDUSTRIES, INC., Tokyo (JP); NATIONAL CANCER CENTER, Tokyo (JP)

(72) Inventors: Satoko KOZONO, Kamakura-shi (JP); Hitoshi NOBUMASA, Kamakura-shi (JP); Satoshi KONDOU, Kamakura-shi (JP); Hiroko SUDO, Kamakura-shi (JP); Junpei KAWAUCHI, Kamakura-shi (JP); Atsushi OCHIAI, Kashiwa-shi (JP); Motohiro KOJIMA, Kashiwa-shi (JP)

(73) Assignees: TORAY INDUSTRIES, INC., Tokyo (JP); NATIONAL CANCER CENTER, Tokyo (JP)

(21) Appl. No.: 19/235,085

(22) Filed: Jun. 11, 2025

Related U.S. Application Data
(62) Division of application No. 18/509,767, filed on Nov. 15, 2023, now Pat. No. 12,359,260.
Application No. 18/509,767 is a division of application No. 17/948,560, filed on Sep. 20, 2022, now Pat. No. 11,859,256.
Application No. 17/948,560 is a division of application No. 16/789,943, filed on Feb. 13, 2020, now Pat. No. 11,486,009.
Application No. 16/789,943 is a division of application No. 15/319,203, now Pat. No. 10,597,727.

(30) Foreign Application Priority Data
Jun. 16, 2014 (JP) 2014-123224
Mar. 31, 2015 (JP) 2015-071485

(51) Int. Cl. C12Q 1/6886; C12Q 1/06; C12Q 1/68; G01N 33/53; G01N 37/00

(52) U.S. Cl. CPC C12Q 1/6886; C12Q 1/06; C12Q 1/68; G01N 33/53; G01N 37/00; C12Q 2600/158; C12Q 2600/178

(57) ABSTRACT

Disclosed is a kit or a device for the detection of stomach cancer and a method for detecting stomach cancer, and provides a kit or a device for the detection of stomach cancer, including a nucleic acid(s) capable of specifically binding to a miRNA(s) in a sample from a subject, and a method for detecting stomach cancer, including measuring the miRNA(s) in vitro.

### CROSS-REFERENCE TO RELATED APPLICATIONS

This application is a Divisional of U.S. application Ser. No. 18/059,767 filed Nov. 15, 2023, which is a Divisional of U.S. application Ser. No. 17/948,560 filed Sep. 20, 2022 (now U.S. Pat. No. 11,859,256), which is a Divisional of U.S. application Ser. No. 16/789,943 filed on Feb. 13, 2020 (now U.S. Pat. No. 11,486,009), which is a Divisional of U.S. application Ser. No. 15/319,203 filed on Dec. 15, 2016 (now U.S. Pat. No. 10,597,727), which is a National Phase of PCT International Application No. PCT/JP2015/067267 filed on Jun. 16, 2015, which claims the benefit under 35 U.S.C. § 119(a) to Patent Application Nos. 2015-071485 and 2014-123224, filed in Japan on Mar. 31, 2015 and Jun. 16, 2014, respectively. All of the above applications are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 14, 2022, is named “PH-6236-PCT-US-DIV1-DIV1.xml” and is 591,826 bytes in size. The sequence listing contained in this .XML file is part of the specification and is hereby incorporated by reference herein in its entirety.

### TECHNICAL FIELD

The present invention relates to a kit or a device for the detection of stomach cancer, comprising a nucleic acid(s) capable of specifically binding to a particular miRNA(s), which is used for examining the presence or absence of stomach cancer in a subject, and a method for detecting stomach cancer, comprising measuring an expression level(s) of the miRNA(s) using the nucleic acid.

### BACKGROUND ART

The stomach is a sac-like digestive organ connected to the esophagus. The stomach temporarily stores food from the esophagus and plays a role in the first step of digestion by secreting gastric juice. The stomach is divided into the cardial end located around the inlet leading to the esophagus, the pyloric end located around the outlet leading to the duodenum, and the other site called the gastric corpus (Non-Patent Literature 1). According to the statistics of the number of cancer type-specific incidences and deaths in Japan disclosed by the Center for Cancer Control and Information Services, National Cancer Center, estimated 125,730 individuals in total involving 86,728 males and 39,002 females were affected by stomach cancer in 2010. The number of stomach cancer deaths was a total of 49,129 people involving 32,206 males and 16,923 females in 2012. Thus, stomach cancer was the second leading cause of cancer death in Japan. Also, 22,220 Americans were affected by stomach cancer in 2014, among which 10,990 people would die of stomach cancer (Non-Patent Literature 1).

The stages of stomach cancer progression are defined in Non-Patent Literature 2 and classified into stages 0, IA, IB, IIA, IIB, IIIA, IIIB, IIIC, and IV according to tumor size, infiltration, lymph node metastasis, distant metastasis, etc. The 5-year relative survival rate of stomach cancer largely depends on the stages of cancer progression and is reportedly 57 to 71% for stage I, 33 to 46% for stage II, 9 to 20% for stage III, and 4% for stage IV (Non-Patent Literature 1). Thus, the early detection of stomach cancer leads to improvement in the survival rate. Therefore, an approach that enables early detection is strongly desired.

The treatment of stomach cancer is performed by the combined use of surgical therapy, drug therapy, and radiotherapy. Particularly, in very early stomach cancer under no suspicion of lymph node metastasis, endoscopic mucosal resection (EMR) or endoscopic submucosal dissection (ESD) is often applicable and the cancer can thus be treated without any burden on patients.

With the aim of detecting stomach cancer early, Japanese men and women aged 40 or older are recommended to take stomach cancer screening once a year. The efficacy of “gastric X-ray examination” as a method for stomach cancer screening has been shown. When detailed examination is required as a result of X-ray examination, gastroscopy is carried out. Alternatively, diagnostic imaging such as CT, PET, or MRI is also utilized for detecting stomach cancer (Non-Patent Literature 1).

On the other hand, no blood marker has been established for the screening of stomach cancer. Although the association of protein tumor markers such as CEA and CA19-9 in serum with stomach cancer has been suggested (Non-Patent Literature 3), there is no enough evidence to recommend using these markers for the purpose of screening. Meanwhile, as shown in Patent Literatures 1 to 3, there are reports, albeit at a research stage, on the detection of stomach cancer using the expression levels of microRNAs (miRNAs) or combinations of the expression levels of miRNAs and the expression levels of additional protein markers in biological samples including blood.

Patent Literature 1 discloses a method for detecting cancers including stomach cancer using hsa-miR-125a-3p in blood.

Patent Literature 2 discloses a method for detecting stomach cancer using hsa-miR-23a-3p, miR-92-1, and miR-92-2 (miR-92a-1-3p and miR-92a-2-3p) and also using miR-128b (miR-128-2-3p), miR-30c (miR-30c-5p), miR-135-1, miR-135-2 (miR-135a-5p), and miR-149 (miR-149-5p), and other miRNAs in blood or tissues.

Patent Literature 3 discloses a method for detecting stomach cancer using hsa-miR-451 and 468 (hsa-miR-468-5p) in blood.

### CITATION LIST

### Patent Literature

Patent Literature 1: International Publication No. WO 2010/062706
Patent Literature 2: JP Patent Publication (Kokai) No. 2014-060993 A (2014)
Patent Literature 3: JP Patent Publication (Kokai) No. 2013-085542 A (2013)

### Non-Patent Literature

Non-Patent Literature 1: American Cancer Society, “Stomach Cancer”, 2013, p. 3, 6, and 18 to 20, http://www.cancer.org/acs/groups/cid/documents/webcontent/003141-pdf.pdf
Non-Patent Literature 2: Sobin, L. et al, “TNM Classification of Malignant Tumours, the 7th edition, Japanese version”, 2010, p. 69 to 73
Non-Patent Literature 3: Kim, H. J. et al., Acta Oncologica, 2009, Vol. 48, p. 385 to 390

### SUMMARY OF INVENTION

### Technical Problem

An object of the present invention is to find a novel tumor marker(s) for stomach cancer and to provide a method that can effectively detect stomach cancer using a nucleic acid(s) capable of specifically binding to the marker(s). Primary tests of stomach cancer include imaging tests such as gastric X-ray examination, which is routinely used in Japan, as well as CT, PET, and MRI (Non-Patent Literature 1). In Japan, however, stomach cancer is still the second leading cause of cancer death. Thus, the imaging tests cannot always work as a deterrent against stomach cancer death.

For example, CEA and CA19-9 are known as tumor markers for the detection of stomach cancer. In general, as shown in Non-Patent Literature 3, 5 ng/mL for CEA and 37 U/mL for CA19-9 are used as reference values. Although these tumor markers may be helpful in confirming the recurrence of or therapeutic effects on stomach cancer, their expression very rarely elevates in early stomach cancer. Therefore, these markers may not be useful for the purpose of stomach cancer screening. The tumor markers such as CEA and CA19-9 may also elevate for reasons other than those due to stomach cancer. Therefore, these markers alone allegedly fail to determine the presence or absence of stomach cancer. The false diagnosis of other cancers as stomach cancer wastes appropriate therapeutic opportunity or places unnecessary economical and physical burdens on patients due to the application of wrong medicine.

As described below, there are reports, albeit at a research stage, on the determination of stomach cancer using the expression levels of microRNAs (miRNAs) in biological samples including blood, none of which, however, have yet been brought into practical use.

Patent Literature 1 discloses a method for detecting cancers including stomach cancer using hsa-miR-125a-3p and other miRNAs in blood. This detection method, however, does not describe specific detection performance such as accuracy, sensitivity, or specificity for determining stomach cancer and is thus industrially less practical.

Patent Literature 2 discloses a method for detecting stomach cancer using hsa-miR-23a-3p, miR-92-1, and miR-92-2 (miR-92a-1-3p and miR-92a-2-3p) and further using miR-128 (miR-128-2-5p), miR-30c (miR-30c-5p), miR-135-1, miR-135-2 (miR-135a-5p), miR-149 (miR-149-5p), and other miRNAs in blood or tissues.

Among them, hsa-miR-23a-3p, miR-92-1, and miR-92-2 (miR-92a-1-3p and miR-92a-2-3p) are particularly described as miRNAs for detecting stomach cancer. According to the description therein, these markers in blood, however, were not validated, and specific detection examples were given for miRNAs in tissues. This is not an easy screening test. Therefore, this detection method is industrially less practical.

As mentioned above, the existing tumor markers exhibit low performance in the detection of stomach cancer, or neither detection methods nor performance is specifically shown as to the markers at a research stage. Therefore, use of these markers might lead to carrying out needless extra examination due to the false detection of healthy subjects as being stomach cancer patients, or might waste therapeutic opportunity because of overlooking stomach cancer patients. In addition, the measurement of dozens to several hundreds of miRNAs increases examination cost and is therefore difficult to use in large-scale screening for medical checkup, etc. Furthermore, the collection of gastric tissues for measuring the tumor markers is highly invasive to patients and is not favorable. Hence, there is a demand for a highly accurate stomach cancer marker that is detectable from blood, which can be collected with limited invasiveness, and is capable of correctly discriminating a stomach cancer patient from a healthy subject. Particularly, screening based on an imaging test, such as gastric X-ray examination, which is currently carried out for the early detection of stomach cancer, presents problems associated with radiation exposure, high cost, etc. Therefore, the provision of a more convenient primary screening test of stomach cancer probably leads to benefits to subjects and the health service.

### Solution to Problem

The present inventors have conducted diligent studies to attain the object and consequently completed the present invention by finding multiple genes usable as markers for the detection of stomach cancer from blood, which can be collected with limited invasiveness, and finding that stomach cancer can be significantly detected by using a nucleic acid(s) capable of specifically binding to any of these markers.

### SUMMARY OF INVENTION

Specifically, the present invention has the following features:
(1) A kit for the detection of stomach cancer, comprising a nucleic acid(s) capable of specifically binding to at least one or more polynucleotides selected from the group consisting of the following stomach cancer markers: miR-4257, miR-6726-5p, miR-1343-3p, miR-1247-3p, miR-6787-5p, miR-6875-5p, miR-1225-3p, miR-8063, miR-6781-5p, miR-4746-3p, miR-1908-5p, miR-6756-5p, miR-204-3p, miR-4651, miR-6757-5p, miR-6825-5p, miR-7108-5p, miR-4792, miR-7641, miR-3188, miR-3131, miR-6780b-5p, miR-8069, miR-6840-3p, miR-8072, miR-1233-5p, miR-6887-5p, miR-1231, miR-5572, miR-6738-5p, miR-6784-5p, miR-6791-5p, miR-6749-5p, miR-6741-5p, miR-128-1-5p, miR-4419b, miR-6746-5p, miR-3184-5p, miR-3679-5p, miR-7110-5p, miR-4516, miR-6717-5p, miR-6826-5p, miR-4433b-3p, miR-3679-3p, miR-3135b, miR-3622a-5p, miR-711, miR-4467, miR-6857-5p, miR-6515-3p, miR-1225-5p, miR-187-5p, miR-3185, miR-642b-3p, miR-1249, miR-744-5p, miR-4442, miR-1228-3p, miR-939-5p, miR-6845-5p, miR-887-3p, miR-7845-5p, miR-6729-5p, miR-4632-5p, miR-615-5p, miR-6724-5p, miR-4728-5p, miR-6732-5p, miR-6816-5p, miR-4695-5p, miR-6088, miR-7975, miR-3197, miR-6125, miR-4433-3p, miR-6727-5p, miR-4706, miR-7847-3p, miR-6805-3p, miR-6766-3p, miR-1913, miR-4649-5p, miR-602, miR-3663-3p, miR-6893-5p, miR-6861-5p, miR-4449, miR-6842-5p, miR-4454, miR-5195-3p, miR-663b, miR-6765-5p, miR-4513, miR-614, miR-6785-5p, miR-6777-5p, miR-940, miR-4741, miR-6870-5p, miR-6131, miR-150-3p, miR-4707-5p, miR-1915-3p, miR-3937, miR-937-5p, miR-4443, miR-1914-3p, miR-3620-5p, miR-1268b, miR-1227-5p, miR-6880-5p, miR-4417, miR-6802-5p, miR-6769a-5p, miR-663a, miR-6721-5p, miR-4532, miR-7977, miR-92b-5p, miR-371a-5p, miR-6126, miR-4734, miR-4665-3p, miR-423-5p, miR-1469, miR-4675, miR-1915-5p, miR-6716-5p, miR-718, miR-4281, miR-6820-5p, miR-6795-5p, miR-6779-5p, miR-7109-5p, miR-6798-5p, miR-4648, miR-8059, miR-6765-3p, miR-6132, miR-4492, miR-7107-5p, miR-3195, miR-3180, miR-296-3p, miR-564, miR-1268a, miR-6848-5p, miR-762, miR-2861, miR-1203, miR-1260b, miR-4476, miR-6885-5p, miR-6769b-5p, miR-23b-3p, miR-1343-5p, miR-3621, miR-4688, miR-4286, miR-4640-5p, miR-4739, miR-1260a, miR-4276, miR-7106-5p, miR-6794-5p, miR-6774-5p, miR-4707-3p, miR-4534, miR-4294, miR-6850-5p, miR-6089 and miR-671-5p.
(2) The kit according to (1), wherein miR-4257 is hsa-miR-4257, miR-6726-5p is hsa-miR-6726-5p, miR-1343-3p is hsa-miR-1343-3p, miR-1247-3p is hsa-miR-1247-3p, miR-6787-5p is hsa-miR-6787-5p, miR-6875-5p is hsa-miR-6875-5p, miR-1225-3p is hsa-miR-1225-3p, miR-8063 is hsa-miR-8063, miR-6781-5p is hsa-miR-6781-5p, miR-4746-3p is hsa-miR-4746-3p, miR-1908-5p is hsa-miR-1908-5p, miR-6756-5p is hsa-miR-6756-5p, miR-204-3p is hsa-miR-204-3p, miR-4651 is hsa-miR-4651, miR-6757-5p is hsa-miR-6757-5p, miR-6825-5p is hsa-miR-6825-5p, miR-7108-5p is hsa-miR-7108-5p, miR-4792 is hsa-miR-4792, miR-7641 is hsa-miR-7641, miR-3188 is hsa-miR-3188, miR-3131 is hsa-miR-3131, miR-6780b-5p is hsa-miR-6780b-5p, miR-8069 is hsa-miR-8069, miR-6840-3p is hsa-miR-6840-3p, miR-8072 is hsa-miR-8072, miR-1233-5p is hsa-miR-1233-5p, miR-6887-5p is hsa-miR-6887-5p, miR-1231 is hsa-miR-1231, miR-5572 is hsa-miR-5572, miR-6738-5p is hsa-miR-6738-5p, miR-6784-5p is hsa-miR-6784-5p, miR-6791-5p is hsa-miR-6791-5p, miR-6749-5p is hsa-miR-6749-5p, miR-6741-5p is hsa-miR-6741-5p, miR-128-1-5p is hsa-miR-128-1-5p, miR-4419b is hsa-miR-4419b, miR-6746-5p is hsa-miR-6746-5p, miR-3184-5p is hsa-miR-3184-5p, miR-3679-5p is hsa-miR-3679-5p, miR-7110-5p is hsa-miR-7110-5p, miR-4516 is hsa-miR-4516, miR-6717-5p is hsa-miR-6717-5p, miR-6826-5p is hsa-miR-6826-5p, miR-4433b-3p is hsa-miR-4433b-3p, miR-3679-3p is hsa-miR-3679-3p, miR-3135b is hsa-miR-3135b, miR-3622a-5p is hsa-miR-3622a-5p, miR-711 is hsa-miR-711, miR-4467 is hsa-miR-4467, miR-6857-5p is hsa-miR-6857-5p, miR-6515-3p is hsa-miR-6515-3p, miR-1225-5p is hsa-miR-1225-5p, miR-187-5p is hsa-miR-187-5p, miR-3185 is hsa-miR-3185, miR-642b-3p is hsa-miR-642b-3p, miR-1249 is hsa-miR-1249, miR-744-5p is hsa-miR-744-5p, miR-4442 is hsa-miR-4442, miR-1228-3p is hsa-miR-1228-3p, miR-939-5p is hsa-miR-939-5p, miR-6845-5p is hsa-miR-6845-5p, miR-887-3p is hsa-miR-887-3p, miR-7845-5p is hsa-miR-7845-5p, miR-6729-5p is hsa-miR-6729-5p, miR-4632-5p is hsa-miR-4632-5p, miR-615-5p is hsa-miR-615-5p, miR-6724-5p is hsa-miR-6724-5p, miR-4728-5p is hsa-miR-4728-5p, miR-6732-5p is hsa-miR-6732-5p, miR-6816-5p is hsa-miR-6816-5p, miR-4695-5p is hsa-miR-4695-5p, miR-6088 is hsa-miR-6088, miR-7975 is hsa-miR-7975, miR-3197 is hsa-miR-3197, miR-6125 is hsa-miR-6125, miR-4433-3p is hsa-miR-4433-3p, miR-6727-5p is hsa-miR-6727-5p, miR-4706 is hsa-miR-4706, miR-7847-3p is hsa-miR-7847-3p, miR-6805-3p is hsa-miR-6805-3p, miR-6766-3p is hsa-miR-6766-3p, miR-1913 is hsa-miR-1913, miR-4649-5p is hsa-miR-4649-5p, miR-602 is hsa-miR-602, miR-3663-3p is hsa-miR-3663-3p, miR-6893-5p is hsa-miR-6893-5p, miR-6861-5p is hsa-miR-6861-5p, miR-4449 is hsa-miR-4449, miR-6842-5p is hsa-miR-6842-5p, miR-4454 is hsa-miR-4454, miR-5195-3p is hsa-miR-5195-3p, miR-663b is hsa-miR-663b, miR-6765-5p is hsa-miR-6765-5p, miR-4513 is hsa-miR-4513, miR-614 is hsa-miR-614, miR-6785-5p is hsa-miR-6785-5p, miR-6777-5p is hsa-miR-6777-5p, miR-940 is hsa-miR-940, miR-4741 is hsa-miR-4741, miR-6870-5p is hsa-miR-6870-5p, miR-6131 is hsa-miR-6131, miR-150-3p is hsa-miR-150-3p, miR-4707-5p is hsa-miR-4707-5p, miR-1915-3p is hsa-miR-1915-3p, miR-3937 is hsa-miR-3937, miR-937-5p is hsa-miR-937-5p, miR-4443 is hsa-miR-4443, miR-1914-3p is hsa-miR-1914-3p, miR-3620-5p is hsa-miR-3620-5p, miR-1268b is hsa-miR-1268b, miR-1227-5p is hsa-miR-1227-5p, miR-6880-5p is hsa-miR-6880-5p, miR-4417 is hsa-miR-4417, miR-6802-5p is hsa-miR-6802-5p, miR-6769a-5p is hsa-miR-6769a-5p, miR-663a is hsa-miR-663a, miR-6721-5p is hsa-miR-6721-5p, miR-4532 is hsa-miR-4532, miR-7977 is hsa-miR-7977, miR-92b-5p is hsa-miR-92b-5p, miR-371a-5p is hsa-miR-371a-5p, miR-6126 is hsa-miR-6126, miR-4734 is hsa-miR-4734, miR-4665-3p is hsa-miR-4665-3p, miR-423-5p is hsa-miR-423-5p, miR-1469 is hsa-miR-1469, miR-4675 is hsa-miR-4675, miR-1915-5p is hsa-miR-1915-5p, miR-6716-5p is hsa-miR-6716-5p, miR-718 is hsa-miR-718, miR-4281 is hsa-miR-4281, miR-6820-5p is hsa-miR-6820-5p, miR-6795-5p is hsa-miR-6795-5p, miR-6779-5p is hsa-miR-6779-5p, miR-7109-5p is hsa-miR-7109-5p, miR-6798-5p is hsa-miR-6798-5p, miR-4648 is hsa-miR-4648, miR-8059 is hsa-miR-8059, miR-6765-3p is hsa-miR-6765-3p, miR-6132 is hsa-miR-6132, miR-4492 is hsa-miR-4492, miR-7107-5p is hsa-miR-7107-5p, miR-3195 is hsa-miR-3195, miR-3180 is hsa-miR-3180, miR-296-3p is hsa-miR-296-3p, miR-564 is hsa-miR-564, miR-1268a is hsa-miR-1268a, miR-6848-5p is hsa-miR-6848-5p, miR-762 is hsa-miR-762, miR-2861 is hsa-miR-2861, miR-1203 is hsa-miR-1203, miR-1260b is hsa-miR-1260b, miR-4476 is hsa-miR-4476, miR-6885-5p is hsa-miR-6885-5p, miR-6769b-5p is hsa-miR-6769b-5p, miR-23b-3p is hsa-miR-23b-3p, miR-1343-5p is hsa-miR-1343-5p, miR-3621 is hsa-miR-3621, miR-4688 is hsa-miR-4688, miR-4286 is hsa-miR-4286, miR-4640-5p is hsa-miR-4640-5p, miR-4739 is hsa-miR-4739, miR-1260a is hsa-miR-1260a, miR-4276 is hsa-miR-4276, miR-7106-5p is hsa-miR-7106-5p, miR-6794-5p is hsa-miR-6794-5p, miR-6774-5p is hsa-miR-6774-5p, miR-4707-3p is hsa-miR-4707-3p, miR-4534 is hsa-miR-4534, miR-4294 is hsa-miR-4294, miR-6850-5p is hsa-miR-6850-5p, miR-6089 is hsa-miR-6089, and miR-671-5p is hsa-miR-671-5p.
(3) The kit according to (1) or (2), wherein the nucleic acid is a polynucleotide selected from the group consisting of the following polynucleotides (a) to (e):
(a) a polynucleotide consisting of a nucleotide sequence represented by any of SEQ ID NOs: 1 to 165 and 635 to 642 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 165 and 635 to 642,
(c) a polynucleotide consisting of a nucleotide sequence complementary to a nucleotide sequence represented by any of SEQ ID NOs: 1 to 165 and 635 to 642 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 165 and 635 to 642 or a nucleotide sequence derived from the nucleotide sequence by the replacement of u with t, and
(e) a polynucleotide hybridizing under stringent conditions to any of the polynucleotides (a) to (d).
(4) The kit according to any of (1) to (3), wherein the kit further comprises a nucleic acid(s) capable of specifically binding to at least one or more polynucleotides selected from the group consisting of the following other stomach cancer markers: miR-128-2-5p, miR-125a-3p, miR-92a-2-5p, and miR-486-3p.
(5) The kit according to (4), wherein miR-128-2-5p is hsa-miR-128-2-5p, miR-125a-3p is hsa-miR-125a-3p, miR-92a-2-5p is hsa-miR-92a-2-5p, and miR-486-3p is hsa-miR-486-3p.
(6) The kit according to (4) or (5), wherein the nucleic acid is a polynucleotide selected from the group consisting of the following polynucleotides (f) to (j):
(f) a polynucleotide consisting of a nucleotide sequence represented by any of SEQ ID NOs: 166 to 169 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 166 to 169,
(h) a polynucleotide consisting of a nucleotide sequence complementary to a nucleotide sequence represented by any of SEQ ID NOs: 166 to 169 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 166 to 169 or a nucleotide sequence derived from the nucleotide sequence by the replacement of u with t, and
j) a polynucleotide hybridizing under stringent conditions to any of the polynucleotides (f) to (i).
(7) The kit according to any of (1) to (6), wherein the kit further comprises a nucleic acid(s) capable of specifically binding to at least one or more polynucleotides selected from the group consisting of the following other stomach cancer markers: miR-3196, miR-211-3p, miR-4271, miR-6851-5p, miR-149-3p, miR-4667-5p, miR-135a-3p, miR-4486, miR-4697-5p, miR-4725-3p, miR-6510-5p, miR-5001-5p, miR-4673, miR-4466, miR-23a-3p, miR-3656, miR-6782-5p, miR-4689, miR-451a, miR-4446-3p, miR-3180-3p, miR-642a-3p, miR-6889-5p, miR-3178, miR-4665-5p, miR-6722-3p, miR-30c-1-3p, miR-4507, miR-3141 and miR-1199-5p.
(8) The kit according to (7), wherein miR-3196 is hsa-miR-3196, miR-211-3p is hsa-miR-211-3p, miR-4271 is hsa-miR-4271, miR-6851-5p is hsa-miR-6851-5p, miR-149-3p is hsa-miR-149-3p, miR-4667-5p is hsa-miR-4667-5p, miR-135a-3p is hsa-miR-135a-3p, miR-4486 is hsa-miR-4486, miR-4697-5p is hsa-miR-4697-5p, miR-4725-3p is hsa-miR-4725-3p, miR-6510-5p is hsa-miR-6510-5p, miR-5001-5p is hsa-miR-5001-5p, miR-4673 is hsa-miR-4673, miR-4466 is hsa-miR-4466, miR-23a-3p is hsa-miR-23a-3p, miR-3656 is hsa-miR-3656, miR-6782-5p is hsa-miR-6782-5p, miR-4689 is hsa-miR-4689, miR-451a is hsa-miR-451a, miR-4446-3p is hsa-miR-4446-3p, miR-3180-3p is hsa-miR-3180-3p, miR-642a-3p is hsa-miR-642a-3p, miR-6889-5p is hsa-miR-6889-5p, miR-3178 is hsa-miR-3178, miR-4665-5p is hsa-miR-4665-5p, miR-6722-3p is hsa-miR-6722-3p, miR-30c-1-3p is hsa-miR-30c-1-3p, miR-4507 is hsa-miR-4507, miR-3141 is hsa-miR-3141, and miR-1199-5p is hsa-miR-1199-5p.
(9) The kit according to (7) or (8), wherein the nucleic acid is a polynucleotide selected from the group consisting of the following polynucleotides (k) to (o):
(k) a polynucleotide consisting of a nucleotide sequence represented by any of SEQ ID NOs: 170 to 199 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 170 to 199,
(m) a polynucleotide consisting of a nucleotide sequence complementary to a nucleotide sequence represented by any of SEQ ID NOs: 170 to 199 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 170 to 199 or a nucleotide sequence derived from the nucleotide sequence by the replacement of u with t, and
(o) a polynucleotide hybridizing under stringent conditions to any of the polynucleotides (k) to (n).
(10) The kit according to any one of (1) to (9), wherein the kit comprises at least two or more nucleic acids capable of specifically binding to at least two or more polynucleotides, respectively, selected from all of the stomach cancer markers according to (1) or (2).
(11) A device for the detection of stomach cancer, comprising a nucleic acid(s) capable of specifically binding to at least one or more polynucleotides selected from the group consisting of the following stomach cancer markers: miR-4257, miR-6726-5p, miR-1343-3p, miR-1247-3p, miR-6787-5p, miR-6875-5p, miR-1225-3p, miR-8063, miR-6781-5p, miR-4746-3p, miR-1908-5p, miR-6756-5p, miR-204-3p, miR-4651, miR-6757-5p, miR-6825-5p, miR-7108-5p, miR-4792, miR-7641, miR-3188, miR-3131, miR-6780b-5p, miR-8069, miR-6840-3p, miR-8072, miR-1233-5p, miR-6887-5p, miR-1231, miR-5572, miR-6738-5p, miR-6784-5p, miR-6791-5p, miR-6749-5p, miR-6741-5p, miR-128-1-5p, miR-4419b, miR-6746-5p, miR-3184-5p, miR-3679-5p, miR-7110-5p, miR-4516, miR-6717-5p, miR-6826-5p, miR-4433b-3p, miR-3679-3p, miR-3135b, miR-3622a-5p, miR-711, miR-4467, miR-6857-5p, miR-6515-3p, miR-1225-5p, miR-187-5p, miR-3185, miR-642b-3p, miR-1249, miR-744-5p, miR-4442, miR-1228-3p, miR-939-5p, miR-6845-5p, miR-887-3p, miR-7845-5p, miR-6729-5p, miR-4632-5p, miR-615-5p, miR-6724-5p, miR-4728-5p, miR-6732-5p, miR-6816-5p, miR-4695-5p, miR-6088, miR-7975, miR-3197, miR-6125, miR-4433-3p, miR-6727-5p, miR-4706, miR-7847-3p, miR-6805-3p, miR-6766-3p, miR-1913, miR-4649-5p, miR-602, miR-3663-3p, miR-6893-5p, miR-6861-5p, miR-4449, miR-6842-5p, miR-4454, miR-5195-3p, miR-663b, miR-6765-5p, miR-4513, miR-614, miR-6785-5p, miR-6777-5p, miR-940, miR-4741, miR-6870-5p, miR-6131, miR-150-3p, miR-4707-5p, miR-1915-3p, miR-3937, miR-937-5p, miR-4443, miR-1914-3p, miR-3620-5p, miR-1268b, miR-1227-5p, miR-6880-5p, miR-4417, miR-6802-5p, miR-6769a-5p, miR-663a, miR-6721-5p, miR-4532, miR-7977, miR-92b-5p, miR-371a-5p, miR-6126, miR-4734, miR-4665-3p, miR-423-5p, miR-1469, miR-4675, miR-1915-5p, miR-6716-5p, miR-718, miR-4281, miR-6820-5p, miR-6795-5p, miR-6779-5p, miR-7109-5p, miR-6798-5p, miR-4648, miR-8059, miR-6765-3p, miR-6132, miR-4492, miR-7107-5p, miR-3195, miR-3180, miR-296-3p, miR-564, miR-1268a, miR-6848-5p, miR-762, miR-2861, miR-1203, miR-1260b, miR-4476, miR-6885-5p, miR-6769b-5p, miR-23b-3p, miR-1343-5p, miR-3621, miR-4688, miR-4286, miR-4640-5p, miR-4739, miR-1260a, miR-4276, miR-7106-5p, miR-6794-5p, miR-6774-5p, miR-4707-3p, miR-4534, miR-4294, miR-6850-5p, miR-6089 and miR-671-5p.
(12) The device according to (11), wherein miR-4257 is hsa-miR-4257, miR-6726-5p is hsa-miR-6726-5p, miR-1343-3p is hsa-miR-1343-3p, miR-1247-3p is hsa-miR-1247-3p, miR-6787-5p is hsa-miR-6787-5p, miR-6875-5p is hsa-miR-6875-5p, miR-1225-3p is hsa-miR-1225-3p, miR-8063 is hsa-miR-8063, miR-6781-5p is hsa-miR-6781-5p, miR-4746-3p is hsa-miR-4746-3p, miR-1908-5p is hsa-miR-1908-5p, miR-6756-5p is hsa-miR-6756-5p, miR-204-3p is hsa-miR-204-3p, miR-4651 is hsa-miR-4651, miR-6757-5p is hsa-miR-6757-5p, miR-6825-5p is hsa-miR-6825-5p, miR-7108-5p is hsa-miR-7108-5p, miR-4792 is hsa-miR-4792, miR-7641 is hsa-miR-7641, miR-3188 is hsa-miR-3188, miR-3131 is hsa-miR-3131, miR-6780b-5p is hsa-miR-6780b-5p, miR-8069 is hsa-miR-8069, miR-6840-3p is hsa-miR-6840-3p, miR-8072 is hsa-miR-8072, miR-1233-5p is hsa-miR-1233-5p, miR-6887-5p is hsa-miR-6887-5p, miR-1231 is hsa-miR-1231, miR-5572 is hsa-miR-5572, miR-6738-5p is hsa-miR-6738-5p, miR-6784-5p is hsa-miR-6784-5p, miR-6791-5p is hsa-miR-6791-5p, miR-6749-5p is hsa-miR-6749-5p, miR-6741-5p is hsa-miR-6741-5p, miR-128-1-5p is hsa-miR-128-1-5p, miR-4419b is hsa-miR-4419b, miR-6746-5p is hsa-miR-6746-5p, miR-3184-5p is hsa-miR-3184-5p, miR-3679-5p is hsa-miR-3679-5p, miR-7110-5p is hsa-miR-7110-5p, miR-4516 is hsa-miR-4516, miR-6717-5p is hsa-miR-6717-5p, miR-6826-5p is hsa-miR-6826-5p, miR-4433b-3p is hsa-miR-4433b-3p, miR-3679-3p is hsa-miR-3679-3p, miR-3135b is hsa-miR-3135b, miR-3622a-5p is hsa-miR-3622a-5p, miR-711 is hsa-miR-711, miR-4467 is hsa-miR-4467, miR-6857-5p is hsa-miR-6857-5p, miR-6515-3p is hsa-miR-6515-3p, miR-1225-5p is hsa-miR-1225-5p, miR-187-5p is hsa-miR-187-5p, miR-3185 is hsa-miR-3185, miR-642b-3p is hsa-miR-642b-3p, miR-1249 is hsa-miR-1249, miR-744-5p is hsa-miR-744-5p, miR-4442 is hsa-miR-4442, miR-1228-3p is hsa-miR-1228-3p, miR-939-5p is hsa-miR-939-5p, miR-6845-5p is hsa-miR-6845-5p, miR-887-3p is hsa-miR-887-3p, miR-7845-5p is hsa-miR-7845-5p, miR-6729-5p is hsa-miR-6729-5p, miR-4632-5p is hsa-miR-4632-5p, miR-615-5p is hsa-miR-615-5p, miR-6724-5p is hsa-miR-6724-5p, miR-4728-5p is hsa-miR-4728-5p, miR-6732-5p is hsa-miR-6732-5p, miR-6816-5p is hsa-miR-6816-5p, miR-4695-5p is hsa-miR-4695-5p, miR-6088 is hsa-miR-6088, miR-7975 is hsa-miR-7975, miR-3197 is hsa-miR-3197, miR-6125 is hsa-miR-6125, miR-4433-3p is hsa-miR-4433-3p, miR-6727-5p is hsa-miR-6727-5p, miR-4706 is hsa-miR-4706, miR-7847-3p is hsa-miR-7847-3p, miR-6805-3p is hsa-miR-6805-3p, miR-6766-3p is hsa-miR-6766-3p, miR-1913 is hsa-miR-1913, miR-4649-5p is hsa-miR-4649-5p, miR-602 is hsa-miR-602, miR-3663-3p is hsa-miR-3663-3p, miR-6893-5p is hsa-miR-6893-5p, miR-6861-5p is hsa-miR-6861-5p, miR-4449 is hsa-miR-4449, miR-6842-5p is hsa-miR-6842-5p, miR-4454 is hsa-miR-4454, miR-5195-3p is hsa-miR-5195-3p, miR-663b is hsa-miR-663b, miR-6765-5p is hsa-miR-6765-5p, miR-4513 is hsa-miR-4513, miR-614 is hsa-miR-614, miR-6785-5p is hsa-miR-6785-5p, miR-6777-5p is hsa-miR-6777-5p, miR-940 is hsa-miR-940, miR-4741 is hsa-miR-4741, miR-6870-5p is hsa-miR-6870-5p, miR-6131 is hsa-miR-6131, miR-150-3p is hsa-miR-150-3p, miR-4707-5p is hsa-miR-4707-5p, miR-1915-3p is hsa-miR-1915-3p, miR-3937 is hsa-miR-3937, miR-937-5p is hsa-miR-937-5p, miR-4443 is hsa-miR-4443, miR-1914-3p is hsa-miR-1914-3p, miR-3620-5p is hsa-miR-3620-5p, miR-1268b is hsa-miR-1268b, miR-1227-5p is hsa-miR-1227-5p, miR-6880-5p is hsa-miR-6880-5p, miR-4417 is hsa-miR-4417, miR-6802-5p is hsa-miR-6802-5p, miR-6769a-5p is hsa-miR-6769a-5p, miR-663a is hsa-miR-663a, miR-6721-5p is hsa-miR-6721-5p, miR-4532 is hsa-miR-4532, miR-7977 is hsa-miR-7977, miR-92b-5p is hsa-miR-92b-5p, miR-371a-5p is hsa-miR-371a-5p, miR-6126 is hsa-miR-6126, miR-4734 is hsa-miR-4734, miR-4665-3p is hsa-miR-4665-3p, miR-423-5p is hsa-miR-423-5p, miR-1469 is hsa-miR-1469, miR-4675 is hsa-miR-4675, miR-1915-5p is hsa-miR-1915-5p, miR-6716-5p is hsa-miR-6716-5p, miR-718 is hsa-miR-718, miR-4281 is hsa-miR-4281, miR-6820-5p is hsa-miR-6820-5p, miR-6795-5p is hsa-miR-6795-5p, miR-6779-5p is hsa-miR-6779-5p, miR-7109-5p is hsa-miR-7109-5p, miR-6798-5p is hsa-miR-6798-5p, miR-4648 is hsa-miR-4648, miR-8059 is hsa-miR-8059, miR-6765-3p is hsa-miR-6765-3p, miR-6132 is hsa-miR-6132, miR-4492 is hsa-miR-4492, miR-7107-5p is hsa-miR-7107-5p, miR-3195 is hsa-miR-3195, miR-3180 is hsa-miR-3180, miR-296-3p is hsa-miR-296-3p, miR-564 is hsa-miR-564, miR-1268a is hsa-miR-1268a, miR-6848-5p is hsa-miR-6848-5p, miR-762 is hsa-miR-762, miR-2861 is hsa-miR-2861, miR-1203 is hsa-miR-1203, miR-1260b is hsa-miR-1260b, miR-4476 is hsa-miR-4476, miR-6885-5p is hsa-miR-6885-5p, miR-6769b-5p is hsa-miR-6769b-5p, miR-23b-3p is hsa-miR-23b-3p, miR-1343-5p is hsa-miR-1343-5p, miR-3621 is hsa-miR-3621, miR-4688 is hsa-miR-4688, miR-4286 is hsa-miR-4286, miR-4640-5p is hsa-miR-4640-5p, miR-4739 is hsa-miR-4739, miR-1260a is hsa-miR-1260a, miR-4276 is hsa-miR-4276, miR-7106-5p is hsa-miR-7106-5p, miR-6794-5p is hsa-miR-6794-5p, miR-6774-5p is hsa-miR-6774-5p, miR-4707-3p is hsa-miR-4707-3p, miR-4534 is hsa-miR-4534, miR-4294 is hsa-miR-4294, miR-6850-5p is hsa-miR-6850-5p, miR-6089 is hsa-miR-6089, and miR-671-5p is hsa-miR-671-5p.
(13) The device according to (11) or (12), wherein the nucleic acid is a polynucleotide selected from the group consisting of the following polynucleotides (a) to (e):
(a) a polynucleotide consisting of a nucleotide sequence represented by any of SEQ ID NOs: 1 to 165 and 635 to 642 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 165 and 635 to 642,
(c) a polynucleotide consisting of a nucleotide sequence complementary to a nucleotide sequence represented by any of SEQ ID NOs: 1 to 165 and 635 to 642 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 165 and 635 to 642 or a nucleotide sequence derived from the nucleotide sequence by the replacement of u with t, and
(e) a polynucleotide hybridizing under stringent conditions to any of the polynucleotides (a) to (d).
(14) The device according to any of (11) to (13), wherein the device further comprises a nucleic acid(s) capable of specifically binding to at least one or more polynucleotides selected from the group consisting of the following other stomach cancer markers: miR-128-2-5p, miR-125a-3p, miR-92a-2-5p, and miR-486-3p.
(15) The device according to (14), wherein miR-128-2-5p is hsa-miR-128-2-5p, miR-125a-3p is hsa-miR-125a-3p, miR-92a-2-5p is hsa-miR-92a-2-5p, and miR-486-3p is hsa-miR-486-3p.
(16) The device according to (14) or (15), wherein the nucleic acid is a polynucleotide selected from the group consisting of the following polynucleotides (f) to (j):
(f) a polynucleotide consisting of a nucleotide sequence represented by any of SEQ ID NOs: 166 to 169 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 166 to 169,
(h) a polynucleotide consisting of a nucleotide sequence complementary to a nucleotide sequence represented by any of SEQ ID NOs: 166 to 169 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 166 to 169 or a nucleotide sequence derived from the nucleotide sequence by the replacement of u with t, and
(j) a polynucleotide hybridizing under stringent conditions to any of the polynucleotides (f) to (i).
(17) The device according to any of (11) to (16), wherein the device further comprises a nucleic acid(s) capable of specifically binding to at least one or more polynucleotides selected from the group consisting of the following other stomach cancer markers: miR-3196, miR-211-3p, miR-4271, miR-6851-5p, miR-149-3p, miR-4667-5p, miR-135a-3p, miR-4486, miR-4697-5p, miR-4725-3p, miR-6510-5p, miR-5001-5p, miR-4673, miR-4466, miR-23a-3p, miR-3656, miR-6782-5p, miR-4689, miR-451a, miR-4446-3p, miR-3180-3p, miR-642a-3p, miR-6889-5p, miR-3178, miR-4665-5p, miR-6722-3p, miR-30c-1-3p, miR-4507, miR-3141 and miR-1199-5p.
(18) The device according to (17), wherein miR-3196 is hsa-miR-3196, miR-211-3p is hsa-miR-211-3p, miR-4271 is hsa-miR-4271, miR-6851-5p is hsa-miR-6851-5p, miR-149-3p is hsa-miR-149-3p, miR-4667-5p is hsa-miR-4667-5p, miR-135a-3p is hsa-miR-135a-3p, miR-4486 is hsa-miR-4486, miR-4697-5p is hsa-miR-4697-5p, miR-4725-3p is hsa-miR-4725-3p, miR-6510-5p is hsa-miR-6510-5p, miR-5001-5p is hsa-miR-5001-5p, miR-4673 is hsa-miR-4673, miR-4466 is hsa-miR-4466, miR-23a-3p is hsa-miR-23a-3p, miR-3656 is hsa-miR-3656, miR-6782-5p is hsa-miR-6782-5p, miR-4689 is hsa-miR-4689, miR-451a is hsa-miR-451a, miR-4446-3p is hsa-miR-4446-3p, miR-3180-3p is hsa-miR-3180-3p, miR-642a-3p is hsa-miR-642a-3p, miR-6889-5p is hsa-miR-6889-5p, miR-3178 is hsa-miR-3178, miR-4665-5p is hsa-miR-4665-5p, miR-6722-3p is hsa-miR-6722-3p, miR-30c-1-3p is hsa-miR-30c-1-3p, miR-4507 is hsa-miR-4507, miR-3141 is hsa-miR-3141, and miR-1199-5p is hsa-miR-1199-5p.
(19) The device according to (17) or (18), wherein the nucleic acid is a polynucleotide selected from the group consisting of the following polynucleotides (k) to (o):
(k) a polynucleotide consisting of a nucleotide sequence represented by any of SEQ ID NOs: 170 to 199 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 170 to 199,
(m) a polynucleotide consisting of a nucleotide sequence complementary to a nucleotide sequence represented by any of SEQ ID NOs: 170 to 199 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 170 to 199 or a nucleotide sequence derived from the nucleotide sequence by the replacement of u with t, and
(o) a polynucleotide hybridizing under stringent conditions to any of the polynucleotides (k) to (n).
(20) The device according to any one of (11) to (19), wherein the device is for measurement based on a hybridization technique.
(21) The device according to (20), wherein the hybridization technique is a nucleic acid array technique.
(22) The device according to any one of (11) to (21), wherein the device comprises at least two or more nucleic acids capable of specifically binding to at least two or more polynucleotides, respectively, selected from all of the stomach cancer markers according to (11) or (12).
(23) A method for detecting stomach cancer, comprising measuring an expression level(s) of a target nucleic acid(s) in a sample from a subject using the kit according to any one of (1) to (10) or the device according to any one of (11) to (22), and evaluating in vitro whether or not the subject has stomach cancer using both of the measured expression level(s) and a control expression level(s) in a sample from a healthy subject measured in the same way.
(24) The method according to (23), wherein the subject is a human.
(25) The method according to (23) or (24), wherein the sample is blood, serum, or plasma.

### DEFINITION OF TERMS

The terms used herein are defined as follows.

Abbreviations or terms such as nucleotide, polynucleotide, DNA, and RNA abide by “Guidelines for the preparation of specification which contain nucleotide and/or amino acid sequences” (edited by Japan Patent Office) and common use in the art.

The term “polynucleotide” used herein refers to a nucleic acid including all of RNA, DNA, and RNA/DNA (chimera). The DNA includes any of cDNA, genomic DNA, and synthetic DNA. The RNA includes any of total RNA, mRNA, rRNA, miRNA, siRNA, snoRNA, snRNA, non-coding RNA and synthetic RNA. Herein, the “synthetic DNA” and the “synthetic RNA” refer to DNA and RNA artificially prepared using, for example, an automatic nucleic acid synthesizer, on the basis of predetermined nucleotide sequences (which may be any of natural and non-natural sequences). The “non-natural sequence” is intended to be used in a broad sense and includes, for example, a sequence containing substitution, deletion, insertion, and/or addition of one or more nucleotides (i.e., a variant sequence) and a sequence containing one or more modified nucleotides (i.e., a modified sequence), which are different from the natural sequence. Herein, the polynucleotide is used interchangeably with a nucleic acid.

The term “fragment” used herein is a polynucleotide having a nucleotide sequence having a consecutive portion of a polynucleotide and desirably has a length of 15 or more nucleotides, preferably 17 or more nucleotides, more preferably 19 or more nucleotides.

The term “gene” used herein is intended to include not only RNA and double-stranded DNA but also each single-stranded DNA such as a plus strand (or a sense strand) or a complementary strand (or an antisense strand) that constitutes a duplex. The gene is not particularly limited by its length.

Thus, herein, the “gene” includes any of double-stranded DNA including human genomic DNA, single-stranded DNA (plus strand), single-stranded DNA having a sequence complementary to the plus strand (complementary strand) including cDNA, microRNA (miRNA), and their fragments, and their transcripts, unless otherwise specified. The “gene” includes not only a “gene” represented by a particular nucleotide sequence (or SEQ ID NO) but “nucleic acids” encoding RNAs having biological functions equivalent to RNA encoded by the gene, for example, a congener (i.e., a homolog or an ortholog), a variant (e.g., a genetic polymorph), and a derivative. Specific examples of such a “nucleic acid” encoding a congener, a variant, or a derivative can include a “nucleic acid” having a nucleotide sequence hybridizing under stringent conditions described later to a complementary sequence of a nucleotide sequence represented by any of SEQ ID NOs: 1 to 657 or a nucleotide sequence derived from the nucleotide sequence by the replacement of u with t. The “gene” is not particularly limited by its functional region and can contain, for example, an expression regulatory region(s), a coding region(s), an exon(s), or an intron(s). The “gene” may be contained in a cell or may exist alone after being released from a cell. Alternatively, the “gene” may be enclosed in a vesicle called exosome.

The term “exosome” used herein is a vesicle that is delimited by a lipid bilayer and secreted from a cell. The exosome is derived from a multivesicular endosome and may incorporate biomaterials such as a “gene” (e.g., RNA or DNA) or a protein when released into an extracellular environment. The exosome is known to be contained in a body fluid such as blood, serum, plasma, or lymph.

The term “transcript” used herein refers to an RNA synthesized from the DNA sequence of a gene as a template. RNA polymerase binds to a site called a promoter located upstream of the gene and adds ribonucleotides complementary to the nucleotide sequence of the DNA to the 3′ end to synthesize RNA. This RNA contains not only the gene itself but also the whole sequence from a transcription initiation site to the end of a polyA sequence, including an expression regulatory region, a coding region, an exon, or an intron.

Unless otherwise specified, the term “microRNA (miRNA)” used herein is intended to mean a 15- to 25-nucleotide non-coding RNA that is involved in the suppression of translation of mRNA, and that transcribed as an RNA precursor having a hairpin-like structure, cleaved by a dsRNA-cleaving enzyme which has RNase III cleavage activity, and integrated into a protein complex called RISC. The term “miRNA” used herein includes not only a “miRNA” represented by a particular nucleotide sequence (or SEQ ID NO) but a precursor of the “miRNA” (pre-miRNA or pri-miRNA), and miRNAs having biological functions equivalent thereto, for example, a congener (i.e., a homolog or an ortholog), a variant (e.g., a genetic polymorph), and a derivative. Such a precursor, a congener, a variant, or a derivative can be specifically identified using miRBase Release 20 (http://www.mirbase.org/), and examples thereof can include a “miRNA” having a nucleotide sequence hybridizing under stringent conditions described later to a complementary sequence of any particular nucleotide sequence represented by any of SEQ ID NOs: 1 to 657. The term “miRNA” used herein may be a gene product of a miR gene. Such a gene product includes a mature miRNA (e.g., a 15- to 25-nucleotide or 19- to 25-nucleotide non-coding RNA involved in the suppression of translation of mRNA as described above) or a miRNA precursor (e.g., pre-miRNA or pri-miRNA as described above).

The term “probe” used herein includes a polynucleotide that is used for specifically detecting RNA resulting from the expression of a gene or a polynucleotide derived from the RNA, and/or a polynucleotide complementary thereto.

The term “primer” used herein includes a polynucleotide that specifically recognizes and amplifies RNA resulting from the expression of a gene or a polynucleotide derived from the RNA, and/or a polynucleotide complementary thereto.

In this context, the complementary polynucleotide (complementary strand or reverse strand) means a polynucleotide in a complementary relationship of A:T (U) and G:C base pairs with the full-length sequence of a polynucleotide consisting of a nucleotide sequence defined by any of SEQ ID NOs: 1 to 657 or a nucleotide sequence derived from the nucleotide sequence by the replacement of u with t, or a partial sequence thereof (here, this full-length or partial sequence is referred to as a plus strand for the sake of convenience). However, such a complementary strand is not limited to a sequence completely complementary to the nucleotide sequence of the target plus strand and may have a complementary relationship to an extent that permits hybridization under stringent conditions to the target plus strand.

The term “stringent conditions” used herein refers to conditions under which a nucleic acid probe hybridizes to its target sequence to a larger extent (e.g., a measurement value equal to or larger than “(a mean of background measurement values)+(a standard deviation of the background measurement values)×2”) than that for other sequences. The stringent conditions are dependent on a sequence and differ depending on an environment where hybridization is performed. A target sequence that is 100% complementary to the nucleic acid probe can be identified by controlling the stringency of hybridization and/or washing conditions. Specific examples of the “stringent conditions” will be mentioned later.

The term “Tm value” used herein means a temperature at which the double-stranded moiety of a polynucleotide is denatured into single strands so that the double strands and the single strands exist at a ratio of 1:1.

The term “variant” used herein means, in the case of a nucleic acid, a natural variant attributed to polymorphism, mutation, or the like; a variant containing the deletion, substitution, addition, or insertion of 1 or 2 or more nucleotides in a nucleotide sequence represented by any of SEQ ID NOs: 1 to 657 or a nucleotide sequence derived from the nucleotide sequence by the replacement of u with t, or a partial sequence thereof; a variant that exhibits percent (%) identity of approximately 90% or higher, approximately 95% or higher, approximately 97% or higher, approximately 98% or higher, approximately 99% or higher to each of these nucleotide sequences or the partial sequences thereof; or a nucleic acid hybridizing under the stringent conditions defined above to a polynucleotide or an oligonucleotide comprising each of these nucleotide sequences or the partial sequences thereof.

The term “several” used herein means an integer of approximately 10, 9, 8, 7, 6, 5, 4, 3, or 2.

The variant used herein can be prepared by use of a well-known technique such as site-directed mutagenesis or PCR-based mutagenesis.

The term “percent (%) identity” used herein can be determined with or without an introduced gap, using a protein or gene search system based on BLAST or FASTA described above (Zheng Zhang et al., 2000, J. Comput. Biol., Vol. 7, p. 203-214; Altschul, S. F. et al., 1990, Journal of Molecular Biology, Vol. 215, p. 403-410; and Pearson, W. R. et al., 1988, Proc. Natl. Acad. Sci. U.S.A., Vol. 85, p. 2444-2448).

The term “derivative” used herein is meant to include a modified nucleic acid, for example, a derivative labeled with a fluorophore or the like, a derivative containing a modified nucleotide (e.g., a nucleotide containing a group such as halogen, alkyl such as methyl, alkoxy such as methoxy, thio, or carboxymethyl, and a nucleotide that has undergone base rearrangement, double bond saturation, deamination, replacement of an oxygen molecule with a sulfur atom, etc.), PNA (peptide nucleic acid; Nielsen, P. E. et al., 1991, Science, Vol. 254, p. 1497-500), and LNA (locked nucleic acid; Obika, S. et al., 1998, Tetrahedron Lett., Vol. 39, p. 5401-5404) without any limitation.

The “nucleic acid” used herein capable of specifically binding to a polynucleotide selected from the stomach cancer marker miRNAs described above is a synthesized or prepared nucleic acid and specifically includes a “nucleic acid probe” or a “primer”. The “nucleic acid” is utilized directly or indirectly for detecting the presence or absence of stomach cancer in a subject, for diagnosing the presence or absence of stomach cancer, the severity of stomach cancer, the presence or absence of amelioration or the degree of amelioration of stomach cancer, or the therapeutic sensitivity of stomach cancer, or for screening for a candidate substance useful in the prevention, amelioration, or treatment of stomach cancer. The “nucleic acid” includes a nucleotide, an oligonucleotide, and a polynucleotide capable of specifically recognizing and binding to a transcript represented by any of SEQ ID NOs: 1 to 657 or a synthetic cDNA nucleic acid thereof in vivo, particularly, in a sample such as a body fluid (e.g., blood or urine), in relation to the development of stomach cancer. The nucleotide, the oligonucleotide, and the polynucleotide can be effectively used as probes for detecting the aforementioned gene expressed in vivo, in tissues, in cells, or the like on the basis of the properties described above, or as primers for amplifying the aforementioned gene expressed in vivo.

The term “detection” used herein is interchangeable with the term “examination”, “measurement”, “detection”, or “decision support”. The term “evaluation” used herein is meant to include diagnosis or evaluation support on the basis of examination results or measurement results.

The term “subject” used herein means a mammal such as a primate including a human and a chimpanzee, a pet animal including a dog and a cat, a livestock animal including cattle, a horse, sheep, and a goat, and a rodent including a mouse and a rat. The term “healthy subject” also means such a mammal without the cancer to be detected.

The term “P” or “P value” used herein refers to a probability at which a more extreme statistic than that actually calculated from data under null hypothesis is observed in a statistical test. Thus, smaller “P” or “P value” is regarded as being a more significant difference between subjects to be compared.

The term “sensitivity” used herein means a value of (the number of true positives)/(the number of true positives+the number of false negatives). High sensitivity allows stomach cancer to be detected early, leading to the complete resection of cancer sites and reduction in the rate of recurrence.

The term “specificity” used herein means a value of (the number of true negatives)/(the number of true negatives+the number of false positives). High specificity prevents needless extra examination for healthy subjects misjudged as being stomach cancer patients, leading to reduction in burden on patients and reduction in medical expense.

The term “accuracy” used herein means a value of (the number of true positives+the number of true negatives)/(the total number of cases). The accuracy indicates the ratio of samples that identified correctly in the discriminant results to all samples, and serves as a primary index for evaluating detection performance.

The “sample” used herein that is subject to determination, detection, or diagnosis refers to a tissue and a biological material in which the expression of the gene of the present invention varies as stomach cancer develops, as stomach cancer progresses, or as therapeutic effects on stomach cancer are exerted. Specifically, the “sample” refers to a gastric tissue, a perigastric vascular channel, lymph node, and organ, an organ suspected of having metastasis, the skin, a body fluid such as blood, urine, saliva, sweat, or tissue exudates, serum or plasma prepared from blood, feces, hair, and the like. The “sample” further refers to a biological sample extracted therefrom, specifically, a gene such as RNA or miRNA.

The term “hsa-miR-4257 gene” or “hsa-miR-4257” used herein includes the hsa-miR-4257 gene (miRBase Accession No. MIMAT0016878) described in SEQ ID NO: 1, a homolog or an ortholog of a different organism species, and the like. The hsa-miR-4257 gene can be obtained by a method described in Goff L A et al., 2009, PLoS One, Vol. 4, e7192. Also, “hsa-mir-4257” (miRBase Accession No. MI0015856, SEQ ID NO: 200) having a hairpin-like structure is known as a precursor of “hsa-miR-4257”.

The term “hsa-miR-6726-5p gene” or “hsa-miR-6726-5p” used herein includes the hsa-miR-6726-5p gene (miRBase Accession No. MIMAT0027353) described in SEQ ID NO: 2, a homolog or an ortholog of a different organism species, and the like. The hsa-miR-6726-5p gene can be obtained by a method described in Ladewig E et al., 2012, Genome Res, Vol. 22, p. 1634-1645. Also, “hsa-mir-6726” (miRBase Accession No. MI0022571, SEQ ID NO: 201) having a hairpin-like structure is known as a precursor of “hsa-miR-6726-5p”.

The term “hsa-miR-1343-3p gene” or “hsa-miR-1343-3p” used herein includes the hsa-miR-1343-3p gene (miRBase Accession No. MIMAT0019776) described in SEQ ID NO: 3, a homolog or an ortholog of a different organism species, and the like. The hsa-miR-1343-3p gene can be obtained by a method described in Persson H et al., 2011, Cancer Res, Vol. 71, p. 78-86. Also, “hsa-mir-1343” (miRBase Accession No. MI0017320, SEQ ID NO: 202) having a hairpin-like structure is known as a precursor of “hsa-miR-1343-3p”.

The term “hsa-miR-1247-3p gene” or “hsa-miR-1247-3p” used herein includes the hsa-miR-1247-3p gene (miRBase Accession No. MIMAT0022721) described in SEQ ID NO: 4, a homolog or an ortholog of a different organism species, and the like. The hsa-miR-1247-3p gene can be obtained by a method described in Morin R D et al., 2008, Genome Res, Vol. 18, p. 610-621. Also, “hsa-mir-1247” (miRBase Accession No. MI0006382, SEQ ID NO: 203) having a hairpin-like structure is known as a precursor of “hsa-miR-1247-3p”.

The term “hsa-miR-6787-5p gene” or “hsa-miR-6787-5p” used herein includes the hsa-miR-6787-5p gene (miRBase Accession No. MIMAT0027474) described in SEQ ID NO: 5, a homolog or an ortholog of a different organism species, and the like. The hsa-miR-6787-5p gene can be obtained by a method described in Ladewig E et al., 2012, Genome Res, Vol. 22, p. 1634-1645. Also, “hsa-mir-6787” (miRBase Accession No. MI0022632, SEQ ID NO: 204) having a hairpin-like structure is known as a precursor of “hsa-miR-6787-5p”.

The term “hsa-miR-6875-5p gene” or “hsa-miR-6875-5p” used herein includes the hsa-miR-6875-5p gene (miRBase Accession No. MIMAT0027650) described in SEQ ID NO: 6, a homolog or an ortholog of a different organism species, and the like. The hsa-miR-6875-5p gene can be obtained by a method described in Ladewig E et al., 2012, Genome Res, Vol. 22, p. 1634-1645. Also, “hsa-mir-6875” (miRBase Accession No. MI0022722, SEQ ID NO: 205) having a hairpin-like structure is known as a precursor of “hsa-miR-6875-5p”.

The term “hsa-miR-1225-3p gene” or “hsa-miR-1225-3p” used herein includes the hsa-miR-1225-3p gene (miRBase Accession No. MIMAT0005573) described in SEQ ID NO: 7, a homolog or an ortholog of a different organism species, and the like. The hsa-miR-1225-3p gene can be obtained by a method described in Berezikov E et al., 2007, Mol Cell, Vol. 28, p. 328-336. Also, “hsa-mir-1225” (miRBase Accession No. MI0006311, SEQ ID NO: 206) having a hairpin-like structure is known as a precursor of “hsa-miR-1225-3p”.

The term “hsa-miR-8063 gene” or “hsa-miR-8063” used herein includes the hsa-miR-8063 gene (miRBase Accession No. MIMAT0030990) described in SEQ ID NO: 8, a homolog or an ortholog of a different organism species, and the like. The hsa-miR-8063 gene can be obtained by a method described in Wang H J et al., 2013, Shock, Vol. 39, p. 480-487. Also, “hsa-mir-8063” (miRBase Accession No. MI0025899, SEQ ID NO: 207) having a hairpin-like structure is known as a precursor of “hsa-miR-8063”.

The term “hsa-miR-6781-5p gene” or “hsa-miR-6781-5p” used herein includes the hsa-miR-6781-5p gene (miRBase Accession No. MIMAT0027462) described in SEQ ID NO: 9, a homolog or an ortholog of a different organism species, and the like. The hsa-miR-6781-5p gene can be obtained by a method described in Ladewig E et al., 2012, Genome Res, Vol. 22, p. 1634-1645. Also, “hsa-mir-6781” (miRBase Accession No. MI0022626, SEQ ID NO: 208) having a hairpin-like structure is known as a precursor of “hsa-miR-6781-5p”.

The term “hsa-miR-4746-3p gene” or “hsa-miR-4746-3p” used herein includes the hsa-miR-4746-3p gene (miRBase Accession No. MIMAT0019881) described in SEQ ID NO: 10, a homolog or an ortholog of a different organism species, and the like. The hsa-miR-4746-3p gene can be obtained by a method described in Persson H et al., 2011, Cancer Res, Vol. 71, p. 78-86. Also, “hsa-mir-4746” (miRBase Accession No. MI0017385, SEQ ID NO: 209) having a hairpin-like structure is known as a precursor of “hsa-miR-4746-3p”.

The term “hsa-miR-1908-5p gene” or “hsa-miR-1908-5p” used herein includes the hsa-miR-1908-5p gene (miRBase Accession No. MIMAT0007881) described in SEQ ID NO: 11, a homolog or an ortholog of a different organism species, and the like. The hsa-miR-1908-5p gene can be obtained by a method described in Bar M et al., 2008, Stem Cells, Vol. 26, p. 2496-2505. Also, “hsa-mir-1908” (miRBase Accession No. MI0008329, SEQ ID NO: 210) having a hairpin-like structure is known as a precursor of “hsa-miR-1908-5p”.

The term “hsa-miR-6756-5p gene” or “hsa-miR-6756-5p” used herein includes the hsa-miR-6756-5p gene (miRBase Accession No. MIMAT0027412) described in SEQ ID NO: 12, a homolog or an ortholog of a different organism species, and the like. The hsa-miR-6756-5p gene can be obtained by a method described in Ladewig E et al., 2012, Genome Res, Vol. 22, p. 1634-1645. Also, “hsa-mir-6756” (miRBase Accession No. MI0022601, SEQ ID NO: 211) having a hairpin-like structure is known as a precursor of “hsa-miR-6756-5p”.

The term “hsa-miR-204-3p gene” or “hsa-miR-204-3p” used herein includes the hsa-miR-204-3p gene (miRBase Accession No. MIMAT0022693) described in SEQ ID NO: 13, a homolog or an ortholog of a different organism species, and the like. The hsa-miR-204-3p gene can be obtained by a method described in Lim L P et al., 2003, Science, Vol. 299, p. 1540. Also, “hsa-mir-204” (miRBase Accession No. MI0000284, SEQ ID NO: 212) having a hairpin-like structure is known as a precursor of “hsa-miR-204-3p”.

The term “hsa-miR-4651 gene” or “hsa-miR-4651” used herein includes the hsa-miR-4651 gene (miRBase Accession No. MIMAT0019715) described in SEQ ID NO: 14, a homolog or an ortholog of a different organism species, and the like. The hsa-miR-4651 gene can be obtained by a method described in Persson H et al., 2011, Cancer Res, Vol. 71, p. 78-86. Also, “hsa-mir-4651” (miRBase Accession No. MI0017279, SEQ ID NO: 213) having a hairpin-like structure is known as a precursor of “hsa-miR-4651”.

The term “hsa-miR-6757-5p gene” or “hsa-miR-6757-5p” used herein includes the hsa-miR-6757-5p gene (miRBase Accession No. MIMAT0027414) described in SEQ ID NO: 15, a homolog or an ortholog of a different organism species, and the like. The hsa-miR-6757-5p gene can be obtained by a method described in Ladewig E et al., 2012, Genome Res, Vol. 22, p. 1634-1645. Also, “hsa-mir-6757” (miRBase Accession No. MI0022602, SEQ ID NO: 214) having a hairpin-like structure is known as a precursor of “hsa-miR-6757-5p”.

The term “hsa-miR-6825-5p gene” or “hsa-miR-6825-5p” used herein includes the hsa-miR-6825-5p gene (miRBase Accession No. MIMAT0027550) described in SEQ ID NO: 16, a homolog or an ortholog of a different organism species, and the like. The hsa-miR-6825-5p gene can be obtained by a method described in Ladewig E et al., 2012, Genome Res, Vol. 22, p. 1634-1645. Also, “hsa-mir-6825” (miRBase Accession No. MI0022670, SEQ ID NO: 215) having a hairpin-like structure is known as a precursor of “hsa-miR-6825-5p”.

The term “hsa-miR-7108-5p gene” or “hsa-miR-7108-5p” used herein includes the hsa-miR-7108-5p gene (miRBase Accession No. MIMAT0028113) described in SEQ ID NO: 17, a homolog or an ortholog of a different organism species, and the like. The hsa-miR-7108-5p gene can be obtained by a method described in Ladewig E et al., 2012, Genome Res, Vol. 22, p. 1634-1645. Also, “hsa-mir-7108” (miRBase Accession No. MI0022959, SEQ ID NO: 216) having a hairpin-like structure is known as a precursor of “hsa-miR-7108-5p”.

The term “hsa-miR-4792 gene” or “hsa-miR-4792” used herein includes the hsa-miR-4792 gene (miRBase Accession No. MIMAT0019964) described in SEQ ID NO: 18, a homolog or an ortholog of a different organism species, and the like. The hsa-miR-4792 gene can be obtained by a method described in Persson H et al., 2011, Cancer Res, Vol. 71, p. 78-86. Also, “hsa-mir-4792” (miRBase Accession No. MI0017439, SEQ ID NO: 217) having a hairpin-like structure is known as a precursor of “hsa-miR-4792”.

The term “hsa-miR-7641 gene” or “hsa-miR-7641” used herein includes the hsa-miR-7641 gene (miRBase Accession No. MIMAT0029782) described in SEQ ID NO: 19, a homolog or an ortholog of a different organism species, and the like. The hsa-miR-7641 gene can be obtained by a method described in Yoo J K et al., 2013, Arch Pharm Res, Vol. 36, p. 353-358. Also, “hsa-mir-7641-1 and hsa-mir-7641-2” (miRBase Accession Nos. MI0024975 and MI0024976, SEQ ID NO: 218 and 219) having a hairpin-like structure are known as precursors of “hsa-miR-7641”.

The term “hsa-miR-3188 gene” or “hsa-miR-3188” used herein includes the hsa-miR-3188 gene (miRBase Accession No. MIMAT0015070) described in SEQ ID NO: 20, a homolog or an ortholog of a different organism species, and the like. The hsa-miR-3188 gene can be obtained by a method described in Stark M S et al., 2010, PLoS One, Vol. 5, e9685. Also, “hsa-mir-3188” (miRBase Accession No. MI0014232, SEQ ID NO: 220) having a hairpin-like structure is known as a precursor of “hsa-miR-3188”.

The term “hsa-miR-3131 gene” or “hsa-miR-3131” used herein includes the hsa-miR-3131 gene (miRBase Accession No. MIMAT0014996) described in SEQ ID NO: 21, a homolog or an ortholog of a different organism species, and the like. The hsa-miR-3131 gene can be obtained by a method described in Stark M S et al., 2010, PLoS One, Vol. 5, e9685. Also, “hsa-mir-3131” (miRBase Accession No. MI0014151, SEQ ID NO: 221) having a hairpin-like structure is known as a precursor of “hsa-miR-3131”.

The term “hsa-miR-6780b-5p gene” or “hsa-miR-6780b-5p” used herein includes the hsa-miR-6780b-5p gene (miRBase Accession No. MIMAT0027572) described in SEQ ID NO: 22, a homolog or an ortholog of a different organism species, and the like. The hsa-miR-6780b-5p gene can be obtained by a method described in Ladewig E et al., 2012, Genome Res, Vol. 22, p. 1634-1645. Also, “hsa-mir-6780b” (miRBase Accession No. MI0022681, SEQ ID NO: 222) having a hairpin-like structure is known as a precursor of “hsa-miR-6780b-5p”.

The term “hsa-miR-8069 gene” or “hsa-miR-8069” used herein includes the hsa-miR-8069 gene (miRBase Accession No. MIMAT0030996) described in SEQ ID NO: 23, a homolog or an ortholog of a different organism species, and the like. The hsa-miR-8069 gene can be obtained by a method described in Wang H J et al., 2013, Shock, Vol. 39, p. 480-487. Also, “hsa-mir-8069” (miRBase Accession No. MI0025905, SEQ ID NO: 223) having a hairpin-like structure is known as a precursor of “hsa-miR-8069”.

The term “hsa-miR-6840-3p gene” or “hsa-miR-6840-3p” used herein includes the hsa-miR-6840-3p gene (miRBase Accession No. MIMAT0027583) described in SEQ ID NO: 24, a homolog or an ortholog of a different organism species, and the like. The hsa-miR-6840-3p gene can be obtained by a method described in Ladewig E et al., 2012, Genome Res, Vol. 22, p. 1634-1645. Also, “hsa-mir-6840” (miRBase Accession No. MI0022686, SEQ ID NO: 224) having a hairpin-like structure is known as a precursor of “hsa-miR-6840-3p”.

The term “hsa-miR-8072 gene” or “hsa-miR-8072” used herein includes the hsa-miR-8072 gene (miRBase Accession No. MIMAT0030999) described in SEQ ID NO: 25, a homolog or an ortholog of a different organism species, and the like. The hsa-miR-8072 gene can be obtained by a method described in Wang H J et al., 2013, Shock, Vol. 39, p. 480-487. Also, “hsa-mir-8072” (miRBase Accession No. MI0025908, SEQ ID NO: 225) having a hairpin-like structure is known as a precursor of “hsa-miR-8072”.

The term “hsa-miR-1233-5p gene” or “hsa-miR-1233-5p” used herein includes the hsa-miR-1233-5p gene (miRBase Accession No. MIMAT0022943) described in SEQ ID NO: 26, a homolog or an ortholog of a different organism species, and the like. The hsa-miR-1233-5p gene can be obtained by a method described in Berezikov E et al., 2007, Mol Cell, Vol. 28, p. 328-336. Also, “hsa-mir-1233-1 and hsa-mir-1233-2” (miRBase Accession Nos. MI0006323 and MI0015973, SEQ ID NOs: 226 and 227) having a hairpin-like structure are known as precursors of “hsa-miR-1233-5p”.

The term “hsa-miR-6887-5p gene” or “hsa-miR-6887-5p” used herein includes the hsa-miR-6887-5p gene (miRBase Accession No. MIMAT0027674) described in SEQ ID NO: 27, a homolog or an ortholog of a different organism species, and the like. The hsa-miR-6887-5p gene can be obtained by a method described in Ladewig E et al., 2012, Genome Res, Vol. 22, p. 1634-1645. Also, “hsa-mir-6887” (miRBase Accession No. MI0022734, SEQ ID NO: 228) having a hairpin-like structure is known as a precursor of “hsa-miR-6887-5p”.

The term “hsa-miR-1231 gene” or “hsa-miR-1231” used herein includes the hsa-miR-1231 gene (miRBase Accession No. MIMAT0005586) described in SEQ ID NO: 28, a homolog or an ortholog of a different organism species, and the like. The hsa-miR-1231 gene can be obtained by a method described in Berezikov E et al., 2007, Mol Cell, Vol. 28, p. 328-336. Also, “hsa-mir-1231” (miRBase Accession No. MI0006321, SEQ ID NO: 229) having a hairpin-like structure is known as a precursor of “hsa-miR-1231”.

The term “hsa-miR-5572 gene” or “hsa-miR-5572” used herein includes the hsa-miR-5572 gene (miRBase Accession No. MIMAT0022260) described in SEQ ID NO: 29, a homolog or an ortholog of a different organism species, and the like. The hsa-miR-5572 gene can be obtained by a method described in Tandon M et al., 2012, Oral Dis, Vol. 18, p. 127-131. Also, “hsa-mir-5572” (miRBase Accession No. MI0019117, SEQ ID NO: 230) having a hairpin-like structure is known as a precursor of “hsa-miR-5572”.

The term “hsa-miR-6738-5p gene” or “hsa-miR-6738-5p” used herein includes the hsa-miR-6738-5p gene (miRBase Accession No. MIMAT0027377) described in SEQ ID NO: 30, a homolog or an ortholog of a different organism species, and the like. The hsa-miR-6738-5p gene can be obtained by a method described in Ladewig E et al., 2012, Genome Res, Vol. 22, p. 1634-1645. Also, “hsa-mir-6738” (miRBase Accession No. MI0022583, SEQ ID NO: 231) having a hairpin-like structure is known as a precursor of “hsa-miR-6738-5p”.

The term “hsa-miR-6784-5p gene” or “hsa-miR-6784-5p” used herein includes the hsa-miR-6784-5p gene (miRBase Accession No. MIMAT0027468) described in SEQ ID NO: 31, a homolog or an ortholog of a different organism species, and the like. The hsa-miR-6784-5p gene can be obtained by a method described in Ladewig E et al., 2012, Genome Res, Vol. 22, p. 1634-1645. Also, “hsa-mir-6784” (miRBase Accession No. MI0022629, SEQ ID NO: 232) having a hairpin-like structure is known as a precursor of “hsa-miR-6784-5p”.

The term “hsa-miR-6791-5p gene” or “hsa-miR-6791-5p” used herein includes the hsa-miR-6791-5p gene (miRBase Accession No. MIMAT0027482) described in SEQ ID NO: 32, a homolog or an ortholog of a different organism species, and the like. The hsa-miR-6791-5p gene can be obtained by a method described in Ladewig E et al., 2012, Genome Res, Vol. 22, p. 1634-1645. Also, “hsa-mir-6791” (miRBase Accession No. MI0022636, SEQ ID NO: 233) having a hairpin-like structure is known as a precursor of “hsa-miR-6791-5p”.

The term “hsa-miR-6749-5p gene” or “hsa-miR-6749-5p” used herein includes the hsa-miR-6749-5p gene (miRBase Accession No. MIMAT0027398) described in SEQ ID NO: 33, a homolog or an ortholog of a different organism species, and the like. The hsa-miR-6749-5p gene can be obtained by a method described in Ladewig E et al., 2012, Genome Res, Vol. 22, p. 1634-1645. Also, “hsa-mir-6749” (miRBase Accession No. MI0022594, SEQ ID NO: 234) having a hairpin-like structure is known as a precursor of “hsa-miR-6749-5p”.

The term “hsa-miR-6741-5p gene” or “hsa-miR-6741-5p” used herein includes the hsa-miR-6741-5p gene (miRBase Accession No. MIMAT0027383) described in SEQ ID NO: 34, a homolog or an ortholog of a different organism species, and the like. The hsa-miR-6741-5p gene can be obtained by a method described in Ladewig E et al., 2012, Genome Res, Vol. 22, p. 1634-1645. Also, “hsa-mir-6741” (miRBase Accession No. MI0022586, SEQ ID NO: 235) having a hairpin-like structure is known as a precursor of “hsa-miR-6741-5p”.

The term “hsa-miR-128-1-5p gene” or “hsa-miR-128-1-5p” used herein includes the hsa-miR-128-1-5p gene (miRBase Accession No. MIMAT0026477) described in SEQ ID NO: 35, a homolog or an ortholog of a different organism species, and the like. The hsa-miR-128-1-5p gene can be obtained by a method described in Lagos-Quintana M et al., 2002, Curr Biol, Vol. 12, p. 735-739. Also, “hsa-mir-128-1” (miRBase Accession No. MI0000447, SEQ ID NO: 236) having a hairpin-like structure is known as a precursor of “hsa-miR-128-1-5p”.

The term “hsa-miR-4419b gene” or “hsa-miR-4419b” used herein includes the hsa-miR-4419b gene (miRBase Accession No. MIMAT0019034) described in SEQ ID NO: 36, a homolog or an ortholog of a different organism species, and the like. The hsa-miR-4419b gene can be obtained by a method described in Jima D D et al., 2010, Blood, Vol. 116, e118-e127. Also, “hsa-mir-4419b” (miRBase Accession No. MI0016861, SEQ ID NO: 237) having a hairpin-like structure is known as a precursor of “hsa-miR-4419b”.

The term “hsa-miR-6746-5p gene” or “hsa-miR-6746-5p” used herein includes the hsa-miR-6746-5p gene (miRBase Accession No. MIMAT0027392) described in SEQ ID NO: 37, a homolog or an ortholog of a different organism species, and the like. The hsa-miR-6746-5p gene can be obtained by a method described in Ladewig E et al., 2012, Genome Res, Vol. 22, p. 1634-1645. Also, “hsa-mir-6746” (miRBase Accession No. MI0022591, SEQ ID NO: 238) having a hairpin-like structure is known as a precursor of “hsa-miR-6746-5p”.

The term “hsa-miR-3184-5p gene” or “hsa-miR-3184-5p” used herein includes the hsa-miR-3184-5p gene (miRBase Accession No. MIMAT0015064) described in SEQ ID NO: 38, a homolog or an ortholog of a different organism species, and the like. The hsa-miR-3184-5p gene can be obtained by a method described in Stark M S et al., 2010, PLoS One, Vol. 5, e9685. Also, “hsa-mir-3184” (miRBase Accession No. MI0014226, SEQ ID NO: 239) having a hairpin-like structure is known as a precursor of “hsa-miR-3184-5p”.

The term “hsa-miR-3679-5p gene” or “hsa-miR-3679-5p” used herein includes the hsa-miR-3679-5p gene (miRBase Accession No. MIMAT0018104) described in SEQ ID NO: 39, a homolog or an ortholog of a different organism species, and the like. The hsa-miR-3679-5p gene can be obtained by a method described in Creighton C J et al., 2010, PLoS One, Vol. 5, e9637. Also, “hsa-mir-3679” (miRBase Accession No. MI0016080, SEQ ID NO: 240) having a hairpin-like structure is known as a precursor of “hsa-miR-3679-5p”.

The term “hsa-miR-7110-5p gene” or “hsa-miR-7110-5p” used herein includes the hsa-miR-7110-5p gene (miRBase Accession No. MIMAT0028117) described in SEQ ID NO: 40, a homolog or an ortholog of a different organism species, and the like. The hsa-miR-7110-5p gene can be obtained by a method described in Ladewig E et al., 2012, Genome Res, Vol. 22, p. 1634-1645. Also, “hsa-mir-7110” (miRBase Accession No. MI0022961, SEQ ID NO: 241) having a hairpin-like structure is known as a precursor of “hsa-miR-7110-5p”.

The term “hsa-miR-4516 gene” or “hsa-miR-4516” used herein includes the hsa-miR-4516 gene (miRBase Accession No. MIMAT0019053) described in SEQ ID NO: 41, a homolog or an ortholog of a different organism species, and the like. The hsa-miR-4516 gene can be obtained by a method described in Jima D D et al., 2010, Blood, Vol. 116, e118-e127. Also, “hsa-mir-4516” (miRBase Accession No. MI0016882, SEQ ID NO: 242) having a hairpin-like structure is known as a precursor of “hsa-miR-4516”.

The term “hsa-miR-6717-5p gene” or “hsa-miR-6717-5p” used herein includes the hsa-miR-6717-5p gene (miRBase Accession No. MIMAT0025846) described in SEQ ID NO: 42, a homolog or an ortholog of a different organism species, and the like. The hsa-miR-6717-5p gene can be obtained by a method described in Li Y et al., 2012, Gene, Vol. 497, p. 330-335. Also, “hsa-mir-6717” (miRBase Accession No. MI0022551, SEQ ID NO: 243) having a hairpin-like structure is known as a precursor of “hsa-miR-6717-5p”.

The term “hsa-miR-6826-5p gene” or “hsa-miR-6826-5p” used herein includes the hsa-miR-6826-5p gene (miRBase Accession No. MIMAT0027552) described in SEQ ID NO: 43, a homolog or an ortholog of a different organism species, and the like. The hsa-miR-6826-5p gene can be obtained by a method described in Ladewig E et al., 2012, Genome Res, Vol. 22, p. 1634-1645. Also, “hsa-mir-6826” (miRBase Accession No. MI0022671, SEQ ID NO: 244) having a hairpin-like structure is known as a precursor of “hsa-miR-6826-5p”.

The term “hsa-miR-4433b-3p gene” or “hsa-miR-4433b-3p” used herein includes the hsa-miR-4433b-3p gene (miRBase Accession No. MIMAT0030414) described in SEQ ID NO: 44, a homolog or an ortholog of a different organism species, and the like. The hsa-miR-4433b-3p gene can be obtained by a method described in Ple H et al., 2012, PLoS One, Vol. 7, e50746. Also, “hsa-mir-4433b” (miRBase Accession No. M10025511, SEQ ID NO: 245) having a hairpin-like structure is known as a precursor of “hsa-miR-4433b-3p”.

The term “hsa-miR-3679-3p gene” or “hsa-miR-3679-3p” used herein includes the hsa-miR-3679-3p gene (miRBase Accession No. MIMAT0018105) described in SEQ ID NO: 45, a homolog or an ortholog of a different organism species, and the like. The hsa-miR-3679-3p gene can be obtained by a method described in Creighton C J et al., 2010, PLoS One, Vol. 5, e9637. Also, “hsa-mir-3679” (miRBase Accession No. MI0016080, SEQ ID NO: 240) having a hairpin-like structure is known as a precursor of “hsa-miR-3679-3p”.

The term “hsa-miR-3135b gene” or “hsa-miR-3135b” used herein includes the hsa-miR-3135b gene (miRBase Accession No. MIMAT0018985) described in SEQ ID NO: 46, a homolog or an ortholog of a different organism species, and the like. The hsa-miR-3135b gene can be obtained by a method described in Jima D D et al., 2010, Blood, Vol. 116, e118-e127. Also, “hsa-mir-3135b” (miRBase Accession No. MI0016809, SEQ ID NO: 246) having a hairpin-like structure is known as a precursor of “hsa-miR-3135b”.

The term “hsa-miR-3622a-5p gene” or “hsa-miR-3622a-5p” used herein includes the hsa-miR-3622a-5p gene (miRBase Accession No. MIMAT0018003) described in SEQ ID NO: 47, a homolog or an ortholog of a different organism species, and the like. The hsa-miR-3622a-5p gene can be obtained by a method described in Witten D et al., 2010, BMC Biol, Vol. 8, p. 58. Also, “hsa-mir-3622a” (miRBase Accession No. MI0016013, SEQ ID NO: 247) having a hairpin-like structure is known as a precursor of “hsa-miR-3622a-5p”.

The term “hsa-miR-711 gene” or “hsa-miR-711” used herein includes the hsa-miR-711 gene (miRBase Accession No. MIMAT0012734) described in SEQ ID NO: 48, a homolog or an ortholog of a different organism species, and the like. The hsa-miR-711 gene can be obtained by a method described in Artzi S et al., 2008, BMC Bioinformatics, Vol. 9, p. 39. Also, “hsa-mir-711” (miRBase Accession No. MI0012488, SEQ ID NO: 248) having a hairpin-like structure is known as a precursor of “hsa-miR-711”.

The term “hsa-miR-4467 gene” or “hsa-miR-4467” used herein includes the hsa-miR-4467 gene (miRBase Accession No. MIMAT0018994) described in SEQ ID NO: 49, a homolog or an ortholog of a different organism species, and the like. The hsa-miR-4467 gene can be obtained by a method described in Jima D D et al., 2010, Blood, Vol. 116, e118-e127. Also, “hsa-mir-4467” (miRBase Accession No. MI0016818, SEQ ID NO: 249) having a hairpin-like structure is known as a precursor of “hsa-miR-4467”.

The term “hsa-miR-6857-5p gene” or “hsa-miR-6857-5p” used herein includes the hsa-miR-6857-5p gene (miRBase Accession No. MIMAT0027614) described in SEQ ID NO: 50, a homolog or an ortholog of a different organism species, and the like. The hsa-miR-6857-5p gene can be obtained by a method described in Ladewig E et al., 2012, Genome Res, Vol. 22, p. 1634-1645. Also, “hsa-mir-6857” (miRBase Accession No. MI0022703, SEQ ID NO: 250) having a hairpin-like structure is known as a precursor of “hsa-miR-6857-5p”.

The term “hsa-miR-6515-3p gene” or “hsa-miR-6515-3p” used herein includes the hsa-miR-6515-3p gene (miRBase Accession No. MIMAT0025487) described in SEQ ID NO: 51, a homolog or an ortholog of a different organism species, and the like. The hsa-miR-6515-3p gene can be obtained by a method described in Joyce C E et al., 2011, Hum Mol Genet, Vol. 20, p. 4025-4040. Also, “hsa-mir-6515” (miRBase Accession No. MI0022227, SEQ ID NO: 251) having a hairpin-like structure is known as a precursor of “hsa-miR-6515-3p”.

The term “hsa-miR-1225-5p gene” or “hsa-miR-1225-5p” used herein includes the hsa-miR-1225-5p gene (miRBase Accession No. MIMAT0005572) described in SEQ ID NO: 52, a homolog or an ortholog of a different organism species, and the like. The hsa-miR-1225-5p gene can be obtained by a method described in Berezikov E et al., 2007, Mol Cell, Vol. 28, p. 328-336. Also, “hsa-mir-1225” (miRBase Accession No. MI0006311, SEQ ID NO: 206) having a hairpin-like structure is known as a precursor of “hsa-miR-1225-5p”.

The term “hsa-miR-187-5p gene” or “hsa-miR-187-5p” used herein includes the hsa-miR-187-5p gene (miRBase Accession No. MIMAT0004561) described in SEQ ID NO: 53, a homolog or an ortholog of a different organism species, and the like. The hsa-miR-187-5p gene can be obtained by a method described in Lim L P et al., 2003, Science, Vol. 299, p. 1540. Also, “hsa-mir-187” (miRBase Accession No. MI0000274, SEQ ID NO: 252) having a hairpin-like structure is known as a precursor of “hsa-miR-187-5p”.

The term “hsa-miR-3185 gene” or “hsa-miR-3185” used herein includes the hsa-miR-3185 gene (miRBase Accession No. MIMAT0015065) described in SEQ ID NO: 54, a homolog or an ortholog of a different organism species, and the like. The hsa-miR-3185 gene can be obtained by a method described in Stark M S et al., 2010, PLoS One, Vol. 5, e9685. Also, “hsa-mir-3185” (miRBase Accession No. MI0014227, SEQ ID NO: 253) having a hairpin-like structure is known as a precursor of “hsa-miR-3185”.

The term “hsa-miR-642b-3p gene” or “hsa-miR-642b-3p” used herein includes the hsa-miR-642b-3p gene (miRBase Accession No. MIMAT0018444) described in SEQ ID NO: 55, a homolog or an ortholog of a different organism species, and the like. The hsa-miR-642b-3p gene can be obtained by a method described in Witten D et al., 2010, BMC Biol, Vol. 8, p. 58. Also, “hsa-mir-642b” (miRBase Accession No. MI0016685, SEQ ID NO: 254) having a hairpin-like structure is known as a precursor of “hsa-miR-642b-3p”.

The term “hsa-miR-1249 gene” or “hsa-miR-1249” used herein includes the hsa-miR-1249 gene (miRBase Accession No. MIMAT0005901) described in SEQ ID NO: 56, a homolog or an ortholog of a different organism species, and the like. The hsa-miR-1249 gene can be obtained by a method described in Morin R D et al., 2008, Genome Res, Vol. 18, p. 610-621. Also, “hsa-mir-1249” (miRBase Accession No. MI0006384, SEQ ID NO: 255) having a hairpin-like structure is known as a precursor of “hsa-miR-1249”.

The term “hsa-miR-744-5p gene” or “hsa-miR-744-5p” used herein includes the hsa-miR-744-5p gene (miRBase Accession No. MIMAT0004945) described in SEQ ID NO: 57, a homolog or an ortholog of a different organism species, and the like. The hsa-miR-744-5p gene can be obtained by a method described in Berezikov E et al., 2006, Genome Res, Vol. 16, p. 1289-1298. Also, “hsa-mir-744” (miRBase Accession No. MI0005559, SEQ ID NO: 256) having a hairpin-like structure is known as a precursor of “hsa-miR-744-5p”.

The term “hsa-miR-4442 gene” or “hsa-miR-4442” used herein includes the hsa-miR-4442 gene (miRBase Accession No. MIMAT0018960) described in SEQ ID NO: 58, a homolog or an ortholog of a different organism species, and the like. The hsa-miR-4442 gene can be obtained by a method described in Jima D D et al., 2010, Blood, Vol. 116, e118-e127. Also, “hsa-mir-4442” (miRBase Accession No. MI0016785, SEQ ID NO: 257) having a hairpin-like structure is known as a precursor of “hsa-miR-4442”.

The term “hsa-miR-1228-3p gene” or “hsa-miR-1228-3p” used herein includes the hsa-miR-1228-3p gene (miRBase Accession No. MIMAT0005583) described in SEQ ID NO: 59, a homolog or an ortholog of a different organism species, and the like. The hsa-miR-1228-3p gene can be obtained by a method described in Berezikov E et al., 2007, Mol Cell, Vol. 28, p. 328-336. Also, “hsa-mir-1228” (miRBase Accession No. MI0006318, SEQ ID NO: 258) having a hairpin-like structure is known as a precursor of “hsa-miR-1228-3p”.

The term “hsa-miR-939-5p gene” or “hsa-miR-939-5p” used herein includes the hsa-miR-939-5p gene (miRBase Accession No. MIMAT0004982) described in SEQ ID NO: 60, a homolog or an ortholog of a different organism species, and the like. The hsa-miR-939-5p gene can be obtained by a method described in Lui W O et al., 2007, Cancer Res, Vol. 67, p. 6031-6043. Also, “hsa-mir-939” (miRBase Accession No. MI0005761, SEQ ID NO: 259) having a hairpin-like structure is known as a precursor of “hsa-miR-939-5p”.

The term “hsa-miR-6845-5p gene” or “hsa-miR-6845-5p” used herein includes the hsa-miR-6845-5p gene (miRBase Accession No. MIMAT0027590) described in SEQ ID NO: 61, a homolog or an ortholog of a different organism species, and the like. The hsa-miR-6845-5p gene can be obtained by a method described in Ladewig E et al., 2012, Genome Res, Vol. 22, p. 1634-1645. Also, “hsa-mir-6845” (miRBase Accession No. MI0022691, SEQ ID NO: 260) having a hairpin-like structure is known as a precursor of “hsa-miR-6845-5p”.

The term “hsa-miR-887-3p gene” or “hsa-miR-887-3p” used herein includes the hsa-miR-887-3p gene (miRBase Accession No. MIMAT0004951) described in SEQ ID NO: 62, a homolog or an ortholog of a different organism species, and the like. The hsa-miR-887-3p gene can be obtained by a method described in Berezikov E et al., 2006, Genome Res, Vol. 16, p. 1289-1298. Also, “hsa-mir-887” (miRBase Accession No. MI0005562, SEQ ID NO: 261) having a hairpin-like structure is known as a precursor of “hsa-miR-887-3p”.

The term “hsa-miR-7845-5p gene” or “hsa-miR-7845-5p” used herein includes the hsa-miR-7845-5p gene (miRBase Accession No. MIMAT0030420) described in SEQ ID NO: 63, a homolog or an ortholog of a different organism species, and the like. The hsa-miR-7845-5p gene can be obtained by a method described in Ple H et al., 2012, PLoS One, Vol. 7, e50746. Also, “hsa-mir-7845” (miRBase Accession No. MI0025515, SEQ ID NO: 262) having a hairpin-like structure is known as a precursor of “hsa-miR-7845-5p”.

The term “hsa-miR-6729-5p gene” or “hsa-miR-6729-5p” used herein includes the hsa-miR-6729-5p gene (miRBase Accession No. MIMAT0027359) described in SEQ ID NO: 64, a homolog or an ortholog of a different organism species, and the like. The hsa-miR-6729-5p gene can be obtained by a method described in Ladewig E et al., 2012, Genome Res, Vol. 22, p. 1634-1645. Also, “hsa-mir-6729” (miRBase Accession No. MI0022574, SEQ ID NO: 263) having a hairpin-like structure is known as a precursor of “hsa-miR-6729-5p”.

The term “hsa-miR-4632-5p gene” or “hsa-miR-4632-5p” used herein includes the hsa-miR-4632-5p gene (miRBase Accession No. MIMAT0022977) described in SEQ ID NO: 65, a homolog or an ortholog of a different organism species, and the like. The hsa-miR-4632-5p gene can be obtained by a method described in Persson H et al., 2011, Cancer Res, Vol. 71, p. 78-86. Also, “hsa-mir-4632” (miRBase Accession No. MI0017259, SEQ ID NO: 264) having a hairpin-like structure is known as a precursor of “hsa-miR-4632-5p”.

The term “hsa-miR-615-5p gene” or “hsa-miR-615-5p” used herein includes the hsa-miR-615-5p gene (miRBase Accession No. MIMAT0004804) described in SEQ ID NO: 66, a homolog or an ortholog of a different organism species, and the like. The hsa-miR-615-5p gene can be obtained by a method described in Cummins J M et al., 2006, Proc Natl Acad Sci USA, Vol. 103, p. 3687-3692. Also, “hsa-mir-615” (miRBase Accession No. MI0003628, SEQ ID NO: 265) having a hairpin-like structure is known as a precursor of “hsa-miR-615-5p”.

The term “hsa-miR-6724-5p gene” or “hsa-miR-6724-5p” used herein includes the hsa-miR-6724-5p gene (miRBase Accession No. MIMAT0025856) described in SEQ ID NO: 67, a homolog or an ortholog of a different organism species, and the like. The hsa-miR-6724-5p gene can be obtained by a method described in Li Y et al., 2012, Gene, Vol. 497, p. 330-335. Also, “hsa-mir-6724” (miRBase Accession No. MI0022559, SEQ ID NO: 266) having a hairpin-like structure is known as a precursor of “hsa-miR-6724-5p”.

The term “hsa-miR-4728-5p gene” or “hsa-miR-4728-5p” used herein includes the hsa-miR-4728-5p gene (miRBase Accession No. MIMAT0019849) described in SEQ ID NO: 68, a homolog or an ortholog of a different organism species, and the like. The hsa-miR-4728-5p gene can be obtained by a method described in Persson H et al., 2011, Cancer Res, Vol. 71, p. 78-86. Also, “hsa-mir-4728” (miRBase Accession No. MI0017365, SEQ ID NO: 267) having a hairpin-like structure is known as a precursor of “hsa-miR-4728-5p”.

The term “hsa-miR-6732-5p gene” or “hsa-miR-6732-5p” used herein includes the hsa-miR-6732-5p gene (miRBase Accession No. MIMAT0027365) described in SEQ ID NO: 69, a homolog or an ortholog of a different organism species, and the like. The hsa-miR-6732-5p gene can be obtained by a method described in Ladewig E et al., 2012, Genome Res, Vol. 22, p. 1634-1645. Also, “hsa-mir-6732” (miRBase Accession No. MI0022577, SEQ ID NO: 268) having a hairpin-like structure is known as a precursor of “hsa-miR-6732-5p”.

The term “hsa-miR-6816-5p gene” or “hsa-miR-6816-5p” used herein includes the hsa-miR-6816-5p gene (miRBase Accession No. MIMAT0027532) described in SEQ ID NO: 70, a homolog or an ortholog of a different organism species, and the like. The hsa-miR-6816-5p gene can be obtained by a method described in Ladewig E et al., 2012, Genome Res, Vol. 22, p. 1634-1645. Also, “hsa-mir-6816” (miRBase Accession No. MI0022661, SEQ ID NO: 269) having a hairpin-like structure is known as a precursor of “hsa-miR-6816-5p”.

The term “hsa-miR-4695-5p gene” or “hsa-miR-4695-5p” used herein includes the hsa-miR-4695-5p gene (miRBase Accession No. MIMAT0019788) described in SEQ ID NO: 71, a homolog or an ortholog of a different organism species, and the like. The hsa-miR-4695-5p gene can be obtained by a method described in Persson H et al., 2011, Cancer Res, Vol. 71, p. 78-86. Also, “hsa-mir-4695” (miRBase Accession No. MI0017328, SEQ ID NO: 270) having a hairpin-like structure is known as a precursor of “hsa-miR-4695-5p”.

The term “hsa-miR-6088 gene” or “hsa-miR-6088” used herein includes the hsa-miR-6088 gene (miRBase Accession No. MIMAT0023713) described in SEQ ID NO: 72, a homolog or an ortholog of a different organism species, and the like. The hsa-miR-6088 gene can be obtained by a method described in Yoo J K et al., 2012, Stem Cells Dev, Vol. 21, p. 2049-2057. Also, “hsa-mir-6088” (miRBase Accession No. MI0020365, SEQ ID NO: 271) having a hairpin-like structure is known as a precursor of “hsa-miR-6088”.

The term “hsa-miR-7975 gene” or “hsa-miR-7975” used herein includes the hsa-miR-7975 gene (miRBase Accession No. MIMAT0031178) described in SEQ ID NO: 73, a homolog or an ortholog of a different organism species, and the like. The hsa-miR-7975 gene can be obtained by a method described in Velthut-Meikas A et al., 2013, Mol Endocrinol, online. Also, “hsa-mir-7975” (miRBase Accession No. MI0025751, SEQ ID NO: 272) having a hairpin-like structure is known as a precursor of “hsa-miR-7975”.

The term “hsa-miR-3197 gene” or “hsa-miR-3197” used herein includes the hsa-miR-3197 gene (miRBase Accession No. MIMAT0015082) described in SEQ ID NO: 74, a homolog or an ortholog of a different organism species, and the like. The hsa-miR-3197 gene can be obtained by a method described in Stark M S et al., 2010, PLoS One, Vol. 5, e9685. Also, “hsa-mir-3197” (miRBase Accession No. MI0014245, SEQ ID NO: 273) having a hairpin-like structure is known as a precursor of “hsa-miR-3197”.

The term “hsa-miR-6125 gene” or “hsa-miR-6125” used herein includes the hsa-miR-6125 gene (miRBase Accession No. MIMAT0024598) described in SEQ ID NO: 75, a homolog or an ortholog of a different organism species, and the like. The hsa-miR-6125 gene can be obtained by a method described in Smith J L et al., 2012, J Virol, Vol. 86, p. 5278-5287. Also, “hsa-mir-6125” (miRBase Accession No. MI0021259, SEQ ID NO: 274) having a hairpin-like structure is known as a precursor of “hsa-miR-6125”.

The term “hsa-miR-4433-3p gene” or “hsa-miR-4433-3p” used herein includes the hsa-miR-4433-3p gene (miRBase Accession No. MIMAT0018949) described in SEQ ID NO: 76, a homolog or an ortholog of a different organism species, and the like. The hsa-miR-4433-3p gene can be obtained by a method described in Jima D D et al., 2010, Blood, Vol. 116, e118-e127. Also, “hsa-mir-4433” (miRBase Accession No. MI0016773, SEQ ID NO: 275) having a hairpin-like structure is known as a precursor of “hsa-miR-4433-3p”.

The term “hsa-miR-6727-5p gene” or “hsa-miR-6727-5p” used herein includes the hsa-miR-6727-5p gene (miRBase Accession No. MIMAT0027355) described in SEQ ID NO: 77, a homolog or an ortholog of a different organism species, and the like. The hsa-miR-6727-5p gene can be obtained by a method described in Ladewig E et al., 2012, Genome Res, Vol. 22, p. 1634-1645. Also, “hsa-mir-6727” (miRBase Accession No. MI0022572, SEQ ID NO: 276) having a hairpin-like structure is known as a precursor of “hsa-miR-6727-5p”.

The term “hsa-miR-4706 gene” or “hsa-miR-4706” used herein includes the hsa-miR-4706 gene (miRBase Accession No. MIMAT0019806) described in SEQ ID NO: 78, a homolog or an ortholog of a different organism species, and the like. The hsa-miR-4706 gene can be obtained by a method described in Persson H et al., 2011, Cancer Res, Vol. 71, p. 78-86. Also, “hsa-mir-4706” (miRBase Accession No. MI0017339, SEQ ID NO: 277) having a hairpin-like structure is known as a precursor of “hsa-miR-4706”.

The term “hsa-miR-7847-3p gene” or “hsa-miR-7847-3p” used herein includes the hsa-miR-7847-3p gene (miRBase Accession No. MIMAT0030422) described in SEQ ID NO: 79, a homolog or an ortholog of a different organism species, and the like. The hsa-miR-7847-3p gene can be obtained by a method described in Ple H et al., 2012, PLoS One, Vol. 7, e50746. Also, “hsa-mir-7847” (miRBase Accession No. MI0025517, SEQ ID NO: 278) having a hairpin-like structure is known as a precursor of “hsa-miR-7847-3p”.

The term “hsa-miR-6805-3p gene” or “hsa-miR-6805-3p” used herein includes the hsa-miR-6805-3p gene (miRBase Accession No. MIMAT0027511) described in SEQ ID NO: 80, a homolog or an ortholog of a different organism species, and the like. The hsa-miR-6805-3p gene can be obtained by a method described in Ladewig E et al., 2012, Genome Res, Vol. 22, p. 1634-1645. Also, “hsa-mir-6805” (miRBase Accession No. MI0022650, SEQ ID NO: 279) having a hairpin-like structure is known as a precursor of “hsa-miR-6805-3p”.

The term “hsa-miR-6766-3p gene” or “hsa-miR-6766-3p” used herein includes the hsa-miR-6766-3p gene (miRBase Accession No. MIMAT0027433) described in SEQ ID NO: 81, a homolog or an ortholog of a different organism species, and the like. The hsa-miR-6766-3p gene can be obtained by a method described in Ladewig E et al., 2012, Genome Res, Vol. 22, p. 1634-1645. Also, “hsa-mir-6766” (miRBase Accession No. MI0022611, SEQ ID NO: 280) having a hairpin-like structure is known as a precursor of “hsa-miR-6766-3p”.

The term “hsa-miR-1913 gene” or “hsa-miR-1913” used herein includes the hsa-miR-1913 gene (miRBase Accession No. MIMAT0007888) described in SEQ ID NO: 82, a homolog or an ortholog of a different organism species, and the like. The hsa-miR-1913 gene can be obtained by a method described in Bar M et al., 2008, Stem Cells, Vol. 26, p. 2496-2505. Also, “hsa-mir-1913” (miRBase Accession No. MI0008334, SEQ ID NO: 281) having a hairpin-like structure is known as a precursor of “hsa-miR-1913”.

The term “hsa-miR-4649-5p gene” or “hsa-miR-4649-5p” used herein includes the hsa-miR-4649-5p gene (miRBase Accession No. MIMAT0019711) described in SEQ ID NO: 83, a homolog or an ortholog of a different organism species, and the like. The hsa-miR-4649-5p gene can be obtained by a method described in Persson H et al., 2011, Cancer Res, Vol. 71, p. 78-86. Also, “hsa-mir-4649” (miRBase Accession No. MI0017276, SEQ ID NO: 282) having a hairpin-like structure is known as a precursor of “hsa-miR-4649-5p”.

The term “hsa-miR-602 gene” or “hsa-miR-602” used herein includes the hsa-miR-602 gene (miRBase Accession No. MIMAT0003270) described in SEQ ID NO: 84, a homolog or an ortholog of a different organism species, and the like. The hsa-miR-602 gene can be obtained by a method described in Cummins J M et al., 2006, Proc Natl Acad Sci USA, Vol. 103, p. 3687-3692. Also, “hsa-mir-602” (miRBase Accession No. MI0003615, SEQ ID NO: 283) having a hairpin-like structure is known as a precursor of “hsa-miR-602”.

The term “hsa-miR-3663-3p gene” or “hsa-miR-3663-3p” used herein includes the hsa-miR-3663-3p gene (miRBase Accession No. MIMAT0018085) described in SEQ ID NO: 85, a homolog or an ortholog of a different organism species, and the like. The hsa-miR-3663-3p gene can be obtained by a method described in Liao J Y et al., 2010, PLoS One, Vol. 5, e10563. Also, “hsa-mir-3663” (miRBase Accession No. MI0016064, SEQ ID NO: 284) having a hairpin-like structure is known as a precursor of “hsa-miR-3663-3p”.

The term “hsa-miR-6893-5p gene” or “hsa-miR-6893-5p” used herein includes the hsa-miR-6893-5p gene (miRBase Accession No. MIMAT0027686) described in SEQ ID NO: 86, a homolog or an ortholog of a different organism species, and the like. The hsa-miR-6893-5p gene can be obtained by a method described in Ladewig E et al., 2012, Genome Res, Vol. 22, p. 1634-1645. Also, “hsa-mir-6893” (miRBase Accession No. MI0022740, SEQ ID NO: 285) having a hairpin-like structure is known as a precursor of “hsa-miR-6893-5p”.

The term “hsa-miR-6861-5p gene” or “hsa-miR-6861-5p” used herein includes the hsa-miR-6861-5p gene (miRBase Accession No. MIMAT0027623) described in SEQ ID NO: 87, a homolog or an ortholog of a different organism species, and the like. The hsa-miR-6861-5p gene can be obtained by a method described in Ladewig E et al., 2012, Genome Res, Vol. 22, p. 1634-1645. Also, “hsa-mir-6861” (miRBase Accession No. MI0022708, SEQ ID NO: 286) having a hairpin-like structure is known as a precursor of “hsa-miR-6861-5p”.

The term “hsa-miR-4449 gene” or “hsa-miR-4449” used herein includes the hsa-miR-4449 gene (miRBase Accession No. MIMAT0018968) described in SEQ ID NO: 88, a homolog or an ortholog of a different organism species, and the like. The hsa-miR-4449 gene can be obtained by a method described in Jima D D et al., 2010, Blood, Vol. 116, e118-e127. Also, “hsa-mir-4449” (miRBase Accession No. MI0016792, SEQ ID NO: 287) having a hairpin-like structure is known as a precursor of “hsa-miR-4449”.

The term “hsa-miR-6842-5p gene” or “hsa-miR-6842-5p” used herein includes the hsa-miR-6842-5p gene (miRBase Accession No. MIMAT0027586) described in SEQ ID NO: 89, a homolog or an ortholog of a different organism species, and the like. The hsa-miR-6842-5p gene can be obtained by a method described in Ladewig E et al., 2012, Genome Res, Vol. 22, p. 1634-1645. Also, “hsa-mir-6842” (miRBase Accession No. MI0022688, SEQ ID NO: 288) having a hairpin-like structure is known as a precursor of “hsa-miR-6842-5p”.

The term “hsa-miR-4454 gene” or “hsa-miR-4454” used herein includes the hsa-miR-4454 gene (miRBase Accession No. MIMAT0018976) described in SEQ ID NO: 90, a homolog or an ortholog of a different organism species, and the like. The hsa-miR-4454 gene can be obtained by a method described in Jima D D et al., 2010, Blood, Vol. 116, e118-e127. Also, “hsa-mir-4454” (miRBase Accession No. MI0016800, SEQ ID NO: 289) having a hairpin-like structure is known as a precursor of “hsa-miR-4454”.

The term “hsa-miR-5195-3p gene” or “hsa-miR-5195-3p” used herein includes the hsa-miR-5195-3p gene (miRBase Accession No. MIMAT0021127) described in SEQ ID NO: 91, a homolog or an ortholog of a different organism species, and the like. The hsa-miR-5195-3p gene can be obtained by a method described in Schotte D et al., 2011, Leukemia, Vol. 25, p. 1389-1399. Also, “hsa-mir-5195” (miRBase Accession No. MI0018174, SEQ ID NO: 290) having a hairpin-like structure is known as a precursor of “hsa-miR-5195-3p”.

The term “hsa-miR-663b gene” or “hsa-miR-663b” used herein includes the hsa-miR-663b gene (miRBase Accession No. MIMAT0005867) described in SEQ ID NO: 92, a homolog or an ortholog of a different organism species, and the like. The hsa-miR-663b gene can be obtained by a method described in Takada S et al., 2008, Leukemia, Vol. 22, p. 1274-1278. Also, “hsa-mir-663b” (miRBase Accession No. MI0006336, SEQ ID NO: 291) having a hairpin-like structure is known as a precursor of “hsa-miR-663b”.

The term “hsa-miR-6765-5p gene” or “hsa-miR-6765-5p” used herein includes the hsa-miR-6765-5p gene (miRBase Accession No. MIMAT0027430) described in SEQ ID NO: 93, a homolog or an ortholog of a different organism species, and the like. The hsa-miR-6765-5p gene can be obtained by a method described in Ladewig E et al., 2012, Genome Res, Vol. 22, p. 1634-1645. Also, “hsa-mir-6765” (miRBase Accession No. MI0022610, SEQ ID NO: 292) having a hairpin-like structure is known as a precursor of “hsa-miR-6765-5p”.

The term “hsa-miR-4513 gene” or “hsa-miR-4513” used herein includes the hsa-miR-4513 gene (miRBase Accession No. MIMAT0019050) described in SEQ ID NO: 94, a homolog or an ortholog of a different organism species, and the like. The hsa-miR-4513 gene can be obtained by a method described in Jima D D et al., 2010, Blood, Vol. 116, e118-e127. Also, “hsa-mir-4513” (miRBase Accession No. MI0016879, SEQ ID NO: 293) having a hairpin-like structure is known as a precursor of “hsa-miR-4513”.

The term “hsa-miR-614 gene” or “hsa-miR-614” used herein includes the hsa-miR-614 gene (miRBase Accession No. MIMAT0003282) described in SEQ ID NO: 95, a homolog or an ortholog of a different organism species, and the like. The hsa-miR-614 gene can be obtained by a method described in Cummins J M et al., 2006, Proc Natl Acad Sci USA, Vol. 103, p. 3687-3692. Also, “hsa-mir-614” (miRBase Accession No. MI0003627, SEQ ID NO: 294) having a hairpin-like structure is known as a precursor of “hsa-miR-614”.

The term “hsa-miR-6785-5p gene” or “hsa-miR-6785-5p” used herein includes the hsa-miR-6785-5p gene (miRBase Accession No. MIMAT0027470) described in SEQ ID NO: 96, a homolog or an ortholog of a different organism species, and the like. The hsa-miR-6785-5p gene can be obtained by a method described in Ladewig E et al., 2012, Genome Res, Vol. 22, p. 1634-1645. Also, “hsa-mir-6785” (miRBase Accession No. MI0022630, SEQ ID NO: 295) having a hairpin-like structure is known as a precursor of “hsa-miR-6785-5p”.

The term “hsa-miR-6777-5p gene” or “hsa-miR-6777-5p” used herein includes the hsa-miR-6777-5p gene (miRBase Accession No. MIMAT0027454) described in SEQ ID NO: 97, a homolog or an ortholog of a different organism species, and the like. The hsa-miR-6777-5p gene can be obtained by a method described in Ladewig E et al., 2012, Genome Res, Vol. 22, p. 1634-1645. Also, “hsa-mir-6777” (miRBase Accession No. MI0022622, SEQ ID NO: 296) having a hairpin-like structure is known as a precursor of “hsa-miR-6777-5p”.

The term “hsa-miR-940 gene” or “hsa-miR-940” used herein includes the hsa-miR-940 gene (miRBase Accession No. MIMAT0004983) described in SEQ ID NO: 98, a homolog or an ortholog of a different organism species, and the like. The hsa-miR-940 gene can be obtained by a method described in Lui W O et al., 2007, Cancer Res, Vol. 67, p. 6031-6043. Also, “hsa-mir-940” (miRBase Accession No. MI0005762, SEQ ID NO: 297) having a hairpin-like structure is known as a precursor of “hsa-miR-940”.

The term “hsa-miR-4741 gene” or “hsa-miR-4741” used herein includes the hsa-miR-4741 gene (miRBase Accession No. MIMAT0019871) described in SEQ ID NO: 99, a homolog or an ortholog of a different organism species, and the like. The hsa-miR-4741 gene can be obtained by a method described in Persson H et al., 2011, Cancer Res, Vol. 71, p. 78-86. Also, “hsa-mir-4741” (miRBase Accession No. MI0017379, SEQ ID NO: 298) having a hairpin-like structure is known as a precursor of “hsa-miR-4741”.

The term “hsa-miR-6870-5p gene” or “hsa-miR-6870-5p” used herein includes the hsa-miR-6870-5p gene (miRBase Accession No. MIMAT0027640) described in SEQ ID NO: 100, a homolog or an ortholog of a different organism species, and the like. The hsa-miR-6870-5p gene can be obtained by a method described in Ladewig E et al., 2012, Genome Res, Vol. 22, p. 1634-1645. Also, “hsa-mir-6870” (miRBase Accession No. MI0022717, SEQ ID NO: 299) having a hairpin-like structure is known as a precursor of “hsa-miR-6870-5p”.

The term “hsa-miR-6131 gene” or “hsa-miR-6131” used herein includes the hsa-miR-6131 gene (miRBase Accession No. MIMAT0024615) described in SEQ ID NO: 101, a homolog or an ortholog of a different organism species, and the like. The hsa-miR-6131 gene can be obtained by a method described in Dannemann M et al., 2012, Genome Biol Evol, Vol. 4, p. 552-564. Also, “hsa-mir-6131” (miRBase Accession No. MI0021276, SEQ ID NO: 300) having a hairpin-like structure is known as a precursor of “hsa-miR-6131”.

The term “hsa-miR-150-3p gene” or “hsa-miR-150-3p” used herein includes the hsa-miR-150-3p gene (miRBase Accession No. MIMAT0004610) described in SEQ ID NO: 102, a homolog or an ortholog of a different organism species, and the like. The hsa-miR-150-3p gene can be obtained by a method described in Lagos-Quintana M et al., 2002, Curr Biol, Vol. 12, p. 735-739. Also, “hsa-mir-150” (miRBase Accession No. MI0000479, SEQ ID NO: 301) having a hairpin-like structure is known as a precursor of “hsa-miR-150-3p”.

The term “hsa-miR-4707-5p gene” or “hsa-miR-4707-5p” used herein includes the hsa-miR-4707-5p gene (miRBase Accession No. MIMAT0019807) described in SEQ ID NO: 103, a homolog or an ortholog of a different organism species, and the like. The hsa-miR-4707-5p gene can be obtained by a method described in Persson H et al., 2011, Cancer Res, Vol. 71, p. 78-86. Also, “hsa-mir-4707” (miRBase Accession No. MI0017340, SEQ ID NO: 302) having a hairpin-like structure is known as a precursor of “hsa-miR-4707-5p”.

The term “hsa-miR-1915-3p gene” or “hsa-miR-1915-3p” used herein includes the hsa-miR-1915-3p gene (miRBase Accession No. MIMAT0007892) described in SEQ ID NO: 104, a homolog or an ortholog of a different organism species, and the like. The hsa-miR-1915-3p gene can be obtained by a method described in Bar M et al., 2008, Stem Cells, Vol. 26, p. 2496-2505. Also, “hsa-mir-1915” (miRBase Accession No. MI0008336, SEQ ID NO: 303) having a hairpin-like structure is known as a precursor of “hsa-miR-1915-3p”.

The term “hsa-miR-3937 gene” or “hsa-miR-3937” used herein includes the hsa-miR-3937 gene (miRBase Accession No. MIMAT0018352) described in SEQ ID NO: 105, a homolog or an ortholog of a different organism species, and the like. The hsa-miR-3937 gene can be obtained by a method described in Liao J Y et al., 2010, PLoS One, Vol. 5, e10563. Also, “hsa-mir-3937” (miRBase Accession No. MI0016593, SEQ ID NO: 304) having a hairpin-like structure is known as a precursor of “hsa-miR-3937”.

The term “hsa-miR-937-5p gene” or “hsa-miR-937-5p” used herein includes the hsa-miR-937-5p gene (miRBase Accession No. MIMAT0022938) described in SEQ ID NO: 106, a homolog or an ortholog of a different organism species, and the like. The hsa-miR-937-5p gene can be obtained by a method described in Lui W O et al., 2007, Cancer Res, Vol. 67, p. 6031-6043. Also, “hsa-mir-937” (miRBase Accession No. MI0005759, SEQ ID NO: 305) having a hairpin-like structure is known as a precursor of “hsa-miR-937-5p”.

The term “hsa-miR-4443 gene” or “hsa-miR-4443” used herein includes the hsa-miR-4443 gene (miRBase Accession No. MIMAT0018961) described in SEQ ID NO: 107, a homolog or an ortholog of a different organism species, and the like. The hsa-miR-4443 gene can be obtained by a method described in Jima D D et al., 2010, Blood, Vol. 116, e118-e127. Also, “hsa-mir-4443” (miRBase Accession No. MI0016786, SEQ ID NO: 306) having a hairpin-like structure is known as a precursor of “hsa-miR-4443”.

The term “hsa-miR-1914-3p gene” or “hsa-miR-1914-3p” used herein includes the hsa-miR-1914-3p gene (miRBase Accession No. MIMAT0007890) described in SEQ ID NO: 108, a homolog or an ortholog of a different organism species, and the like. The hsa-miR-1914-3p gene can be obtained by a method described in Bar M et al., 2008, Stem Cells, Vol. 26, p. 2496-2505. Also, “hsa-mir-1914” (miRBase Accession No. MI0008335, SEQ ID NO: 307) having a hairpin-like structure is known as a precursor of “hsa-miR-1914-3p”.

The term “hsa-miR-3620-5p gene” or “hsa-miR-3620-5p” used herein includes the hsa-miR-3620-5p gene (miRBase Accession No. MIMAT0022967) described in SEQ ID NO: 109, a homolog or an ortholog of a different organism species, and the like. The hsa-miR-3620-5p gene can be obtained by a method described in Witten D et al., 2010, BMC Biol, Vol. 8, p. 58. Also, “hsa-mir-3620” (miRBase Accession No. MI0016011, SEQ ID NO: 308) having a hairpin-like structure is known as a precursor of “hsa-miR-3620-5p”.

The term “hsa-miR-1268b gene” or “hsa-miR-1268b” used herein includes the hsa-miR-1268b gene (miRBase Accession No. MIMAT0018925) described in SEQ ID NO: 110, a homolog or an ortholog of a different organism species, and the like. The hsa-miR-1268b gene can be obtained by a method described in Jima D D et al., 2010, Blood, Vol. 116, e118-e127. Also, “hsa-mir-1268b” (miRBase Accession No. MI0016748, SEQ ID NO: 309) having a hairpin-like structure is known as a precursor of “hsa-miR-1268b”.

The term “hsa-miR-1227-5p gene” or “hsa-miR-1227-5p” used herein includes the hsa-miR-1227-5p gene (miRBase Accession No. MIMAT0022941) described in SEQ ID NO: 111, a homolog or an ortholog of a different organism species, and the like. The hsa-miR-1227-5p gene can be obtained by a method described in Berezikov E et al., 2007, Mol Cell, Vol. 28, p. 328-336. Also, “hsa-mir-1227” (miRBase Accession No. MI0006316, SEQ ID NO: 310) having a hairpin-like structure is known as a precursor of “hsa-miR-1227-5p”.

The term “hsa-miR-6880-5p gene” or “hsa-miR-6880-5p” used herein includes the hsa-miR-6880-5p gene (miRBase Accession No. MIMAT0027660) described in SEQ ID NO: 112, a homolog or an ortholog of a different organism species, and the like. The hsa-miR-6880-5p gene can be obtained by a method described in Ladewig E et al., 2012, Genome Res, Vol. 22, p. 1634-1645. Also, “hsa-mir-6880” (miRBase Accession No. MI0022727, SEQ ID NO: 311) having a hairpin-like structure is known as a precursor of “hsa-miR-6880-5p”.

The term “hsa-miR-4417 gene” or “hsa-miR-4417” used herein includes the hsa-miR-4417 gene (miRBase Accession No. MIMAT0018929) described in SEQ ID NO: 113, a homolog or an ortholog of a different organism species, and the like. The hsa-miR-4417 gene can be obtained by a method described in Jima D D et al., 2010, Blood, Vol. 116, e118-e127. Also, “hsa-mir-4417” (miRBase Accession No. MI0016753, SEQ ID NO: 312) having a hairpin-like structure is known as a precursor of “hsa-miR-4417”.

The term “hsa-miR-6802-5p gene” or “hsa-miR-6802-5p” used herein includes the hsa-miR-6802-5p gene (miRBase Accession No. MIMAT0027504) described in SEQ ID NO: 114, a homolog or an ortholog of a different organism species, and the like. The hsa-miR-6802-5p gene can be obtained by a method described in Ladewig E et al., 2012, Genome Res, Vol. 22, p. 1634-1645. Also, “hsa-mir-6802” (miRBase Accession No. MI0022647, SEQ ID NO: 313) having a hairpin-like structure is known as a precursor of “hsa-miR-6802-5p”.

The term “hsa-miR-6769a-5p gene” or “hsa-miR-6769a-5p” used herein includes the hsa-miR-6769a-5p gene (miRBase Accession No. MIMAT0027438) described in SEQ ID NO: 115, a homolog or an ortholog of a different organism species, and the like. The hsa-miR-6769a-5p gene can be obtained by a method described in Ladewig E et al., 2012, Genome Res, Vol. 22, p. 1634-1645. Also, “hsa-mir-6769a” (miRBase Accession No. MI0022614, SEQ ID NO: 314) having a hairpin-like structure is known as a precursor of “hsa-miR-6769a-5p”.

The term “hsa-miR-663a gene” or “hsa-miR-663a” used herein includes the hsa-miR-663a gene (miRBase Accession No. MIMAT0003326) described in SEQ ID NO: 116, a homolog or an ortholog of a different organism species, and the like. The hsa-miR-663a gene can be obtained by a method described in Cummins J M et al., 2006, Proc Natl Acad Sci USA, Vol. 103, p. 3687-3692. Also, “hsa-mir-663a” (miRBase Accession No. MI0003672, SEQ ID NO: 315) having a hairpin-like structure is known as a precursor of “hsa-miR-663a”.

The term “hsa-miR-6721-5p gene” or “hsa-miR-6721-5p” used herein includes the hsa-miR-6721-5p gene (miRBase Accession No. MIMAT0025852) described in SEQ ID NO: 117, a homolog or an ortholog of a different organism species, and the like. The hsa-miR-6721-5p gene can be obtained by a method described in Li Y et al., 2012, Gene, Vol. 497, p. 330-335. Also, “hsa-mir-6721” (miRBase Accession No. MI0022556, SEQ ID NO: 316) having a hairpin-like structure is known as a precursor of “hsa-miR-6721-5p”.

The term “hsa-miR-4532 gene” or “hsa-miR-4532” used herein includes the hsa-miR-4532 gene (miRBase Accession No. MIMAT0019071) described in SEQ ID NO: 118, a homolog or an ortholog of a different organism species, and the like. The hsa-miR-4532 gene can be obtained by a method described in Jima D D et al., 2010, Blood, Vol. 116, e118-e127. Also, “hsa-mir-4532” (miRBase Accession No. MI0016899, SEQ ID NO: 317) having a hairpin-like structure is known as a precursor of “hsa-miR-4532”.

The term “hsa-miR-7977 gene” or “hsa-miR-7977” used herein includes the hsa-miR-7977 gene (miRBase Accession No. MIMAT0031180) described in SEQ ID NO: 119, a homolog or an ortholog of a different organism species, and the like. The hsa-miR-7977 gene can be obtained by a method described in Velthut-Meikas A et al., 2013, Mol Endocrinol, online. Also, “hsa-mir-7977” (miRBase Accession No. MI0025753, SEQ ID NO: 318) having a hairpin-like structure is known as a precursor of “hsa-miR-7977”.

The term “hsa-miR-92b-5p gene” or “hsa-miR-92b-5p” used herein includes the hsa-miR-92b-5p gene (miRBase Accession No. MIMAT0004792) described in SEQ ID NO: 120, a homolog or an ortholog of a different organism species, and the like. The hsa-miR-92b-5p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 319) having a hairpin-like structure is known as a precursor of “hsa-miR-92b-5p”.

The term “hsa-miR-371a-5p gene” or “hsa-miR-371a-5p” used herein includes the hsa-miR-371a-5p gene (miRBase Accession No. MIMAT0004687) described in SEQ ID NO: 121, a homolog or an ortholog of a different organism species, and the like. The hsa-miR-371a-5p gene can be obtained by a method described in Suh M R et al., 2004, Dev Biol, Vol. 270, p. 488-498. Also, “hsa-mir-371a” (miRBase Accession No. MI0000779, SEQ ID NO: 320) having a hairpin-like structure is known as a precursor of “hsa-miR-371a-5p”.

The term “hsa-miR-6126 gene” or “hsa-miR-6126” used herein includes the hsa-miR-6126 gene (miRBase Accession No. MIMAT0024599) described in SEQ ID NO: 122, a homolog or an ortholog of a different organism species, and the like. The hsa-miR-6126 gene can be obtained by a method described in Smith J L et al., 2012, J Virol, Vol. 86, p. 5278-5287. Also, “hsa-mir-6126” (miRBase Accession No. MI0021260, SEQ ID NO: 321) having a hairpin-like structure is known as a precursor of “hsa-miR-6126”.

The term “hsa-miR-4734 gene” or “hsa-miR-4734” used herein includes the hsa-miR-4734 gene (miRBase Accession No. MIMAT0019859) described in SEQ ID NO: 123, a homolog or an ortholog of a different organism species, and the like. The hsa-miR-4734 gene can be obtained by a method described in Persson H et al., 2011, Cancer Res, Vol. 71, p. 78-86. Also, “hsa-mir-4734” (miRBase Accession No. MI0017371, SEQ ID NO: 322) having a hairpin-like structure is known as a precursor of “hsa-miR-4734”.

The term “hsa-miR-4665-3p gene” or “hsa-miR-4665-3p” used herein includes the hsa-miR-4665-3p gene (miRBase Accession No. MIMAT0019740) described in SEQ ID NO: 124, a homolog or an ortholog of a different organism species, and the like. The hsa-miR-4665-3p gene can be obtained by a method described in Persson H et al., 2011, Cancer Res, Vol. 71, p. 78-86. Also, “hsa-mir-4665” (miRBase Accession No. MI0017295, SEQ ID NO: 323) having a hairpin-like structure is known as a precursor of “hsa-miR-4665-3p”.

The term “hsa-miR-423-5p gene” or “hsa-miR-423-5p” used herein includes the hsa-miR-423-5p gene (miRBase Accession No. MIMAT0004748) described in SEQ ID NO: 125, a homolog or an ortholog of a different organism species, and the like. The hsa-miR-423-5p gene can be obtained by a method described in Kasashima K et al., 2004, Biochem Biophys Res Commun, Vol. 322, p. 403-410. Also, “hsa-mir-423” (miRBase Accession No. MI0001445, SEQ ID NO: 324) having a hairpin-like structure is known as a precursor of “hsa-miR-423-5p”.

The term “hsa-miR-1469 gene” or “hsa-miR-1469” used herein includes the hsa-miR-1469 gene (miRBase Accession No. MIMAT0007347) described in SEQ ID NO: 126, a homolog or an ortholog of a different organism species, and the like. The hsa-miR-1469 gene can be obtained by a method described in Kawaji H et al., 2008, BMC Genomics, Vol. 9, p. 157. Also, “hsa-mir-1469” (miRBase Accession No. MI0007074, SEQ ID NO: 325) having a hairpin-like structure is known as a precursor of “hsa-miR-1469”.

The term “hsa-miR-4675 gene” or “hsa-miR-4675” used herein includes the hsa-miR-4675 gene (miRBase Accession No. MIMAT0019757) described in SEQ ID NO: 127, a homolog or an ortholog of a different organism species, and the like. The hsa-miR-4675 gene can be obtained by a method described in Persson H et al., 2011, Cancer Res, Vol. 71, p. 78-86. Also, “hsa-mir-4675” (miRBase Accession No. MI0017306, SEQ ID NO: 326) having a hairpin-like structure is known as a precursor of “hsa-miR-4675”.

The term “hsa-miR-1915-5p gene” or “hsa-miR-1915-5p” used herein includes the hsa-miR-1915-5p gene (miRBase Accession No. MIMAT0007891) described in SEQ ID NO: 128, a homolog or an ortholog of a different organism species, and the like. The hsa-miR-1915-5p gene can be obtained by a method described in Bar M et al., 2008, Stem Cells, Vol. 26, p. 2496-2505. Also, “hsa-mir-1915” (miRBase Accession No. MI0008336, SEQ ID NO: 303) having a hairpin-like structure is known as a precursor of “hsa-miR-1915-5p”.

The term “hsa-miR-6716-5p gene” or “hsa-miR-6716-5p” used herein includes the hsa-miR-6716-5p gene (miRBase Accession No. MIMAT0025844) described in SEQ ID NO: 129, a homolog or an ortholog of a different organism species, and the like. The hsa-miR-6716-5p gene can be obtained by a method described in Li Y et al., 2012, Gene, Vol. 497, p. 330-335. Also, “hsa-mir-6716” (miRBase Accession No. MI0022550, SEQ ID NO: 327) having a hairpin-like structure is known as a precursor of “hsa-miR-6716-5p”.

The term “hsa-miR-718 gene” or “hsa-miR-718” used herein includes the hsa-miR-718 gene (miRBase Accession No. MIMAT0012735) described in SEQ ID NO: 130, a homolog or an ortholog of a different organism species, and the like. The hsa-miR-718 gene can be obtained by a method described in Artzi S et al., 2008, BMC Bioinformatics, Vol. 9, p. 39. Also, “hsa-mir-718” (miRBase Accession No. MI0012489, SEQ ID NO: 328) having a hairpin-like structure is known as a precursor of “hsa-miR-718”.

The term “hsa-miR-4281 gene” or “hsa-miR-4281” used herein includes the hsa-miR-4281 gene (miRBase Accession No. MIMAT0016907) described in SEQ ID NO: 131, a homolog or an ortholog of a different organism species, and the like. The hsa-miR-4281 gene can be obtained by a method described in Goff L A et al., 2009, PLoS One, Vol. 4, e7192. Also, “hsa-mir-4281” (miRBase Accession No. MI0015885, SEQ ID NO: 329) having a hairpin-like structure is known as a precursor of “hsa-miR-4281”.

The term “hsa-miR-6820-5p gene” or “hsa-miR-6820-5p” used herein includes the hsa-miR-6820-5p gene (miRBase Accession No. MIMAT0027540) described in SEQ ID NO: 132, a homolog or an ortholog of a different organism species, and the like. The hsa-miR-6820-5p gene can be obtained by a method described in Ladewig E et al., 2012, Genome Res, Vol. 22, p. 1634-1645. Also, “hsa-mir-6820” (miRBase Accession No. MI0022665, SEQ ID NO: 330) having a hairpin-like structure is known as a precursor of “hsa-miR-6820-5p”.

The term “hsa-miR-6795-5p gene” or “hsa-miR-6795-5p” used herein includes the hsa-miR-6795-5p gene (miRBase Accession No. MIMAT0027490) described in SEQ ID NO: 133, a homolog or an ortholog of a different organism species, and the like. The hsa-miR-6795-5p gene can be obtained by a method described in Ladewig E et al., 2012, Genome Res, Vol. 22, p. 1634-1645. Also, “hsa-mir-6795” (miRBase Accession No. MI0022640, SEQ ID NO: 331) having a hairpin-like structure is known as a precursor of “hsa-miR-6795-5p”.

The term “hsa-miR-6779-5p gene” or “hsa-miR-6779-5p” used herein includes the hsa-miR-6779-5p gene (miRBase Accession No. MIMAT0027458) described in SEQ ID NO: 134, a homolog or an ortholog of a different organism species, and the like. The hsa-miR-6779-5p gene can be obtained by a method described in Ladewig E et al., 2012, Genome Res, Vol. 22, p. 1634-1645. Also, “hsa-mir-6779” (miRBase Accession No. MI0022624, SEQ ID NO: 332) having a hairpin-like structure is known as a precursor of “hsa-miR-6779-5p”.

The term “hsa-miR-7109-5p gene” or “hsa-miR-7109-5p” used herein includes the hsa-miR-7109-5p gene (miRBase Accession No. MIMAT0028115) described in SEQ ID NO: 135, a homolog or an ortholog of a different organism species, and the like. The hsa-miR-7109-5p gene can be obtained by a method described in Ladewig E et al., 2012, Genome Res, Vol. 22, p. 1634-1645. Also, “hsa-mir-7109” (miRBase Accession No. MI0022960, SEQ ID NO: 333) having a hairpin-like structure is known as a precursor of “hsa-miR-7109-5p”.

The term “hsa-miR-6798-5p gene” or “hsa-miR-6798-5p” used herein includes the hsa-miR-6798-5p gene (miRBase Accession No. MIMAT0027496) described in SEQ ID NO: 136, a homolog or an ortholog of a different organism species, and the like. The hsa-miR-6798-5p gene can be obtained by a method described in Ladewig E et al., 2012, Genome Res, Vol. 22, p. 1634-1645. Also, “hsa-mir-6798” (miRBase Accession No. MI0022643, SEQ ID NO: 334) having a hairpin-like structure is known as a precursor of “hsa-miR-6798-5p”.

The term “hsa-miR-4648 gene” or “hsa-miR-4648” used herein includes the hsa-miR-4648 gene (miRBase Accession No. MIMAT0019710) described in SEQ ID NO: 137, a homolog or an ortholog of a different organism species, and the like. The hsa-miR-4648 gene can be obtained by a method described in Persson H et al., 2011, Cancer Res, Vol. 71, p. 78-86. Also, “hsa-mir-4648” (miRBase Accession No. MI0017275, SEQ ID NO: 335) having a hairpin-like structure is known as a precursor of “hsa-miR-4648”.

The term “hsa-miR-8059 gene” or “hsa-miR-8059” used herein includes the hsa-miR-8059 gene (miRBase Accession No. MIMAT0030986) described in SEQ ID NO: 138, a homolog or an ortholog of a different organism species, and the like. The hsa-miR-8059 gene can be obtained by a method described in Wang H J et al., 2013, Shock, Vol. 39, p. 480-487. Also, “hsa-mir-8059” (miRBase Accession No. MI0025895, SEQ ID NO: 336) having a hairpin-like structure is known as a precursor of “hsa-miR-8059”.

The term “hsa-miR-6765-3p gene” or “hsa-miR-6765-3p” used herein includes the hsa-miR-6765-3p gene (miRBase Accession No. MIMAT0027431) described in SEQ ID NO: 139, a homolog or an ortholog of a different organism species, and the like. The hsa-miR-6765-3p gene can be obtained by a method described in Ladewig E et al., 2012, Genome Res, Vol. 22, p. 1634-1645. Also, “hsa-mir-6765” (miRBase Accession No. MI0022610, SEQ ID NO: 292) having a hairpin-like structure is known as a precursor of “hsa-miR-6765-3p”.

The term “hsa-miR-6132 gene” or “hsa-miR-6132” used herein includes the hsa-miR-6132 gene (miRBase Accession No. MIMAT0024616) described in SEQ ID NO: 140, a homolog or an ortholog of a different organism species, and the like. The hsa-miR-6132 gene can be obtained by a method described in Dannemann M et al., 2012, Genome Biol Evol, Vol. 4, p. 552-564. Also, “hsa-mir-6132” (miRBase Accession No. MI0021277, SEQ ID NO: 337) having a hairpin-like structure is known as a precursor of “hsa-miR-6132”.

The term “hsa-miR-4492 gene” or “hsa-miR-4492” used herein includes the hsa-miR-4492 gene (miRBase Accession No. MIMAT0019027) described in SEQ ID NO: 141, a homolog or an ortholog of a different organism species, and the like. The hsa-miR-4492 gene can be obtained by a method described in Jima D D et al., 2010, Blood, Vol. 116, e118-e127. Also, “hsa-mir-4492” (miRBase Accession No. MI0016854, SEQ ID NO: 338) having a hairpin-like structure is known as a precursor of “hsa-miR-4492”.

The term “hsa-miR-7107-5p gene” or “hsa-miR-7107-5p” used herein includes the hsa-miR-7107-5p gene (miRBase Accession No. MIMAT0028111) described in SEQ ID NO: 142, a homolog or an ortholog of a different organism species, and the like. The hsa-miR-7107-5p gene can be obtained by a method described in Ladewig E et al., 2012, Genome Res, Vol. 22, p. 1634-1645. Also, “hsa-mir-7107” (miRBase Accession No. MI0022958, SEQ ID NO: 339) having a hairpin-like structure is known as a precursor of “hsa-miR-7107-5p”.

The term “hsa-miR-3195 gene” or “hsa-miR-3195” used herein includes the hsa-miR-3195 gene (miRBase Accession No. MIMAT0015079) described in SEQ ID NO: 143, a homolog or an ortholog of a different organism species, and the like. The hsa-miR-3195 gene can be obtained by a method described in Stark M S et al., 2010, PLoS One, Vol. 5, e9685. Also, “hsa-mir-3195” (miRBase Accession No. MI0014240, SEQ ID NO: 340) having a hairpin-like structure is known as a precursor of “hsa-miR-3195”.

The term “hsa-miR-3180 gene” or “hsa-miR-3180” used herein includes the hsa-miR-3180 gene (miRBase Accession No. MIMAT0018178) described in SEQ ID NO: 144, a homolog or an ortholog of a different organism species, and the like. The hsa-miR-3180 gene can be obtained by a method described in Creighton C J et al., 2010, PLoS One, Vol. 5, e9637. Also, “hsa-mir-3180-4 and hsa-mir-3180-5” (miRBase Accession Nos. MI0016408 and MI0016409, SEQ ID NOs: 341 and 342) having a hairpin-like structure are known as precursors of “hsa-miR-3180”.

The term “hsa-miR-296-3p gene” or “hsa-miR-296-3p” used herein includes the hsa-miR-296-3p gene (miRBase Accession No. MIMAT0004679) described in SEQ ID NO: 145, a homolog or an ortholog of a different organism species, and the like. The hsa-miR-296-3p gene can be obtained by a method described in Houbaviy H B et al., 2003, Dev Cell, Vol. 5, p. 351-358. Also, “hsa-mir-296” (miRBase Accession No. MI0000747, SEQ ID NO: 343) having a hairpin-like structure is known as a precursor of “hsa-miR-296-3p”.

The term “hsa-miR-564 gene” or “hsa-miR-564” used herein includes the hsa-miR-564 gene (miRBase Accession No. MIMAT0003228) described in SEQ ID NO: 146, a homolog or an ortholog of a different organism species, and the like. The hsa-miR-564 gene can be obtained by a method described in Cummins J M et al., 2006, Proc Natl Acad Sci USA, Vol. 103, p. 3687-3692. Also, “hsa-mir-564” (miRBase Accession No. MI0003570, SEQ ID NO: 344) having a hairpin-like structure is known as a precursor of “hsa-miR-564”.

The term “hsa-miR-1268a gene” or “hsa-miR-1268a” used herein includes the hsa-miR-1268a gene (miRBase Accession No. MIMAT0005922) described in SEQ ID NO: 147, a homolog or an ortholog of a different organism species, and the like. The hsa-miR-1268a gene can be obtained by a method described in Morin R D et al., 2008, Genome Res, Vol. 18, p. 610-621. Also, “hsa-mir-1268a” (miRBase Accession No. MI0006405, SEQ ID NO: 345) having a hairpin-like structure is known as a precursor of “hsa-miR-1268a”.

The term “hsa-miR-6848-5p gene” or “hsa-miR-6848-5p” used herein includes the hsa-miR-6848-5p gene (miRBase Accession No. MIMAT0027596) described in SEQ ID NO: 148, a homolog or an ortholog of a different organism species, and the like. The hsa-miR-6848-5p gene can be obtained by a method described in Ladewig E et al., 2012, Genome Res, Vol. 22, p. 1634-1645. Also, “hsa-mir-6848” (miRBase Accession No. MI0022694, SEQ ID NO: 346) having a hairpin-like structure is known as a precursor of “hsa-miR-6848-5p”.

The term “hsa-miR-762 gene” or “hsa-miR-762” used herein includes the hsa-miR-762 gene (miRBase Accession No. MIMAT0010313) described in SEQ ID NO: 149, a homolog or an ortholog of a different organism species, and the like. The hsa-miR-762 gene can be obtained by a method described in Berezikov E et al., 2006, Genome Res, Vol. 16, p. 1289-1298. Also, “hsa-mir-762” (miRBase Accession No. MI0003892, SEQ ID NO: 347) having a hairpin-like structure is known as a precursor of “hsa-miR-762”.

The term “hsa-miR-2861 gene” or “hsa-miR-2861” used herein includes the hsa-miR-2861 gene (miRBase Accession No. MIMAT0013802) described in SEQ ID NO: 150, a homolog or an ortholog of a different organism species, and the like. The hsa-miR-2861 gene can be obtained by a method described in Li H et al., 2009, J Clin Invest, Vol. 119, p. 3666-3677. Also, “hsa-mir-2861” (miRBase Accession No. MI0013006, SEQ ID NO: 348) having a hairpin-like structure is known as a precursor of “hsa-miR-2861”.

The term “hsa-miR-1203 gene” or “hsa-miR-1203” used herein includes the hsa-miR-1203 gene (miRBase Accession No. MIMAT0005866) described in SEQ ID NO: 151, a homolog or an ortholog of a different organism species, and the like. The hsa-miR-1203 gene can be obtained by a method described in Marton S et al., 2008, Leukemia, Vol. 22, p. 330-338. Also, “hsa-mir-1203” (miRBase Accession No. MI0006335, SEQ ID NO: 349) having a hairpin-like structure is known as a precursor of “hsa-miR-1203”.

The term “hsa-miR-1260b gene” or “hsa-miR-1260b” used herein includes the hsa-miR-1260b gene (miRBase Accession No. MIMAT0015041) described in SEQ ID NO: 152, a homolog or an ortholog of a different organism species, and the like. The hsa-miR-1260b gene can be obtained by a method described in Stark M S et al., 2010, PLoS One, Vol. 5, e9685. Also, “hsa-mir-1260b” (miRBase Accession No. MI0014197, SEQ ID NO: 350) having a hairpin-like structure is known as a precursor of “hsa-miR-1260b”.

The term “hsa-miR-4476 gene” or “hsa-miR-4476” used herein includes the hsa-miR-4476 gene (miRBase Accession No. MIMAT0019003) described in SEQ ID NO: 153, a homolog or an ortholog of a different organism species, and the like. The hsa-miR-4476 gene can be obtained by a method described in Jima D D et al., 2010, Blood, Vol. 116, e118-e127. Also, “hsa-mir-4476” (miRBase Accession No. MI0016828, SEQ ID NO: 351) having a hairpin-like structure is known as a precursor of “hsa-miR-4476”.

The term “hsa-miR-6885-5p gene” or “hsa-miR-6885-5p” used herein includes the hsa-miR-6885-5p gene (miRBase Accession No. MIMAT0027670) described in SEQ ID NO: 154, a homolog or an ortholog of a different organism species, and the like. The hsa-miR-6885-5p gene can be obtained by a method described in Ladewig E et al., 2012, Genome Res, Vol. 22, p. 1634-1645. Also, “hsa-mir-6885” (miRBase Accession No. MI0022732, SEQ ID NO: 352) having a hairpin-like structure is known as a precursor of “hsa-miR-6885-5p”.

The term “hsa-miR-6769b-5p gene” or “hsa-miR-6769b-5p” used herein includes the hsa-miR-6769b-5p gene (miRBase Accession No. MIMAT0027620) described in SEQ ID NO: 155, a homolog or an ortholog of a different organism species, and the like. The hsa-miR-6769b-5p gene can be obtained by a method described in Ladewig E et al., 2012, Genome Res, Vol. 22, p. 1634-1645. Also, “hsa-mir-6769b” (miRBase Accession No. MI0022706, SEQ ID NO: 353) having a hairpin-like structure is known as a precursor of “hsa-miR-6769b-5p”.

The term “hsa-miR-23b-3p gene” or “hsa-miR-23b-3p” used herein includes the hsa-miR-23b-3p gene (miRBase Accession No. MIMAT0000418) described in SEQ ID NO: 156, a homolog or an ortholog of a different organism species, and the like. The hsa-miR-23b-3p gene can be obtained by a method described in Lagos-Quintana M et al., 2002, Curr Biol, Vol. 12, p. 735-739. Also, “hsa-mir-23b” (miRBase Accession No. MI0000439, SEQ ID NO: 354) having a hairpin-like structure is known as a precursor of “hsa-miR-23b-3p”.

The term “hsa-miR-1343-5p gene” or “hsa-miR-1343-5p” used herein includes the hsa-miR-1343-5p gene (miRBase Accession No. MIMAT0027038) described in SEQ ID NO: 157, a homolog or an ortholog of a different organism species, and the like. The hsa-miR-1343-5p gene can be obtained by a method described in Persson H et al., 2011, Cancer Res, Vol. 71, p. 78-86. Also, “hsa-mir-1343” (miRBase Accession No. MI0017320, SEQ ID NO: 202) having a hairpin-like structure is known as a precursor of “hsa-miR-1343-5p”.

The term “hsa-miR-3621 gene” or “hsa-miR-3621” used herein includes the hsa-miR-3621 gene (miRBase Accession No. MIMAT0018002) described in SEQ ID NO: 158, a homolog or an ortholog of a different organism species, and the like. The hsa-miR-3621 gene can be obtained by a method described in Witten D et al., 2010, BMC Biol, Vol. 8, p. 58. Also, “hsa-mir-3621” (miRBase Accession No. MI0016012, SEQ ID NO: 355) having a hairpin-like structure is known as a precursor of “hsa-miR-3621”.

The term “hsa-miR-4688 gene” or “hsa-miR-4688” used herein includes the hsa-miR-4688 gene (miRBase Accession No. MIMAT0019777) described in SEQ ID NO: 159, a homolog or an ortholog of a different organism species, and the like. The hsa-miR-4688 gene can be obtained by a method described in Persson H et al., 2011, Cancer Res, Vol. 71, p. 78-86. Also, “hsa-mir-4688” (miRBase Accession No. MI0017321, SEQ ID NO: 356) having a hairpin-like structure is known as a precursor of “hsa-miR-4688”.

The term “hsa-miR-4286 gene” or “hsa-miR-4286” used herein includes the hsa-miR-4286 gene (miRBase Accession No. MIMAT0016916) described in SEQ ID NO: 160, a homolog or an ortholog of a different organism species, and the like. The hsa-miR-4286 gene can be obtained by a method described in Goff L A et al., 2009, PLoS One, Vol. 4, e7192. Also, “hsa-mir-4286” (miRBase Accession No. MI0015894, SEQ ID NO: 357) having a hairpin-like structure is known as a precursor of “hsa-miR-4286”.

The term “hsa-miR-4640-5p gene” or “hsa-miR-4640-5p” used herein includes the hsa-miR-4640-5p gene (miRBase Accession No. MIMAT0019699) described in SEQ ID NO: 161, a homolog or an ortholog of a different organism species, and the like. The hsa-miR-4640-5p gene can be obtained by a method described in Persson H et al., 2011, Cancer Res, Vol. 71, p. 78-86. Also, “hsa-mir-4640” (miRBase Accession No. MI0017267, SEQ ID NO: 358) having a hairpin-like structure is known as a precursor of “hsa-miR-4640-5p”.

The term “hsa-miR-4739 gene” or “hsa-miR-4739” used herein includes the hsa-miR-4739 gene (miRBase Accession No. MIMAT0019868) described in SEQ ID NO: 162, a homolog or an ortholog of a different organism species, and the like. The hsa-miR-4739 gene can be obtained by a method described in Persson H et al., 2011, Cancer Res, Vol. 71, p. 78-86. Also, “hsa-mir-4739” (miRBase Accession No. MI0017377, SEQ ID NO: 359) having a hairpin-like structure is known as a precursor of “hsa-miR-4739”.

The term “hsa-miR-1260a gene” or “hsa-miR-1260a” used herein includes the hsa-miR-1260a gene (miRBase Accession No. MIMAT0005911) described in SEQ ID NO: 163, a homolog or an ortholog of a different organism species, and the like. The hsa-miR-1260a gene can be obtained by a method described in Morin R D et al., 2008, Genome Res, Vol. 18, p. 610-621. Also, “hsa-mir-1260a” (miRBase Accession No. MI0006394, SEQ ID NO: 360) having a hairpin-like structure is known as a precursor of “hsa-miR-1260a”.

The term “hsa-miR-4276 gene” or “hsa-miR-4276” used herein includes the hsa-miR-4276 gene (miRBase Accession No. MIMAT0016904) described in SEQ ID NO: 164, a homolog or an ortholog of a different organism species, and the like. The hsa-miR-4276 gene can be obtained by a method described in Goff L A et al., 2009, PLoS One, Vol. 4, e7192. Also, “hsa-mir-4276” (miRBase Accession No. MI0015882, SEQ ID NO: 361) having a hairpin-like structure is known as a precursor of “hsa-miR-4276”.

The term “hsa-miR-7106-5p gene” or “hsa-miR-7106-5p” used herein includes the hsa-miR-7106-5p gene (miRBase Accession No. MIMAT0028109) described in SEQ ID NO: 165, a homolog or an ortholog of a different organism species, and the like. The hsa-miR-7106-5p gene can be obtained by a method described in Ladewig E et al., 2012, Genome Res, Vol. 22, p. 1634-1645. Also, “hsa-mir-7106” (miRBase Accession No. MI0022957, SEQ ID NO: 362) having a hairpin-like structure is known as a precursor of “hsa-miR-7106-5p”.

The term “hsa-miR-128-2-5p gene” or “hsa-miR-128-2-5p” used herein includes the hsa-miR-128-2-5p gene (miRBase Accession No. MIMAT0031095) described in SEQ ID NO: 166, a homolog or an ortholog of a different organism species, and the like. The hsa-miR-128-2-5p gene can be obtained by a method described in Lagos-Quintana M et al., 2002, Curr Biol, Vol. 12, p. 735-739. Also, “hsa-mir-128-2” (miRBase Accession No. MI0000727, SEQ ID NO: 363) having a hairpin-like structure is known as a precursor of “hsa-miR-128-2-5p”.

The term “hsa-miR-125a-3p gene” or “hsa-miR-125a-3p” used herein includes the hsa-miR-125a-3p gene (miRBase Accession No. MIMAT0004602) described in SEQ ID NO: 167, a homolog or an ortholog of a different organism species, and the like. The hsa-miR-125a-3p gene can be obtained by a method described in Lagos-Quintana M et al., 2002, Curr Biol, Vol. 12, p. 735-739. Also, “hsa-mir-125a” (miRBase Accession No. MI0000469, SEQ ID NO: 364) having a hairpin-like structure is known as a precursor of “hsa-miR-125a-3p”.

The term “hsa-miR-92a-2-5p gene” or “hsa-miR-92a-2-5p” used herein includes the hsa-miR-92a-2-5p gene (miRBase Accession No. MIMAT0004508) described in SEQ ID NO: 168, a homolog or an ortholog of a different organism species, and the like. The hsa-miR-92a-2-5p gene can be obtained by a method described in Mourelatos Z et al., 2002, Genes Dev, Vol. 16, p. 720-728. Also, “hsa-mir-92a-2” (miRBase Accession No. MI0000094, SEQ ID NO: 365) having a hairpin-like structure is known as a precursor of “hsa-miR-92a-2-5p”.

The term “hsa-miR-486-3p gene” or “hsa-miR-486-3p” used herein includes the hsa-miR-486-3p gene (miRBase Accession No. MIMAT0004762) described in SEQ ID NO: 169, a homolog or an ortholog of a different organism species, and the like. The hsa-miR-486-3p gene can be obtained by a method described in Fu H et al., 2005, FEBS Lett, Vol. 579, p. 3849-3854. Also, “hsa-mir-486 and hsa-mir-486-2” (miRBase Accession Nos. MI0002470 and MI0023622, SEQ ID NOs: 366 and 367) having a hairpin-like structure are known as precursors of “hsa-miR-486-3p”.

The term “hsa-miR-3196 gene” or “hsa-miR-3196” used herein includes the hsa-miR-3196 gene (miRBase Accession No. MIMAT0015080) described in SEQ ID NO: 170, a homolog or an ortholog of a different organism species, and the like. The hsa-miR-3196 gene can be obtained by a method described in Stark M S et al., 2010, PLoS One, Vol. 5, e9685. Also, “hsa-mir-3196” (miRBase Accession No. MI0014241, SEQ ID NO: 368) having a hairpin-like structure is known as a precursor of “hsa-miR-3196”.

The term “hsa-miR-211-3p gene” or “hsa-miR-211-3p” used herein includes the hsa-miR-211-3p gene (miRBase Accession No. MIMAT0022694) described in SEQ ID NO: 171, a homolog or an ortholog of a different organism species, and the like. The hsa-miR-211-3p gene can be obtained by a method described in Lim L P et al., 2003, Science, Vol. 299, p. 1540. Also, “hsa-mir-211” (miRBase Accession No. MI0000287, SEQ ID NO: 369) having a hairpin-like structure is known as a precursor of “hsa-miR-211-3p”.

The term “hsa-miR-4271 gene” or “hsa-miR-4271” used herein includes the hsa-miR-4271 gene (miRBase Accession No. MIMAT0016901) described in SEQ ID NO: 172, a homolog or an ortholog of a different organism species, and the like. The hsa-miR-4271 gene can be obtained by a method described in Goff L A et al., 2009, PLoS One, Vol. 4, e7192. Also, “hsa-mir-4271” (miRBase Accession No. MI0015879, SEQ ID NO: 370) having a hairpin-like structure is known as a precursor of “hsa-miR-4271”.

The term “hsa-miR-6851-5p gene” or “hsa-miR-6851-5p” used herein includes the hsa-miR-6851-5p gene (miRBase Accession No. MIMAT0027602) described in SEQ ID NO: 173, a homolog or an ortholog of a different organism species, and the like. The hsa-miR-6851-5p gene can be obtained by a method described in Ladewig E et al., 2012, Genome Res, Vol. 22, p. 1634-1645. Also, “hsa-mir-6851” (miRBase Accession No. MI0022697, SEQ ID NO: 371) having a hairpin-like structure is known as a precursor of “hsa-miR-6851-5p”.

The term “hsa-miR-149-3p gene” or “hsa-miR-149-3p” used herein includes the hsa-miR-149-3p gene (miRBase Accession No. MIMAT0004609) described in SEQ ID NO: 174, a homolog or an ortholog of a different organism species, and the like. The hsa-miR-149-3p gene can be obtained by a method described in Lagos-Quintana M et al., 2002, Curr Biol, Vol. 12, p. 735-739. Also, “hsa-mir-149” (miRBase Accession No. MI0000478, SEQ ID NO: 372) having a hairpin-like structure is known as a precursor of “hsa-miR-149-3p”.

The term “hsa-miR-4667-5p gene” or “hsa-miR-4667-5p” used herein includes the hsa-miR-4667-5p gene (miRBase Accession No. MIMAT0019743) described in SEQ ID NO: 175, a homolog or an ortholog of a different organism species, and the like. The hsa-miR-4667-5p gene can be obtained by a method described in Persson H et al., 2011, Cancer Res, Vol. 71, p. 78-86. Also, “hsa-mir-4667” (miRBase Accession No. MI0017297, SEQ ID NO: 373) having a hairpin-like structure is known as a precursor of “hsa-miR-4667-5p”.

The term “hsa-miR-135a-3p gene” or “hsa-miR-135a-3p” used herein includes the hsa-miR-135a-3p gene (miRBase Accession No. MIMAT0004595) described in SEQ ID NO: 176, a homolog or an ortholog of a different organism species, and the like. The hsa-miR-135a-3p gene can be obtained by a method described in Lagos-Quintana M et al., 2002, Curr Biol, Vol. 12, p. 735-739. Also, “hsa-mir-135a-1” (miRBase Accession No. MI0000452, SEQ ID NO: 374) having a hairpin-like structure is known as a precursor of “hsa-miR-135a-3p”.

The term “hsa-miR-4486 gene” or “hsa-miR-4486” used herein includes the hsa-miR-4486 gene (miRBase Accession No. MIMAT0019020) described in SEQ ID NO: 177, a homolog or an ortholog of a different organism species, and the like. The hsa-miR-4486 gene can be obtained by a method described in Jima D D et al., 2010, Blood, Vol. 116, e118-e127. Also, “hsa-mir-4486” (miRBase Accession No. MI0016847, SEQ ID NO: 375) having a hairpin-like structure is known as a precursor of “hsa-miR-4486”.

The term “hsa-miR-4697-5p gene” or “hsa-miR-4697-5p” used herein includes the hsa-miR-4697-5p gene (miRBase Accession No. MIMAT0019791) described in SEQ ID NO: 178, a homolog or an ortholog of a different organism species, and the like. The hsa-miR-4697-5p gene can be obtained by a method described in Persson H et al., 2011, Cancer Res, Vol. 71, p. 78-86. Also, “hsa-mir-4697” (miRBase Accession No. MI0017330, SEQ ID NO: 376) having a hairpin-like structure is known as a precursor of “hsa-miR-4697-5p”.

The term “hsa-miR-4725-3p gene” or “hsa-miR-4725-3p” used herein includes the hsa-miR-4725-3p gene (miRBase Accession No. MIMAT0019844) described in SEQ ID NO: 179, a homolog or an ortholog of a different organism species, and the like. The hsa-miR-4725-3p gene can be obtained by a method described in Persson H et al., 2011, Cancer Res, Vol. 71, p. 78-86. Also, “hsa-mir-4725” (miRBase Accession No. MI0017362, SEQ ID NO: 377) having a hairpin-like structure is known as a precursor of “hsa-miR-4725-3p”.

The term “hsa-miR-6510-5p gene” or “hsa-miR-6510-5p” used herein includes the hsa-miR-6510-5p gene (miRBase Accession No. MIMAT0025476) described in SEQ ID NO: 180, a homolog or an ortholog of a different organism species, and the like. The hsa-miR-6510-5p gene can be obtained by a method described in Joyce C E et al., 2011, Hum Mol Genet, Vol. 20, p. 4025-4040. Also, “hsa-mir-6510” (miRBase Accession No. MI0022222, SEQ ID NO: 378) having a hairpin-like structure is known as a precursor of “hsa-miR-6510-5p”.

The term “hsa-miR-5001-5p gene” or “hsa-miR-5001-5p” used herein includes the hsa-miR-5001-5p gene (miRBase Accession No. MIMAT0021021) described in SEQ ID NO: 181, a homolog or an ortholog of a different organism species, and the like. The hsa-miR-5001-5p gene can be obtained by a method described in Hansen T B et al., 2011, RNA Biol, Vol. 8, p. 378-383. Also, “hsa-mir-5001” (miRBase Accession No. MI0017867, SEQ ID NO: 379) having a hairpin-like structure is known as a precursor of “hsa-miR-5001-5p”.

The term “hsa-miR-4673 gene” or “hsa-miR-4673” used herein includes the hsa-miR-4673 gene (miRBase Accession No. MIMAT0019755) described in SEQ ID NO: 182, a homolog or an ortholog of a different organism species, and the like. The hsa-miR-4673 gene can be obtained by a method described in Persson H et al., 2011, Cancer Res, Vol. 71, p. 78-86. Also, “hsa-mir-4673” (miRBase Accession No. MI0017304, SEQ ID NO: 380) having a hairpin-like structure is known as a precursor of “hsa-miR-4673”.

The term “hsa-miR-4466 gene” or “hsa-miR-4466” used herein includes the hsa-miR-4466 gene (miRBase Accession No. MIMAT0018993) described in SEQ ID NO: 183, a homolog or an ortholog of a different organism species, and the like. The hsa-miR-4466 gene can be obtained by a method described in Jima D D et al., 2010, Blood, Vol. 116, e118-e127. Also, “hsa-mir-4466” (miRBase Accession No. MI0016817, SEQ ID NO: 381) having a hairpin-like structure is known as a precursor of “hsa-miR-4466”.

The term “hsa-miR-23a-3p gene” or “hsa-miR-23a-3p” used herein includes the hsa-miR-23a-3p gene (miRBase Accession No. MIMAT0000078) described in SEQ ID NO: 184, a homolog or an ortholog of a different organism species, and the like. The hsa-miR-23a-3p gene can be obtained by a method described in Lagos-Quintana M et al., 2001, Science, Vol. 294, p. 853-858. Also, “hsa-mir-23a” (miRBase Accession No. MI0000079, SEQ ID NO: 382) having a hairpin-like structure is known as a precursor of “hsa-miR-23a-3p”.

The term “hsa-miR-3656 gene” or “hsa-miR-3656” used herein includes the hsa-miR-3656 gene (miRBase Accession No. MIMAT0018076) described in SEQ ID NO: 185, a homolog or an ortholog of a different organism species, and the like. The hsa-miR-3656 gene can be obtained by a method described in Meiri E et al., 2010, Nucleic Acids Res, Vol. 38, p. 6234-6246. Also, “hsa-mir-3656” (miRBase Accession No. MI0016056, SEQ ID NO: 383) having a hairpin-like structure is known as a precursor of “hsa-miR-3656”.

The term “hsa-miR-6782-5p gene” or “hsa-miR-6782-5p” used herein includes the hsa-miR-6782-5p gene (miRBase Accession No. MIMAT0027464) described in SEQ ID NO: 186, a homolog or an ortholog of a different organism species, and the like. The hsa-miR-6782-5p gene can be obtained by a method described in Ladewig E et al., 2012, Genome Res, Vol. 22, p. 1634-1645. Also, “hsa-mir-6782” (miRBase Accession No. MI0022627, SEQ ID NO: 384) having a hairpin-like structure is known as a precursor of “hsa-miR-6782-5p”.

The term “hsa-miR-4689 gene” or “hsa-miR-4689” used herein includes the hsa-miR-4689 gene (miRBase Accession No. MIMAT0019778) described in SEQ ID NO: 187, a homolog or an ortholog of a different organism species, and the like. The hsa-miR-4689 gene can be obtained by a method described in Persson H et al., 2011, Cancer Res, Vol. 71, p. 78-86. Also, “hsa-mir-4689” (miRBase Accession No. MI0017322, SEQ ID NO: 385) having a hairpin-like structure is known as a precursor of “hsa-miR-4689”.

The term “hsa-miR-451a gene” or “hsa-miR-451a” used herein includes the hsa-miR-451a gene (miRBase Accession No. MIMAT0001631) described in SEQ ID NO: 188, a homolog or an ortholog of a different organism species, and the like. The hsa-miR-451a gene can be obtained by a method described in Altuvia Y et al., 2005, Nucleic Acids Res, Vol. 33, p. 2697-2706. Also, “hsa-mir-451a” (miRBase Accession No. MI0001729, SEQ ID NO: 386) having a hairpin-like structure is known as a precursor of “hsa-miR-451a”.

The term “hsa-miR-4446-3p gene” or “hsa-miR-4446-3p” used herein includes the hsa-miR-4446-3p gene (miRBase Accession No. MIMAT0018965) described in SEQ ID NO: 189, a homolog or an ortholog of a different organism species, and the like. The hsa-miR-4446-3p gene can be obtained by a method described in Jima D D et al., 2010, Blood, Vol. 116, e118-e127. Also, “hsa-mir-4446” (miRBase Accession No. MI0016789, SEQ ID NO: 387) having a hairpin-like structure is known as a precursor of “hsa-miR-4446-3p”.

The term “hsa-miR-3180-3p gene” or “hsa-miR-3180-3p” used herein includes the hsa-miR-3180-3p gene (miRBase Accession No. MIMAT0015058) described in SEQ ID NO: 190, a homolog or an ortholog of a different organism species, and the like. The hsa-miR-3180-3p gene can be obtained by a method described in Creighton C J et al., 2010, PLoS One, Vol. 5, e9637. Also, “hsa-mir-3180-1, hsa-mir-3180-2, and hsa-mir-3180-3” (miRBase Accession Nos. MI0014214, MI0014215, and MI0014217, SEQ ID NOs: 388, 389, and 390) having a hairpin-like structure are known as precursors of “hsa-miR-3180-3p”.

The term “hsa-miR-642a-3p gene” or “hsa-miR-642a-3p” used herein includes the hsa-miR-642a-3p gene (miRBase Accession No. MIMAT0020924) described in SEQ ID NO: 191, a homolog or an ortholog of a different organism species, and the like. The hsa-miR-642a-3p gene can be obtained by a method described in Cummins J M et al., 2006, Proc Natl Acad Sci USA, Vol. 103, p. 3687-3692. Also, “hsa-mir-642a” (miRBase Accession No. MI0003657, SEQ ID NO: 391) having a hairpin-like structure is known as a precursor of “hsa-miR-642a-3p”.

The term “hsa-miR-6889-5p gene” or “hsa-miR-6889-5p” used herein includes the hsa-miR-6889-5p gene (miRBase Accession No. MIMAT0027678) described in SEQ ID NO: 192, a homolog or an ortholog of a different organism species, and the like. The hsa-miR-6889-5p gene can be obtained by a method described in Ladewig E et al., 2012, Genome Res, Vol. 22, p. 1634-1645. Also, “hsa-mir-6889” (miRBase Accession No. MI0022736, SEQ ID NO: 392) having a hairpin-like structure is known as a precursor of “hsa-miR-6889-5p”.

The term “hsa-miR-3178 gene” or “hsa-miR-3178” used herein includes the hsa-miR-3178 gene (miRBase Accession No. MIMAT0015055) described in SEQ ID NO: 193, a homolog or an ortholog of a different organism species, and the like. The hsa-miR-3178 gene can be obtained by a method described in Stark M S et al., 2010, PLoS One, Vol. 5, e9685. Also, “hsa-mir-3178” (miRBase Accession No. MI0014212, SEQ ID NO: 393) having a hairpin-like structure is known as a precursor of “hsa-miR-3178”.

The term “hsa-miR-4665-5p gene” or “hsa-miR-4665-5p” used herein includes the hsa-miR-4665-5p gene (miRBase Accession No. MIMAT0019739) described in SEQ ID NO: 194, a homolog or an ortholog of a different organism species, and the like. The hsa-miR-4665-5p gene can be obtained by a method described in Persson H et al., 2011, Cancer Res, Vol. 71, p. 78-86. Also, “hsa-mir-4665” (miRBase Accession No. MI0017295, SEQ ID NO: 323) having a hairpin-like structure is known as a precursor of “hsa-miR-4665-5p”.

The term “hsa-miR-6722-3p gene” or “hsa-miR-6722-3p” used herein includes the hsa-miR-6722-3p gene (miRBase Accession No. MIMAT0025854) described in SEQ ID NO: 195, a homolog or an ortholog of a different organism species, and the like. The hsa-miR-6722-3p gene can be obtained by a method described in Li Y et al., 2012, Gene, Vol. 497, p. 330-335. Also, “hsa-mir-6722” (miRBase Accession No. MI0022557, SEQ ID NO: 394) having a hairpin-like structure is known as a precursor of “hsa-miR-6722-3p”.

The term “hsa-miR-30c-1-3p gene” or “hsa-miR-30c-1-3p” used herein includes the hsa-miR-30c-1-3p gene (miRBase Accession No. MIMAT0004674) described in SEQ ID NO: 196, a homolog or an ortholog of a different organism species, and the like. The hsa-miR-30c-1-3p gene can be obtained by a method described in Lagos-Quintana M et al., 2002, Curr Biol, Vol. 12, p. 735-739. Also, “hsa-mir-30c-1” (miRBase Accession No. MI0000736, SEQ ID NO: 395) having a hairpin-like structure is known as a precursor of “hsa-miR-30c-1-3p”.

The term “hsa-miR-4507 gene” or “hsa-miR-4507” used herein includes the hsa-miR-4507 gene (miRBase Accession No. MIMAT0019044) described in SEQ ID NO: 197, a homolog or an ortholog of a different organism species, and the like. The hsa-miR-4507 gene can be obtained by a method described in Jima D D et al., 2010, Blood, Vol. 116, e118-e127. Also, “hsa-mir-4507” (miRBase Accession No. MI0016871, SEQ ID NO: 396) having a hairpin-like structure is known as a precursor of “hsa-miR-4507”.

The term “hsa-miR-3141 gene” or “hsa-miR-3141” used herein includes the hsa-miR-3141 gene (miRBase Accession No. MIMAT0015010) described in SEQ ID NO: 198, a homolog or an ortholog of a different organism species, and the like. The hsa-miR-3141 gene can be obtained by a method described in Stark M S et al., 2010, PLoS One, Vol. 5, e9685. Also, “hsa-mir-3141” (miRBase Accession No. MI0014165, SEQ ID NO: 397) having a hairpin-like structure is known as a precursor of “hsa-miR-3141”.

The term “hsa-miR-1199-5p gene” or “hsa-miR-1199-5p” used herein includes the hsa-miR-1199-5p gene (miRBase Accession No. MIMAT0031119) described in SEQ ID NO: 199, a homolog or an ortholog of a different organism species, and the like. The hsa-miR-1199-5p gene can be obtained by a method described in Salvi A et al., 2013, Int J Oncol, Vol. 42, p. 391-402. Also, “hsa-mir-1199” (miRBase Accession No. MI0020340, SEQ ID NO: 398) having a hairpin-like structure is known as a precursor of “hsa-miR-1199-5p”.

The term “hsa-miR-6794-5p gene” or “hsa-miR-6794-5p” used herein includes the hsa-miR-6794-5p gene (miRBase Accession No. MIMAT0027488) described in SEQ ID NO: 635, a homolog or an ortholog of a different organism species, and the like. The hsa-miR-6794-5p gene can be obtained by a method described in Ladewig E et al., 2012, Genome Res, Vol. 22, p. 1634-1645. Also, “hsa-mir-6794” (miRBase Accession No. MI0022639, SEQ ID NO: 643) having a hairpin-like structure is known as a precursor of “hsa-miR-6794-5p”.

The term “hsa-miR-6774-5p gene” or “hsa-miR-6774-5p” used herein includes the hsa-miR-6774-5p gene (miRBase Accession No. MIMAT0027448) described in SEQ ID NO: 636, a homolog or an ortholog of a different organism species, and the like. The hsa-miR-6774-5p gene can be obtained by a method described in Ladewig E et al., 2012, Genome Res, Vol. 22, p. 1634-1645. Also, “hsa-mir-6774” (miRBase Accession No. MI0022619, SEQ ID NO: 644) having a hairpin-like structure is known as a precursor of “hsa-miR-6774-5p”.

The term “hsa-miR-4707-3p gene” or “hsa-miR-4707-3p” used herein includes the hsa-miR-4707-3p gene (miRBase Accession No. MIMAT0019808) described in SEQ ID NO: 637, a homolog or an ortholog of a different organism species, and the like. The hsa-miR-4707-3p gene can be obtained by a method described in Persson H et al., 2011, Cancer Res, Vol. 71, p. 78-86. Also, “hsa-mir-4707” (miRBase Accession No. MI0017340, SEQ ID NO: 645) having a hairpin-like structure is known as a precursor of “hsa-miR-4707-3p”.

The term “hsa-miR-4534 gene” or “hsa-miR-4534” used herein includes the hsa-miR-4534 gene (miRBase Accession No. MIMAT0019073) described in SEQ ID NO: 638, a homolog or an ortholog of a different organism species, and the like. The hsa-miR-4534 gene can be obtained by a method described in Jima D D et al., 2010, Blood., Vol. 116, e118-e127. Also, “hsa-mir-4534” (miRBase Accession No. MI0016901, SEQ ID NO: 646) having a hairpin-like structure is known as a precursor of “hsa-miR-4534”.

The term “hsa-miR-4294 gene” or “hsa-miR-4294” used herein includes the hsa-miR-4294 gene (miRBase Accession No. MIMAT0016849) described in SEQ ID NO: 639, a homolog or an ortholog of a different organism species, and the like. The hsa-miR-4294 gene can be obtained by a method described in Goff L A et al., 2009, PLoS One., Vol. 4, e7192. Also, “hsa-mir-4294” (miRBase Accession No. MI0015827, SEQ ID NO: 647) having a hairpin-like structure is known as a precursor of “hsa-miR-4294”.

The term “hsa-miR-6850-5p gene” or “hsa-miR-6850-5p” used herein includes the hsa-miR-6850-5p gene (miRBase Accession No. MIMAT0027600) described in SEQ ID NO: 640, a homolog or an ortholog of a different organism species, and the like. The hsa-miR-6850-5p gene can be obtained by a method described in Ladewig E et al., 2012, Genome Res., Vol. 22, p. 1634-1645. Also, “hsa-mir-6850” (miRBase Accession No. MI0022696, SEQ ID NO: 648) having a hairpin-like structure is known as a precursor of “hsa-miR-6850-5p”.

The term “hsa-miR-6089 gene” or “hsa-miR-6089” used herein includes the hsa-miR-6089 gene (miRBase Accession No. MIMAT0023714) described in SEQ ID NO: 641, a homolog or an ortholog of a different organism species, and the like. The hsa-miR-6089 gene can be obtained by a method described in Yoo J K et al., 2012, Stem Cells Dev, Vol. 21, p. 2049-2057. Also, “hsa-mir-6089-1 and hsa-mir-6089-2” (miRBase Accession Nos. MI0020366 and MI0023563, SEQ ID NOs: 649 and 650) having a hairpin-like structure are known as precursors of “hsa-miR-6089”.

The term “hsa-miR-671-5p gene” or “hsa-miR-671-5p” used herein includes the hsa-miR-671-5p gene (miRBase Accession No. MIMAT0003880) described in SEQ ID NO: 642, a homolog or an ortholog of a different organism species, and the like. The hsa-miR-671-5p gene can be obtained by a method described in Berezikov E et al., 2006, Genome Res, Vol. 16, p. 1289-1298. Also, “hsa-mir-671” (miRBase Accession No. MI0003760, SEQ ID NO: 651) having a hairpin-like structure is known as a precursor of “hsa-miR-671-5p”.

A mature miRNA may become a variant due to the sequence cleaved shorter or longer by one to several flanking nucleotides or due to substitution of nucleotides when cleaved as the mature miRNA from its RNA precursor having a hairpin-like structure. This variant is called isomiR (Morin R D. et al., 2008, Genome Res., Vol. 18, p. 610-621). The miRBase Release 20 shows the nucleotide sequences represented by SEQ ID NOs: 1 to 199 and 635 to 642 as well as a large number of the nucleotide sequence variants and fragments represented by SEQ ID NOs: 399 to 634 and 652 to 657, called isomiRs. These variants can also be obtained as miRNAs having a nucleotide sequence represented by any of SEQ ID NOs: 1 to 199 and 635 to 642.

Specifically, among the variants of polynucleotides consisting of the nucleotide sequence represented by any of SEQ ID NOs: 3, 4, 11, 13, 14, 18, 20, 21, 26, 29, 35, 36, 39, 41, 42, 45, 46, 47, 48, 49, 51, 53, 54, 55, 56, 57, 58, 59, 60, 62, 65, 66, 67, 68, 71, 72, 73, 74, 75, 76, 78, 82, 83, 88, 90, 91, 92, 94, 95, 98, 99, 101, 102, 103, 104, 106, 107, 108, 109, 110, 113, 116, 117, 118, 120, 121, 122, 123, 125, 128, 129, 130, 131, 137, 140, 141, 143, 144, 145, 146, 147, 150, 152, 153, 156, 159, 160, 161, 162, 163, 166, 167, 168, 169, 170, 171, 172, 174, 175, 176, 177, 179, 180, 181, 182, 183,184, 185, 187, 188, 189, 190, 191, 193, 194, 196, 197, 198, 637, 641 and 642 or a nucleotide sequence derived from the nucleotide sequence by the replacement of u with t according to the present invention, examples of the longest variants registered in the miRBase Release 20 include polynucleotides represented by SEQ ID NOs: 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 652, 654 and 656, respectively.

Also, among the variants of polynucleotides consisting of a nucleotide sequence represented by any of SEQ ID NOs: 3, 4, 11, 13, 14, 18, 20, 21, 26, 29, 35, 36, 39, 41, 42, 45, 46, 47, 48, 49, 51, 53, 54, 55, 56, 57, 58, 59, 60, 62, 65, 66, 67, 68, 71, 72, 73, 74, 75, 76, 78, 82, 83, 88, 90, 91, 92, 94, 95, 98, 99, 101, 102, 103, 104, 106, 107, 108, 109, 110, 113, 116, 117, 118, 120, 121, 122, 123, 125, 128, 129, 130, 131, 137, 140, 141, 143, 144, 145, 146, 147, 150, 152, 153, 156, 159, 160, 161, 162, 163, 166, 167, 168, 169, 170, 171, 172, 174, 175, 176, 177, 179, 180, 181, 182, 183,184, 185, 187, 188, 189, 190, 191, 193, 194, 196, 197, 198, 637, 641 and 642 or a nucleotide sequence derived from the nucleotide sequence by the replacement of u with t according to the present invention, examples of the shortest variants registered in the miRBase Release 20 include polynucleotides having sequences represented by SEQ ID NOs: 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 653, 655 and 657, respectively. In addition to these variants and fragments, examples thereof include a large number of isomiR polynucleotides of SEQ ID NOs: 1 to 199 and 635 to 642 registered in the miRBase. Examples of the polynucleotide comprising a nucleotide sequence represented by any of SEQ ID NOs: 1 to 199 and 635 to 642 include a polynucleotide represented by any of SEQ ID NOs: 200 to 398 and 643 to 651, which are their respective precursors.

The terms and miRBase Accession Nos. (registration numbers) of the genes represented by SEQ ID NOs: 1 to 657 are shown in Table 1.

The term “capable of specifically binding” used herein means that the nucleic acid probe or the primer used in the present invention binds to a particular target nucleic acid and cannot substantially bind to other nucleic acids.

TABLE 1

| SEQ | | miRBase |
|---|---|---|
| ID NO: | Gene name | registration No. |
| 1 | hsa-miR-4257 | MIMAT0016878 |
| 2 | hsa-miR-6726-5p | MIMAT0027353 |
| 3 | hsa-miR-1343-3p | MIMAT0019776 |
| 4 | hsa-miR-1247-3p | MIMAT0022721 |
| 5 | hsa-miR-6787-5p | MIMAT0027474 |
| 6 | hsa-miR-6875-5p | MIMAT0027650 |
| 7 | hsa-miR-1225-3p | MIMAT0005573 |
| 8 | hsa-miR-8063 | MIMAT0030990 |
| 9 | hsa-miR-6781-5p | MIMAT0027462 |
| 10 | hsa-miR-4746-3p | MIMAT0019881 |
| 11 | hsa-miR-1908-5p | MIMAT0007881 |
| 12 | hsa-miR-6756-5p | MIMAT0027412 |
| 13 | hsa-miR-204-3p | MIMAT0022693 |
| 14 | hsa-miR-4651 | MIMAT0019715 |
| 15 | hsa-miR-6757-5p | MIMAT0027414 |
| 16 | hsa-miR-6825-5p | MIMAT0027550 |
| 17 | hsa-miR-7108-5p | MIMAT0028113 |
| 18 | hsa-miR-4792 | MIMAT0019964 |
| 19 | hsa-miR-7641 | MIMAT0029782 |
| 20 | hsa-miR-3188 | MIMAT0015070 |
| 21 | hsa-miR-3131 | MIMAT0014996 |
| 22 | hsa-miR-6780b-5p | MIMAT0027572 |
| 23 | hsa-miR-8069 | MIMAT0030996 |
| 24 | hsa-miR-6840-3p | MIMAT0027583 |
| 25 | hsa-miR-8072 | MIMAT0030999 |
| 26 | hsa-niiR-1233-5p | MIMAT0022943 |
| 27 | hsa-miR-6887-5p | MIMAT0027674 |
| 28 | hsa-miR-1231 | MIMAT0005586 |
| 29 | hsa-miR-5572 | MIMAT0022260 |
| 30 | hsa-miR-6738-5p | MIMAT0027377 |
| 31 | hsa-miR-6784-5p | MIMAT0027468 |
| 32 | hsa-miR-6791-5p | MIMAT0027482 |
| 33 | hsa-miR-6749-5p | MIMAT0027398 |
| 34 | hsa-miR-6741-5p | MIMAT0027383 |
| 35 | hsa-miR-128-1-5p | MIMAT0026477 |
| 36 | hsa-miR-4419b | MIMAT0019034 |
| 37 | hsa-miR-6746-5p | MIMAT0027392 |
| 38 | hsa-miR-3184-5p | MIMAT0015064 |
| 39 | hsa-miR-3679-5p | MIMAT0018104 |
| 40 | hsa-miR-7110-5p | MIMAT0028117 |
| 41 | hsa-miR-4516 | MIMAT0019053 |
| 42 | hsa-miR-6717-5p | MIMAT0025846 |
| 43 | hsa-miR-6826-5p | MIMAT0027552 |
| 44 | hsa-miR-4433b-3p | MIMAT0030414 |
| 45 | hsa-miR-3679-3p | MIMAT0018105 |
| 46 | hsa-miR-3135b | MIMAT0018985 |
| 47 | hsa-miR-3622a-5p | MIMAT0018003 |
| 48 | hsa-miR-711 | MIMAT0012734 |
| 49 | hsa-miR-4467 | MIMAT0018994 |
| 50 | hsa-miR-6857-5p | MIMAT0027614 |
| 51 | hsa-miR-6515-3p | MIMAT0025487 |
| 52 | hsa-miR-1225-5p | MIMAT0005572 |
| 53 | hsa-miR-187-5p | MIMAT0004561 |
| 54 | hsa-miR-3185 | MIMAT0015065 |
| 55 | hsa-miR-642b-3p | MIMAT0018444 |
| 56 | hsa-miR-1249 | MIMAT0005901 |
| 57 | hsa-miR-744-5p | MIMAT0004945 |
| 58 | hsa-miR-4442 | MIMAT0018960 |
| 59 | hsa-miR-1228-3p | MIMAT0005583 |
| 60 | hsa-miR-939-5p | MIMAT0004982 |
| 61 | hsa-miR-6845-5p | MIMAT0027590 |
| 62 | hsa-miR-887-3p | MIMAT0004951 |
| 63 | hsa-miR-7845-5p | MIMAT0030420 |
| 64 | hsa-miR-6729-5p | MIMAT0027359 |
| 65 | hsa-miR-4632-5p | MIMAT0022977 |
| 66 | hsa-miR-615-5p | MIMAT0004804 |
| 67 | hsa-miR-6724-5p | MIMAT0025856 |
| 68 | hsa-miR-4728-5p | MIMAT0019849 |
| 69 | hsa-miR-6732-5p | MIMAT0027365 |
| 70 | hsa-miR-6816-5p | MIMAT0027532 |
| 71 | hsa-miR-4695-5p | MIMAT0019788 |
| 72 | hsa-miR-6088 | MIMAT0023713 |
| 73 | hsa-miR-7975 | MIMAT0031178 |
| 74 | hsa-miR-3197 | MIMAT0015082 |
| 75 | hsa-miR-6125 | MIMAT0024598 |
| 76 | hsa-miR-4433-3p | MIMAT0018949 |
| 77 | hsa-miR-6727-5p | MIMAT0027355 |
| 78 | hsa-miR-4706 | MIMAT0019806 |
| 79 | hsa-miR-7847-3p | MIMAT0030422 |
| 80 | hsa-miR-6805-3p | MIMAT0027511 |
| 81 | hsa-miR-6766-3p | MIMAT0027433 |
| 82 | hsa-miR-1913 | MIMAT0007888 |
| 83 | hsa-miR-4649-5p | MIMAT0019711 |
| 84 | hsa-miR-602 | MIMAT0003270 |
| 85 | hsa-miR-3663-3p | MIMAT0018085 |
| 86 | hsa-miR-6893-5p | MIMAT0027686 |
| 87 | hsa-miR-6861-5p | MIMAT0027623 |
| 88 | hsa-miR-4449 | MIMAT0018968 |
| 89 | hsa-miR-6842-5p | MIMAT0027586 |
| 90 | hsa-miR-4454 | MIMAT0018976 |
| 91 | hsa-miR-5195-3p | MIMAT0021127 |
| 92 | hsa-miR-663b | MIMAT0005867 |
| 93 | hsa-miR-6765-5p | MIMAT0027430 |
| 94 | hsa-miR-4513 | MIMAT0019050 |
| 95 | hsa-miR-614 | MIMAT0003282 |
| 96 | hsa-miR-6785-5p | MIMAT0027470 |
| 97 | hsa-miR-6777-5p | MIMAT0027454 |
| 98 | hsa-miR-940 | MIMAT0004983 |
| 99 | hsa-miR-4741 | MIMAT0019871 |
| 100 | hsa-miR-6870-5p | MIMAT0027640 |
| 101 | hsa-miR-6131 | MIMAT0024615 |
| 102 | hsa-miR-150-3p | MIMAT0004610 |
| 103 | hsa-miR-4707-5p | MIMAT0019807 |
| 104 | hsa-miR-1915-3p | MIMAT0007892 |
| 105 | hsa-miR-3937 | MIMAT0018352 |
| 106 | hsa-miR-937-5p | MIMAT0022938 |
| 107 | hsa-miR-4443 | MIMAT0018961 |
| 108 | hsa-miR-1914-3p | MIMAT0007890 |
| 109 | hsa-miR-3620-5p | MIMAT0022967 |
| 110 | hsa-miR-1268b | MIMAT0018925 |
| 111 | hsa-miR-1227-5p | MIMAT0022941 |
| 112 | hsa-miR-6880-5p | MIMAT0027660 |
| 113 | hsa-miR-4417 | MIMAT0018929 |
| 114 | hsa-miR-6802-5p | MIMAT0027504 |
| 115 | hsa-miR-6769a-5p | MIMAT0027438 |
| 116 | hsa-miR-663a | MIMAT0003326 |
| 117 | hsa-miR-6721-5p | MIMAT0025852 |
| 118 | hsa-miR-4532 | MIMAT0019071 |
| 119 | hsa-miR-7977 | MIMAT0031180 |
| 120 | hsa-miR-92b-5p | MIMAT0004792 |
| 121 | hsa-miR-371a-5p | MIMAT0004687 |
| 122 | hsa-miR-6126 | MIMAT0024599 |
| 123 | hsa-miR-4734 | MIMAT0019859 |
| 124 | hsa-miR-4665-3p | MIMAT0019740 |
| 125 | hsa-miR-423-5p | MIMAT0004748 |
| 126 | hsa-miR-1469 | MIMAT0007347 |
| 127 | hsa-miR-4675 | MIMAT0019757 |
| 128 | hsa-miR-1915-5p | MIMAT0007891 |
| 129 | hsa-miR-6716-5p | MIMAT0025844 |
| 130 | hsa-miR-718 | MIMAT0012735 |
| 131 | hsa-miR-4281 | MIMAT0016907 |
| 132 | hsa-miR-6820-5p | MIMAT0027540 |
| 133 | hsa-miR-6795-5p | MIMAT0027490 |
| 134 | hsa-miR-6779-5p | MIMAT0027458 |
| 135 | hsa-miR-7109-5p | MIMAT0028115 |
| 136 | hsa-miR-6798-5p | MIMAT0027496 |
| 137 | hsa-miR-4648 | MIMAT0019710 |
| 138 | hsa-miR-8059 | MIMAT0030986 |
| 139 | hsa-miR-6765-3p | MIMAT0027431 |
| 140 | hsa-miR-6132 | MIMAT0024616 |
| 141 | hsa-miR-4492 | MIMAT0019027 |
| 142 | hsa-miR-7107-5p | MIMAT0028111 |
| 143 | hsa-miR-3195 | MIMAT0015079 |
| 144 | hsa-miR-3180 | MIMAT0018178 |
| 145 | hsa-miR-296-3p | MIMAT0004679 |
| 146 | hsa-miR-564 | MIMAT0003228 |
| 147 | hsa-miR-1268a | MIMAT0005922 |
| 148 | hsa-miR-6848-5p | MIMAT0027596 |
| 149 | hsa-miR-762 | MIMAT0010313 |
| 150 | hsa-miR-2861 | MIMAT0013802 |
| 151 | hsa-miR-1203 | MIMAT0005866 |
| 152 | hsa-miR-1260b | MIMAT0015041 |
| 153 | hsa-miR-4476 | MIMAT0019003 |
| 154 | hsa-miR-6885-5p | MIMAT0027670 |
| 155 | hsa-miR-6769b-5p | MIMAT0027620 |
| 156 | hsa-miR-23b-3p | MIMAT0000418 |
| 157 | hsa-miR-1343-5p | MIMAT0027038 |
| 158 | hsa-miR-3621 | MIMAT0018002 |
| 159 | hsa-miR-4688 | MIMAT0019777 |
| 160 | hsa-miR-4286 | MIMAT0016916 |
| 161 | hsa-miR-4640-5p | MIMAT0019699 |
| 162 | hsa-miR-4739 | MIMAT0019868 |
| 163 | hsa-miR-1260a | MIMAT0005911 |
| 164 | hsa-miR-4276 | MIMAT0016904 |
| 165 | hsa-miR-7106-5p | MIMAT0028109 |
| 166 | hsa-miR-128-2-5p | MIMAT0031095 |
| 167 | hsa-miR-125a-3p | MIMAT0004602 |
| 168 | hsa-miR-92a-2-5p | MIMAT0004508 |
| 169 | hsa-miR-486-3p | MIMAT0004762 |
| 170 | hsa-miR-3196 | MIMAT0015080 |
| 171 | hsa-miR-211-3p | MIMAT0022694 |
| 172 | hsa-miR-4271 | MIMAT0016901 |
| 173 | hsa-miR-6851-5p | MIMAT0027602 |
| 174 | hsa-miR-149-3p | MIMAT0004609 |
| 175 | hsa-miR-4667-5p | MIMAT0019743 |
| 176 | hsa-miR-135a-3p | MIMAT0004595 |
| 177 | hsa-miR-4486 | MIMAT0019020 |
| 178 | hsa-miR-4697-5p | MIMAT0019791 |
| 179 | hsa-miR-4725-3p | MIMAT0019844 |
| 180 | hsa-miR-6510-5p | MIMAT0025476 |
| 181 | hsa-miR-5001-5p | MIMAT0021021 |
| 182 | hsa-miR-4673 | MIMAT0019755 |
| 183 | hsa-miR-4466 | MIMAT0018993 |
| 184 | hsa-miR-23a-3p | MIMAT0000078 |
| 185 | hsa-miR-3656 | MIMAT0018076 |
| 186 | hsa-miR-6782-5p | MIMAT0027464 |
| 187 | hsa-miR-4689 | MIMAT0019778 |
| 188 | hsa-miR-451a | MIMAT0001631 |
| 189 | hsa-miR-4446-3p | MIMAT0018965 |
| 190 | hsa-miR-3180-3p | MIMAT0015058 |
| 191 | hsa-miR-642a-3p | MIMAT0020924 |
| 192 | hsa-miR-6889-5p | MIMAT0027678 |
| 193 | hsa-miR-3178 | MIMAT0015055 |
| 194 | hsa-miR-4665-5p | MIMAT0019739 |
| 195 | hsa-miR-6722-3p | MIMAT0025854 |
| 196 | hsa-miR-30c-1-3p | MIMAT0004674 |
| 197 | hsa-miR-4507 | MIMAT0019044 |
| 198 | hsa-miR-3141 | MIMAT0015010 |
| 199 | hsa-miR-1199-5p | MIMAT0031119 |
| 200 | hsa-mir-4257 | MI0015856 |
| 201 | hsa-mir-6726 | MI0022571 |
| 202 | hsa-mir-1343 | MI0017320 |
| 203 | hsa-mir-1247 | MI0006382 |
| 204 | hsa-mir-6787 | MI0022632 |
| 205 | hsa-mir-6875 | MI0022722 |
| 206 | hsa-mir-1225 | MI0006311 |
| 207 | hsa-mir-8063 | MI0025899 |
| 208 | hsa-mir-6781 | MI0022626 |
| 209 | hsa-mir-4746 | MI0017385 |
| 210 | hsa-mir-1908 | MI0008329 |
| 211 | hsa-mir-6756 | MI0022601 |
| 212 | hsa-mir-204 | MI0000284 |
| 213 | hsa-mir-4651 | MI0017279 |
| 214 | hsa-mir-6757 | MI0022602 |
| 215 | hsa-mir-6825 | MI0022670 |
| 216 | hsa-mir-7108 | MI0022959 |
| 217 | hsa-mir-4792 | MI0017439 |
| 218 | hsa-mir-7641-1 | MI0024975 |
| 219 | hsa-mir-7641-2 | MI0024976 |
| 220 | hsa-miR-3188 | MI0014232 |
| 221 | hsa-miR-3131 | MI0014151 |
| 222 | hsa-mir-6780b | MI0022681 |
| 223 | hsa-mir-8069 | MI0025905 |
| 224 | hsa-mir-6840 | MI0022686 |
| 225 | hsa-mir-8072 | MI0025908 |
| 226 | hsa-mir-1233-1 | MI0006323 |
| 227 | hsa-mir-1233-2 | MI0015973 |
| 228 | hsa-mir-6887 | MI0022734 |
| 229 | hsa-mir-1231 | MI0006321 |
| 230 | hsa-mir-5572 | MI0019117 |
| 231 | hsa-mir-6738 | MI0022583 |
| 232 | hsa-mir-6784 | MI0022629 |
| 233 | hsa-mir-6791 | MI0022636 |
| 234 | hsa-mir-6749 | MI0022594 |
| 235 | hsa-mir-6741 | MI0022586 |
| 236 | hsa-mir-128-1 | MI0000447 |
| 237 | hsa-mir-4419b | MI0016861 |
| 238 | hsa-mir-6746 | MI0022591 |
| 239 | hsa-mir-3184 | MI0014226 |
| 240 | hsa-mir-3679 | MI0016080 |
| 241 | hsa-mir-7110 | MI0022961 |
| 242 | hsa-mir-4516 | MI0016882 |
| 243 | hsa-mir-6717 | MI0022551 |
| 244 | hsa-mir-6826 | MI0022671 |
| 245 | hsa-mir-4433b | MI0025511 |
| 246 | hsa-mir-3135b | MI0016809 |
| 247 | hsa-mir-3622a | MI0016013 |
| 248 | hsa-mir-711 | MI0012488 |
| 249 | hsa-mir-4467 | MI0016818 |
| 250 | hsa-mir-6857 | MI0022703 |
| 251 | hsa-mir-6515 | MI0022227 |
| 252 | hsa-mir-187 | MI0000274 |
| 253 | hsa-mir-3185 | MI0014227 |
| 254 | hsa-mir-642b | MI0016685 |
| 255 | hsa-mir-1249 | MI0006384 |
| 256 | hsa-mir-744 | MI0005559 |
| 257 | hsa-mir-4442 | MI0016785 |
| 258 | hsa-mir-1228 | MI0006318 |
| 259 | hsa-mir-939 | MI0005761 |
| 260 | hsa-mir-6845 | MI0022691 |
| 261 | hsa-mir-887 | MI0005562 |
| 262 | hsa-mir-7845 | MI0025515 |
| 263 | hsa-mir-6729 | MI0022574 |
| 264 | hsa-mir-4632 | MI0017259 |
| 265 | hsa-mir-615 | MI0003628 |
| 266 | hsa-mir-6724 | MI0022559 |
| 267 | hsa-mir-4728 | MI0017365 |
| 268 | hsa-mir-6732 | MI0022577 |
| 269 | hsa-mir-6816 | MI0022661 |
| 270 | hsa-mir-4695 | MI0017328 |
| 271 | hsa-mir-6088 | MI0020365 |
| 272 | hsa-mir-7975 | MI0025751 |
| 273 | hsa-mir-3197 | MI0014245 |
| 274 | hsa-mir-6125 | MI0021259 |
| 275 | hsa-mir-4433 | MI0016773 |
| 276 | hsa-mir-6727 | MI0022572 |
| 277 | hsa-mir-4706 | MI0017339 |
| 278 | hsa-mir-7847 | MI0025517 |
| 279 | hsa-mir-6805 | MI0022650 |
| 280 | hsa-mir-6766 | MI0022611 |
| 281 | hsa-mir-1913 | MI0008334 |
| 282 | hsa-mir-4649 | MI0017276 |
| 283 | hsa-mir-602 | MI0003615 |
| 284 | hsa-mir-3663 | MI0016064 |
| 285 | hsa-mir-6893 | MI0022740 |
| 286 | hsa-mir-6861 | MI0022708 |
| 287 | hsa-mir-4449 | MI0016792 |
| 288 | hsa-mir-6842 | MI0022688 |
| 289 | hsa-mir-4454 | MI0016800 |
| 290 | hsa-mir-5195 | MI0018174 |
| 291 | hsa-mir-663b | MI0006336 |
| 292 | hsa-mir-6765 | MI0022610 |
| 293 | hsa-mir-4513 | MI0016879 |
| 294 | hsa-mir-614 | MI0003627 |
| 295 | hsa-mir-6785 | MI0022630 |
| 296 | hsa-mir-6777 | MI0022622 |
| 297 | hsa-mir-940 | MI0005762 |
| 298 | hsa-mir-4741 | MI0017379 |
| 299 | hsa-mir-6870 | MI0022717 |
| 300 | hsa-mir-6131 | MI0021276 |
| 301 | hsa-mir-150 | MI0000479 |
| 302 | hsa-mir-4707 | MI0017340 |
| 303 | hsa-mir-1915 | MI0008336 |
| 304 | hsa-mir-3937 | MI0016593 |
| 305 | hsa-mir-937 | MI0005759 |
| 306 | hsa-mir-4443 | MI0016786 |
| 307 | hsa-mir-1914 | MI0008335 |
| 308 | hsa-mir-3620 | MI0016011 |
| 309 | hsa-mir-1268b | MI0016748 |
| 310 | hsa-mir-1227 | MI0006316 |
| 311 | hsa-mir-6880 | MI0022727 |
| 312 | hsa-mir-4417 | MI0016753 |
| 313 | hsa-mir-6802 | MI0022647 |
| 314 | hsa-mir-6769a | MI0022614 |
| 315 | hsa-mir-663a | MI0003672 |
| 316 | hsa-mir-6721 | MI0022556 |
| 317 | hsa-mir-4532 | MI0016899 |
| 318 | hsa-mir-7977 | MI0025753 |
| 319 | hsa-mir-92b | MI0003560 |
| 320 | hsa-mir-371a | MI0000779 |
| 321 | hsa-mir-6126 | MI0021260 |
| 322 | hsa-mir-4734 | MI0017371 |
| 323 | hsa-mir-4665 | MI0017295 |
| 324 | hsa-mir-423 | MI0001445 |
| 325 | hsa-mir-1469 | MI0007074 |
| 326 | hsa-mir-4675 | MI0017306 |
| 327 | hsa-mir-6716 | MI0022550 |
| 328 | hsa-mir-718 | MI0012489 |
| 329 | hsa-mir-4281 | MI0015885 |
| 330 | hsa-mir-6820 | MI0022665 |
| 331 | hsa-mir-6795 | MI0022640 |
| 332 | hsa-mir-6779 | MI0022624 |
| 333 | hsa-mir-7109 | MI0022960 |
| 334 | hsa-mir-6798 | MI0022643 |
| 335 | hsa-mir-4648 | MI0017275 |
| 336 | hsa-mir-8059 | MI0025895 |
| 337 | hsa-mir-6132 | MI0021277 |
| 338 | hsa-mir-4492 | MI0016854 |
| 339 | hsa-mir-7107 | MI0022958 |
| 340 | hsa-mir-3195 | MI0014240 |
| 341 | hsa-mir-3180-4 | MI0016408 |
| 342 | hsa-mir-3180-5 | MI0016409 |
| 343 | hsa-mir-296 | MI0000747 |
| 344 | hsa-mir-564 | MI0003570 |
| 345 | hsa-mir-1268a | MI0006405 |
| 346 | hsa-mir-6848 | MI0022694 |
| 347 | hsa-mir-762 | MI0003892 |
| 348 | hsa-mir-2861 | MI0013006 |
| 349 | hsa-mir-1203 | MI0006335 |
| 350 | hsa-mir-1260b | MI0014197 |
| 351 | hsa-mir-4476 | MI0016828 |
| 352 | hsa-mir-6885 | MI0022732 |
| 353 | hsa-mir-6769b | MI0022706 |
| 354 | hsa-mir-23b | MI0000439 |
| 355 | hsa-mir-3621 | MI0016012 |
| 356 | hsa-mir-4688 | MI0017321 |
| 357 | hsa-mir-4286 | MI0015894 |
| 358 | hsa-mir-4640 | MI0017267 |
| 359 | hsa-mir-4739 | MI0017377 |
| 360 | hsa-mir-1260a | MI0006394 |
| 361 | hsa-mir-4276 | MI0015882 |
| 362 | hsa-mir-7106 | MI0022957 |
| 363 | hsa-mir-128-2 | MI0000727 |
| 364 | hsa-mir-125a | MI0000469 |
| 365 | hsa-mir-92a-2 | MI0000094 |
| 366 | hsa-mir-486 | MI0002470 |
| 367 | hsa-mir-486-2 | MI0023622 |
| 368 | hsa-mir-3196 | MI0014241 |
| 369 | hsa-mir-211 | MI0000287 |
| 370 | hsa-mir-4271 | MI0015879 |
| 371 | hsa-mir-6851 | MI0022697 |
| 372 | hsa-mir-149 | MI0000478 |
| 373 | hsa-mir-4667 | MI0017297 |
| 374 | hsa-mir-135a-1 | MI0000452 |
| 375 | hsa-mir-4486 | MI0016847 |
| 376 | hsa-mir-4697 | MI0017330 |
| 377 | hsa-mir-4725 | MI0017362 |
| 378 | hsa-mir-6510 | MI0022222 |
| 379 | hsa-mir-5001 | MI0017867 |
| 380 | hsa-mir-4673 | MI0017304 |
| 381 | hsa-mir-4466 | MI0016817 |
| 382 | hsa-mir-23a | MI0000079 |
| 383 | hsa-mir-3656 | MI0016056 |
| 384 | hsa-mir-6782 | MI0022627 |
| 385 | hsa-mir-4689 | MI0017322 |
| 386 | hsa-mir-451a | MI0001729 |
| 387 | hsa-mir-4446 | MI0016789 |
| 388 | hsa-mir-3180-1 | MI0014214 |
| 389 | hsa-mir-3180-2 | MI0014215 |
| 390 | hsa-mir-3180-3 | MI0014217 |
| 391 | hsa-mir-642a | MI0003657 |
| 392 | hsa-mir-6889 | MI0022736 |
| 393 | hsa-mir-3178 | MI0014212 |
| 394 | hsa-mir-6722 | MI0022557 |
| 395 | hsa-mir-30c-1 | MI0000736 |
| 396 | hsa-mir-4507 | MI0016871 |
| 397 | hsa-mir-3141 | MI0014165 |
| 398 | hsa-mir-1199 | MI0020340 |
| 399 | isomiR example 1 of SEQ ID NO: 3 | — |
| 400 | isomiR example 2 of SEQ ID NO: 3 | — |
| 401 | isomiR example 1 of SEQ ID NO: 4 | — |
| 402 | isomiR example 2 of SEQ ID NO: 4 | — |
| 403 | isomiR example 1 of SEQ ID NO: 11 | — |
| 404 | isomiR example 2 of SEQ ID NO: 11 | — |
| 405 | isomiR example 1 of SEQ ID NO: 13 | — |
| 406 | isomiR example 2 of SEQ ID NO: 13 | — |
| 407 | isomiR example 1 of SEQ ID NO: 14 | — |
| 408 | isomiR example 2 of SEQ ID NO: 14 | — |
| 409 | isomiR example 1 of SEQ ID NO: 18 | — |
| 410 | isomiR example 2 of SEQ ID NO: 18 | — |
| 411 | isomiR example 1 of SEQ ID NO: 20 | — |
| 412 | isomiR example 2 of SEQ ID NO: 20 | — |
| 413 | isomiR example 1 of SEQ ID NO: 21 | — |
| 414 | isomiR example 2 of SEQ ID NO: 21 | — |
| 415 | isomiR example 1 of SEQ ID NO: 26 | — |
| 416 | isomiR example 2 of SEQ ID NO: 26 | — |
| 417 | isomiR example 1 of SEQ ID NO: 29 | — |
| 418 | isomiR example 2 of SEQ ID NO: 29 | — |
| 419 | isomiR example 1 of SEQ ID NO: 35 | — |
| 420 | isomiR example 2 of SEQ ID NO: 35 | — |
| 421 | isomiR example 1 of SEQ ID NO: 36 | — |
| 422 | isomiR example 2 of SEQ ID NO: 36 | — |
| 423 | isomiR example 1 of SEQ ID NO: 39 | — |
| 424 | isomiR example 2 of SEQ ID NO: 39 | — |
| 425 | isomiR example 1 of SEQ ID NO: 41 | — |
| 426 | isomiR example 2 of SEQ ID NO: 41 | — |
| 427 | isomiR example 1 of SEQ ID NO: 42 | — |
| 428 | isomiR example 2 of SEQ ID NO: 42 | — |
| 429 | isomiR example 1 of SEQ ID NO: 45 | — |
| 430 | isomiR example 2 of SEQ ID NO: 45 | — |
| 431 | isomiR example 1 of SEQ ID NO: 46 | — |
| 432 | isomiR example 2 of SEQ ID NO: 46 | — |
| 433 | isomiR example 1 of SEQ ID NO: 47 | — |
| 434 | isomiR example 2 of SEQ ID NO: 47 | — |
| 435 | isomiR example 1 of SEQ ID NO: 48 | — |
| 436 | isomiR example 2 of SEQ ID NO: 48 | — |
| 437 | isomiR example 1 of SEQ ID NO: 49 | — |
| 438 | isomiR example 2 of SEQ ID NO: 49 | — |
| 439 | isomiR example 1 of SEQ ID NO: 51 | — |
| 440 | isomiR example 2 of SEQ ID NO: 51 | — |
| 441 | isomiR example 1 of SEQ ID NO: 53 | — |
| 442 | isomiR example 2 of SEQ ID NO: 53 | — |
| 443 | isomiR example 1 of SEQ ID NO: 54 | — |
| 444 | isomiR example 2 of SEQ ID NO: 54 | — |
| 445 | isomiR example 1 of SEQ ID NO: 55 | — |
| 446 | isomiR example 2 of SEQ ID NO: 55 | — |
| 447 | isomiR example 1 of SEQ ID NO: 56 | — |
| 448 | isomiR example 2 of SEQ ID NO: 56 | — |
| 449 | isomiR example 1 of SEQ ID NO: 57 | — |
| 450 | isomiR example 2 of SEQ ID NO: 57 | — |
| 451 | isomiR example 1 of SEQ ID NO: 58 | — |
| 452 | isomiR example 2 of SEQ ID NO: 58 | — |
| 453 | isomiR example 1 of SEQ ID NO: 59 | — |
| 454 | isomiR example 2 of SEQ ID NO: 59 | — |
| 455 | isomiR example 1 of SEQ ID NO: 60 | — |
| 456 | isomiR example 2 of SEQ ID NO: 60 | — |
| 457 | isomiR example 1 of SEQ ID NO: 62 | — |
| 458 | isomiR example 2 of SEQ ID NO: 62 | — |
| 459 | isomiR example 1 of SEQ ID NO: 65 | — |
| 460 | isomiR example 2 of SEQ ID NO: 65 | — |
| 461 | isomiR example 1 of SEQ ID NO: 66 | — |
| 462 | isomiR example 2 of SEQ ID NO: 66 | — |
| 463 | isomiR example 1 of SEQ ID NO: 67 | — |
| 464 | isomiR example 2 of SEQ ID NO: 67 | — |
| 465 | isomiR example 1 of SEQ ID NO: 68 | — |
| 466 | isomiR example 2 of SEQ ID NO: 68 | — |
| 467 | isomiR example 1 of SEQ ID NO: 71 | — |
| 468 | isomiR example 2 of SEQ ID NO: 71 | — |
| 469 | isomiR example 1 of SEQ ID NO: 72 | — |
| 470 | isomiR example 2 of SEQ ID NO: 72 | — |
| 471 | isomiR example 1 of SEQ ID NO: 73 | — |
| 472 | isomiR example 2 of SEQ ID NO: 73 | — |
| 473 | isomiR example 1 of SEQ ID NO: 74 | — |
| 474 | isomiR example 2 of SEQ ID NO: 74 | — |
| 475 | isomiR example 1 of SEQ ID NO: 75 | — |
| 476 | isomiR example 2 of SEQ ID NO: 75 | — |
| 477 | isomiR example 1 of SEQ ID NO: 76 | — |
| 478 | isomiR example 2 of SEQ ID NO: 76 | — |
| 479 | isomiR example 1 of SEQ ID NO: 78 | — |
| 480 | isomiR example 2 of SEQ ID NO: 78 | — |
| 481 | isomiR example 1 of SEQ ID NO: 82 | — |
| 482 | isomiR example 2 of SEQ ID NO: 82 | — |
| 483 | isomiR example 1 of SEQ ID NO: 83 | — |
| 484 | isomiR example 2 of SEQ ID NO: 83 | — |
| 485 | isomiR example 1 of SEQ ID NO: 88 | — |
| 486 | isomiR example 2 of SEQ ID NO: 88 | — |
| 487 | isomiR example 1 of SEQ ID NO: 90 | — |
| 488 | isomiR example 2 of SEQ ID NO: 90 | — |
| 489 | isomiR example 1 of SEQ ID NO: 91 | — |
| 490 | isomiR example 2 of SEQ ID NO: 91 | — |
| 491 | isomiR example 1 of SEQ ID NO: 92 | — |
| 492 | isomiR example 2 of SEQ ID NO: 92 | — |
| 493 | isomiR example 1 of SEQ ID NO: 94 | — |
| 494 | isomiR example 2 of SEQ ID NO: 94 | — |
| 495 | isomiR example 1 of SEQ ID NO: 95 | — |
| 496 | isomiR example 2 of SEQ ID NO: 95 | — |
| 497 | isomiR example 1 of SEQ ID NO: 98 | — |
| 498 | isomiR example 2 of SEQ ID NO: 98 | — |
| 499 | isomiR example 1 of SEQ ID NO: 99 | — |
| 500 | isomiR example 2 of SEQ ID NO: 99 | — |
| 501 | isomiR example 1 of SEQ ID NO: 101 | — |
| 502 | isomiR example 2 of SEQ ID NO: 101 | — |
| 503 | isomiR example 1 of SEQ ID NO: 102 | — |
| 504 | isomiR example 2 of SEQ ID NO: 102 | — |
| 505 | isomiR example 1 of SEQ ID NO: 103 | — |
| 506 | isomiR example 2 of SEQ ID NO: 103 | — |
| 507 | isomiR example 1 of SEQ ID NO: 104 | — |
| 508 | isomiR example 2 of SEQ ID NO: 104 | — |
| 509 | isomiR example 1 of SEQ ID NO: 106 | — |
| 510 | isomiR example 2 of SEQ ID NO: 106 | — |
| 511 | isomiR example 1 of SEQ ID NO: 107 | — |
| 512 | isomiR example 2 of SEQ ID NO: 107 | — |
| 513 | isomiR example 1 of SEQ ID NO: 108 | — |
| 514 | isomiR example 2 of SEQ ID NO: 108 | — |
| 515 | isomiR example 1 of SEQ ID NO: 109 | — |
| 516 | isomiR example 2 of SEQ ID NO: 109 | — |
| 517 | isomiR example 1 of SEQ ID NO: 110 | — |
| 518 | isomiR example 2 of SEQ ID NO: 110 | — |
| 519 | isomiR example 1 of SEQ ID NO: 113 | — |
| 520 | isomiR example 2 of SEQ ID NO: 113 | — |
| 521 | isomiR example 1 of SEQ ID NO: 116 | — |
| 522 | isomiR example 2 of SEQ ID NO: 116 | — |
| 523 | isomiR example 1 of SEQ ID NO: 117 | — |
| 524 | isomiR example 2 of SEQ ID NO: 117 | — |
| 525 | isomiR example 1 of SEQ ID NO: 118 | — |
| 526 | isomiR example 2 of SEQ ID NO: 118 | — |
| 527 | isomiR example 1 of SEQ ID NO: 120 | — |
| 528 | isomiR example 2 of SEQ ID NO: 120 | — |
| 529 | isomiR example 1 of SEQ E) NO: 121 | — |
| 530 | isomiR example 2 of SEQ E) NO: 121 | — |
| 531 | isomiR example 1 of SEQ ID NO: 122 | — |
| 532 | isomiR example 2 of SEQ ID NO: 122 | — |
| 533 | isomiR example 1 of SEQ ID NO: 123 | — |
| 534 | isomiR example 2 of SEQ ID NO: 123 | — |
| 535 | isomiR example 1 of SEQ ID NO: 125 | — |
| 536 | isomiR example 2 of SEQ ID NO: 125 | — |
| 537 | isomiR example 1 of SEQ ID NO: 128 | — |
| 538 | isomiR example 2 of SEQ ID NO: 128 | — |
| 539 | isomiR example 1 of SEQ ID NO: 129 | — |
| 540 | isomiR example 2 of SEQ ID NO: 129 | — |
| 541 | isomiR example 1 of SEQ ID NO: 130 | — |
| 542 | isomiR example 2 of SEQ ID NO: 130 | — |
| 543 | isomiR example 1 of SEQ ID NO: 131 | — |
| 544 | isomiR example 2 of SEQ ID NO: 131 | — |
| 545 | isomiR example 1 of SEQ ID NO: 137 | — |
| 546 | isomiR example 2 of SEQ ID NO: 137 | — |
| 547 | isomiR example 1 of SEQ ID NO: 140 | — |
| 548 | isomiR example 2 of SEQ ID NO: 140 | — |
| 549 | isomiR example 1 of SEQ ID NO: 141 | — |
| 550 | isomiR example 2 of SEQ ID NO: 141 | — |
| 551 | isomiR example 1 of SEQ ID NO: 143 | — |
| 552 | isomiR example 2 of SEQ ID NO: 143 | — |
| 553 | isomiR example 1 of SEQ ID NO: 144 | — |
| 554 | isomiR example 2 of SEQ ID NO: 144 | — |
| 555 | isomiR example 1 of SEQ ID NO: 145 | — |
| 556 | isomiR example 2 of SEQ ID NO: 145 | — |
| 557 | isomiR example 1 of SEQ ID NO: 146 | — |
| 558 | isomiR example 2 of SEQ ID NO: 146 | — |
| 559 | isomiR example 1 of SEQ ID NO: 147 | — |
| 560 | isomiR example 2 of SEQ ID NO: 147 | — |
| 561 | isomiR example 1 of SEQ ID NO: 150 | — |
| 562 | isomiR example 2 of SEQ ID NO: 150 | — |
| 563 | isomiR example 1 of SEQ ID NO: 152 | — |
| 564 | isomiR example 2 of SEQ ID NO: 152 | — |
| 565 | isomiR example 1 of SEQ ID NO: 153 | — |
| 566 | isomiR example 2 of SEQ ID NO: 153 | — |
| 567 | isomiR example 1 of SEQ ID NO: 156 | — |
| 568 | isomiR example 2 of SEQ ID NO: 156 | — |
| 569 | isomiR example 1 of SEQ ID NO: 159 | — |
| 570 | isomiR example 2 of SEQ ID NO: 159 | — |
| 571 | isomiR example 1 of SEQ ID NO: 160 | — |
| 572 | isomiR example 2 of SEQ ID NO: 160 | — |
| 573 | isomiR example 1 of SEQ ID NO: 161 | — |
| 574 | isomiR example 2 of SEQ ID NO: 161 | — |
| 575 | isomiR example 1 of SEQ ID NO: 162 | — |
| 576 | isomiR example 2 of SEQ ID NO: 162 | — |
| 577 | isomiR example 1 of SEQ ID NO: 163 | — |
| 578 | isomiR example 2 of SEQ ID NO: 163 | — |
| 579 | isomiR example 1 of SEQ ID NO: 166 | — |
| 580 | isomiR example 2 of SEQ ID NO: 166 | — |
| 581 | isomiR example 1 of SEQ ID NO: 167 | — |
| 582 | isomiR example 2 of SEQ ID NO: 167 | — |
| 583 | isomiR example 1 of SEQ ID NO: 168 | — |
| 584 | isomiR example 2 of SEQ ID NO: 168 | — |
| 585 | isomiR example 1 of SEQ ID NO: 169 | — |
| 586 | isomiR example 2 of SEQ ID NO: 169 | — |
| 587 | isomiR example 1 of SEQ ID NO: 170 | — |
| 588 | isomiR example 2 of SEQ ID NO: 170 | — |
| 589 | isomiR example 1 of SEQ ID NO: 171 | — |
| 590 | isomiR example 2 of SEQ ID NO: 171 | — |
| 591 | isomiR example 1 of SEQ ID NO: 172 | — |
| 592 | isomiR example 2 of SEQ ID NO: 172 | — |
| 593 | isomiR example 1 of SEQ ID NO: 174 | — |
| 594 | isomiR example 2 of SEQ ID NO: 174 | — |
| 595 | isomiR example 1 of SEQ ID NO: 175 | — |
| 596 | isomiR example 2 of SEQ ID NO: 175 | — |
| 597 | isomiR example 1 of SEQ ID NO: 176 | — |
| 598 | isomiR example 2 of SEQ ID NO: 176 | — |
| 599 | isomiR example 1 of SEQ ID NO: 177 | — |
| 600 | isomiR example 2 of SEQ ID NO: 177 | — |
| 601 | isomiR example 1 of SEQ ID NO: 179 | — |
| 602 | isomiR example 2 of SEQ ID NO: 179 | — |
| 603 | isomiR example 1 of SEQ ID NO: 180 | — |
| 604 | isomiR example 2 of SEQ ID NO: 180 | — |
| 605 | isomiR example 1 of SEQ ID NO: 181 | — |
| 606 | isomiR example 2 of SEQ ID NO: 181 | — |
| 607 | isomiR example 1 of SEQ ID NO: 182 | — |
| 608 | isomiR example 2 of SEQ ID NO: 182 | — |
| 609 | isomiR example 1 of SEQ ID NO: 183 | — |
| 610 | isomiR example 2 of SEQ ID NO: 183 | — |
| 611 | isomiR example 1 of SEQ ID NO: 184 | — |
| 612 | isomiR example 2 of SEQ ID NO: 184 | — |
| 613 | isomiR example 1 of SEQ ID NO: 185 | — |
| 614 | isomiR example 2 of SEQ ID NO: 185 | — |
| 615 | isomiR example 1 of SEQ ID NO: 187 | — |
| 616 | isomiR example 2 of SEQ ID NO: 187 | — |
| 617 | isomiR example 1 of SEQ ID NO: 188 | — |
| 618 | isomiR example 2 of SEQ ID NO: 188 | — |
| 619 | isomiR example 1 of SEQ ID NO: 189 | — |
| 620 | isomiR example 2 of SEQ ID NO: 189 | — |
| 621 | isomiR example 1 of SEQ ID NO: 190 | — |
| 622 | isomiR example 2 of SEQ ID NO: 190 | — |
| 623 | isomiR example 1 of SEQ ID NO: 191 | — |
| 624 | isomiR example 2 of SEQ ID NO: 191 | — |
| 625 | isomiR example 1 of SEQ ID NO: 193 | — |
| 626 | isomiR example 2 of SEQ ID NO: 193 | — |
| 627 | isomiR example 1 of SEQ ID NO: 194 | — |
| 628 | isomiR example 2 of SEQ ID NO: 194 | — |
| 629 | isomiR example 1 of SEQ ID NO: 196 | — |
| 630 | isomiR example 2 of SEQ ID NO: 196 | — |
| 631 | isomiR example 1 of SEQ ID NO: 197 | — |
| 632 | isomiR example 2 of SEQ ID NO: 197 | — |
| 633 | isomiR example 1 of SEQ ID NO: 198 | — |
| 634 | isomiR example 2 of SEQ ID NO: 198 | — |
| 635 | hsa-miR-6794-5p | MIMAT0027488 |
| 636 | hsa-miR-6774-5p | MIMAT0027448 |
| 637 | hsa-miR-4707-3p | MIMAT0019808 |
| 638 | hsa-miR-4534 | MIMAT0019073 |
| 639 | hsa-miR-4294 | MIMAT0016849 |
| 640 | hsa-miR-6850-5p | MIMAT0027600 |
| 641 | hsa-miR-6089 | MIMAT0023714 |
| 642 | hsa-miR-671-5p | MIMAT0003880 |
| 643 | hsa-mir-6794 | MI0022639 |
| 644 | hsa-mir-6774 | MI0022619 |
| 645 | hsa-mir-4707 | MI0017340 |
| 646 | hsa-mir-4534 | MI0016901 |
| 647 | hsa-mir-4294 | MI0015827 |
| 648 | hsa-mir-6850 | MI0022696 |
| 649 | hsa-mir-6089-1 | MI0020366 |
| 650 | hsa-mir-6089-2 | MI0023563 |
| 651 | hsa-mir-671 | MI0003760 |
| 652 | isomiR example 1 of SEQ ID NO: 637 | — |
| 653 | isomiR example 2 of SEQ ID NO: 637 | — |
| 654 | isomiR example 1 of SEQ ID NO: 641 | — |
| 655 | isomiR example 2 of SEQ ID NO: 641 | — |
| 656 | isomiR example 1 of SEQ ID NO: 642 | — |
| 657 | isomiR example 2 of SEQ ID NO: 642 | — |

The present specification encompasses the contents described in the specifications and/or drawings of Japanese Patent Application Nos. 2014-123224 and 2015-071485 from which the present application claims priority.

### Advantageous Effects of Invention

According to the present invention, stomach cancer can be detected easily and in high accuracy.

For example, the presence or absence of stomach cancer in a patient(s) can be easily detected by using, as an indicator(s), the measurement values of several miRNAs in blood, serum, and/or plasma of the patient(s), which can be collected with limited invasiveness.

### BRIEF DESCRIPTION OF DRAWINGS

FIG. 1 This figure shows the relationship between the nucleotide sequences of hsa-miR-1225-3p represented by SEQ ID NO: 7 and hsa-miR-1225-5p represented by SEQ ID NO: 52, which are produced from a precursor hsa-mir-1225 represented by SEQ ID NO: 206.

FIG. 2 Left diagram: the measurement values of hsa-miR-4257 (SEQ ID NO: 1) in healthy subjects (100 persons) and stomach cancer patients (34 persons) selected as a training cohort were each plotted on the ordinate. The horizontal line in the diagram depicts a threshold (6.29) that was optimized by Fisher's discriminant analysis and discriminated between the two groups. Right diagram: the measurement values of hsa-miR-4257 (SEQ ID NO: 1) in healthy subjects (50 persons) and stomach cancer patients (16 persons) selected as a validation cohort were each plotted on the ordinate. The horizontal line in the diagram depicts the threshold (6.29) that was set in the training cohort and discriminated between the two groups.

FIG. 3 Left diagram: the measurement values of hsa-miR-4257 (SEQ ID NO: 1) in healthy subjects (100 persons, circles) and stomach cancer patients (34 persons, triangles) selected as a training cohort were each plotted on the abscissa against their measurement values of hsa-miR-6726-5p (SEQ ID NO: 2) on the ordinate. The line in the diagram depicts a discriminant function (0=0.83x+y−14.78) that was optimized by Fisher's discriminant analysis and discriminated between the two groups. Right diagram: the measurement values of hsa-miR-4257 (SEQ ID NO: 1) in healthy subjects (50 persons, circles) and stomach cancer patients (16 persons, triangles) selected as a validation cohort were each plotted on the abscissa against their measurement values of hsa-miR-6726-5p (SEQ ID NO: 2) on the ordinate. The line in the diagram depicts the threshold (0=0.83x+y−14.78) that was set for the training cohort and discriminated between the two groups.

FIG. 4 Upper diagram: a discriminant (2.51×hsa-miR-6781-5p−0.63×hsa-miR-4419b+0.98×hsa-miR-940+0.63×hsa-miR-4294−0.70×hsa-miR-6769b-5p+0.85×hsa-miR-1914-3p−37.81) was prepared by use of Fisher's discriminant analysis from the measurement values of hsa-miR-6781-5p (SEQ ID NO: 9), hsa-miR-204-3p (SEQ ID NO: 13), hsa-miR-3195 (SEQ ID NO: 143), hsa-miR-6769b-5p (SEQ ID NO: 155), hsa-miR-4665-5p (SEQ ID NO: 194), and hsa-miR-4294 (SEQ ID NO: 639) in 34 stomach cancer patients, 102 healthy subjects, 63 pancreatic cancer patients, 65 bile duct cancer patients, 35 colorectal cancer patients, 32 liver cancer patients, and 17 benign pancreaticobiliary disease patients selected as a training cohort, and discriminant scores obtained from the discriminant were plotted on the ordinate against the sample groups on the abscissa. The dotted line in the diagram depicts a discriminant boundary that offered a discriminant score of 0 and discriminated between the groups. Lower diagram: discriminant scores obtained from the discriminant prepared from the training cohort as to the measurement values of hsa-miR-6781-5p (SEQ ID NO: 9), hsa-miR-204-3p (SEQ ID NO: 13), hsa-miR-3195 (SEQ ID NO: 143), hsa-miR-6769b-5p (SEQ ID NO: 155), hsa-miR-4665-5p (SEQ ID NO: 194), and hsa-miR-4294 (SEQ ID NO: 639) in 16 stomach cancer patients, 48 healthy subjects, 37 pancreatic cancer patients, 33 bile duct cancer patients, 15 colorectal cancer patients, 20 liver cancer patients, and 4 benign pancreaticobiliary disease patients selected as a validation cohort were plotted on the ordinate against the sample groups on the abscissa. The dotted line in the diagram depicts the discriminant boundary that offered a discriminant score of 0 and discriminated between both of the groups.

### DESCRIPTION OF EMBODIMENTS

Hereinafter, the present invention will be further described in detail.

### 1. Target Nucleic Acid for Stomach Cancer

Primary target nucleic acids as a stomach cancer marker(s) for detecting the presence and/or absence of stomach cancer or stomach cancer cells using the nucleic acid probe(s) or the primer(s) for the detection of stomach cancer defined above according to the present invention comprises at least one or more miRNAs selected from the group consisting of hsa-miR-4257, hsa-miR-6726-5p, hsa-miR-1343-3p, hsa-miR-1247-3p, hsa-miR-6787-5p, hsa-miR-6875-5p, hsa-miR-1225-3p, hsa-miR-8063, hsa-miR-6781-5p, hsa-miR-4746-3p, hsa-miR-1908-5p, hsa-miR-6756-5p, hsa-miR-204-3p, hsa-miR-4651, hsa-miR-6757-5p, hsa-miR-6825-5p, hsa-miR-7108-5p, hsa-miR-4792, hsa-miR-7641, hsa-miR-3188, hsa-miR-3131, hsa-miR-6780b-5p, hsa-miR-8069, hsa-miR-6840-3p, hsa-miR-8072, hsa-miR-1233-5p, hsa-miR-6887-5p, hsa-miR-1231, hsa-miR-5572, hsa-miR-6738-5p, hsa-miR-6784-5p, hsa-miR-6791-5p, hsa-miR-6749-5p, hsa-miR-6741-5p, hsa-miR-128-1-5p, hsa-miR-4419b, hsa-miR-6746-5p, hsa-miR-3184-5p, hsa-miR-3679-5p, hsa-miR-7110-5p, hsa-miR-4516, hsa-miR-6717-5p, hsa-miR-6826-5p, hsa-miR-4433b-3p, hsa-miR-3679-3p, hsa-miR-3135b, hsa-miR-3622a-5p, hsa-miR-711, hsa-miR-4467, hsa-miR-6857-5p, hsa-miR-6515-3p, hsa-miR-1225-5p, hsa-miR-187-5p, hsa-miR-3185, hsa-miR-642b-3p, hsa-miR-1249, hsa-miR-744-5p, hsa-miR-4442, hsa-miR-1228-3p, hsa-miR-939-5p, hsa-miR-6845-5p, hsa-miR-887-3p, hsa-miR-7845-5p, hsa-miR-6729-5p, hsa-miR-4632-5p, hsa-miR-615-5p, hsa-miR-6724-5p, hsa-miR-4728-5p, hsa-miR-6732-5p, hsa-miR-6816-5p, hsa-miR-4695-5p, hsa-miR-6088, hsa-miR-7975, hsa-miR-3197, hsa-miR-6125, hsa-miR-4433-3p, hsa-miR-6727-5p, hsa-miR-4706, hsa-miR-7847-3p, hsa-miR-6805-3p, hsa-miR-6766-3p, hsa-miR-1913, hsa-miR-4649-5p, hsa-miR-602, hsa-miR-3663-3p, hsa-miR-6893-5p, hsa-miR-6861-5p, hsa-miR-4449, hsa-miR-6842-5p, hsa-miR-4454, hsa-miR-5195-3p, hsa-miR-663b, hsa-miR-6765-5p, hsa-miR-4513, hsa-miR-614, hsa-miR-6785-5p, hsa-miR-6777-5p, hsa-miR-940, hsa-miR-4741, hsa-miR-6870-5p, hsa-miR-6131, hsa-miR-150-3p, hsa-miR-4707-5p, hsa-miR-1915-3p, hsa-miR-3937, hsa-miR-937-5p, hsa-miR-4443, hsa-miR-1914-3p, hsa-miR-3620-5p, hsa-miR-1268b, hsa-miR-1227-5p, hsa-miR-6880-5p, hsa-miR-4417, hsa-miR-6802-5p, hsa-miR-6769a-5p, hsa-miR-663a, hsa-miR-6721-5p, hsa-miR-4532, hsa-miR-7977, hsa-miR-92b-5p, hsa-miR-371a-5p, hsa-miR-6126, hsa-miR-4734, hsa-miR-4665-3p, hsa-miR-423-5p, hsa-miR-1469, hsa-miR-4675, hsa-miR-1915-5p, hsa-miR-6716-5p, hsa-miR-718, hsa-miR-4281, hsa-miR-6820-5p, hsa-miR-6795-5p, hsa-miR-6779-5p, hsa-miR-7109-5p, hsa-miR-6798-5p, hsa-miR-4648, hsa-miR-8059, hsa-miR-6765-3p, hsa-miR-6132, hsa-miR-4492, hsa-miR-7107-5p, hsa-miR-3195, hsa-miR-3180, hsa-miR-296-3p, hsa-miR-564, hsa-miR-1268a, hsa-miR-6848-5p, hsa-miR-762, hsa-miR-2861, hsa-miR-1203, hsa-miR-1260b, hsa-miR-4476, hsa-miR-6885-5p, hsa-miR-6769b-5p, hsa-miR-23b-3p, hsa-miR-1343-5p, hsa-miR-3621, hsa-miR-4688, hsa-miR-4286, hsa-miR-4640-5p, hsa-miR-4739, hsa-miR-1260a, hsa-miR-4276, hsa-miR-7106, hsa-miR-6794-5p, hsa-miR-6774-5p, hsa-miR-4707-3p, hsa-miR-4534, hsa-miR-4294, hsa-miR-6850-5p, hsa-miR-6089 and hsa-miR-671-5p. Furthermore, at least one or more miRNAs selected from the group consisting of other stomach cancer markers that can be combined with these miRNAs, i.e., hsa-miR-128-2-5p, hsa-miR-125a-3p, hsa-miR-92a-2-5p, and hsa-miR-486-3p can also be preferably used as a target nucleic acid. Moreover, at least one or more miRNAs selected from the group consisting of other stomach cancer markers that can be combined with these miRNAs, i.e., hsa-miR-3196, hsa-miR-211-3p, hsa-miR-4271, hsa-miR-6851-5p, hsa-miR-149-3p, hsa-miR-4667-5p, hsa-miR-135a-3p, hsa-miR-4486, hsa-miR-4697-5p, hsa-miR-4725-3p, hsa-miR-6510-5p, hsa-miR-5001-5p, hsa-miR-4673, hsa-miR-4466, hsa-miR-23a-3p, hsa-miR-3656, hsa-miR-6782-5p, hsa-miR-4689, hsa-miR-451a, hsa-miR-4446-3p, hsa-miR-3180-3p, hsa-miR-642a-3p, hsa-miR-6889-5p, hsa-miR-3178, hsa-miR-4665-5p, hsa-miR-6722-3p, hsa-miR-30c-1-3p, hsa-miR-4507, hsa-miR-3141 and hsa-miR-1199-5p can also be preferably used as a target nucleic acid(s).

These miRNAs include, for example, a human gene comprising a nucleotide sequence represented by any of SEQ ID NOs: 1 to 199 and 635 to 642 (i.e., hsa-miR-4257, hsa-miR-6726-5p, hsa-miR-1343-3p, hsa-miR-1247-3p, hsa-miR-6787-5p, hsa-miR-6875-5p, hsa-miR-1225-3p, hsa-miR-8063, hsa-miR-6781-5p, hsa-miR-4746-3p, hsa-miR-1908-5p, hsa-miR-6756-5p, hsa-miR-204-3p, hsa-miR-4651, hsa-miR-6757-5p, hsa-miR-6825-5p, hsa-miR-7108-5p, hsa-miR-4792, hsa-miR-7641, hsa-miR-3188, hsa-miR-3131, hsa-miR-6780b-5p, hsa-miR-8069, hsa-miR-6840-3p, hsa-miR-8072, hsa-miR-1233-5p, hsa-miR-6887-5p, hsa-miR-1231, hsa-miR-5572, hsa-miR-6738-5p, hsa-miR-6784-5p, hsa-miR-6791-5p, hsa-miR-6749-5p, hsa-miR-6741-5p, hsa-miR-128-1-5p, hsa-miR-4419b, hsa-miR-6746-5p, hsa-miR-3184-5p, hsa-miR-3679-5p, hsa-miR-7110-5p, hsa-miR-4516, hsa-miR-6717-5p, hsa-miR-6826-5p, hsa-miR-4433b-3p, hsa-miR-3679-3p, hsa-miR-3135b, hsa-miR-3622a-5p, hsa-miR-711, hsa-miR-4467, hsa-miR-6857-5p, hsa-miR-6515-3p, hsa-miR-1225-5p, hsa-miR-187-5p, hsa-miR-3185, hsa-miR-642b-3p, hsa-miR-1249, hsa-miR-744-5p, hsa-miR-4442, hsa-miR-1228-3p, hsa-miR-939-5p, hsa-miR-6845-5p, hsa-miR-887-3p, hsa-miR-7845-5p, hsa-miR-6729-5p, hsa-miR-4632-5p, hsa-miR-615-5p, hsa-miR-6724-5p, hsa-miR-4728-5p, hsa-miR-6732-5p, hsa-miR-6816-5p, hsa-miR-4695-5p, hsa-miR-6088, hsa-miR-7975, hsa-miR-3197, hsa-miR-6125, hsa-miR-4433-3p, hsa-miR-6727-5p, hsa-miR-4706, hsa-miR-7847-3p, hsa-miR-6805-3p, hsa-miR-6766-3p, hsa-miR-1913, hsa-miR-4649-5p, hsa-miR-602, hsa-miR-3663-3p, hsa-miR-6893-5p, hsa-miR-6861-5p, hsa-miR-4449, hsa-miR-6842-5p, hsa-miR-4454, hsa-miR-5195-3p, hsa-miR-663b, hsa-miR-6765-5p, hsa-miR-4513, hsa-miR-614, hsa-miR-6785-5p, hsa-miR-6777-5p, hsa-miR-940, hsa-miR-4741, hsa-miR-6870-5p, hsa-miR-6131, hsa-miR-150-3p, hsa-miR-4707-5p, hsa-miR-1915-3p, hsa-miR-3937, hsa-miR-937-5p, hsa-miR-4443, hsa-miR-1914-3p, hsa-miR-3620-5p, hsa-miR-1268b, hsa-miR-1227-5p, hsa-miR-6880-5p, hsa-miR-4417, hsa-miR-6802-5p, hsa-miR-6769a-5p, hsa-miR-663a, hsa-miR-6721-5p, hsa-miR-4532, hsa-miR-7977, hsa-miR-92b-5p, hsa-miR-371a-5p, hsa-miR-6126, hsa-miR-4734, hsa-miR-4665-3p, hsa-miR-423-5p, hsa-miR-1469, hsa-miR-4675, hsa-miR-1915-5p, hsa-miR-6716-5p, hsa-miR-718, hsa-miR-4281, hsa-miR-6820-5p, hsa-miR-6795-5p, hsa-miR-6779-5p, hsa-miR-7109-5p, hsa-miR-6798-5p, hsa-miR-4648, hsa-miR-8059, hsa-miR-6765-3p, hsa-miR-6132, hsa-miR-4492, hsa-miR-7107-5p, hsa-miR-3195, hsa-miR-3180, hsa-miR-296-3p, hsa-miR-564, hsa-miR-1268a, hsa-miR-6848-5p, hsa-miR-762, hsa-miR-2861, hsa-miR-1203, hsa-miR-1260b, hsa-miR-4476, hsa-miR-6885-5p, hsa-miR-6769b-5p, hsa-miR-23b-3p, hsa-miR-1343-5p, hsa-miR-3621, hsa-miR-4688, hsa-miR-4286, hsa-miR-4640-5p, hsa-miR-4739, hsa-miR-1260a, hsa-miR-4276, hsa-miR-7106, hsa-miR-6794-5p, hsa-miR-6774-5p, hsa-miR-4707-3p, hsa-miR-4534, hsa-miR-4294, hsa-miR-6850-5p, hsa-miR-6089, hsa-miR-671-5p, hsa-miR-128-2-5p, hsa-miR-125a-3p, hsa-miR-92a-2-5p, hsa-miR-486-3p, hsa-miR-3196, hsa-miR-211-3p, hsa-miR-4271, hsa-miR-6851-5p, hsa-miR-149-3p, hsa-miR-4667-5p, hsa-miR-135a-3p, hsa-miR-4486, hsa-miR-4697-5p, hsa-miR-4725-3p, hsa-miR-6510-5p, hsa-miR-5001-5p, hsa-miR-4673, hsa-miR-4466, hsa-miR-23a-3p, hsa-miR-3656, hsa-miR-6782-5p, hsa-miR-4689, hsa-miR-451a, hsa-miR-4446-3p, hsa-miR-3180-3p, hsa-miR-642a-3p, hsa-miR-6889-5p, hsa-miR-3178, hsa-miR-4665-5p, hsa-miR-6722-3p, hsa-miR-30c-1-3p, hsa-miR-4507, hsa-miR-3141 and hsa-miR-1199-5p, respectively), a congener thereof, a transcript thereof, and a variant or a derivative thereof. In this context, the gene, the congener, the transcript, the variant, and the derivative are as defined above.

The target nucleic acid is preferably a human gene comprising a nucleotide sequence represented by any of SEQ ID NOs: 1 to 657 or a transcript thereof, more preferably the transcript, i.e., a miRNA or its precursor RNA (pri-miRNA or pre-miRNA).

The first target gene is the hsa-miR-4257 gene, a congener thereof, a transcript thereof, or a variant or a derivative thereof. None of the previously known reports show that change in the expression of the gene or the transcript thereof can serve as a marker for stomach cancer.

The second target gene is the hsa-miR-6726-5p gene, a congener thereof, a transcript thereof, or a variant or a derivative thereof. None of the previously known reports show that change in the expression of the gene or the transcript thereof can serve as a marker for stomach cancer.

The third target gene is the hsa-miR-1343-3p gene, a congener thereof, a transcript thereof, or a variant or a derivative thereof. None of the previously known reports show that change in the expression of the gene or the transcript thereof can serve as a marker for stomach cancer.

The fourth target gene is the hsa-miR-1247-3p gene, a congener thereof, a transcript thereof, or a variant or a derivative thereof. None of the previously known reports show that change in the expression of the gene or the transcript thereof can serve as a marker for stomach cancer.

The fifth target gene is the hsa-miR-6787-5p gene, a congener thereof, a transcript thereof, or a variant or a derivative thereof. None of the previously known reports show that change in the expression of the gene or the transcript thereof can serve as a marker for stomach cancer.

The sixth target gene is the hsa-miR-6875-5p gene, a congener thereof, a transcript thereof, or a variant or a derivative thereof. None of the previously known reports show that change in the expression of the gene or the transcript thereof can serve as a marker for stomach cancer.

The seventh target gene is the hsa-miR-1225-3p gene, a congener thereof, a transcript thereof, or a variant or a derivative thereof. None of the previously known reports show that change in the expression of the gene or the transcript thereof can serve as a marker for stomach cancer.

The eighth target gene is the hsa-miR-8063 gene, a congener thereof, a transcript thereof, or a variant or a derivative thereof. None of the previously known reports show that change in the expression of the gene or the transcript thereof can serve as a marker for stomach cancer.

The ninth target gene is the hsa-miR-6781-5p gene, a congener thereof, a transcript thereof, or a variant or a derivative thereof. None of the previously known reports show that change in the expression of the gene or the transcript thereof can serve as a marker for stomach cancer.

The 10th target gene is the hsa-miR-4746-3p gene, a congener thereof, a transcript thereof, or a variant or a derivative thereof. None of the previously known reports show that change in the expression of the gene or the transcript thereof can serve as a marker for stomach cancer.

The 11th target gene is the hsa-miR-1908-5p gene, a congener thereof, a transcript thereof, or a variant or a derivative thereof. None of the previously known reports show that change in the expression of the gene or the transcript thereof can serve as a marker for stomach cancer.

The 12th target gene is the hsa-miR-6756-5p gene, a congener thereof, a transcript thereof, or a variant or a derivative thereof. None of the previously known reports show that change in the expression of the gene or the transcript thereof can serve as a marker for stomach cancer.

The 13th target gene is the hsa-miR-204-3p gene, a congener thereof, a transcript thereof, or a variant or a derivative thereof. None of the previously known reports show that change in the expression of the gene or the transcript thereof can serve as a marker for stomach cancer.

The 14th target gene is the hsa-miR-4651 gene, a congener thereof, a transcript thereof, or a variant or a derivative thereof. None of the previously known reports show that change in the expression of the gene or the transcript thereof can serve as a marker for stomach cancer.

The 15th target gene is the hsa-miR-6757-5p gene, a congener thereof, a transcript thereof, or a variant or a derivative thereof. None of the previously known reports show that change in the expression of the gene or the transcript thereof can serve as a marker for stomach cancer.

The 16th target gene is the hsa-miR-6825-5p gene, a congener thereof, a transcript thereof, or a variant or a derivative thereof. None of the previously known reports show that change in the expression of the gene or the transcript thereof can serve as a marker for stomach cancer.

The 17th target gene is the hsa-miR-7108-5p gene, a congener thereof, a transcript thereof, or a variant or a derivative thereof. None of the previously known reports show that change in the expression of the gene or the transcript thereof can serve as a marker for stomach cancer.

The 18th target gene is the hsa-miR-4792 gene, a congener thereof, a transcript thereof, or a variant or a derivative thereof. None of the previously known reports show that change in the expression of the gene or the transcript thereof can serve as a marker for stomach cancer.

The 19th target gene is the hsa-miR-7641 gene, a congener thereof, a transcript thereof, or a variant or a derivative thereof. None of the previously known reports show that change in the expression of the gene or the transcript thereof can serve as a marker for stomach cancer.

The 20th target gene is the hsa-miR-3188 gene, a congener thereof, a transcript thereof, or a variant or a derivative thereof. None of the previously known reports show that change in the expression of the gene or the transcript thereof can serve as a marker for stomach cancer.

The 21st target gene is the hsa-miR-3131 gene, a congener thereof, a transcript thereof, or a variant or a derivative thereof. None of the previously known reports show that change in the expression of the gene or the transcript thereof can serve as a marker for stomach cancer.

The 22nd target gene is the hsa-miR-6780b-5p gene, a congener thereof, a transcript thereof, or a variant or a derivative thereof. None of the previously known reports show that change in the expression of the gene or the transcript thereof can serve as a marker for stomach cancer.

The 23rd target gene is the hsa-miR-8069 gene, a congener thereof, a transcript thereof, or a variant or a derivative thereof. None of the previously known reports show that change in the expression of the gene or the transcript thereof can serve as a marker for stomach cancer.

The 24th target gene is the hsa-miR-6840-3p gene, a congener thereof, a transcript thereof, or a variant or a derivative thereof. None of the previously known reports show that change in the expression of the gene or the transcript thereof can serve as a marker for stomach cancer.

The 25th target gene is the hsa-miR-8072 gene, a congener thereof, a transcript thereof, or a variant or a derivative thereof. None of the previously known reports show that change in the expression of the gene or the transcript thereof can serve as a marker for stomach cancer.

The 26th target gene is the hsa-miR-1233-5p gene, a congener thereof, a transcript thereof, or a variant or a derivative thereof. None of the previously known reports show that change in the expression of the gene or the transcript thereof can serve as a marker for stomach cancer.

The 27th target gene is the hsa-miR-6887-5p gene, a congener thereof, a transcript thereof, or a variant or a derivative thereof. None of the previously known reports show that change in the expression of the gene or the transcript thereof can serve as a marker for stomach cancer.

The 28th target gene is the hsa-miR-1231 gene, a congener thereof, a transcript thereof, or a variant or a derivative thereof. None of the previously known reports show that change in the expression of the gene or the transcript thereof can serve as a marker for stomach cancer.

The 29th target gene is the hsa-miR-5572 gene, a congener thereof, a transcript thereof, or a variant or a derivative thereof. None of the previously known reports show that change in the expression of the gene or the transcript thereof can serve as a marker for stomach cancer.

The 30th target gene is the hsa-miR-6738-5p gene, a congener thereof, a transcript thereof, or a variant or a derivative thereof. None of the previously known reports show that change in the expression of the gene or the transcript thereof can serve as a marker for stomach cancer.

The 31st target gene is the hsa-miR-6784-5p gene, a congener thereof, a transcript thereof, or a variant or a derivative thereof. None of the previously known reports show that change in the expression of the gene or the transcript thereof can serve as a marker for stomach cancer.

The 32nd target gene is the hsa-miR-6791-5p gene, a congener thereof, a transcript thereof, or a variant or a derivative thereof. None of the previously known reports show that change in the expression of the gene or the transcript thereof can serve as a marker for stomach cancer.

The 33rd target gene is the hsa-miR-6749-5p gene, a congener thereof, a transcript thereof, or a variant or a derivative thereof. None of the previously known reports show that change in the expression of the gene or the transcript thereof can serve as a marker for stomach cancer.

The 34th target gene is the hsa-miR-6741-5p gene, a congener thereof, a transcript thereof, or a variant or a derivative thereof. None of the previously known reports show that change in the expression of the gene or the transcript thereof can serve as a marker for stomach cancer.

The 35th target gene is the hsa-miR-128-1-5p gene, a congener thereof, a transcript thereof, or a variant or a derivative thereof. None of the previously known reports show that change in the expression of the gene or the transcript thereof can serve as a marker for stomach cancer.

The 36th target gene is the hsa-miR-4419b gene, a congener thereof, a transcript thereof, or a variant or a derivative thereof. None of the previously known reports show that change in the expression of the gene or the transcript thereof can serve as a marker for stomach cancer.

The 37th target gene is the hsa-miR-6746-5p gene, a congener thereof, a transcript thereof, or a variant or a derivative thereof. None of the previously known reports show that change in the expression of the gene or the transcript thereof can serve as a marker for stomach cancer.

The 38th target gene is the hsa-miR-3184-5p gene, a congener thereof, a transcript thereof, or a variant or a derivative thereof. None of the previously known reports show that change in the expression of the gene or the transcript thereof can serve as a marker for stomach cancer.

The 39th target gene is the hsa-miR-3679-5p gene, a congener thereof, a transcript thereof, or a variant or a derivative thereof. None of the previously known reports show that change in the expression of the gene or the transcript thereof can serve as a marker for stomach cancer.

The 40th target gene is the hsa-miR-7110-5p gene, a congener thereof, a transcript thereof, or a variant or a derivative thereof. None of the previously known reports show that change in the expression of the gene or the transcript thereof can serve as a marker for stomach cancer.

The 41st target gene is the hsa-miR-4516 gene, a congener thereof, a transcript thereof, or a variant or a derivative thereof. None of the previously known reports show that change in the expression of the gene or the transcript thereof can serve as a marker for stomach cancer.

The 42nd target gene is the hsa-miR-6717-5p gene, a congener thereof, a transcript thereof, or a variant or a derivative thereof. None of the previously known reports show that change in the expression of the gene or the transcript thereof can serve as a marker for stomach cancer.

The 43rd target gene is the hsa-miR-6826-5p gene, a congener thereof, a transcript thereof, or a variant or a derivative thereof. None of the previously known reports show that change in the expression of the gene or the transcript thereof can serve as a marker for stomach cancer.

The 44th target gene is the hsa-miR-4433b-3p gene, a congener thereof, a transcript thereof, or a variant or a derivative thereof. None of the previously known reports show that change in the expression of the gene or the transcript thereof can serve as a marker for stomach cancer.

The 45th target gene is the hsa-miR-3679-3p gene, a congener thereof, a transcript thereof, or a variant or a derivative thereof. None of the previously known reports show that change in the expression of the gene or the transcript thereof can serve as a marker for stomach cancer.

The 46th target gene is the hsa-miR-3135b gene, a congener thereof, a transcript thereof, or a variant or a derivative thereof. None of the previously known reports show that change in the expression of the gene or the transcript thereof can serve as a marker for stomach cancer.

The 47th target gene is the hsa-miR-3622a-5p gene, a congener thereof, a transcript thereof, or a variant or a derivative thereof. None of the previously known reports show that change in the expression of the gene or the transcript thereof can serve as a marker for stomach cancer.

The 48th target gene is the hsa-miR-711 gene, a congener thereof, a transcript thereof, or a variant or a derivative thereof. None of the previously known reports show that change in the expression of the gene or the transcript thereof can serve as a marker for stomach cancer.

The 49th target gene is the hsa-miR-4467 gene, a congener thereof, a transcript thereof, or a variant or a derivative thereof. None of the previously known reports show that change in the expression of the gene or the transcript thereof can serve as a marker for stomach cancer.

The 50th target gene is the hsa-miR-6857-5p gene, a congener thereof, a transcript thereof, or a variant or a derivative thereof. None of the previously known reports show that change in the expression of the gene or the transcript thereof can serve as a marker for stomach cancer.

The 51st target gene is the hsa-miR-6515-3p gene, a congener thereof, a transcript thereof, or a variant or a derivative thereof. None of the previously known reports show that change in the expression of the gene or the transcript thereof can serve as a marker for stomach cancer.

The 52nd target gene is the hsa-miR-1225-5p gene, a congener thereof, a transcript thereof, or a variant or a derivative thereof. None of the previously known reports show that change in the expression of the gene or the transcript thereof can serve as a marker for stomach cancer.

The 53rd target gene is the hsa-miR-187-5p gene, a congener thereof, a transcript thereof, or a variant or a derivative thereof. None of the previously known reports show that change in the expression of the gene or the transcript thereof can serve as a marker for stomach cancer.

The 54th target gene is the hsa-miR-3185 gene, a congener thereof, a transcript thereof, or a variant or a derivative thereof. None of the previously known reports show that change in the expression of the gene or the transcript thereof can serve as a marker for stomach cancer.

The 55th target gene is the hsa-miR-642b-3p gene, a congener thereof, a transcript thereof, or a variant or a derivative thereof. None of the previously known reports show that change in the expression of the gene or the transcript thereof can serve as a marker for stomach cancer.

The 56th target gene is the hsa-miR-1249 gene, a congener thereof, a transcript thereof, or a variant or a derivative thereof. None of the previously known reports show that change in the expression of the gene or the transcript thereof can serve as a marker for stomach cancer.

The 57th target gene is the hsa-miR-744-5p gene, a congener thereof, a transcript thereof, or a variant or a derivative thereof. None of the previously known reports show that change in the expression of the gene or the transcript thereof can serve as a marker for stomach cancer.

The 58th target gene is the hsa-miR-4442 gene, a congener thereof, a transcript thereof, or a variant or a derivative thereof. None of the previously known reports show that change in the expression of the gene or the transcript thereof can serve as a marker for stomach cancer.

The 59th target gene is the hsa-miR-1228-3p gene, a congener thereof, a transcript thereof, or a variant or a derivative thereof. None of the previously known reports show that change in the expression of the gene or the transcript thereof can serve as a marker for stomach cancer.

The 60th target gene is the hsa-miR-939-5p gene, a congener thereof, a transcript thereof, or a variant or a derivative thereof. None of the previously known reports show that change in the expression of the gene or the transcript thereof can serve as a marker for stomach cancer.

The 61st target gene is the hsa-miR-6845-5p gene, a congener thereof, a transcript thereof, or a variant or a derivative thereof. None of the previously known reports show that change in the expression of the gene or the transcript thereof can serve as a marker for stomach cancer.

The 62nd target gene is the hsa-miR-887-3p gene, a congener thereof, a transcript thereof, or a variant or a derivative thereof. None of the previously known reports show that change in the expression of the gene or the transcript thereof can serve as a marker for stomach cancer.

The 63rd target gene is the hsa-miR-7845-5p gene, a congener thereof, a transcript thereof, or a variant or a derivative thereof. None of the previously known reports show that change in the expression of the gene or the transcript thereof can serve as a marker for stomach cancer.

The 64th target gene is the hsa-miR-6729-5p gene, a congener thereof, a transcript thereof, or a variant or a derivative thereof. None of the previously known reports show that change in the expression of the gene or the transcript thereof can serve as a marker for stomach cancer.

The 65th target gene is the hsa-miR-4632-5p gene, a congener thereof, a transcript thereof, or a variant or a derivative thereof. None of the previously known reports show that change in the expression of the gene or the transcript thereof can serve as a marker for stomach cancer.

The 66th target gene is the hsa-miR-615-5p gene, a congener thereof, a transcript thereof, or a variant or a derivative thereof. None of the previously known reports show that change in the expression of the gene or the transcript thereof can serve as a marker for stomach cancer.

The 67th target gene is the hsa-miR-6724-5p gene, a congener thereof, a transcript thereof, or a variant or a derivative thereof. None of the previously known reports show that change in the expression of the gene or the transcript thereof can serve as a marker for stomach cancer.

The 68th target gene is the hsa-miR-4728-5p gene, a congener thereof, a transcript thereof, or a variant or a derivative thereof. None of the previously known reports show that change in the expression of the gene or the transcript thereof can serve as a marker for stomach cancer.

The 69th target gene is the hsa-miR-6732-5p gene, a congener thereof, a transcript thereof, or a variant or a derivative thereof. None of the previously known reports show that change in the expression of the gene or the transcript thereof can serve as a marker for stomach cancer.

The 70th target gene is the hsa-miR-6816-5p gene, a congener thereof, a transcript thereof, or a variant or a derivative thereof. None of the previously known reports show that change in the expression of the gene or the transcript thereof can serve as a marker for stomach cancer.

The 71st target gene is the hsa-miR-4695-5p gene, a congener thereof, a transcript thereof, or a variant or a derivative thereof. None of the previously known reports show that change in the expression of the gene or the transcript thereof can serve as a marker for stomach cancer.

The 72nd target gene is the hsa-miR-6088 gene, a congener thereof, a transcript thereof, or a variant or a derivative thereof. None of the previously known reports show that change in the expression of the gene or the transcript thereof can serve as a marker for stomach cancer.

The 73rd target gene is the hsa-miR-7975 gene, a congener thereof, a transcript thereof, or a variant or a derivative thereof. None of the previously known reports show that change in the expression of the gene or the transcript thereof can serve as a marker for stomach cancer.

The 74th target gene is the hsa-miR-3197 gene, a congener thereof, a transcript thereof, or a variant or a derivative thereof. None of the previously known reports show that change in the expression of the gene or the transcript thereof can serve as a marker for stomach cancer.

The 75th target gene is the hsa-miR-6125 gene, a congener thereof, a transcript thereof, or a variant or a derivative thereof. None of the previously known reports show that change in the expression of the gene or the transcript thereof can serve as a marker for stomach cancer.

The 76th target gene is the hsa-miR-4433-3p gene, a congener thereof, a transcript thereof, or a variant or a derivative thereof. None of the previously known reports show that change in the expression of the gene or the transcript thereof can serve as a marker for stomach cancer.

The 77th target gene is the hsa-miR-6727-5p gene, a congener thereof, a transcript thereof, or a variant or a derivative thereof. None of the previously known reports show that change in the expression of the gene or the transcript thereof can serve as a marker for stomach cancer.

The 78th target gene is the hsa-miR-4706 gene, a congener thereof, a transcript thereof, or a variant or a derivative thereof. None of the previously known reports show that change in the expression of the gene or the transcript thereof can serve as a marker for stomach cancer.

The 79th target gene is the hsa-miR-7847-3p gene, a congener thereof, a transcript thereof, or a variant or a derivative thereof. None of the previously known reports show that change in the expression of the gene or the transcript thereof can serve as a marker for stomach cancer.

The 80th target gene is the hsa-miR-6805-3p gene, a congener thereof, a transcript thereof, or a variant or a derivative thereof. None of the previously known reports show that change in the expression of the gene or the transcript thereof can serve as a marker for stomach cancer.

The 81st target gene is the hsa-miR-6766-3p gene, a congener thereof, a transcript thereof, or a variant or a derivative thereof. None of the previously known reports show that change in the expression of the gene or the transcript thereof can serve as a marker for stomach cancer.

The 82nd target gene is the hsa-miR-1913 gene, a congener thereof, a transcript thereof, or a variant or a derivative thereof. None of the previously known reports show that change in the expression of the gene or the transcript thereof can serve as a marker for stomach cancer.

The 83rd target gene is the hsa-miR-4649-5p gene, a congener thereof, a transcript thereof, or a variant or a derivative thereof. None of the previously known reports show that change in the expression of the gene or the transcript thereof can serve as a marker for stomach cancer.

The 84th target gene is the hsa-miR-602 gene, a congener thereof, a transcript thereof, or a variant or a derivative thereof. None of the previously known reports show that change in the expression of the gene or the transcript thereof can serve as a marker for stomach cancer.

The 85th target gene is the hsa-miR-3663-3p gene, a congener thereof, a transcript thereof, or a variant or a derivative thereof. None of the previously known reports show that change in the expression of the gene or the transcript thereof can serve as a marker for stomach cancer.

The 86th target gene is the hsa-miR-6893-5p gene, a congener thereof, a transcript thereof, or a variant or a derivative thereof. None of the previously known reports show that change in the expression of the gene or the transcript thereof can serve as a marker for stomach cancer.

The 87th target gene is the hsa-miR-6861-5p gene, a congener thereof, a transcript thereof, or a variant or a derivative thereof. None of the previously known reports show that change in the expression of the gene or the transcript thereof can serve as a marker for stomach cancer.

The 88th target gene is the hsa-miR-4449 gene, a congener thereof, a transcript thereof, or a variant or a derivative thereof. None of the previously known reports show that change in the expression of the gene or the transcript thereof can serve as a marker for stomach cancer.

The 89th target gene is the hsa-miR-6842-5p gene, a congener thereof, a transcript thereof, or a variant or a derivative thereof. None of the previously known reports show that change in the expression of the gene or the transcript thereof can serve as a marker for stomach cancer.

The 90th target gene is the hsa-miR-4454 gene, a congener thereof, a transcript thereof, or a variant or a derivative thereof. None of the previously known reports show that change in the expression of the gene or the transcript thereof can serve as a marker for stomach cancer.

The 91st target gene is the hsa-miR-5195-3p gene, a congener thereof, a transcript thereof, or a variant or a derivative thereof. None of the previously known reports show that change in the expression of the gene or the transcript thereof can serve as a marker for stomach cancer.

The 92nd target gene is the hsa-miR-663b gene, a congener thereof, a transcript thereof, or a variant or a derivative thereof. None of the previously known reports show that change in the expression of the gene or the transcript thereof can serve as a marker for stomach cancer.

The 93rd target gene is the hsa-miR-6765-5p gene, a congener thereof, a transcript thereof, or a variant or a derivative thereof. None of the previously known reports show that change in the expression of the gene or the transcript thereof can serve as a marker for stomach cancer.

The 94th target gene is the hsa-miR-4513 gene, a congener thereof, a transcript thereof, or a variant or a derivative thereof. None of the previously known reports show that change in the expression of the gene or the transcript thereof can serve as a marker for stomach cancer.

The 95th target gene is the hsa-miR-614 gene, a congener thereof, a transcript thereof, or a variant or a derivative thereof. None of the previously known reports show that change in the expression of the gene or the transcript thereof can serve as a marker for stomach cancer.

The 96th target gene is the hsa-miR-6785-5p gene, a congener thereof, a transcript thereof, or a variant or a derivative thereof. None of the previously known reports show that change in the expression of the gene or the transcript thereof can serve as a marker for stomach cancer.

The 97th target gene is the hsa-miR-6777-5p gene, a congener thereof, a transcript thereof, or a variant or a derivative thereof. None of the previously known reports show that change in the expression of the gene or the transcript thereof can serve as a marker for stomach cancer.

The 98th target gene is the hsa-miR-940 gene, a congener thereof, a transcript thereof, or a variant or a derivative thereof. None of the previously known reports show that change in the expression of the gene or the transcript thereof can serve as a marker for stomach cancer.

The 99th target gene is the hsa-miR-4741 gene, a congener thereof, a transcript thereof, or a variant or a derivative thereof. None of the previously known reports show that change in the expression of the gene or the transcript thereof can serve as a marker for stomach cancer.

The 100th target gene is the hsa-miR-6870-5p gene, a congener thereof, a transcript thereof, or a variant or a derivative thereof. None of the previously known reports show that change in the expression of the gene or the transcript thereof can serve as a marker for stomach cancer.

The 101st target gene is the hsa-miR-6131 gene, a congener thereof, a transcript thereof, or a variant or a derivative thereof. None of the previously known reports show that change in the expression of the gene or the transcript thereof can serve as a marker for stomach cancer.

The 102nd target gene is the hsa-miR-150-3p gene, a congener thereof, a transcript thereof, or a variant or a derivative thereof. None of the previously known reports show that change in the expression of the gene or the transcript thereof can serve as a marker for stomach cancer.

The 103rd target gene is the hsa-miR-4707-5p gene, a congener thereof, a transcript thereof, or a variant or a derivative thereof. None of the previously known reports show that change in the expression of the gene or the transcript thereof can serve as a marker for stomach cancer.

The 104th target gene is the hsa-miR-1915-3p gene, a congener thereof, a transcript thereof, or a variant or a derivative thereof. None of the previously known reports show that change in the expression of the gene or the transcript thereof can serve as a marker for stomach cancer.

The 105th target gene is the hsa-miR-3937 gene, a congener thereof, a transcript thereof, or a variant or a derivative thereof. None of the previously known reports show that change in the expression of the gene or the transcript thereof can serve as a marker for stomach cancer.

The 106th target gene is the hsa-miR-937-5p gene, a congener thereof, a transcript thereof, or a variant or a derivative thereof. None of the previously known reports show that change in the expression of the gene or the transcript thereof can serve as a marker for stomach cancer.

The 107th target gene is the hsa-miR-4443 gene, a congener thereof, a transcript thereof, or a variant or a derivative thereof. None of the previously known reports show that change in the expression of the gene or the transcript thereof can serve as a marker for stomach cancer.

The 108th target gene is the hsa-miR-1914-3p gene, a congener thereof, a transcript thereof, or a variant or a derivative thereof. None of the previously known reports show that change in the expression of the gene or the transcript thereof can serve as a marker for stomach cancer.

The 109th target gene is the hsa-miR-3620-5p gene, a congener thereof, a transcript thereof, or a variant or a derivative thereof. None of the previously known reports show that change in the expression of the gene or the transcript thereof can serve as a marker for stomach cancer.

The 110th target gene is the hsa-miR-1268b gene, a congener thereof, a transcript thereof, or a variant or a derivative thereof. None of the previously known reports show that change in the expression of the gene or the transcript thereof can serve as a marker for stomach cancer.

The 111th target gene is the hsa-miR-1227-5p gene, a congener thereof, a transcript thereof, or a variant or a derivative thereof. None of the previously known reports show that change in the expression of the gene or the transcript thereof can serve as a marker for stomach cancer.

The 112th target gene is the hsa-miR-6880-5p gene, a congener thereof, a transcript thereof, or a variant or a derivative thereof. None of the previously known reports show that change in the expression of the gene or the transcript thereof can serve as a marker for stomach cancer.

The 113th target gene is the hsa-miR-4417 gene, a congener thereof, a transcript thereof, or a variant or a derivative thereof. None of the previously known reports show that change in the expression of the gene or the transcript thereof can serve as a marker for stomach cancer.

The 114th target gene is the hsa-miR-6802-5p gene, a congener thereof, a transcript thereof, or a variant or a derivative thereof. None of the previously known reports show that change in the expression of the gene or the transcript thereof can serve as a marker for stomach cancer.

The 115th target gene is the hsa-miR-6769a-5p gene, a congener thereof, a transcript thereof, or a variant or a derivative thereof. None of the previously known reports show that change in the expression of the gene or the transcript thereof can serve as a marker for stomach cancer.

The 116th target gene is the hsa-miR-663a gene, a congener thereof, a transcript thereof, or a variant or a derivative thereof. None of the previously known reports show that change in the expression of the gene or the transcript thereof can serve as a marker for stomach cancer.

The 117th target gene is the hsa-miR-6721-5p gene, a congener thereof, a transcript thereof, or a variant or a derivative thereof. None of the previously known reports show that change in the expression of the gene or the transcript thereof can serve as a marker for stomach cancer.

The 118th target gene is the hsa-miR-4532 gene, a congener thereof, a transcript thereof, or a variant or a derivative thereof. None of the previously known reports show that change in the expression of the gene or the transcript thereof can serve as a marker for stomach cancer.

The 119th target gene is the hsa-miR-7977 gene, a congener thereof, a transcript thereof, or a variant or a derivative thereof. None of the previously known reports show that change in the expression of the gene or the transcript thereof can serve as a marker for stomach cancer.

The 120th target gene is the hsa-miR-92b-5p gene, a congener thereof, a transcript thereof, or a variant or a derivative thereof. None of the previously known reports show that change in the expression of the gene or the transcript thereof can serve as a marker for stomach cancer.

The 121st target gene is the hsa-miR-371a-5p gene, a congener thereof, a transcript thereof, or a variant or a derivative thereof. None of the previously known reports show that change in the expression of the gene or the transcript thereof can serve as a marker for stomach cancer.

The 122nd target gene is the hsa-miR-6126 gene, a congener thereof, a transcript thereof, or a variant or a derivative thereof. None of the previously known reports show that change in the expression of the gene or the transcript thereof can serve as a marker for stomach cancer.

The 123rd target gene is the hsa-miR-4734 gene, a congener thereof, a transcript thereof, or a variant or a derivative thereof. None of the previously known reports show that change in the expression of the gene or the transcript thereof can serve as a marker for stomach cancer.

The 124th target gene is the hsa-miR-4665-3p gene, a congener thereof, a transcript thereof, or a variant or a derivative thereof. None of the previously known reports show that change in the expression of the gene or the transcript thereof can serve as a marker for stomach cancer.

The 125th target gene is the hsa-miR-423-5p gene, a congener thereof, a transcript thereof, or a variant or a derivative thereof. None of the previously known reports show that change in the expression of the gene or the transcript thereof can serve as a marker for stomach cancer.

The 126th target gene is the hsa-miR-1469 gene, a congener thereof, a transcript thereof, or a variant or a derivative thereof. None of the previously known reports show that change in the expression of the gene or the transcript thereof can serve as a marker for stomach cancer.

The 127th target gene is the hsa-miR-4675 gene, a congener thereof, a transcript thereof, or a variant or a derivative thereof. None of the previously known reports show that change in the expression of the gene or the transcript thereof can serve as a marker for stomach cancer.

The 128th target gene is the hsa-miR-1915-5p gene, a congener thereof, a transcript thereof, or a variant or a derivative thereof. None of the previously known reports show that change in the expression of the gene or the transcript thereof can serve as a marker for stomach cancer.

The 129th target gene is the hsa-miR-6716-5p gene, a congener thereof, a transcript thereof, or a variant or a derivative thereof. None of the previously known reports show that change in the expression of the gene or the transcript thereof can serve as a marker for stomach cancer.

The 130th target gene is the hsa-miR-718 gene, a congener thereof, a transcript thereof, or a variant or a derivative thereof. None of the previously known reports show that change in the expression of the gene or the transcript thereof can serve as a marker for stomach cancer.

The 131st target gene is the hsa-miR-4281 gene, a congener thereof, a transcript thereof, or a variant or a derivative thereof. None of the previously known reports show that change in the expression of the gene or the transcript thereof can serve as a marker for stomach cancer.

The 132nd target gene is the hsa-miR-6820-5p gene, a congener thereof, a transcript thereof, or a variant or a derivative thereof. None of the previously known reports show that change in the expression of the gene or the transcript thereof can serve as a marker for stomach cancer.

The 133rd target gene is the hsa-miR-6795-5p gene, a congener thereof, a transcript thereof, or a variant or a derivative thereof. None of the previously known reports show that change in the expression of the gene or the transcript thereof can serve as a marker for stomach cancer.

The 134th target gene is the hsa-miR-6779-5p gene, a congener thereof, a transcript thereof, or a variant or a derivative thereof. None of the previously known reports show that change in the expression of the gene or the transcript thereof can serve as a marker for stomach cancer.

The 135th target gene is the hsa-miR-7109-5p gene, a congener thereof, a transcript thereof, or a variant or a derivative thereof. None of the previously known reports show that change in the expression of the gene or the transcript thereof can serve as a marker for stomach cancer.

The 136th target gene is the hsa-miR-6798-5p gene, a congener thereof, a transcript thereof, or a variant or a derivative thereof. None of the previously known reports show that change in the expression of the gene or the transcript thereof can serve as a marker for stomach cancer.

The 137th target gene is the hsa-miR-4648 gene, a congener thereof, a transcript thereof, or a variant or a derivative thereof. None of the previously known reports show that change in the expression of the gene or the transcript thereof can serve as a marker for stomach cancer.

The 138th target gene is the hsa-miR-8059 gene, a congener thereof, a transcript thereof, or a variant or a derivative thereof. None of the previously known reports show that change in the expression of the gene or the transcript thereof can serve as a marker for stomach cancer.

The 139th target gene is the hsa-miR-6765-3p gene, a congener thereof, a transcript thereof, or a variant or a derivative thereof. None of the previously known reports show that change in the expression of the gene or the transcript thereof can serve as a marker for stomach cancer.

The 140th target gene is the hsa-miR-6132 gene, a congener thereof, a transcript thereof, or a variant or a derivative thereof. None of the previously known reports show that change in the expression of the gene or the transcript thereof can serve as a marker for stomach cancer.

The 141st target gene is the hsa-miR-4492 gene, a congener thereof, a transcript thereof, or a variant or a derivative thereof. None of the previously known reports show that change in the expression of the gene or the transcript thereof can serve as a marker for stomach cancer.

The 142nd target gene is the hsa-miR-7107-5p gene, a congener thereof, a transcript thereof, or a variant or a derivative thereof. None of the previously known reports show that change in the expression of the gene or the transcript thereof can serve as a marker for stomach cancer.

The 143rd target gene is the hsa-miR-3195 gene, a congener thereof, a transcript thereof, or a variant or a derivative thereof. None of the previously known reports show that change in the expression of the gene or the transcript thereof can serve as a marker for stomach cancer.

The 144th target gene is the hsa-miR-3180 gene, a congener thereof, a transcript thereof, or a variant or a derivative thereof. None of the previously known reports show that change in the expression of the gene or the transcript thereof can serve as a marker for stomach cancer.

The 145th target gene is the hsa-miR-296-3p gene, a congener thereof, a transcript thereof, or a variant or a derivative thereof. None of the previously known reports show that change in the expression of the gene or the transcript thereof can serve as a marker for stomach cancer.

The 146th target gene is the hsa-miR-564 gene, a congener thereof, a transcript thereof, or a variant or a derivative thereof. None of the previously known reports show that change in the expression of the gene or the transcript thereof can serve as a marker for stomach cancer.

The 147th target gene is the hsa-miR-1268a gene, a congener thereof, a transcript thereof, or a variant or a derivative thereof. None of the previously known reports show that change in the expression of the gene or the transcript thereof can serve as a marker for stomach cancer.

The 148th target gene is the hsa-miR-6848-5p gene, a congener thereof, a transcript thereof, or a variant or a derivative thereof. None of the previously known reports show that change in the expression of the gene or the transcript thereof can serve as a marker for stomach cancer.

The 149th target gene is the hsa-miR-762 gene, a congener thereof, a transcript thereof, or a variant or a derivative thereof. None of the previously known reports show that change in the expression of the gene or the transcript thereof can serve as a marker for stomach cancer.

The 150th target gene is the hsa-miR-2861 gene, a congener thereof, a transcript thereof, or a variant or a derivative thereof. None of the previously known reports show that change in the expression of the gene or the transcript thereof can serve as a marker for stomach cancer.

The 151st target gene is the hsa-miR-1203 gene, a congener thereof, a transcript thereof, or a variant or a derivative thereof. None of the previously known reports show that change in the expression of the gene or the transcript thereof can serve as a marker for stomach cancer.

The 152nd target gene is the hsa-miR-1260b gene, a congener thereof, a transcript thereof, or a variant or a derivative thereof. None of the previously known reports show that change in the expression of the gene or the transcript thereof can serve as a marker for stomach cancer.

The 153rd target gene is the hsa-miR-4476 gene, a congener thereof, a transcript thereof, or a variant or a derivative thereof. None of the previously known reports show that change in the expression of the gene or the transcript thereof can serve as a marker for stomach cancer.

The 154th target gene is the hsa-miR-6885-5p gene, a congener thereof, a transcript thereof, or a variant or a derivative thereof. None of the previously known reports show that change in the expression of the gene or the transcript thereof can serve as a marker for stomach cancer.

The 155th target gene is the hsa-miR-6769b-5p gene, a congener thereof, a transcript thereof, or a variant or a derivative thereof. None of the previously known reports show that change in the expression of the gene or the transcript thereof can serve as a marker for stomach cancer.

The 156th target gene is the hsa-miR-23b-3p gene, a congener thereof, a transcript thereof, or a variant or a derivative thereof. None of the previously known reports show that change in the expression of the gene or the transcript thereof can serve as a marker for stomach cancer.

The 157th target gene is the hsa-miR-1343-5p gene, a congener thereof, a transcript thereof, or a variant or a derivative thereof. None of the previously known reports show that change in the expression of the gene or the transcript thereof can serve as a marker for stomach cancer.

The 158th target gene is the hsa-miR-3621 gene, a congener thereof, a transcript thereof, or a variant or a derivative thereof. None of the previously known reports show that change in the expression of the gene or the transcript thereof can serve as a marker for stomach cancer.

The 159th target gene is the hsa-miR-4688 gene, a congener thereof, a transcript thereof, or a variant or a derivative thereof. None of the previously known reports show that change in the expression of the gene or the transcript thereof can serve as a marker for stomach cancer.

The 160th target gene is the hsa-miR-4286 gene, a congener thereof, a transcript thereof, or a variant or a derivative thereof. None of the previously known reports show that change in the expression of the gene or the transcript thereof can serve as a marker for stomach cancer.

The 161st target gene is the hsa-miR-4640-5p gene, a congener thereof, a transcript thereof, or a variant or a derivative thereof. None of the previously known reports show that change in the expression of the gene or the transcript thereof can serve as a marker for stomach cancer.

The 162nd target gene is the hsa-miR-4739 gene, a congener thereof, a transcript thereof, or a variant or a derivative thereof. None of the previously known reports show that change in the expression of the gene or the transcript thereof can serve as a marker for stomach cancer.

The 163rd target gene is the hsa-miR-1260a gene, a congener thereof, a transcript thereof, or a variant or a derivative thereof. None of the previously known reports show that change in the expression of the gene or the transcript thereof can serve as a marker for stomach cancer.

The 164th target gene is the hsa-miR-4276 gene, a congener thereof, a transcript thereof, or a variant or a derivative thereof. None of the previously known reports show that change in the expression of the gene or the transcript thereof can serve as a marker for stomach cancer.

The 165th target gene is the hsa-miR-7106-5p gene, a congener thereof, a transcript thereof, or a variant or a derivative thereof. None of the previously known reports show that change in the expression of the gene or the transcript thereof can serve as a marker for stomach cancer.

The 166th target gene is the hsa-miR-128-2-5p gene, a congener thereof, a transcript thereof, or a variant or a derivative thereof. The previously known report shows that change in the expression of the hsa-miR-128b (hsa-miR-128-2-3p) gene, which is derived from the same precursor, or the transcript thereof can serve as a marker for stomach cancer (Patent Literature 2).

The 167th target gene is the hsa-miR-125a-3p gene, a congener thereof, a transcript thereof, or a variant or a derivative thereof. The previously known report shows that change in the expression of the gene or the transcript thereof can serve as a marker for stomach cancer (Patent Literature 1).

The 168th target gene is the hsa-miR-92a-2-5p gene, a congener thereof, a transcript thereof, or a variant or a derivative thereof. The previously known report shows that change in the expression of the hsa-miR-92-2 (hsa-miR-92a-2-3p) gene, which is derived from the same precursor, or the transcript thereof can serve as a marker for stomach cancer (Patent Literature 2).

The 169th target gene is the hsa-miR-486-3p gene, a congener thereof, a transcript thereof, or a variant or a derivative thereof. The previously known report shows that change in the expression of the hsa-miR-486-5p gene, which is derived from the same precursor, or the transcript thereof can serve as a marker for stomach cancer (Patent Literature 3).

The 170th target gene is the hsa-miR-3196 gene, a congener thereof, a transcript thereof, or a variant or a derivative thereof. None of the previously known reports show that change in the expression of the gene or the transcript thereof can serve as a marker for stomach cancer.

The 171st target gene is the hsa-miR-211-3p gene, a congener thereof, a transcript thereof, or a variant or a derivative thereof. The previously known report shows that change in the expression of the hsa-miR-211 (hsa-miR-211-5p) gene, which is derived from the same precursor, or the transcript thereof can serve as a marker for stomach cancer (Patent Literature 2).

The 172nd target gene is the hsa-miR-4271 gene, a congener thereof, a transcript thereof, or a variant or a derivative thereof. None of the previously known reports show that change in the expression of the gene or the transcript thereof can serve as a marker for stomach cancer.

The 173rd target gene is the hsa-miR-6851-5p gene, a congener thereof, a transcript thereof, or a variant or a derivative thereof. None of the previously known reports show that change in the expression of the gene or the transcript thereof can serve as a marker for stomach cancer.

The 174th target gene is the hsa-miR-149-3p gene, a congener thereof, a transcript thereof, or a variant or a derivative thereof. None of the previously known reports show that change in the expression of the gene or the transcript thereof can serve as a marker for stomach cancer.

The 175th target gene is the hsa-miR-4667-5p gene, a congener thereof, a transcript thereof, or a variant or a derivative thereof. None of the previously known reports show that change in the expression of the gene or the transcript thereof can serve as a marker for stomach cancer.

The 176th target gene is the hsa-miR-135a-3p gene, a congener thereof, a transcript thereof, or a variant or a derivative thereof. None of the previously known reports show that change in the expression of the gene or the transcript thereof can serve as a marker for stomach cancer.

The 177th target gene is the hsa-miR-4486 gene, a congener thereof, a transcript thereof, or a variant or a derivative thereof. None of the previously known reports show that change in the expression of the gene or the transcript thereof can serve as a marker for stomach cancer.

The 178th target gene is the hsa-miR-4697-5p gene, a congener thereof, a transcript thereof, or a variant or a derivative thereof. None of the previously known reports show that change in the expression of the gene or the transcript thereof can serve as a marker for stomach cancer.

The 179th target gene is the hsa-miR-4725-3p gene, a congener thereof, a transcript thereof, or a variant or a derivative thereof. None of the previously known reports show that change in the expression of the gene or the transcript thereof can serve as a marker for stomach cancer.

The 180th target gene is the hsa-miR-6510-5p gene, a congener thereof, a transcript thereof, or a variant or a derivative thereof. None of the previously known reports show that change in the expression of the gene or the transcript thereof can serve as a marker for stomach cancer.

The 181st target gene is the hsa-miR-5001-5p gene, a congener thereof, a transcript thereof, or a variant or a derivative thereof. None of the previously known reports show that change in the expression of the gene or the transcript thereof can serve as a marker for stomach cancer.

The 182nd target gene is the hsa-miR-4673 gene, a congener thereof, a transcript thereof, or a variant or a derivative thereof. None of the previously known reports show that change in the expression of the gene or the transcript thereof can serve as a marker for stomach cancer.

The 183rd target gene is the hsa-miR-4466 gene, a congener thereof, a transcript thereof, or a variant or a derivative thereof. None of the previously known reports show that change in the expression of the gene or the transcript thereof can serve as a marker for stomach cancer.

The 184th target gene is the hsa-miR-23a-3p gene, a congener thereof, a transcript thereof, or a variant or a derivative thereof. The previously known report shows that change in the expression of the gene or the transcript thereof can serve as a marker for stomach cancer (Patent Literature 2).

The 185th target gene is the hsa-miR-3656 gene, a congener thereof, a transcript thereof, or a variant or a derivative thereof. None of the previously known reports show that change in the expression of the gene or the transcript thereof can serve as a marker for stomach cancer.

The 186th target gene is the hsa-miR-6782-5p gene, a congener thereof, a transcript thereof, or a variant or a derivative thereof. None of the previously known reports show that change in the expression of the gene or the transcript thereof can serve as a marker for stomach cancer.

The 187th target gene is the hsa-miR-4689 gene, a congener thereof, a transcript thereof, or a variant or a derivative thereof. None of the previously known reports show that change in the expression of the gene or the transcript thereof can serve as a marker for stomach cancer.

The 188th target gene is the hsa-miR-451a gene, a congener thereof, a transcript thereof, or a variant or a derivative thereof. The previously known report shows that change in the expression of the gene or the transcript thereof can serve as a marker for stomach cancer (Patent Literature 3).

The 189th target gene is the hsa-miR-4446-3p gene, a congener thereof, a transcript thereof, or a variant or a derivative thereof. None of the previously known reports show that change in the expression of the gene or the transcript thereof can serve as a marker for stomach cancer.

The 190th target gene is the hsa-miR-3180-3p gene, a congener thereof, a transcript thereof, or a variant or a derivative thereof. None of the previously known reports show that change in the expression of the gene or the transcript thereof can serve as a marker for stomach cancer.

The 191st target gene is the hsa-miR-642a-3p gene, a congener thereof, a transcript thereof, or a variant or a derivative thereof. None of the previously known reports show that change in the expression of the gene or the transcript thereof can serve as a marker for stomach cancer.

The 192nd target gene is the hsa-miR-6889-5p gene, a congener thereof, a transcript thereof, or a variant or a derivative thereof. None of the previously known reports show that change in the expression of the gene or the transcript thereof can serve as a marker for stomach cancer.

The 193rd target gene is the hsa-miR-3178 gene, a congener thereof, a transcript thereof, or a variant or a derivative thereof. None of the previously known reports show that change in the expression of the gene or the transcript thereof can serve as a marker for stomach cancer.

The 194th target gene is the hsa-miR-4665-5p gene, a congener thereof, a transcript thereof, or a variant or a derivative thereof. None of the previously known reports show that change in the expression of the gene or the transcript thereof can serve as a marker for stomach cancer.

The 195th target gene is the hsa-miR-6722-3p gene, a congener thereof, a transcript thereof, or a variant or a derivative thereof. None of the previously known reports show that change in the expression of the gene or the transcript thereof can serve as a marker for stomach cancer.

The 196th target gene is the hsa-miR-30c-1-3p gene, a congener thereof, a transcript thereof, or a variant or a derivative thereof. The previously known report shows that change in the expression of the hsa-miR-30c (hsa-miR-30c-1-5p) gene, which is derived from the same precursor, or the transcript thereof can serve as a marker for stomach cancer (Patent Literature 2).

The 197th target gene is the hsa-miR-4507 gene, a congener thereof, a transcript thereof, or a variant or a derivative thereof. None of the previously known reports show that change in the expression of the gene or the transcript thereof can serve as a marker for stomach cancer.

The 198th target gene is the hsa-miR-3141 gene, a congener thereof, a transcript thereof, or a variant or a derivative thereof. None of the previously known reports show that change in the expression of the gene or the transcript thereof can serve as a marker for stomach cancer.

The 199th target gene is the hsa-miR-1199-5p gene, a congener thereof, a transcript thereof, or a variant or a derivative thereof. None of the previously known reports show that change in the expression of the gene or the transcript thereof can serve as a marker for stomach cancer.

The 200th target gene is the hsa-miR-6794-5p gene, a congener thereof, a transcript thereof, or a variant or a derivative thereof. None of the previously known reports show that change in the expression of the gene or the transcript thereof can serve as a marker for stomach cancer.

The 201st target gene is the hsa-miR-6774-5p gene, a congener thereof, a transcript thereof, or a variant or a derivative thereof. None of the previously known reports show that change in the expression of the gene or the transcript thereof can serve as a marker for stomach cancer.

The 202nd target gene is the hsa-miR-4707-3p gene, a congener thereof, a transcript thereof, or a variant or a derivative thereof. None of the previously known reports show that change in the expression of the gene or the transcript thereof can serve as a marker for stomach cancer.

The 203rd target gene is the hsa-miR-4534 gene, a congener thereof, a transcript thereof, or a variant or a derivative thereof. None of the previously known reports show that change in the expression of the gene or the transcript thereof can serve as a marker for stomach cancer.

The 204th target gene is the hsa-miR-4294 gene, a congener thereof, a transcript thereof, or a variant or a derivative thereof. None of the previously known reports show that change in the expression of the gene or the transcript thereof can serve as a marker for stomach cancer.

The 205th target gene is the hsa-miR-6850-5p gene, a congener thereof, a transcript thereof, or a variant or a derivative thereof. None of the previously known reports show that change in the expression of the gene or the transcript thereof can serve as a marker for stomach cancer.

The 206th target gene is the hsa-miR-6089 gene, a congener thereof, a transcript thereof, or a variant or a derivative thereof. None of the previously known reports show that change in the expression of the gene or the transcript thereof can serve as a marker for stomach cancer.

The 207th target gene is the hsa-miR-671-5p gene, a congener thereof, a transcript thereof, or a variant or a derivative thereof. None of the previously known reports show that change in the expression of the gene or the transcript thereof can serve as a marker for stomach cancer.

### 2. Nucleic Acid Probe or Primer for Detection of Stomach Cancer

In the present invention, a nucleic acid(s) capable of specifically binding to any of the target nucleic acid(s) as the stomach cancer marker(s) described above can be used as a nucleic acid(s), for example, a nucleic acid probe(s) or a primer(s), for the detection or diagnosis of stomach cancer.

In the present invention, the nucleic acid probe(s) or the primer(s) that can be used for detecting stomach cancer or for diagnosing stomach cancer enables qualitative and/or quantitative measurement of the presence, expression level, or abundance of any of the target nucleic acids as the stomach cancer markers described above, for example: human-derived hsa-miR-4257, hsa-miR-6726-5p, hsa-miR-1343-3p, hsa-miR-1247-3p, hsa-miR-6787-5p, hsa-miR-6875-5p, hsa-miR-1225-3p, hsa-miR-8063, hsa-miR-6781-5p, hsa-miR-4746-3p, hsa-miR-1908-5p, hsa-miR-6756-5p, hsa-miR-204-3p, hsa-miR-4651, hsa-miR-6757-5p, hsa-miR-6825-5p, hsa-miR-7108-5p, hsa-miR-4792, hsa-miR-7641, hsa-miR-3188, hsa-miR-3131, hsa-miR-6780b-5p, hsa-miR-8069, hsa-miR-6840-3p, hsa-miR-8072, hsa-miR-1233-5p, hsa-miR-6887-5p, hsa-miR-1231, hsa-miR-5572, hsa-miR-6738-5p, hsa-miR-6784-5p, hsa-miR-6791-5p, hsa-miR-6749-5p, hsa-miR-6741-5p, hsa-miR-128-1-5p, hsa-miR-4419b, hsa-miR-6746-5p, hsa-miR-3184-5p, hsa-miR-3679-5p, hsa-miR-7110-5p, hsa-miR-4516, hsa-miR-6717-5p, hsa-miR-6826-5p, hsa-miR-4433b-3p, hsa-miR-3679-3p, hsa-miR-3135b, hsa-miR-3622a-5p, hsa-miR-711, hsa-miR-4467, hsa-miR-6857-5p, hsa-miR-6515-3p, hsa-miR-1225-5p, hsa-miR-187-5p, hsa-miR-3185, hsa-miR-642b-3p, hsa-miR-1249, hsa-miR-744-5p, hsa-miR-4442, hsa-miR-1228-3p, hsa-miR-939-5p, hsa-miR-6845-5p, hsa-miR-887-3p, hsa-miR-7845-5p, hsa-miR-6729-5p, hsa-miR-4632-5p, hsa-miR-615-5p, hsa-miR-6724-5p, hsa-miR-4728-5p, hsa-miR-6732-5p, hsa-miR-6816-5p, hsa-miR-4695-5p, hsa-miR-6088, hsa-miR-7975, hsa-miR-3197, hsa-miR-6125, hsa-miR-4433-3p, hsa-miR-6727-5p, hsa-miR-4706, hsa-miR-7847-3p, hsa-miR-6805-3p, hsa-miR-6766-3p, hsa-miR-1913, hsa-miR-4649-5p, hsa-miR-602, hsa-miR-3663-3p, hsa-miR-6893-5p, hsa-miR-6861-5p, hsa-miR-4449, hsa-miR-6842-5p, hsa-miR-4454, hsa-miR-5195-3p, hsa-miR-663b, hsa-miR-6765-5p, hsa-miR-4513, hsa-miR-614, hsa-miR-6785-5p, hsa-miR-6777-5p, hsa-miR-940, hsa-miR-4741, hsa-miR-6870-5p, hsa-miR-6131, hsa-miR-150-3p, hsa-miR-4707-5p, hsa-miR-1915-3p, hsa-miR-3937, hsa-miR-937-5p, hsa-miR-4443, hsa-miR-1914-3p, hsa-miR-3620-5p, hsa-miR-1268b, hsa-miR-1227-5p, hsa-miR-6880-5p, hsa-miR-4417, hsa-miR-6802-5p, hsa-miR-6769a-5p, hsa-miR-663a, hsa-miR-6721-5p, hsa-miR-4532, hsa-miR-7977, hsa-miR-92b-5p, hsa-miR-371a-5p, hsa-miR-6126, hsa-miR-4734, hsa-miR-4665-3p, hsa-miR-423-5p, hsa-miR-1469, hsa-miR-4675, hsa-miR-1915-5p, hsa-miR-6716-5p, hsa-miR-718, hsa-miR-4281, hsa-miR-6820-5p, hsa-miR-6795-5p, hsa-miR-6779-5p, hsa-miR-7109-5p, hsa-miR-6798-5p, hsa-miR-4648, hsa-miR-8059, hsa-miR-6765-3p, hsa-miR-6132, hsa-miR-4492, hsa-miR-7107-5p, hsa-miR-3195, hsa-miR-3180, hsa-miR-296-3p, hsa-miR-564, hsa-miR-1268a, hsa-miR-6848-5p, hsa-miR-762, hsa-miR-2861, hsa-miR-1203, hsa-miR-1260b, hsa-miR-4476, hsa-miR-6885-5p, hsa-miR-6769b-5p, hsa-miR-23b-3p, hsa-miR-1343-5p, hsa-miR-3621, hsa-miR-4688, hsa-miR-4286, hsa-miR-4640-5p, hsa-miR-4739, hsa-miR-1260a, hsa-miR-4276, hsa-miR-7106, hsa-miR-6794-5p, hsa-miR-6774-5p, hsa-miR-4707-3p, hsa-miR-4534, hsa-miR-4294, hsa-miR-6850-5p, hsa-miR-6089 and hsa-miR-671-5p or a combination thereof, congeners thereof, transcripts thereof, or variants or derivatives thereof; and, optionally combined therewith, hsa-miR-128-2-5p, hsa-miR-125a-3p, hsa-miR-92a-2-5p, and hsa-miR-486-3p or a combination thereof, congeners thereof, transcripts thereof, or variants or derivatives thereof; and, optionally combined therewith, hsa-miR-3196, hsa-miR-211-3p, hsa-miR-4271, hsa-miR-6851-5p, hsa-miR-149-3p, hsa-miR-4667-5p, hsa-miR-135a-3p, hsa-miR-4486, hsa-miR-4697-5p, hsa-miR-4725-3p, hsa-miR-6510-5p, hsa-miR-5001-5p, hsa-miR-4673, hsa-miR-4466, hsa-miR-23a-3p, hsa-miR-3656, hsa-miR-6782-5p, hsa-miR-4689, hsa-miR-451a, hsa-miR-4446-3p, hsa-miR-3180-3p, hsa-miR-642a-3p, hsa-miR-6889-5p, hsa-miR-3178, hsa-miR-4665-5p, hsa-miR-6722-3p, hsa-miR-30c-1-3p, hsa-miR-4507, hsa-miR-3141 and hsa-miR-1199-5p or a combination thereof, congeners thereof, transcripts thereof, or variants or derivatives thereof.

The expression level of each target nucleic acid described above is increased or decreased (hereinafter, referred to as “increased/decreased”) depending on the type of the target nucleic acid in a subject having stomach cancer as compared with a healthy subject. Hence, the nucleic acid of the present invention can be effectively used for measuring the expression level of the target nucleic acid described above in a body fluid derived from a subject (e.g., a human) suspected of having stomach cancer and a body fluid derived from a healthy subject and thereby detecting stomach cancer by the comparison thereof.

The nucleic acid probe(s) or the primer(s) that can be used in the present invention is a nucleic acid probe(s) capable of specifically binding to at least one polynucleotide consisting of a nucleotide sequence represented by any of SEQ ID NOs: 1 to 165 and 635 to 642, or a primer for amplifying at least one polynucleotide consisting of a nucleotide sequence represented by any of SEQ ID NOs: 1 to 165 and 635 to 642.

The nucleic acid probe(s) or the primer(s) that can be used in the present invention may further comprise a nucleic acid probe capable of specifically binding to at least one polynucleotide consisting of a nucleotide sequence represented by any of SEQ ID NOs: 166 to 169, or a primer for amplifying at least one polynucleotide consisting of a nucleotide sequence represented by any of SEQ ID NOs: 166 to 169.

The nucleic acid probe(s) or the primer(s) that can be used in the present invention may further comprise a nucleic acid probe capable of specifically binding to at least one polynucleotide consisting of a nucleotide sequence represented by any of SEQ ID NOs: 170 to 199, or a primer for amplifying at least one polynucleotide consisting of a nucleotide sequence represented by any of SEQ ID NOs: 170 to 199.

Specifically, these nucleic acid probes or primers comprise a combination of one or more polynucleotides selected from a group of polynucleotides comprising nucleotide sequences represented by any of SEQ ID NOs: 1 to 657, or nucleotide sequences derived from the nucleotide sequences by the replacement of u with t, and a group of complementary polynucleotides thereof, a group of polynucleotides respectively hybridizing under stringent conditions (mentioned later) to DNAs consisting of nucleotide sequences complementary to these nucleotide sequences, and a group of complementary polynucleotides thereof, and a group of polynucleotides comprising 15 or more, preferably 17 or more consecutive nucleotides in the nucleotide sequences of these polynucleotide groups. These polynucleotides can be used as nucleic acid probes and primers for detecting the stomach cancer markers as target nucleic acids.

More specifically, examples of the nucleic acid probe(s) or the primer(s) that can be used in the present invention include one or more polynucleotides selected from the group consisting of the following polynucleotides (a) to (e):
(a) a polynucleotide consisting of a nucleotide sequence represented by any of SEQ ID NOs: 1 to 165 and 635 to 642 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 165 and 635 to 642,
(c) a polynucleotide consisting of a nucleotide sequence complementary to a nucleotide sequence represented by any of SEQ ID NOs: 1 to 165 and 635 to 642, or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 165 and 635 to 642, or a nucleotide sequence derived from the nucleotide sequence by the replacement of u with t, and
(e) a polynucleotide hybridizing under stringent conditions to any of the polynucleotides (a) to (d).

In addition to at least one or more polynucleotides selected from the group consisting of the polynucleotides (a) to (e), the nucleic acid probe(s) or the primer(s) that can be used in the present invention may further comprise polynucleotides selected from the group consisting of the following polynucleotides (f) to (j):
(f) a polynucleotide consisting of a nucleotide sequence represented by any of SEQ ID NOs: 166 to 169 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 166 to 169,
(h) a polynucleotide consisting of a nucleotide sequence complementary to a nucleotide sequence represented by any of SEQ ID NOs: 166 to 169 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 166 to 169 or a nucleotide sequence derived from the nucleotide sequence by the replacement of u with t, and
(j) a polynucleotide hybridizing under stringent conditions to any of the polynucleotides (f) to (i).

In addition to at least one or more polynucleotide(s) selected from the group consisting of the polynucleotides (a) to (j), the nucleic acid probe(s) or the primer(s) that can be used in the present invention may further comprise polynucleotides selected from the group consisting of the following polynucleotides (k) to (o):
(k) a polynucleotide consisting of a nucleotide sequence represented by any of SEQ ID NOs: 170 to 199 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 170 to 199,
(m) a polynucleotide consisting of a nucleotide sequence complementary to a nucleotide sequence represented by any of SEQ ID NOs: 170 to 199 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 170 to 199 or a nucleotide sequence derived from the nucleotide sequence by the replacement of u with t, and
(o) a polynucleotide hybridizing under stringent conditions to any of the polynucleotides (k) to (n).

For the above-mentioned polynucleotides, the “fragment thereof comprising 15 or more consecutive nucleotides” can comprise, but not limited to, the number of nucleotides in the range from, for example, 15 consecutive nucleotides to less than the total number of nucleotides of the sequence, from 17 consecutive nucleotides to less than the total number of nucleotides of the sequence, or from 19 consecutive nucleotides to less than the total number of nucleotides of the sequence, or the like, in the nucleotide sequence of each polynucleotide.

These polynucleotides or fragments thereof used in the present invention may each be DNA or may each be RNA.

The polynucleotides that can be used in the present invention can each be prepared by use of a general technique such as a DNA recombination technique, PCR, or a method using an automatic DNA/RNA synthesizer.

The DNA recombination technique and the PCR method may employ a technique described in, for example, Ausubel et al., Current Protocols in Molecular Biology, John Willey & Sons, US (1993); and Sambrook et al., Molecular Cloning—A Laboratory Manual, Cold Spring Harbor Laboratory Press, US (1989).

The human-derived hsa-miR-4257, hsa-miR-6726-5p, hsa-miR-1343-3p, hsa-miR-1247-3p, hsa-miR-6787-5p, hsa-miR-6875-5p, hsa-miR-1225-3p, hsa-miR-8063, hsa-miR-6781-5p, hsa-miR-4746-3p, hsa-miR-1908-5p, hsa-miR-6756-5p, hsa-miR-204-3p, hsa-miR-4651, hsa-miR-6757-5p, hsa-miR-6825-5p, hsa-miR-7108-5p, hsa-miR-4792, hsa-miR-7641, hsa-miR-3188, hsa-miR-3131, hsa-miR-6780b-5p, hsa-miR-8069, hsa-miR-6840-3p, hsa-miR-8072, hsa-miR-1233-5p, hsa-miR-6887-5p, hsa-miR-1231, hsa-miR-5572, hsa-miR-6738-5p, hsa-miR-6784-5p, hsa-miR-6791-5p, hsa-miR-6749-5p, hsa-miR-6741-5p, hsa-miR-128-1-5p, hsa-miR-4419b, hsa-miR-6746-5p, hsa-miR-3184-5p, hsa-miR-3679-5p, hsa-miR-7110-5p, hsa-miR-4516, hsa-miR-6717-5p, hsa-miR-6826-5p, hsa-miR-4433b-3p, hsa-miR-3679-3p, hsa-miR-3135b, hsa-miR-3622a-5p, hsa-miR-711, hsa-miR-4467, hsa-miR-6857-5p, hsa-miR-6515-3p, hsa-miR-1225-5p, hsa-miR-187-5p, hsa-miR-3185, hsa-miR-642b-3p, hsa-miR-1249, hsa-miR-744-5p, hsa-miR-4442, hsa-miR-1228-3p, hsa-miR-939-5p, hsa-miR-6845-5p, hsa-miR-887-3p, hsa-miR-7845-5p, hsa-miR-6729-5p, hsa-miR-4632-5p, hsa-miR-615-5p, hsa-miR-6724-5p, hsa-miR-4728-5p, hsa-miR-6732-5p, hsa-miR-6816-5p, hsa-miR-4695-5p, hsa-miR-6088, hsa-miR-7975, hsa-miR-3197, hsa-miR-6125, hsa-miR-4433-3p, hsa-miR-6727-5p, hsa-miR-4706, hsa-miR-7847-3p, hsa-miR-6805-3p, hsa-miR-6766-3p, hsa-miR-1913, hsa-miR-4649-5p, hsa-miR-602, hsa-miR-3663-3p, hsa-miR-6893-5p, hsa-miR-6861-5p, hsa-miR-4449, hsa-miR-6842-5p, hsa-miR-4454, hsa-miR-5195-3p, hsa-miR-663b, hsa-miR-6765-5p, hsa-miR-4513, hsa-miR-614, hsa-miR-6785-5p, hsa-miR-6777-5p, hsa-miR-940, hsa-miR-4741, hsa-miR-6870-5p, hsa-miR-6131, hsa-miR-150-3p, hsa-miR-4707-5p, hsa-miR-1915-3p, hsa-miR-3937, hsa-miR-937-5p, hsa-miR-4443, hsa-miR-1914-3p, hsa-miR-3620-5p, hsa-miR-1268b, hsa-miR-1227-5p, hsa-miR-6880-5p, hsa-miR-4417, hsa-miR-6802-5p, hsa-miR-6769a-5p, hsa-miR-663a, hsa-miR-6721-5p, hsa-miR-4532, hsa-miR-7977, hsa-miR-92b-5p, hsa-miR-371a-5p, hsa-miR-6126, hsa-miR-4734, hsa-miR-4665-3p, hsa-miR-423-5p, hsa-miR-1469, hsa-miR-4675, hsa-miR-1915-5p, hsa-miR-6716-5p, hsa-miR-718, hsa-miR-4281, hsa-miR-6820-5p, hsa-miR-6795-5p, hsa-miR-6779-5p, hsa-miR-7109-5p, hsa-miR-6798-5p, hsa-miR-4648, hsa-miR-8059, hsa-miR-6765-3p, hsa-miR-6132, hsa-miR-4492, hsa-miR-7107-5p, hsa-miR-3195, hsa-miR-3180, hsa-miR-296-3p, hsa-miR-564, hsa-miR-1268a, hsa-miR-6848-5p, hsa-miR-762, hsa-miR-2861, hsa-miR-1203, hsa-miR-1260b, hsa-miR-4476, hsa-miR-6885-5p, hsa-miR-6769b-5p, hsa-miR-23b-3p, hsa-miR-1343-5p, hsa-miR-3621, hsa-miR-4688, hsa-miR-4286, hsa-miR-4640-5p, hsa-miR-4739, hsa-miR-1260a, hsa-miR-4276, hsa-miR-7106, hsa-miR-6794-5p, hsa-miR-6774-5p, hsa-miR-4707-3p, hsa-miR-4534, hsa-miR-4294, hsa-miR-6850-5p, hsa-miR-6089 and hsa-miR-671-5p, hsa-miR-128-2-5p, hsa-miR-125a-3p, hsa-miR-92a-2-5p, hsa-miR-486-3p, hsa-miR-3196, hsa-miR-211-3p, hsa-miR-4271, hsa-miR-6851-5p, hsa-miR-149-3p, hsa-miR-4667-5p, hsa-miR-135a-3p, hsa-miR-4486, hsa-miR-4697-5p, hsa-miR-4725-3p, hsa-miR-6510-5p, hsa-miR-5001-5p, hsa-miR-4673, hsa-miR-4466, hsa-miR-23a-3p, hsa-miR-3656, hsa-miR-6782-5p, hsa-miR-4689, hsa-miR-451a, hsa-miR-4446-3p, hsa-miR-3180-3p, hsa-miR-642a-3p, hsa-miR-6889-5p, hsa-miR-3178, hsa-miR-4665-5p, hsa-miR-6722-3p, hsa-miR-30c-1-3p, hsa-miR-4507, hsa-miR-3141 and hsa-miR-1199-5p represented by SEQ ID NOs: 1 to 199 and 635 to 642 are known in the art, and their acquisition methods are also known as mentioned above. Therefore, each polynucleotide that can be used as a nucleic acid probe(s) or a primer(s) in the present invention can be prepared by cloning the gene.

Such a nucleic acid probe(s) or a primer(s) can be chemically synthesized using an automatic DNA synthesizer. In general, a phosphoramidite method is used in this synthesis, and single-stranded DNA up to approximately 100 nucleotides can be automatically synthesized by this method. The automatic DNA synthesizer is commercially available from, for example, Polygen GmbH, ABI, or Applied Biosystems, Inc.

Alternatively, the polynucleotide of the present invention can also be prepared by a cDNA cloning method. The cDNA cloning technique may employ, for example, microRNA Cloning Kit Wako.

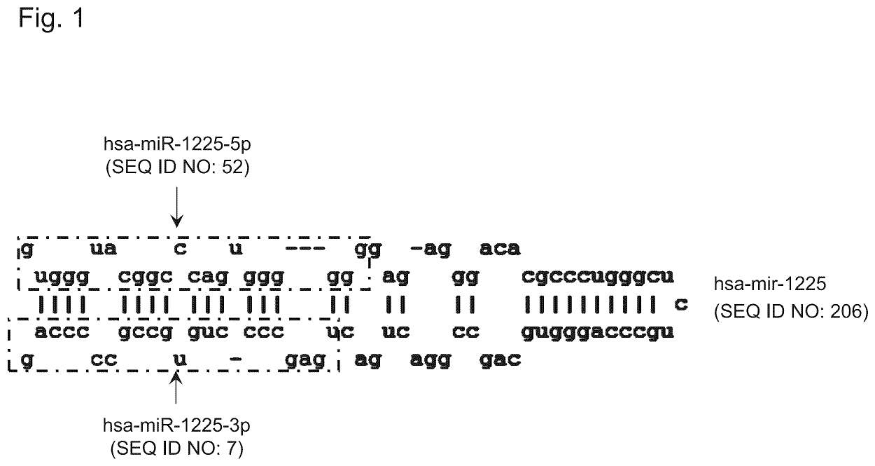

In this context, the sequences of the nucleic acid probe(s) and the primer(s) for detecting the polynucleotide consisting of a nucleotide sequence represented by any of SEQ ID NOs: 1 to 199 and 635 to 642 do not exist as miRNAs or precursors thereof in the living body or in vivo. For example, the nucleotide sequences represented by SEQ ID NO: 7 and SEQ ID NO: 52 are produced from the precursor represented by SEQ ID NO: 206. This precursor has a hairpin-like structure as shown in FIG. 1, and the nucleotide sequences represented by SEQ ID NO: 7 and SEQ ID NO: 52 have mismatch sequences with each other. As such, a nucleotide sequence completely complementary to the nucleotide sequence represented by SEQ ID NO: 7 or SEQ ID NO: 52 is not naturally produced in vivo. Therefore, the nucleic acid probe and the primer for detecting the nucleotide sequence represented by any of SEQ ID NOs: 1 to 199 and 635 to 642 each have an artificial nucleotide sequence that does not exist in the living body or in vivo.

### 3. Kit or Device for Detection of Stomach Cancer

The present invention also provides a kit or a device for the detection of stomach cancer, comprising one or more polynucleotides (which may include a variant, a fragment, or a derivative thereof; hereinafter, also referred to as a polynucleotide for detection) that can be used as a nucleic acid probe(s) or a primer(s) in the present invention for measuring a target nucleic acid(s) as a stomach cancer marker(s).

The target nucleic acid(s) as a stomach cancer marker(s) according to the present invention is selected from the following group 1: miR-4257, miR-6726-5p, miR-1343-3p, miR-1247-3p, miR-6787-5p, miR-6875-5p, miR-1225-3p, miR-8063, miR-6781-5p, miR-4746-3p, miR-1908-5p, miR-6756-5p, miR-204-3p, miR-4651, miR-6757-5p, miR-6825-5p, miR-7108-5p, miR-4792, miR-7641, miR-3188, miR-3131, miR-6780b-5p, miR-8069, miR-6840-3p, miR-8072, miR-1233-5p, miR-6887-5p, miR-1231, miR-5572, miR-6738-5p, miR-6784-5p, miR-6791-5p, miR-6749-5p, miR-6741-5p, miR-128-1-5p, miR-4419b, miR-6746-5p, miR-3184-5p, miR-3679-5p, miR-7110-5p, miR-4516, miR-6717-5p, miR-6826-5p, miR-4433b-3p, miR-3679-3p, miR-3135b, miR-3622a-5p, miR-711, miR-4467, miR-6857-5p, miR-6515-3p, miR-1225-5p, miR-187-5p, miR-3185, miR-642b-3p, miR-1249, miR-744-5p, miR-4442, miR-1228-3p, miR-939-5p, miR-6845-5p, miR-887-3p, miR-7845-5p, miR-6729-5p, miR-4632-5p, miR-615-5p, miR-6724-5p, miR-4728-5p, miR-6732-5p, miR-6816-5p, miR-4695-5p, miR-6088, miR-7975, miR-3197, miR-6125, miR-4433-3p, miR-6727-5p, miR-4706, miR-7847-3p, miR-6805-3p, miR-6766-3p, miR-1913, miR-4649-5p, miR-602, miR-3663-3p, miR-6893-5p, miR-6861-5p, miR-4449, miR-6842-5p, miR-4454, miR-5195-3p, miR-663b, miR-6765-5p, miR-4513, miR-614, miR-6785-5p, miR-6777-5p, miR-940, miR-4741, miR-6870-5p, miR-6131, miR-150-3p, miR-4707-5p, miR-1915-3p, miR-3937, miR-937-5p, miR-4443, miR-1914-3p, miR-3620-5p, miR-1268b, miR-1227-5p, miR-6880-5p, miR-4417, miR-6802-5p, miR-6769a-5p, miR-663a, miR-6721-5p, miR-4532, miR-7977, miR-92b-5p, miR-371a-5p, miR-6126, miR-4734, miR-4665-3p, miR-423-5p, miR-1469, miR-4675, miR-1915-5p, miR-6716-5p, miR-718, miR-4281, miR-6820-5p, miR-6795-5p, miR-6779-5p, miR-7109-5p, miR-6798-5p, miR-4648, miR-8059, miR-6765-3p, miR-6132, miR-4492, miR-7107-5p, miR-3195, miR-3180, miR-296-3p, miR-564, miR-1268a, miR-6848-5p, miR-762, miR-2861, miR-1203, miR-1260b, miR-4476, miR-6885-5p, miR-6769b-5p, miR-23b-3p, miR-1343-5p, miR-3621, miR-4688, miR-4286, miR-4640-5p, miR-4739, miR-1260a, miR-4276, miR-7106-5p, miR-6794-5p, miR-6774-5p, miR-4707-3p, miR-4534, miR-4294, miR-6850-5p, miR-6089 and miR-671-5p.

Additional target nucleic acid(s) that may be optionally used in the measurement is preferably selected from the following group 2: miR-128-2-5p, miR-125a-3p, miR-92a-2-5p, and miR-486-3p. Additional target nucleic acid(s) that may be optionally further used in the measurement is preferably selected from the following group 3: miR-3196, miR-211-3p, miR-4271, miR-6851-5p, miR-149-3p, miR-4667-5p, miR-135a-3p, miR-4486, miR-4697-5p, miR-4725-3p, miR-6510-5p, miR-5001-5p, miR-4673, miR-4466, miR-23a-3p, miR-3656, miR-6782-5p, miR-4689, miR-451a, miR-4446-3p, miR-3180-3p, miR-642a-3p, miR-6889-5p, miR-3178, miR-4665-5p, miR-6722-3p, miR-30c-1-3p, miR-4507, miR-3141 and miR-1199-5p.

The kit or the device of the present invention comprises a nucleic acid(s) capable of specifically binding to any of the target nucleic acids as the stomach cancer markers described above, preferably one or more polynucleotides selected from the nucleic acid probes or the primers described in the preceding Section 2, specifically, the polynucleotides described in the preceding Section 2 or variant(s) thereof.

Specifically, the kit or the device of the present invention can comprise at least one or more polynucleotides comprising (or consisting of) a nucleotide sequence represented by any of SEQ ID NOs: 1 to 165 and 635 to 642 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, or variant(s) or fragment(s) comprising 15 or more consecutive nucleotides of any of these polynucleotide sequences.

The kit or the device of the present invention can further comprise one or more polynucleotides comprising (or consisting of) a nucleotide sequence represented by any of SEQ ID NOs: 166 to 169 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, variant(s) or fragment(s) comprising 15 or more consecutive nucleotides of any of these polynucleotide sequences.

The kit or the device of the present invention can further comprise one or more polynucleotides comprising (or consisting of) a nucleotide sequence represented by any of SEQ ID NOs: 170 to 199 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, variant(s) or fragment(s) comprising 15 or more consecutive nucleotides of any of these polynucleotide sequences.

The fragment(s) that can be contained in the kit or the device of the present invention is, for example, one or more, preferably two or more polynucleotides selected from the group consisting of the following polynucleotides (1) to (3):
(1) a polynucleotide comprising 15 or more consecutive nucleotides that are from a nucleotide sequence derived from a nucleotide sequence represented by any of SEQ ID NOs: 1 to 165 and 635 to 642 by the replacement of u with t, or a complementary sequence thereof;
(2) a polynucleotide comprising 15 or more consecutive nucleotides that are from a nucleotide sequence derived from a nucleotide sequence represented by any of SEQ ID NOs: 166 to 169 by the replacement of u with t, or a complementary sequence thereof; and
(3) a polynucleotide comprising 15 or more consecutive nucleotides that are from a nucleotide sequence derived from a nucleotide sequence represented by any of SEQ ID NOs: 170 to 199 by the replacement of u with t, or a complementary sequence thereof.

In a preferred embodiment, the polynucleotide is a polynucleotide consisting of a nucleotide sequence represented by any of SEQ ID NOs: 1 to 165 and 635 to 642 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 166 to 169 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 170 to 199 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the fragment can be a polynucleotide comprising 15 or more, preferably 17 or more, more preferably 19 or more consecutive nucleotides.

In the present invention, the size of the polynucleotide fragment is the number of nucleotides in the range from, for example, 15 consecutive nucleotides to less than the total number of nucleotides of the sequence, from 17 consecutive nucleotides to less than the total number of nucleotides of the sequence, or from 19 consecutive nucleotides to less than the total number of nucleotides of the sequence, in the nucleotide sequence of each polynucleotide.

Specific examples of the aforementioned polynucleotide combination as target nucleic acids for the kit or the device of the present invention can include combinations of the polynucleotides consisting of nucleotide sequences represented by SEQ ID NOs shown in Table 1 (SEQ ID NOs: 1 to 199 and 635 to 642 corresponding to the miRNA markers in Table 1). However, these are given merely for illustrative purposes, and all of various other possible combinations are included in the present invention.

The combination of the target nucleic acids for the kit or the device for discriminating a stomach cancer patient from a healthy subject according to the present invention is desirably, for example, a combination of two or more polynucleotides consisting of the nucleotide sequences represented by SEQ ID NOs shown in Table 1. Usually, a combination of two of these polynucleotides can produce adequate performance.

The specific combination of two polynucleotides consisting of the nucleotide sequences or the complementary sequences thereof for discriminating a stomach cancer patient from a healthy subject is preferably a combination comprising at least one or more of the newly found polynucleotides consisting of the nucleotide sequences represented by SEQ ID NOs: 1 to 165 and 635 to 642, among the combinations of two of the aforementioned polynucleotides consisting of the nucleotide sequences represented by SEQ ID NOs: 1 to 199 and 635 to 642.

The combination of polynucleotides with cancer type specificity capable of discriminating a stomach cancer patient not only from a healthy subject but also from other cancer patients is preferably, for example, a combination of multiple polynucleotides comprising at least one polynucleotide selected from the group consisting of polynucleotides of SEQ ID NOs: 9, 13, 21, 27, 34, 36, 66, 75, 95, 98, 108, 130, 135, 143, 155, 183, 185, 187, 191, 193, 194, 635, 636, 637, 638, 639, 640, 641 and 642 (hereinafter, this group is referred to as “cancer type-specific polynucleotide group 1”), with any of the polynucleotides of the other SEQ ID NOs.

The combination of polynucleotides with cancer type specificity capable of discriminating a stomach cancer patient not only from a healthy subject but also from other cancer patients is more preferably a combination of multiple polynucleotides selected from the cancer type-specific polynucleotide group 1.

The combination of polynucleotides with cancer type specificity capable of discriminating a stomach cancer patient not only from a healthy subject but also from other cancer patients is more preferably a combination comprising at least one or more polynucleotides selected from the group consisting of polynucleotides of SEQ ID NOs: 21, 34, 36, 98, and 155 (hereinafter, this group is referred to as “cancer type-specific polynucleotide group 2”) included in the cancer type-specific polynucleotide group 1, among the combinations of multiple polynucleotides selected from the cancer type-specific polynucleotide group 1.

The number of the polynucleotides with cancer type specificity may be 2, 3, 4, 5, 6, 7, 8, 9, 10 or more in the combination and is more preferably 6 or more in the combination. Usually, the combination of 6 of these polynucleotides can produce adequate performance.

Non-limiting examples of the combination of the polynucleotide consisting of the nucleotide sequence represented by SEQ ID NO: 21 or a complementary sequence thereof with polynucleotides consisting of nucleotide sequences represented by SEQ ID NOs of five polynucleotides selected from the cancer type-specific polynucleotide group 1 or complementary sequences thereof are listed below.
(1) a combination of SEQ ID NOs: 9, 21, 36, 98, 130, and 637 (markers: hsa-miR-6781-5p, hsa-miR-3131, hsa-miR-4419b, hsa-miR-940, hsa-miR-718, and hsa-miR-4707-3p);
(2) a combination of SEQ ID NOs: 9, 21, 34, 36, 98, and 637 (markers: hsa-miR-6781-5p, hsa-miR-3131, hsa-miR-6741-5p, hsa-miR-4419b, hsa-miR-940, and hsa-miR-4707-3p);
(3) a combination of SEQ ID NOs: 9, 21, 34, 36, 98, and 155 (markers: hsa-miR-6781-5p, hsa-miR-3131, hsa-miR-6741-5p, hsa-miR-4419b, hsa-miR-940, and hsa-miR-6769b-5p);
(4) a combination of SEQ ID NOs: 21, 36, 75, 98, 155, and 635 (markers: hsa-miR-3131, hsa-miR-4419b, hsa-miR-6125, hsa-miR-940, hsa-miR-6769b-5p, and hsa-miR-6794-5p); and
(5) a combination of SEQ ID NOs: 9, 21, 36, 98, 108, and 155 (markers: hsa-miR-6781-5p, hsa-miR-3131, hsa-miR-4419b, hsa-miR-940, hsa-miR-1914-3p, and hsa-miR-6769b-5p).

Non-limiting examples of the combination of the polynucleotide consisting of the nucleotide sequence represented by SEQ ID NO: 34 or a complementary sequence thereof with polynucleotides consisting of nucleotide sequences represented by SEQ ID NOs of five polynucleotides selected from the cancer type-specific polynucleotide group 1 or complementary sequences thereof are further listed.
(1) a combination of SEQ ID NOs: 34, 36, 143, 155, 187, and 635 (markers: hsa-miR-6741-5p, hsa-miR-4419b, hsa-miR-3195, hsa-miR-6769b-5p, hsa-miR-4689, and hsa-miR-6794-5p);
(2) a combination of SEQ ID NOs: 9, 34, 36, 66, 98, and 187 (markers: hsa-miR-6781-5p, hsa-miR-6741-5p, hsa-miR-4419b, hsa-miR-615-5p, hsa-miR-940, and hsa-miR-4689);
(3) a combination of SEQ ID NOs: 9, 34, 36, 98, 187, and 637 (markers: hsa-miR-6781-5p, hsa-miR-6741-5p, hsa-miR-4419b, hsa-miR-940, hsa-miR-4689, and hsa-miR-4707-3p);
(4) a combination of SEQ ID NOs: 9, 34, 36, 98, 185, and 637 (markers: hsa-miR-6781-5p, hsa-miR-6741-5p, hsa-miR-4419b, hsa-miR-940, hsa-miR-3656, and hsa-miR-4707-3p); and
(5) a combination of SEQ ID NOs: 9, 34, 36, 98, 637, and 639 (markers: hsa-miR-6781-5p, hsa-miR-6741-5p, hsa-miR-4419b, hsa-miR-940, hsa-miR-4707-3p, and hsa-miR-4294).

Non-limiting examples of the combination of the polynucleotide consisting of the nucleotide sequence represented by SEQ ID NO: 36 or a complementary sequence thereof with polynucleotides consisting of nucleotide sequences represented by SEQ ID NOs of five polynucleotides selected from the cancer type-specific polynucleotide group 1 or complementary sequences thereof are further listed.
(1) a combination of SEQ ID NOs: 9, 36, 98, 108, 638, and 639 (markers: hsa-miR-6781-5p, hsa-miR-4419b, hsa-miR-940, hsa-miR-1914-3p, hsa-miR-4534, and hsa-miR-4294);
(2) a combination of SEQ ID NOs: 36, 98, 155, 194, 635, and 642 (markers: hsa-miR-4419b, hsa-miR-940, hsa-miR-6769b-5p, hsa-miR-4665-5p, hsa-miR-6794-5p, and hsa-miR-671-5p);
(3) a combination of SEQ ID NOs: 9, 34, 36, 75, 98, and 637 (markers: hsa-miR-6781-5p, hsa-miR-6741-5p, hsa-miR-4419b, hsa-miR-6125, hsa-miR-940, and hsa-miR-4707-3p);
(4) a combination of SEQ ID NOs: 21, 36, 98, 155, 185, and 635 (markers: hsa-miR-3131, hsa-miR-4419b, hsa-miR-940, hsa-miR-6769b-5p, hsa-miR-3656, and hsa-miR-6794-5p); and
(5) a combination of SEQ ID NOs: 9, 36, 98, 108, 155, and 635 (markers: hsa-miR-6781-5p, hsa-miR-4419b, hsa-miR-940, hsa-miR-1914-3p, hsa-miR-6769b-5p, and hsa-miR-6794-5p).

Non-limiting examples of the combination of the polynucleotide consisting of the nucleotide sequence represented by SEQ ID NO: 98 or a complementary sequence thereof with polynucleotides consisting of nucleotide sequences represented by SEQ ID NOs of five polynucleotides selected from the cancer type-specific polynucleotide group 1 or complementary sequences thereof are further listed.
(1) a combination of SEQ ID NOs: 9, 36, 98, 130, 194, and 637 (markers: hsa-miR-6781-5p, hsa-miR-4419b, hsa-miR-940, hsa-miR-718, hsa-miR-4665-5p, and hsa-miR-4707-3p);
(2) a combination of SEQ ID NOs: 21, 36, 98, 108, 155, and 635 (markers: hsa-miR-3131, hsa-miR-4419b, hsa-miR-940, hsa-miR-1914-3p, hsa-miR-6769b-5p, and hsa-miR-6794-5p);
(3) a combination of SEQ ID NOs: 9, 36, 98, 108, 155, and 639 (markers: hsa-miR-6781-5p, hsa-miR-4419b, hsa-miR-940, hsa-miR-1914-3p, hsa-miR-6769b-5p, and hsa-miR-4294);
(4) a combination of SEQ ID NOs: 9, 36, 98, 155, 187, and 639 (markers: hsa-miR-6781-5p, hsa-miR-4419b, hsa-miR-940, hsa-miR-6769b-5p, hsa-miR-4689, and hsa-miR-4294); and
(5) a combination of SEQ ID NOs: 9, 36, 98, 155, 187, and 637 (markers: hsa-miR-6781-5p, hsa-miR-4419b, hsa-miR-940, hsa-miR-6769b-5p, hsa-miR-4689, and hsa-miR-4707-3p).

Non-limiting examples of the combination of the polynucleotide consisting of the nucleotide sequence represented by SEQ ID NO: 155 or a complementary sequence thereof with polynucleotides consisting of nucleotide sequences represented by SEQ ID NOs of five polynucleotides selected from the cancer type-specific polynucleotide group 1 or complementary sequences thereof are further listed.
(1) a combination of SEQ ID NOs: 9, 36, 75, 98, 155, and 635 (markers: hsa-miR-6781-5p, hsa-miR-4419b, hsa-miR-6125, hsa-miR-940, hsa-miR-6769b-5p, and hsa-miR-6794-5p);
(2) a combination of SEQ ID NOs: 36, 98, 130, 155, 185, and 635 (markers: hsa-miR-4419b, hsa-miR-940, hsa-miR-718, hsa-miR-6769b-5p, hsa-miR-3656, and hsa-miR-6794-5p);
(3) a combination of SEQ ID NOs: 9, 13, 143, 155, 194, and 639 (markers: hsa-miR-6781-5p, hsa-miR-204-3p, hsa-miR-3195, hsa-miR-6769b-5p, hsa-miR-4665-5p, and hsa-miR-4294);
(4) a combination of SEQ ID NOs: 9, 13, 34, 36, 98, and 155 (markers: hsa-miR-6781-5p, hsa-miR-204-3p, hsa-miR-6741-5p, hsa-miR-4419b, hsa-miR-940, and hsa-miR-6769b-5p); and
(5) a combination of SEQ ID NOs: 36, 98, 108, 155, 193, and 635 (markers: hsa-miR-4419b, hsa-miR-940, hsa-miR-1914-3p, hsa-miR-6769b-5p, hsa-miR-3178, and hsa-miR-6794-5p).

The kit or the device of the present invention can also comprise a polynucleotide(s) that is already known or that will be found in the future, to enable detection of stomach cancer, in addition to the polynucleotide(s) (which may include a variant(s), a fragment(s), and a derivative(s)) as described above according to the present invention.

The kit of the present invention can also comprise an antibody for measuring a marker(s) for stomach cancer examination known in the art, such as CEA, or CA19-9, in addition to the polynucleotide(s) according to the present invention as described above, and a variant(s) thereof or a fragment(s) thereof.

These polynucleotides contained in the kit of the present invention may be packaged in different containers either individually or in any combination.

The kit of the present invention may comprise a kit for extracting a nucleic acid(s) (e.g., total RNA) from body fluids, cells, or tissues, a fluorescent material for labeling, an enzyme and a medium for nucleic acid amplification, an instruction manual, etc.

The device of the present invention is a device for cancer marker measurement in which nucleic acids such as the polynucleotides according to the present invention described above are bonded or attached to, for example, a solid phase. Examples of the material for the solid phase include plastics, paper, glass, and silicon. The material for the solid phase is preferably a plastic from the viewpoint of easy processability. The solid phase has any shape and is, for example, square, round, reed-shaped, or film-shaped. The device of the present invention includes, for example, a device for measurement by a hybridization technique. Specific examples thereof include blotting devices and nucleic acid arrays (e.g., microarrays, DNA chips, and RNA chips).

The nucleic acid array technique is a technique which involves bonding or attaching the nucleic acids one by one by use of a method [e.g., a method of spotting the nucleic acids using a high-density dispenser called spotter or arrayer onto the surface of the solid phase surface-treated, if necessary, by coating with L-lysine or the introduction of a functional group such as an amino group or a carboxyl group, a method of spraying the nucleic acids onto the solid phase using an inkjet which injects very small liquid droplets by a piezoelectric element or the like from a nozzle, or a method of sequentially synthesizing nucleotides on the solid phase] to prepare an array such as a chip and measuring target nucleic acids through the use of hybridization using this array.

The kit or the device of the present invention comprises nucleic acids capable of specifically binding to the polynucleotides of at least one or more, preferably at least two or more, more preferably at least three or more, most preferably at least five or more to all of the stomach cancer marker miRNAs, respectively, of the group 1 described above. The kit or the device of the present invention can optionally further comprise nucleic acids capable of specifically binding to the polynucleotides of at least one or more, preferably at least two or more, more preferably at least three or more, most preferably at least five or more to all of the stomach cancer marker miRNAs, respectively, of the group 2 described above. The kit or the device of the present invention can optionally further comprise nucleic acids capable of specifically binding to the polynucleotides of at least one or more, preferably at least two or more, more preferably at least three or more, most preferably at least five or more to all of the stomach cancer marker miRNAs, respectively, of the group 3 described above.

The kit or the device of the present invention can be used for detecting stomach cancer as described in the Section 4 below.

### 4. Method for Detecting Stomach Cancer

The present invention further provides a method for detecting stomach cancer, comprising using the kit or the device of the present invention (comprising the nucleic acid(s) that can be used in the present invention) described in the preceding Section 3 above to measure an expression level(s) of one or more stomach cancer-derived genes represented by: an expression level(s) of stomach cancer-derived gene(s) selected from the following group: miR-4257, miR-6726-5p, miR-1343-3p, miR-1247-3p, miR-6787-5p, miR-6875-5p, miR-1225-3p, miR-8063, miR-6781-5p, miR-4746-3p, miR-1908-5p, miR-6756-5p, miR-204-3p, miR-4651, miR-6757-5p, miR-6825-5p, miR-7108-5p, miR-4792, miR-7641, miR-3188, miR-3131, miR-6780b-5p, miR-8069, miR-6840-3p, miR-8072, miR-1233-5p, miR-6887-5p, miR-1231, miR-5572, miR-6738-5p, miR-6784-5p, miR-6791-5p, miR-6749-5p, miR-6741-5p, miR-128-1-5p, miR-4419b, miR-6746-5p, miR-3184-5p, miR-3679-5p, miR-7110-5p, miR-4516, miR-6717-5p, miR-6826-5p, miR-4433b-3p, miR-3679-3p, miR-3135b, miR-3622a-5p, miR-711, miR-4467, miR-6857-5p, miR-6515-3p, miR-1225-5p, miR-187-5p, miR-3185, miR-642b-3p, miR-1249, miR-744-5p, miR-4442, miR-1228-3p, miR-939-5p, miR-6845-5p, miR-887-3p, miR-7845-5p, miR-6729-5p, miR-4632-5p, miR-615-5p, miR-6724-5p, miR-4728-5p, miR-6732-5p, miR-6816-5p, miR-4695-5p, miR-6088, miR-7975, miR-3197, miR-6125, miR-4433-3p, miR-6727-5p, miR-4706, miR-7847-3p, miR-6805-3p, miR-6766-3p, miR-1913, miR-4649-5p, miR-602, miR-3663-3p, miR-6893-5p, miR-6861-5p, miR-4449, miR-6842-5p, miR-4454, miR-5195-3p, miR-663b, miR-6765-5p, miR-4513, miR-614, miR-6785-5p, miR-6777-5p, miR-940, miR-4741, miR-6870-5p, miR-6131, miR-150-3p, miR-4707-5p, miR-1915-3p, miR-3937, miR-937-5p, miR-4443, miR-1914-3p, miR-3620-5p, miR-1268b, miR-1227-5p, miR-6880-5p, miR-4417, miR-6802-5p, miR-6769a-5p, miR-663a, miR-6721-5p, miR-4532, miR-7977, miR-92b-5p, miR-371a-5p, miR-6126, miR-4734, miR-4665-3p, miR-423-5p, miR-1469, miR-4675, miR-1915-5p, miR-6716-5p, miR-718, miR-4281, miR-6820-5p, miR-6795-5p, miR-6779-5p, miR-7109-5p, miR-6798-5p, miR-4648, miR-8059, miR-6765-3p, miR-6132, miR-4492, miR-7107-5p, miR-3195, miR-3180, miR-296-3p, miR-564, miR-1268a, miR-6848-5p, miR-762, miR-2861, miR-1203, miR-1260b, miR-4476, miR-6885-5p, miR-6769b-5p, miR-23b-3p, miR-1343-5p, miR-3621, miR-4688, miR-4286, miR-4640-5p, miR-4739, miR-1260a, miR-4276, miR-7106-5p, miR-6794-5p, miR-6774-5p, miR-4707-3p, miR-4534, miR-4294, miR-6850-5p, miR-6089 and miR-671-5p; and optionally an expression level(s) of stomach cancer-derived gene(s) selected from the following group: miR-128-2-5p, miR-125a-3p, miR-92a-2-5p, and miR-486-3p; and optionally an expression level(s) of stomach cancer-derived gene(s) selected from the following group: miR-3196, miR-211-3p, miR-4271, miR-6851-5p, miR-149-3p, miR-4667-5p, miR-135a-3p, miR-4486, miR-4697-5p, miR-4725-3p, miR-6510-5p, miR-5001-5p, miR-4673, miR-4466, miR-23a-3p, miR-3656, miR-6782-5p, miR-4689, miR-451a, miR-4446-3p, miR-3180-3p, miR-642a-3p, miR-6889-5p, miR-3178, miR-4665-5p, miR-6722-3p, miR-30c-1-3p, miR-4507, miR-3141 and miR-1199-5p in a sample in vitro, further comparing, for example, the expression level(s) of the gene(s) in the sample (e.g., blood, serum, or plasma) collected from a subject suspected of having stomach cancer with a control expression level in the sample collected from a healthy subject (including a non-stomach cancer patient), and evaluating the subject as having stomach cancer when the expression level of the target nucleic acid is statistically significantly different between the samples.

This method of the present invention enables a limitedly invasive, early diagnosis of cancer with high sensitivity and specificity and thereby brings about early treatment and improved prognosis. In addition, exacerbation of the disease or the effectiveness of surgical, radiotherapeutic, and chemotherapeutic treatments can be monitored.

The method for extracting the stomach cancer-derived gene(s) from the sample such as blood, serum, or plasma according to the present invention is particularly preferably prepared by the addition of a reagent for RNA extraction in 3D-Gene(™) RNA extraction reagent from liquid sample kit (Toray Industries, Inc.). A general acidic phenol method (acid guanidinium-phenol-chloroform (AGPC)) may be used, or Trizol(™) (Life Technologies Corp.) may be used. The stomach cancer-derived gene(s) may be prepared by the addition of a reagent for RNA extraction containing acidic phenol, such as Trizol (Life Technologies Corp.) or Isogen (Nippon Gene Co., Ltd.). Alternatively, a kit such as miRNeasy(™) Mini Kit (Qiagen N.V.) may be used, though the method is not limited thereto.

The present invention also provides use of the kit or the device of the present invention for detecting in vitro an expression product(s) of a stomach cancer-derived miRNA gene(s) in a sample derived from a subject.

In the method of the present invention, the kit or the device described above comprises a single polynucleotide or any possible combination of polynucleotides that can be used in the present invention as described above.

In the detection or (genetic) diagnosis of stomach cancer according to the present invention, each polynucleotide contained in the kit or the device of the present invention can be used as a probe or a primer. In the case of using the polynucleotide as a primer, TaqMan(™) MicroRNA Assays from Life Technologies Corp., miScript PCR System from Qiagen N.V., or the like can be used, though the method is not limited thereto.

The polynucleotide contained in the kit or the device of the present invention can be used as a primer or a probe according to a routine method in a method known in the art for specifically detecting the particular gene, for example, a hybridization technique such as Northern blot, Southern blot, in situ hybridization, Northern hybridization, or Southern hybridization, or a quantitative amplification technique such as quantitative RT-PCR. A body fluid such as blood, serum, plasma, or urine from a subject is collected as a sample to be assayed according to the type of the detection method used. Alternatively, total RNA prepared from such a body fluid by the method described above may be used, and various polynucleotides including cDNA prepared on the basis of the RNA may be used.

The kit or the device of the present invention is useful for the diagnosis of stomach cancer or the detection of the presence or absence of stomach cancer. Specifically, the detection of stomach cancer using the kit or the device can be performed by detecting in vitro an expression level(s) of a gene(s) using the nucleic acid probe(s) or the primer(s) contained in the kit or the device in a sample such as blood, serum, plasma, or urine from a subject suspected of having stomach cancer. The subject suspected of having stomach cancer can be evaluated as having stomach cancer when the expression level(s) of a target miRNA marker(s) measured using polynucleotide(s) (including a variant(s), a fragment(s), and a derivative(s) thereof) consisting of a nucleotide sequence(s) represented by at least one or more of SEQ ID NOs: 1 to 165 and 635 to 642 or a complementary sequence(s) thereof, optionally a nucleotide sequence(s) represented by one or more of SEQ ID NOs: 166 to 169 or a complementary sequence(s) thereof, and optionally a nucleotide sequence(s) represented by one or more of SEQ ID NOs: 170 to 199 or a complementary sequence(s) thereof in the sample such as blood, serum, plasma, or urine of the subject is statistically significantly different from the expression level(s) thereof in the sample such as blood, serum, or plasma, or urine of a healthy subject.

The method of the present invention can be combined with gastric X-ray examination and gastroscopy as well as a diagnostic imaging method such as CT, PET, or MRI. The method of the present invention is capable of specifically detecting stomach cancer and can substantially discriminate stomach cancer from other cancers.

The method for detecting the absence of an expression product(s) of a stomach cancer-derived gene(s) or the presence of the expression product(s) of a stomach cancer-derived gene(s) in a sample using the kit or the device of the present invention comprises collecting a body fluid such as blood, serum, plasma, or urine of a subject, and measuring the expression level(s) of the target gene(s) contained therein using one or more polynucleotide(s) (including a variant(s), a fragment(s), and a derivative(s)) selected from the polynucleotide group of the present invention, to evaluate the presence or absence of stomach cancer or to detect stomach cancer. Using the method for detecting stomach cancer according to the present invention, for example, the presence or absence of amelioration of the disease or the degree of amelioration thereof in a stomach cancer patient to whom a therapeutic drug for the amelioration of the disease is administered can be also evaluated or diagnosed.

The method of the present invention can comprise, for example, the following steps (a), (b), and (c):
(a) contacting a sample derived from a subject with a polynucleotide(s) in the kit or the device of the present invention in vitro;
(b) measuring an expression level(s) of the target nucleic acid(s) in the sample using the polynucleotide(s) as a nucleic acid probe(s) or a primer(s); and
(c) evaluating the presence or absence of stomach cancer (cells) in the subject on the basis of the result in the step (b).

Specifically, the present invention provides a method for detecting stomach cancer, comprising measuring an expression level(s) of a target nucleic acid(s) in a sample of a subject using a nucleic acid(s) capable of specifically binding to at least one or more (preferably at least two or more) polynucleotide(s) selected from the group consisting of miR-4257, miR-6726-5p, miR-1343-3p, miR-1247-3p, miR-6787-5p, miR-6875-5p, miR-1225-3p, miR-8063, miR-6781-5p, miR-4746-3p, miR-1908-5p, miR-6756-5p, miR-204-3p, miR-4651, miR-6757-5p, miR-6825-5p, miR-7108-5p, miR-4792, miR-7641, miR-3188, miR-3131, miR-6780b-5p, miR-8069, miR-6840-3p, miR-8072, miR-1233-5p, miR-6887-5p, miR-1231, miR-5572, miR-6738-5p, miR-6784-5p, miR-6791-5p, miR-6749-5p, miR-6741-5p, miR-128-1-5p, miR-4419b, miR-6746-5p, miR-3184-5p, miR-3679-5p, miR-7110-5p, miR-4516, miR-6717-5p, miR-6826-5p, miR-4433b-3p, miR-3679-3p, miR-3135b, miR-3622a-5p, miR-711, miR-4467, miR-6857-5p, miR-6515-3p, miR-1225-5p, miR-187-5p, miR-3185, miR-642b-3p, miR-1249, miR-744-5p, miR-4442, miR-1228-3p, miR-939-5p, miR-6845-5p, miR-887-3p, miR-7845-5p, miR-6729-5p, miR-4632-5p, miR-615-5p, miR-6724-5p, miR-4728-5p, miR-6732-5p, miR-6816-5p, miR-4695-5p, miR-6088, miR-7975, miR-3197, miR-6125, miR-4433-3p, miR-6727-5p, miR-4706, miR-7847-3p, miR-6805-3p, miR-6766-3p, miR-1913, miR-4649-5p, miR-602, miR-3663-3p, miR-6893-5p, miR-6861-5p, miR-4449, miR-6842-5p, miR-4454, miR-5195-3p, miR-663b, miR-6765-5p, miR-4513, miR-614, miR-6785-5p, miR-6777-5p, miR-940, miR-4741, miR-6870-5p, miR-6131, miR-150-3p, miR-4707-5p, miR-1915-3p, miR-3937, miR-937-5p, miR-4443, miR-1914-3p, miR-3620-5p, miR-1268b, miR-1227-5p, miR-6880-5p, miR-4417, miR-6802-5p, miR-6769a-5p, miR-663a, miR-6721-5p, miR-4532, miR-7977, miR-92b-5p, miR-371a-5p, miR-6126, miR-4734, miR-4665-3p, miR-423-5p, miR-1469, miR-4675, miR-1915-5p, miR-6716-5p, miR-718, miR-4281, miR-6820-5p, miR-6795-5p, miR-6779-5p, miR-7109-5p, miR-6798-5p, miR-4648, miR-8059, miR-6765-3p, miR-6132, miR-4492, miR-7107-5p, miR-3195, miR-3180, miR-296-3p, miR-564, miR-1268a, miR-6848-5p, miR-762, miR-2861, miR-1203, miR-1260b, miR-4476, miR-6885-5p, miR-6769b-5p, miR-23b-3p, miR-1343-5p, miR-3621, miR-4688, miR-4286, miR-4640-5p, miR-4739, miR-1260a, miR-4276, miR-7106-5p, miR-6794-5p, miR-6774-5p, miR-4707-3p, miR-4534, miR-4294, miR-6850-5p, miR-6089 and miR-671-5p and evaluating in vitro whether or not the subject has stomach cancer using the measured expression level(s) and a control expression level(s) of a healthy subject measured in the same way as above.

The term “evaluation” used herein is evaluation support based on results of in vitro examination, not physician's judgment.

As described above, as for the target nucleic acids in a preferred embodiment of the method of the present invention, specifically, miR-4257 is hsa-miR-4257, miR-6726-5p is hsa-miR-6726-5p, miR-1343-3p is hsa-miR-1343-3p, miR-1247-3p is hsa-miR-1247-3p, miR-6787-5p is hsa-miR-6787-5p, miR-6875-5p is hsa-miR-6875-5p, miR-1225-3p is hsa-miR-1225-3p, miR-8063 is hsa-miR-8063, miR-6781-5p is hsa-miR-6781-5p, miR-4746-3p is hsa-miR-4746-3p, miR-1908-5p is hsa-miR-1908-5p, miR-6756-5p is hsa-miR-6756-5p, miR-204-3p is hsa-miR-204-3p, miR-4651 is hsa-miR-4651, miR-6757-5p is hsa-miR-6757-5p, miR-6825-5p is hsa-miR-6825-5p, miR-7108-5p is hsa-miR-7108-5p, miR-4792 is hsa-miR-4792, miR-7641 is hsa-miR-7641, miR-3188 is hsa-miR-3188, miR-3131 is hsa-miR-3131, miR-6780b-5p is hsa-miR-6780b-5p, miR-8069 is hsa-miR-8069, miR-6840-3p is hsa-miR-6840-3p, miR-8072 is hsa-miR-8072, miR-1233-5p is hsa-miR-1233-5p, miR-6887-5p is hsa-miR-6887-5p, miR-1231 is hsa-miR-1231, miR-5572 is hsa-miR-5572, miR-6738-5p is hsa-miR-6738-5p, miR-6784-5p is hsa-miR-6784-5p, miR-6791-5p is hsa-miR-6791-5p, miR-6749-5p is hsa-miR-6749-5p, miR-6741-5p is hsa-miR-6741-5p, miR-128-1-5p is hsa-miR-128-1-5p, miR-4419b is hsa-miR-4419b, miR-6746-5p is hsa-miR-6746-5p, miR-3184-5p is hsa-miR-3184-5p, miR-3679-5p is hsa-miR-3679-5p, miR-7110-5p is hsa-miR-7110-5p, miR-4516 is hsa-miR-4516, miR-6717-5p is hsa-miR-6717-5p, miR-6826-5p is hsa-miR-6826-5p, miR-4433b-3p is hsa-miR-4433b-3p, miR-3679-3p is hsa-miR-3679-3p, miR-3135b is hsa-miR-3135b, miR-3622a-5p is hsa-miR-3622a-5p, miR-711 is hsa-miR-711, miR-4467 is hsa-miR-4467, miR-6857-5p is hsa-miR-6857-5p, miR-6515-3p is hsa-miR-6515-3p, miR-1225-5p is hsa-miR-1225-5p, miR-187-5p is hsa-miR-187-5p, miR-3185 is hsa-miR-3185, miR-642b-3p is hsa-miR-642b-3p, miR-1249 is hsa-miR-1249, miR-744-5p is hsa-miR-744-5p, miR-4442 is hsa-miR-4442, miR-1228-3p is hsa-miR-1228-3p, miR-939-5p is hsa-miR-939-5p, miR-6845-5p is hsa-miR-6845-5p, miR-887-3p is hsa-miR-887-3p, miR-7845-5p is hsa-miR-7845-5p, miR-6729-5p is hsa-miR-6729-5p, miR-4632-5p is hsa-miR-4632-5p, miR-615-5p is hsa-miR-615-5p, miR-6724-5p is hsa-miR-6724-5p, miR-4728-5p is hsa-miR-4728-5p, miR-6732-5p is hsa-miR-6732-5p, miR-6816-5p is hsa-miR-6816-5p, miR-4695-5p is hsa-miR-4695-5p, miR-6088 is hsa-miR-6088, miR-7975 is hsa-miR-7975, miR-3197 is hsa-miR-3197, miR-6125 is hsa-miR-6125, miR-4433-3p is hsa-miR-4433-3p, miR-6727-5p is hsa-miR-6727-5p, miR-4706 is hsa-miR-4706, miR-7847-3p is hsa-miR-7847-3p, miR-6805-3p is hsa-miR-6805-3p, miR-6766-3p is hsa-miR-6766-3p, miR-1913 is hsa-miR-1913, miR-4649-5p is hsa-miR-4649-5p, miR-602 is hsa-miR-602, miR-3663-3p is hsa-miR-3663-3p, miR-6893-5p is hsa-miR-6893-5p, miR-6861-5p is hsa-miR-6861-5p, miR-4449 is hsa-miR-4449, miR-6842-5p is hsa-miR-6842-5p, miR-4454 is hsa-miR-4454, miR-5195-3p is hsa-miR-5195-3p, miR-663b is hsa-miR-663b, miR-6765-5p is hsa-miR-6765-5p, miR-4513 is hsa-miR-4513, miR-614 is hsa-miR-614, miR-6785-5p is hsa-miR-6785-5p, miR-6777-5p is hsa-miR-6777-5p, miR-940 is hsa-miR-940, miR-4741 is hsa-miR-4741, miR-6870-5p is hsa-miR-6870-5p, miR-6131 is hsa-miR-6131, miR-150-3p is hsa-miR-150-3p, miR-4707-5p is hsa-miR-4707-5p, miR-1915-3p is hsa-miR-1915-3p, miR-3937 is hsa-miR-3937, miR-937-5p is hsa-miR-937-5p, miR-4443 is hsa-miR-4443, miR-1914-3p is hsa-miR-1914-3p, miR-3620-5p is hsa-miR-3620-5p, miR-1268b is hsa-miR-1268b, miR-1227-5p is hsa-miR-1227-5p, miR-6880-5p is hsa-miR-6880-5p, miR-4417 is hsa-miR-4417, miR-6802-5p is hsa-miR-6802-5p, miR-6769a-5p is hsa-miR-6769a-5p, miR-663a is hsa-miR-663a, miR-6721-5p is hsa-miR-6721-5p, miR-4532 is hsa-miR-4532, miR-7977 is hsa-miR-7977, miR-92b-5p is hsa-miR-92b-5p, miR-371a-5p is hsa-miR-371a-5p, miR-6126 is hsa-miR-6126, miR-4734 is hsa-miR-4734, miR-4665-3p is hsa-miR-4665-3p, miR-423-5p is hsa-miR-423-5p, miR-1469 is hsa-miR-1469, miR-4675 is hsa-miR-4675, miR-1915-5p is hsa-miR-1915-5p, miR-6716-5p is hsa-miR-6716-5p, miR-718 is hsa-miR-718, miR-4281 is hsa-miR-4281, miR-6820-5p is hsa-miR-6820-5p, miR-6795-5p is hsa-miR-6795-5p, miR-6779-5p is hsa-miR-6779-5p, miR-7109-5p is hsa-miR-7109-5p, miR-6798-5p is hsa-miR-6798-5p, miR-4648 is hsa-miR-4648, miR-8059 is hsa-miR-8059, miR-6765-3p is hsa-miR-6765-3p, miR-6132 is hsa-miR-6132, miR-4492 is hsa-miR-4492, miR-7107-5p is hsa-miR-7107-5p, miR-3195 is hsa-miR-3195, miR-3180 is hsa-miR-3180, miR-296-3p is hsa-miR-296-3p, miR-564 is hsa-miR-564, miR-1268a is hsa-miR-1268a, miR-6848-5p is hsa-miR-6848-5p, miR-762 is hsa-miR-762, miR-2861 is hsa-miR-2861, miR-1203 is hsa-miR-1203, miR-1260b is hsa-miR-1260b, miR-4476 is hsa-miR-4476, miR-6885-5p is hsa-miR-6885-5p, miR-6769b-5p is hsa-miR-6769b-5p, miR-23b-3p is hsa-miR-23b-3p, miR-1343-5p is hsa-miR-1343-5p, miR-3621 is hsa-miR-3621, miR-4688 is hsa-miR-4688, miR-4286 is hsa-miR-4286, miR-4640-5p is hsa-miR-4640-5p, miR-4739 is hsa-miR-4739, miR-1260a is hsa-miR-1260a, miR-4276 is hsa-miR-4276, miR-7106-5p is hsa-miR-7106-5p, miR-6794-5p is hsa-miR-6794-5p, miR-6774-5p is hsa-miR-6774-5p, miR-4707-3p is hsa-miR-4707-3p, miR-4534 is hsa-miR-4534, miR-4294 is hsa-miR-4294, miR-6850-5p is hsa-miR-6850-5p, miR-6089 is hsa-miR-6089, and miR-671-5p is hsa-miR-671-5p.

In a preferred embodiment of the method of the present invention, specifically, the nucleic acid(s) (specifically, probe(s) or primer(s)) is selected from the group consisting of the following polynucleotides (a) to (e):
(a) a polynucleotide consisting of a nucleotide sequence represented by any of SEQ ID NOs: 1 to 165 and 635 to 642 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 165 and 635 to 642,
(c) a polynucleotide consisting of a nucleotide sequence complementary to a nucleotide sequence represented by any of SEQ ID NOs: 1 to 165 and 635 to 642 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 165 and 635 to 642 or a nucleotide sequence derived from the nucleotide sequence by the replacement of u with t, and
(e) a polynucleotide hybridizing under stringent conditions to any of the polynucleotides (a) to (d).

The method of the present invention can further employ a nucleic acid(s) capable of specifically binding to at least one or more polynucleotides selected from miR-128-2-5p, miR-125a-3p, miR-92a-2-5p, and miR-486-3p.

In a preferred embodiment, as for such a nucleic acid, specifically, miR-128-2-5p is hsa-miR-128-2-5p, miR-125a-3p is hsa-miR-125a-3p, miR-92a-2-5p is hsa-miR-92a-2-5p, and miR-486-3p is hsa-miR-486-3p.

In a preferred embodiment, such a nucleic acid(s) is specifically selected from the group consisting of the following polynucleotides (f) to (j):
(f) a polynucleotide consisting of a nucleotide sequence represented by any of SEQ ID NOs: 166 to 169 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 166 to 169,
(h) a polynucleotide consisting of a nucleotide sequence complementary to a nucleotide sequence represented by any of SEQ ID NOs: 166 to 169 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 166 to 169 or a nucleotide sequence derived from the nucleotide sequence by the replacement of u with t, and
(j) a polynucleotide hybridizing under stringent conditions to any of the polynucleotides (f) to (i).

The method of the present invention can further employ a nucleic acid(s) capable of specifically binding to at least one or more polynucleotides selected from the group consisting of miR-3196, miR-211-3p, miR-4271, miR-6851-5p, miR-149-3p, miR-4667-5p, miR-135a-3p, miR-4486, miR-4697-5p, miR-4725-3p, miR-6510-5p, miR-5001-5p, miR-4673, miR-4466, miR-23a-3p, miR-3656, miR-6782-5p, miR-4689, miR-451a, miR-4446-3p, miR-3180-3p, miR-642a-3p, miR-6889-5p, miR-3178, miR-4665-5p, miR-6722-3p, miR-30c-1-3p, miR-4507, miR-3141, and miR-1199-5p.

In a preferred embodiment, as for such a nucleic acid, specifically, miR-3196 is hsa-miR-3196, miR-211-3p is hsa-miR-211-3p, miR-4271 is hsa-miR-4271, miR-6851-5p is hsa-miR-6851-5p, miR-149-3p is hsa-miR-149-3p, miR-4667-5p is hsa-miR-4667-5p, miR-135a-3p is hsa-miR-135a-3p, miR-4486 is hsa-miR-4486, miR-4697-5p is hsa-miR-4697-5p, miR-4725-3p is hsa-miR-4725-3p, miR-6510-5p is hsa-miR-6510-5p, miR-5001-5p is hsa-miR-5001-5p, miR-4673 is hsa-miR-4673, miR-4466 is hsa-miR-4466, miR-23a-3p is hsa-miR-23a-3p, miR-3656 is hsa-miR-3656, miR-6782-5p is hsa-miR-6782-5p, miR-4689 is hsa-miR-4689, miR-451a is hsa-miR-451a, miR-4446-3p is hsa-miR-4446-3p, miR-3180-3p is hsa-miR-3180-3p, miR-642a-3p is hsa-miR-642a-3p, miR-6889-5p is hsa-miR-6889-5p, miR-3178 is hsa-miR-3178, miR-4665-5p is hsa-miR-4665-5p, miR-6722-3p is hsa-miR-6722-3p, miR-30c-1-3p is hsa-miR-30c-1-3p, miR-4507 is hsa-miR-4507, miR-3141 is hsa-miR-3141, and miR-1199-5p is hsa-miR-1199-5p.

Specifically, the nucleic acid(s) further used is a polynucleotide(s) selected from the group consisting of the following polynucleotides (k) to (o):
(k) a polynucleotide consisting of a nucleotide sequence represented by any of SEQ ID NOs: 170 to 199 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 170 to 199,
(m) a polynucleotide consisting of a nucleotide sequence complementary to a nucleotide sequence represented by any of SEQ ID NOs: 170 to 199 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 170 to 199 or a nucleotide sequence derived from the nucleotide sequence by the replacement of u with t, and
(o) a polynucleotide hybridizing under stringent conditions to any of the polynucleotides (k) to (n).

Examples of the sample used in the method of the present invention can include samples prepared from a living tissue (preferably a stomach tissue) or a body fluid such as blood, serum, plasma, or urine from the subject. Specifically, for example, an RNA-containing sample prepared from the tissue, a polynucleotide-containing sample further prepared therefrom, a body fluid such as blood, serum, plasma, or urine, a portion or the whole of a living tissue collected from the subject by biopsy or the like, or a living tissue excised by surgery can be used, and the sample for measurement can be prepared therefrom.

The subject used herein refers to a mammal, for example, a human, a monkey, a mouse or a rat without any limitation, and is preferably a human.

The steps of the method of the present invention can be changed according to the type of the sample to be assayed.

In the case of using RNA as an analyte, the detection of stomach cancer (cells) can comprise, for example, the following steps (a), (b), and (c):
(a) binding RNA(s) prepared from the sample of the subject or a complementary polynucleotide(s) (cDNA(s)) transcribed therefrom to a polynucleotide(s) in the kit or the device of the present invention;
(b) measuring the sample-derived RNA or the cDNA(s) synthesized from the RNA, bound with the polynucleotide by hybridization using the polynucleotide as a nucleic acid probe(s) or by quantitative RT-PCR using the polynucleotide(s) as a primer(s); and
(c) evaluating the presence or absence of stomach cancer (or stomach cancer-derived gene expression) on the basis of the measurement results of the step (b).

For example, various hybridization methods can be used for detecting, examining, evaluating, or diagnosing stomach cancer (or stomach cancer-derived gene expression) in vitro according to the present invention. For example, Northern blot, Southern blot, RT-PCR, DNA chip analysis, in situ hybridization, Northern hybridization, or Southern hybridization can be used as such a hybridization method.

In the case of using the Northern blot, the presence or absence of expression of each gene or the expression level thereof in the RNA can be detected or measured by use of the nucleic acid probe(s) that can be used in the present invention. Specific examples thereof can include a method which comprises labeling the nucleic acid probe (or a complementary strand) with a radioisotope (³²P, ³³P, ³¹S, etc.), a fluorescent material, or the like, hybridizing the labeled product with the tissue-derived RNA of the subject, which is transferred to a nylon membrane or the like according to a routine method, and then detecting and measuring a signal derived from the label (radioisotope or fluorescent material) on the formed DNA/RNA duplex using a radiation detector (examples thereof can include BAS-1800 II (Fujifilm Corp.)) or a fluorescence detector (examples thereof can include STORM 865 (GE Healthcare Japan Corp.)).

In the case of using the quantitative RT-PCR, the presence or absence of expression of each gene or the expression level thereof in the RNA can be detected or measured by use of the primer that can be used in the present invention. Specific examples thereof can include a method which comprises preparing cDNA from the tissue-derived RNA of the subject according to a routine method, hybridizing a pair of primers (consisting of a plus strand and a reverse strand binding to the cDNA) of the present invention with the cDNA such that the region of each target gene can be amplified with the cDNA as a template, and performing PCR according to a routine method to detect the obtained double-stranded DNA. The method for detecting the double-stranded DNA can include a method of performing the PCR using the primers labeled in advance with a radioisotope or a fluorescent material, a method of electrophoresing the PCR product on an agarose gel and staining the double-stranded DNA with ethidium bromide or the like for detection, and a method of transferring the produced double-stranded DNA to a nylon membrane or the like according to a routine method and hybridizing the double-stranded DNA to a labeled nucleic acid probe for detection.

In the case of using the nucleic acid array analysis, an RNA chip or a DNA chip in which the nucleic acid probes (single-stranded or double-stranded) of the present invention is attached to a substrate (solid phase) is used. Regions having the attached nucleic acid probes are referred to as probe spots, and regions having no attached nucleic acid probe are referred to as blank spots. A group of genes immobilized on a solid-phase substrate is generally called a nucleic acid chip, a nucleic acid array, a microarray, or the like. The DNA or RNA array includes a DNA or RNA macroarray and a DNA or RNA microarray. The term “chip” used herein includes these arrays. 3D-Gene(™) Human miRNA Oligo chip (Toray Industries, Inc.) can be used as the DNA chip, though the DNA chip is not limited thereto.

Examples of the measurement using the DNA chip can include, but are not limited to, a method of detecting and measuring a signal derived from the label on nucleic acid probes using an image detector (examples thereof can include Typhoon 9410 (GE Healthcare) and 3D-Gene(™) scanner (Toray Industries, Inc.)).

The “stringent conditions” used herein are, as mentioned above, conditions under which a nucleic acid probe hybridizes to its target sequence to a larger extent (e.g., a measurement value equal to or larger than “(a mean of background measurement values)+(a standard deviation of the background measurement values)×2”) than that for other sequences.

The stringent conditions are defined by hybridization and subsequent washing conditions. Examples of the hybridization conditions include, but not limited to, 30° C. to 60° C. for 1 to 24 hours in a solution containing SSC, a surfactant, formamide, dextran sulfate, a blocking agent(s), etc. In this context, 1×SSC is an aqueous solution (pH 7.0) containing 150 mM sodium chloride and 15 mM sodium citrate. The surfactant includes, for example, SDS (sodium dodecyl sulfate), Triton, or Tween. The hybridization conditions more preferably involve 3 to 10×SSC and 0.1 to 1% SDS. Examples of the conditions for the washing, following the hybridization, which is another condition to define the stringent conditions, can include conditions involving continuous washing at 30° C. in a solution containing 0.5×SSC and 0.1% SDS, at 30° C. in a solution containing 0.2×SSC and 0.1% SDS, and at 30° C. in a 0.05×SSC solution. It is desirable that the complementary strand should maintain its hybridized state with a target plus strand even by washing under such conditions. Specifically, examples of such a complementary strand can include a strand consisting of a nucleotide sequence in a completely complementary relationship with the nucleotide sequence of the target plus strand, and a strand consisting of a nucleotide sequence having at least 80%, preferably at least 85%, more preferably at least 90% or at least 95%, for example, at least 98% or at least 99% identity to the strand.

Other examples of the “stringent conditions” for the hybridization are described in, for example, Sambrook, J. & Russel, D., Molecular Cloning, A LABORATORY MANUAL, Cold Spring Harbor Laboratory Press, published on Jan. 15, 2001, Vol. 1, 7.42 to 7.45 and Vol. 2, 8.9 to 8.17, and can be used in the present invention.

Examples of the conditions for carrying out PCR using a polynucleotide fragment(s) in the kit of the present invention as a primer(s) include treatment for approximately 15 seconds to 1 minute at 5 to 10° C. plus a Tm value calculated from the sequence(s) of the primer(s), using a PCR buffer having composition such as 10 mM Tris-HCL (pH 8.3), 50 mM KCL, and 1 to 2 mM MgCl₂. Examples of the method for calculating such a Tm value include Tm value=2×(the number of adenine residues+the number of thymine residues)+4×(the number of guanine residues+the number of cytosine residues).

In the case of using the quantitative RT-PCR, a commercially available kit for measurement specially designed for quantitatively measuring miRNA, such as TaqMan(™) MicroRNA Assays (Life Technologies Corp.); LNA(™)-based MicroRNA PCR (Exiqon); or Ncode(™) miRNA qRT-PCT kit (Invitrogen Corp.) may be used.

For the calculation of gene expression levels according to the present invention, statistical treatment described in, for example, Statistical analysis of gene expression microarray data (Speed T., Chapman and Hall/CRC), and A beginner's guide Microarray gene expression data analysis (Causton H. C. et al., Blackwell publishing) can be used, though the calculation method is not limited thereto. For example, twice, preferably 3 times, more preferably 6 times the standard deviation of the measurement values of the blank spots are added to the average measurement value of the blank spots on the DNA chip, and probe spots having a signal value equal to or larger than the resulting value can be regarded as detection spots. Alternatively, the average measurement value of the blank spots is regarded as a background and can be subtracted from the measurement values of the probe spots to determine gene expression levels. A missing value for a gene expression level can be excluded from the analyte, preferably replaced with the smallest value of the gene expression level in each DNA chip, or more preferably replaced with a value obtained by subtracting 0.1 from a logarithmic value of the smallest value of the gene expression level. In order to eliminate low-signal genes, only a gene having a gene expression level of 2⁶, preferably 28, more preferably 2¹⁰ or larger in 20% or more, preferably 50% or more, more preferably 80% or more of the number of measurement samples can be selected as the analyte. Examples of the normalization of the gene expression level include, but are not limited to, global normalization and quantile normalization (Bolstad, B. M. et al., 2003, Bioinformatics, Vol. 19, p. 185-193).

The present invention also provides a method comprising measuring a target gene or gene expression level(s) in a sample derived from a subject using the polynucleotide(s), the kit, or the device (e.g., chip) for detection of the present invention, or a combination thereof, preparing a discriminant (discriminant function) with gene expression levels in a sample derived from a stomach cancer patient and a sample derived from a healthy subject as supervising samples, and determining or evaluating the presence and/or absence of the stomach cancer-derived gene(s) in the sample.

Specifically, the present invention further provides the method comprising: a first step of measuring in vitro an expression level(s) of a target gene(s) (target nucleic acid) in multiple samples that were known to be able to determine or evaluate the presence and/or absence of the stomach cancer-derived gene(s) in the samples, using the polynucleotide(s), the kit, or the device (e.g., chip) for detection of the present invention, or a combination thereof; a second step of constructing a discriminant with the measurement values of the expression level(s) of the target gene(s) that was obtained in the first step as supervising samples; a third step of measuring in vitro an expression level(s) of the target gene(s) in a sample derived from a subject in the same way as in the first step; and a fourth step of substituting the measurement value(s) of the expression level(s) of the target gene(s) obtained in the third step into the discriminant obtained in the second step, and determining or evaluating the presence or absence of the stomach cancer-derived gene(s) in the sample on the basis of the results obtained from the discriminant, wherein the target gene(s) can be detected using the polynucleotide(s) or using a polynucleotide(s) for the detection, that was contained in the polynucleotide, the kit or the device (e.g., chip). In this context, the discriminant can be prepared by use of Fisher's discriminant analysis, nonlinear discriminant analysis based on Mahalanobis' distance, neural network, Support Vector Machine (SVM), or the like, though the method is not limited thereto.

When a clustering boundary is a straight line or a hyperplane, the linear discriminant analysis is a method for determining the association of a cluster using Formula 1 as a discriminant. In Formula 1, x represents an explanatory variable, w represents a coefficient of the explanatory variable, and w0 represents a constant term.

$$f ( x ) = {w}_{0} + {\sum }_{i = 1}^{n} {w}_{i} {x}_{i} \qquad Formula 1$$

Values obtained from the discriminant are referred to as discriminant scores. The measurement values of a newly offered data set can be substituted as explanatory variables into the discriminant to determine clusters on the basis of the signs of the discriminant scores.

The Fisher's discriminant analysis, one type of linear discriminant analysis, is a dimension reduction method for selecting a dimension suitable for discriminating classes, and constructs a highly discriminating synthetic variable by focusing on the variance of the synthetic variables and minimizing the variance of data having the same label (Venables, W. N. et al., Modern Applied Statistics with S. Fourth edition. Springer., 2002). In the Fisher's discriminant analysis, direction w of projection is determined so as to maximize Formula 2. In Formula 2, μ represents an average input, ng represents the number of data associate with class g, and μg represents an average input of the data associate with class g. The numerator and the denominator are the interclass variance and the intraclass variance, respectively, when each data is projected in the direction of the vector w. Discriminant coefficient wi is determined by maximizing this ratio (Takafumi Kanamori et al., “Pattern Recognition”, Kyoritsu Shuppan Co., Ltd. (2009); and Richard O. et al., Pattern Classification Second Edition., Wiley-Interscience, 2000).

$$J ( w ) = \frac{{\sum }_{g = 1}^{G} {n}_{g} ( {w}^{T} {μ}_{g} - {w}^{T} μ ) {( {w}^{T} {μ}_{g} - {w}^{T} μ )}^{T}}{{\sum }_{g = 1}^{G} {\sum }_{i : {y}_{i} = g} ( {w}^{T} {x}_{i} - {w}^{T} {μ}_{g} ) ( {w}^{T} {x}_{i} - {w}^{T} {μ}_{g} )} \\ subject to μ = {\sum }_{i = 1}^{n} \frac{{x}_{i}}{n} , {μ}_{g} = {\sum }_{i : {u}_{i} = g}^{n} \frac{{x}_{i}}{{n}_{g}} \qquad Formula 2$$

The Mahalanobis' distance is calculated according to Formula 3 in consideration of data correlation and can be used as nonlinear discriminant analysis for determining a cluster in which a data point belongs to, based on a short Mahalanobis' distance from the data point to that cluster. In Formula 3, p represents a central vector of each cluster, and S-1 represents an inverse matrix of the variance-covariance matrix of the cluster. The central vector is calculated from explanatory variable x, and an average vector, a median value vector, or the like can be used.

$$D ( x , μ ) = {\{ {( x - μ )}^{t} {S}^{- 1} ( x - μ ) \}}^{\frac{1}{2}} \qquad Formula 3$$

SVM is a discriminant analysis method devised by V. Vapnik (The Nature of Statistical Leaning Theory, Springer, 1995). Particular data points of a data set having known classes are defined as explanatory variables, and classes are defined as objective variables. A boundary plane called hyperplane for correctly classifying the data set into the known classes is determined, and a discriminant for data classification is determined using the boundary plane. Then, the measurement values of a newly offered data set can be substituted as explanatory variables into the discriminant to determine classes. In this respect, the result of the discriminant analysis may be classes, may be a probability of data to be classified into correct classes, or may be the distance from the hyperplane. In SVM, a method of nonlinearly converting a feature vector to a high dimension and performing linear discriminant analysis in the space is known as a method for tackling nonlinear problems. An expression in which an inner product of two factors in a nonlinearly mapped space is expressed only by inputs in their original spaces is called kernel. Examples of the kernel can include a linear kernel, a RBF (Radial Basis Function) kernel, and a Gaussian kernel. While highly dimensional mapping is performed according to the kernel, the optimum discriminant, i.e., a discriminant, can be actually constructed by mere calculation according to the kernel, which avoids calculating features in the mapped space (e.g., Hideki Aso et al., Frontier of Statistical Science 6 “Statistics of pattern recognition and learning—New concepts and approaches”, Iwanami Shoten, Publishers (2004); Nello Cristianini et al., Introduction to SVM, Kyoritsu Shuppan Co., Ltd. (2008)).

C-support vector classification (C-SVC), one type of SVM, comprises preparing a hyperplane by supervising a data set with the explanatory variables of two groups and classifying an unknown data set into either of the groups (C. Cortes et al., 1995, Machine Learning, Vol. 20, p. 273-297).

Exemplary calculation of the C-SVC discriminant that can be used in the method of the present invention will be given below. First, all subjects are divided into two groups, i.e., a stomach cancer patient group and a healthy subject group. For example, stomach tissue examination can be used for each subject to be confirmed either as a stomach cancer patient or a healthy subject.

Next, a data set consisting of comprehensive gene expression levels of serum-derived samples of the two divided groups (hereinafter, this data set is referred to as a training cohort) is prepared, and a C-SVC discriminant is determined by using genes that were found to differ clearly in their gene expression levels between the two groups as explanatory variables and using this grouping as objective variables (e.g., −1 and +1). An optimizing objective function is represented by Formula 4 wherein e represents all input vectors, y represents an objective variable, a represents a Lagrange's undetermined multiplier vector, Q represents a positive definite matrix, and C represents a parameter for adjusting constrained conditions.

$${min}_{a} \frac{1}{2} {a}^{T} Qa - {e}^{T} a \\ subject to {y}^{T} a = 0 , 0 \leq {a}_{i} \leq C , i = 1 , … , l , \qquad Formula 4$$

Formula 5 is a finally obtained discriminant, and a group in which the data point belongs to can be determined on the basis of the sign of a value obtained according to the discriminant. In this formula, x represents a support vector, y represents a label indicating the association of a group, a represents the corresponding coefficient, b represents a constant term, and K represents a kernel function.

$$f ( x ) = sgn ( {\sum }_{i = 1}^{l} {y}_{i} {a}_{i} K ( {x}_{i} , x ) + b ) \qquad Formula 5$$

For example, a RBF kernel defined by Formula 6 can be used as the kernel function. In this formula, x represents a support vector, and y represents a kernel parameter for adjusting the complexity of the hyperplane.

$$K ( {x}_{i} , {x}_{j} ) = exp ( - r {‖ {x}_{i} - {x}_{j} ‖}^{2} ) , r < 0 \qquad Formula 6$$

In addition, an approach such as neural network, k-nearest neighbor algorithms, decision trees, or logistic regression analysis can be selected as a method for determining or evaluating the presence and/or absence of expression of a stomach cancer-derived target gene(s) in a sample derived from a subject, or for evaluating the expression level thereof by comparison with a control derived from a healthy subject.

The method of the present invention can comprise, for example, the following steps (a), (b), and (c):
(a) measuring an expression level(s) of a target gene(s) in tissues containing stomach cancer-derived genes derived from stomach cancer patients and/or samples already known to be tissues containing no stomach cancer-derived gene(s) derived from healthy subjects, using the polynucleotide(s), the kit, or the device (e.g., DNA chip) for detection according to the present invention;
(b) preparing the discriminants of Formulas 1 to 3, 5, and 6 described above from the measurement values of the expression level measured in the step (a); and
(c) measuring an expression level(s) of the target gene(s) in a sample derived from a subject using the polynucleotide(s), the kit, or the device (e.g., DNA chip) for diagnosis (detection) according to the present invention, substituting the obtained measurement value(s) into the discriminants prepared in the step (b), and determining or evaluating the presence and/or absence of the stomach cancer-derived target gene(s) in the sample, or evaluating the expression level(s) thereof by comparison with a healthy subject-derived control, on the basis of the obtained results. In this context, in the discriminants of Formulas 1 to 3, 5, and 6, x represents an explanatory variable and includes a value obtained by measuring a polynucleotide(s) selected from the polynucleotides described in the Section 2 above, or any fragment thereof, etc. Specifically, the explanatory variable for discriminating a stomach cancer patient from a healthy subject according to the present invention is a gene expression level(s) selected from, for example, the following expression levels (1) to (3):
(1) a gene expression level(s) in the serum of a stomach cancer patient or a healthy subject measured by any DNA comprising 15 or more consecutive nucleotides in a nucleotide sequence represented by any of SEQ ID NOs: 1 to 165 and 635 to 642 or a complementary sequence thereof,
(2) a gene expression level(s) in the serum of a stomach cancer patient or a healthy subject measured by any DNA comprising 15 or more consecutive nucleotides in a nucleotide sequence represented by any of SEQ ID NOs: 166 to 169 or a complementary sequence thereof, and
(3) a gene expression level(s) in the serum of a stomach cancer patient or a healthy subject measured by any DNA comprising 15 or more consecutive nucleotides in a nucleotide sequence represented by any of SEQ ID NOs: 170 to 199 or a complementary sequence thereof.

As described above, for the method for determining or evaluating the presence and/or absence of a stomach cancer-derived gene(s) in a sample derived from a subject, the preparation of a discriminant requires a discriminant prepared in a training cohort. For enhancing the accuracy of the discriminant, it is necessary for the discriminant to use genes that show clear difference between two groups in the training cohort.

Each gene that is used for an explanatory variable in a discriminant is preferably determined as follows. First, comprehensive gene expression levels of a stomach cancer patient group and comprehensive gene expression levels of a healthy subject group in a training cohort are used as a data set, the degree of difference in the expression level of each gene between the two groups is determined through the use of, for example, the P value of t test, which is parametric analysis, or the P value of Mann-Whitney's U test or Wilcoxon test, which is nonparametric analysis.

The gene can be regarded as being statistically significant when the critical rate (significance level) as the P value obtained by the test is smaller than, for example, 5%, 1%, or 0.01%.

In order to correct an increased probability of type I error attributed to the repetition of a test, a method known in the art, for example, Bonferroni or Holm method, can be used for the correction (e.g., Yasushi Nagata et al., “Basics of statistical multiple comparison methods”, Scientist Press Co., Ltd. (2007)). As an example of the Bonferroni correction, for example, the P value obtained by a test is multiplied by the number of repetitions of the test, i.e., the number of genes used in the analysis, and the obtained value can be compared with a desired significance level to suppress a probability of causing type I error in the whole test.

Instead of the statistical test, the absolute value (fold change) of an expression ratio of a median value of each gene expression level between gene expression levels of a stomach cancer patient group and gene expression levels of a healthy subject group may be calculated to select a gene that is used for an explanatory variable in a discriminant. Alternatively, ROC curves may be prepared using gene expression levels of a stomach cancer patient group and a healthy subject group, and a gene that is used for an explanatory variable in a discriminant can be selected on the basis of an AUROC value.

Next, a discriminant that can be calculated by various methods described above is prepared using any number of genes having large difference in their gene expression levels determined here. Examples of the method for constructing a discriminant that produces the largest discriminant accuracy include a method of constructing a discriminant in every combination of genes that satisfy the significance level of P value, and a method of repetitively evaluating a discriminant while increasing the number of genes for use one by one in a descending order of difference in gene expression level (Furey T S. et al., 2000, Bioinformatics., Vol. 16, p. 906-14). A gene expression level of another independent stomach cancer patient or healthy subject is substituted as an explanatory variable into this discriminant to calculate discriminant results of the group to which this independent stomach cancer patient or healthy subject associate. Specifically, the found gene set for diagnosis and the discriminant constructed using the gene set for diagnosis can be evaluated in an independent sample cohort to find a more universal gene set for diagnosis capable of detecting stomach cancer and a more universal method for discriminating stomach cancer.

Split-sample method is preferably used for evaluating the discriminant performance (generality) of the discriminant. Specifically, a data set is divided into a training cohort and a validation cohort, and gene selection by a statistical test and discriminant preparation are performed in the training cohort. Accuracy, sensitivity, and specificity are calculated using a result of discriminating a validation cohort according to the discriminant and a true group to which the validation cohort associate, to evaluate the performance of the discriminant. On the other hand, instead of dividing a data set, the gene selection by a statistical test and discriminant preparation may be performed using all of samples, and accuracy, sensitivity, and specificity can be calculated by the discriminant analysis of a newly prepared samples for evaluation of the performance of the discriminant.

The present invention provides a polynucleotide(s) for detection or for disease diagnosis useful in the diagnosis and treatment of stomach cancer, a method for detecting stomach cancer using the polynucleotide(s), and a kit and a device for the detection of stomach cancer, comprising the polynucleotide(s). Particularly, in order to select a gene(s) for diagnosis and prepare a discriminant so as to exhibit accuracy beyond a stomach cancer diagnosis method using existing tumor markers CEA and CA19-9, a gene set for diagnosis and a discriminant for the method of the present invention, that exhibit accuracy beyond CEA and CA19-9, can be constructed, for example, by comparing expressed genes in serum derived from a patient confirmed to be negative using CEA and CA19-9 but finally found to have stomach cancer by detailed examination such as computed tomography using a contrast medium, with genes expressed in serum derived from a patient having no stomach cancer.

For example, the gene set for diagnosis is set to any combination selected from one or two or more of the polynucleotides based on a nucleotide sequence(s) represented by any of SEQ ID NOs: 1 to 165 and 635 to 642 or a complementary sequence(s) thereof as described above; and optionally one or two or more of the polynucleotides based on a nucleotide sequence(s) represented by any of SEQ ID NOs: 166 to 169 or a complementary sequence(s) thereof; and optionally one or two or more of the polynucleotides based on a nucleotide sequence represented by any of SEQ ID NOs: 170 to 199 or a complementary sequence thereof. Further, a discriminant is constructed using expression levels of the gene set for diagnosis in samples derived from class I stomach cancer patients and samples derived from class II healthy subjects as a result of tissue diagnosis. As a result, the presence or absence of stomach cancer-derived genes in an unknown sample can be determined with 100% accuracy at the maximum by measuring expression levels of the gene set for diagnosis in an unknown sample.

### EXAMPLES

Hereinafter, the present invention will be described further specifically with reference to Examples below. However, the scope of the present invention is not intended to be limited by these Examples.

### Reference Example 1

<Collection of Samples from Stomach Cancer Patient and Healthy Subject>

Serum was collected using VENOJECT II vacuum blood collecting tube VP-AS109K60 (Terumo Corp.) from each of 100 healthy subjects and 34 stomach cancer patients (19 cases with stage IA, 5 cases with stage IB, 2 cases with stage IIA, 2 cases with stage IIB, 3 cases with stage IIIA, and 3 cases with stage IIIC) with no primary cancer found in areas other than stomach cancer after acquisition of informed consent, and used as a training cohort. Likewise, serum was collected using VENOJECT II vacuum blood collecting tube VP-AS109K60 (Terumo Corp.) from each of 50 healthy subjects and 16 stomach cancer patients (9 cases with stage IA, 2 cases with stage IB, 2 cases with stage IIA, 1 case with stage IIB, 1 case with stage IIIA, and 1 case with stage IIIC) with no primary cancer found in areas other than stomach cancer after acquisition of informed consent, and used as a validation cohort.

### <Extraction of Total RNA>

Total RNA was obtained from 300 μL of the serum sample obtained from each of 200 persons in total of 150 healthy subjects and 50 stomach cancer patients included in the training cohort and the validation cohort, using a reagent for RNA extraction in 3D-Gene(™) RNA extraction reagent from liquid sample kit (Toray Industries, Inc.) according to the protocol provided by the manufacturer.

### <Measurement of Gene Expression Level>

miRNAs in the total RNA obtained from the serum samples of each of 200 persons in total of 150 healthy subjects and 50 stomach cancer patients included in the training cohort and the validation cohort were fluorescently labeled using 3D-Gene(™) miRNA Labeling kit (Toray Industries, Inc.) according to the protocol (ver 2.20) provided by the manufacturer. The oligo DNA chip used was 3D-Gene(™) Human miRNA Oligo chip (Toray Industries, Inc.) with attached probes having sequences complementary to 2,555 miRNAs among the miRNAs registered in miRBase Release 20. Hybridization between the miRNAs in the total RNA and the probes on the DNA chip under stringent conditions and washing following the hybridization were performed according to the protocol provided by the manufacturer. The DNA chip was scanned using 3D-Gene(™) scanner (Toray Industries, Inc.) to obtain images. Fluorescence intensity was digitized using 3D-Gene(™) Extraction (Toray Industries, Inc.). The digitized fluorescence intensity was converted to a logarithmic value having a base of 2 and used as a gene expression level, from which a blank value was subtracted. A missing value was replaced with a value obtained by subtracting 0.1 from a logarithmic value of the smallest value of the gene expression level in each DNA chip. As a result, the comprehensive gene expression levels of the miRNAs in the sera were obtained in the 50 stomach cancer patients and the 150 healthy subjects. Calculation and statistical analysis using the digitized gene expression levels of the miRNAs were carried out using R language 3.0.2 (R Development Core Team (2013). R: A language and environment for statistical computing. R Foundation for Statistical Computing, URL http://www.R-project.org/) and MASS package 7.3-30 (Venables, W. N. & Ripley, B. D. (2002) Modern Applied Statistics with S. Fourth Edition. Springer, New York. ISBN 0-387-95457-0).

### Reference Example 2

<Collection of Sample from Patient with Cancer Other than Stomach Cancers>

Serum was collected using VENOJECT II vacuum blood collecting tube VP-AS109K60 (Terumo Corp.) from each of 63 pancreatic cancer patients, 65 bile duct cancer patients, 35 colorectal cancer patients, 32 liver cancer patients, and 17 benign pancreaticobiliary disease patients with no cancer found in other organs after acquisition of informed consent, and used as a training cohort together with the samples of 34 stomach cancer patients and 102 healthy subjects of Reference Example 1. Likewise, serum was collected using VENOJECT II vacuum blood collecting tube VP-AS109K60 (Terumo Corp.) from each of 37 pancreatic cancer patients, 33 bile duct cancer patients, 15 colorectal cancer patients, 20 liver cancer patients, and 4 benign pancreaticobiliary disease patients with no cancer found in other organs after acquisition of informed consent, and used as a validation cohort together with the samples of 16 stomach cancer patients with no cancer found in areas other than stomach cancer and 48 healthy subjects of Reference Example 1. Subsequent operations were conducted in the same way as in Reference Example 1.

### Example 1

### <Selection of Gene Markers Using the Training Cohort, and Method for Evaluating Stomach Cancer Discriminant Performance of Single Gene Marker Using the Validation Cohort>

In this Example, a gene marker for discriminating a stomach cancer patient from a healthy subject was selected from the training cohort and studied in samples of the validation cohort independent of the training cohort, for a method for evaluating the stomach cancer discriminant performance of each selected gene marker alone.

Specifically, first, the miRNA expression levels of the training cohort and the validation cohort obtained in the preceding Reference Examples were combined and normalized by quantile normalization.

Next, genes for diagnosis were selected using the training cohort. Here, in order to acquire diagnostic markers with higher reliability, only genes having the expression level of 2⁶ or higher in 50% or more of the samples in either of the stomach cancer patient group of the training cohort or the healthy subject group of the training cohort were selected. In order to further acquire statistically significant genes for discriminating a stomach cancer patient group from a healthy subject group, the P value obtained by two-tailed t-test assuming equal variance as to each gene expression level was corrected by the Bonferroni method, and genes that satisfied p<0.01 were acquired as gene markers for use in explanatory variables of a discriminant. The result is described in Table 2.

In this way, polynucleotides consisting of hsa-miR-4257, hsa-miR-6726-5p, hsa-miR-1343-3p, hsa-miR-1247-3p, hsa-miR-6787-5p, hsa-miR-6875-5p, hsa-miR-1225-3p, hsa-miR-8063, hsa-miR-6781-5p, hsa-miR-4746-3p, hsa-miR-1908-5p, hsa-miR-6756-5p, hsa-miR-204-3p, hsa-miR-4651, hsa-miR-6757-5p, hsa-miR-6825-5p, hsa-miR-7108-5p, hsa-miR-4792, hsa-miR-7641, hsa-miR-3188, hsa-miR-3131, hsa-miR-6780b-5p, hsa-miR-8069, hsa-miR-6840-3p, hsa-miR-8072, hsa-miR-1233-5p, hsa-miR-6887-5p, hsa-miR-1231, hsa-miR-5572, hsa-miR-6738-5p, hsa-miR-6784-5p, hsa-miR-6791-5p, hsa-miR-6749-5p, hsa-miR-6741-5p, hsa-miR-128-1-5p, hsa-miR-4419b, hsa-miR-6746-5p, hsa-miR-3184-5p, hsa-miR-3679-5p, hsa-miR-7110-5p, hsa-miR-4516, hsa-miR-6717-5p, hsa-miR-6826-5p, hsa-miR-4433b-3p, hsa-miR-3679-3p, hsa-miR-3135b, hsa-miR-3622a-5p, hsa-miR-711, hsa-miR-4467, hsa-miR-6857-5p, hsa-miR-6515-3p, hsa-miR-1225-5p, hsa-miR-187-5p, hsa-miR-3185, hsa-miR-642b-3p, hsa-miR-1249, hsa-miR-744-5p, hsa-miR-4442, hsa-miR-1228-3p, hsa-miR-939-5p, hsa-miR-6845-5p, hsa-miR-887-3p, hsa-miR-7845-5p, hsa-miR-6729-5p, hsa-miR-4632-5p, hsa-miR-615-5p, hsa-miR-6724-5p, hsa-miR-4728-5p, hsa-miR-6732-5p, hsa-miR-6816-5p, hsa-miR-4695-5p, hsa-miR-6088, hsa-miR-7975, hsa-miR-3197, hsa-miR-6125, hsa-miR-4433-3p, hsa-miR-6727-5p, hsa-miR-4706, hsa-miR-7847-3p, hsa-miR-6805-3p, hsa-miR-6766-3p, hsa-miR-1913, hsa-miR-4649-5p, hsa-miR-602, hsa-miR-3663-3p, hsa-miR-6893-5p, hsa-miR-6861-5p, hsa-miR-4449, hsa-miR-6842-5p, hsa-miR-4454, hsa-miR-5195-3p, hsa-miR-663b, hsa-miR-6765-5p, hsa-miR-4513, hsa-miR-614, hsa-miR-6785-5p, hsa-miR-6777-5p, hsa-miR-940, hsa-miR-4741, hsa-miR-6870-5p, hsa-miR-6131, hsa-miR-150-3p, hsa-miR-4707-5p, hsa-miR-1915-3p, hsa-miR-3937, hsa-miR-937-5p, hsa-miR-4443, hsa-miR-1914-3p, hsa-miR-3620-5p, hsa-miR-1268b, hsa-miR-1227-5p, hsa-miR-6880-5p, hsa-miR-4417, hsa-miR-6802-5p, hsa-miR-6769a-5p, hsa-miR-663a, hsa-miR-6721-5p, hsa-miR-4532, hsa-miR-7977, hsa-miR-92b-5p, hsa-miR-371a-5p, hsa-miR-6126, hsa-miR-4734, hsa-miR-4665-3p, hsa-miR-423-5p, hsa-miR-1469, hsa-miR-4675, hsa-miR-1915-5p, hsa-miR-6716-5p, hsa-miR-718, hsa-miR-4281, hsa-miR-6820-5p, hsa-miR-6795-5p, hsa-miR-6779-5p, hsa-miR-7109-5p, hsa-miR-6798-5p, hsa-miR-4648, hsa-miR-8059, hsa-miR-6765-3p, hsa-miR-6132, hsa-miR-4492, hsa-miR-7107-5p, hsa-miR-3195, hsa-miR-3180, hsa-miR-296-3p, hsa-miR-564, hsa-miR-1268a, hsa-miR-6848-5p, hsa-miR-762, hsa-miR-2861, hsa-miR-1203, hsa-miR-1260b, hsa-miR-4476, hsa-miR-6885-5p, hsa-miR-6769b-5p, hsa-miR-23b-3p, hsa-miR-1343-5p, hsa-miR-3621, hsa-miR-4688, hsa-miR-4286, hsa-miR-4640-5p, hsa-miR-4739, hsa-miR-1260a, hsa-miR-4276, hsa-miR-7106, hsa-miR-128-2-5p, hsa-miR-125a-3p, hsa-miR-92a-2-5p and hsa-miR-486-3p genes, and the nucleotide sequences of SEQ ID NOs: 1 to 169 related thereto were found.

Among them, genes newly found as markers for examining the presence or absence of stomach cancer are polynucleotides consisting of the nucleotide sequences represented by SEQ ID NOs: 1 to 165.

A discriminant for determining the presence or absence of stomach cancer was further prepared by Fisher's discriminant analysis with the expression levels of these genes as indicators. Specifically, any newly found polynucleotide consisting of a nucleotide sequence represented by any of SEQ ID NOs: 1 to 169 in the training cohort was applied for Formula 2 to construct a discriminant. Calculated accuracy, sensitivity, and specificity are shown in Table 3. In this respect, a discriminant coefficient and a constant term are shown in Table 4. In this context, all of the polynucleotides consisting of the nucleotide sequences represented by SEQ ID NOs: 1 to 169 were selected as markers capable of determining all of papillary adenocarcinoma, tubular adenocarcinoma (3 cases), low differentiated adenocarcinoma, signet-ring cell carcinoma, and mucinous carcinoma, which are main types of stomach cancer.

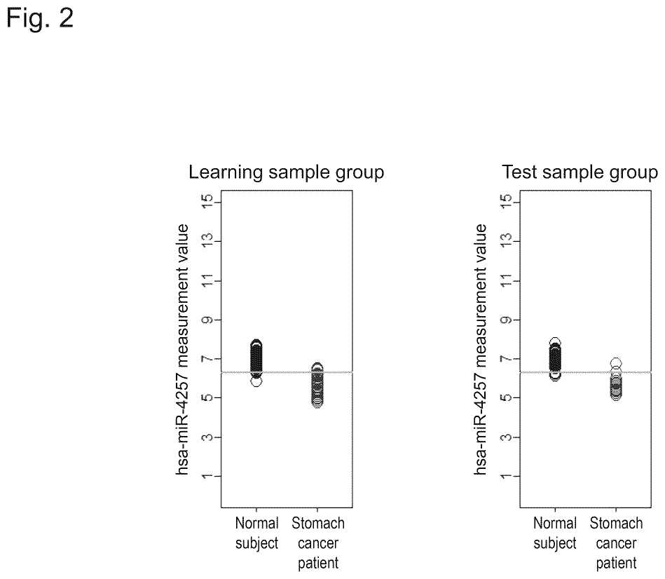

Next, accuracy, sensitivity, and specificity in the validation cohort were calculated using the discriminant thus prepared, and the discriminant performance of the selected polynucleotides was validated using independent samples (Table 3). For example, the expression level measurement value of the nucleotide sequence represented by SEQ ID NO: 1 was compared between the healthy subjects (100 persons) and the stomach cancer patients (34 persons) in the training cohort. As a result, the gene expression level measurement values were found to be significantly lower in the stomach cancer patient group than in the healthy subject group (see the left diagram of FIG. 2). These results were also reproducible in the healthy subjects (50 persons) and the stomach cancer patients (16 persons) in the validation cohort (see the right diagram of FIG. 2). Likewise, the results obtained about the other polynucleotides shown in SEQ ID NOs: 2 to 169 showed that the gene expression level measurement values were significantly lower (−) or higher (+) in the stomach cancer patient group than in the healthy subject group (Table 2). These results were able to be validated in the validation cohort. For example, as for this nucleotide sequence represented by SEQ ID NO: 1, the number of correctly or incorrectly identified samples in the detection of stomach cancer was calculated using the threshold (6.29) that was set in the training cohort and discriminated between the groups. As a result, 14 true positives, 49 true negatives, 1 false positive, and 2 false negatives were obtained. From these values, 95.5% accuracy, 87.5% sensitivity, and 98% specificity were obtained as the detection performance. In this way, the detection performance was calculated as to all of the polynucleotides shown in SEQ ID NOs: 1 to 169, and described in Table 3. The polynucleotides consisting of the nucleotide sequences represented by SEQ ID NOs: 1 to 169 shown in Table 3, exhibited sensitivity of 87.5%, 93.8%, 93.8%, 81.2%, 93.8%, 87.5%, 87.5%, 81.2%, 68.8%, 87.5%, 75.0%, 81.2%, 87.5%, 75.0%, 81.2%, 93.8%, 68.8%, 81.2%, 56.2%, 68.8%, 87.5%, 56.2%, 62.5%, 62.5%, 62.5%, 75.0%, 56.2%, 87.5%, 93.8%, 62.5%, 87.5%, 62.5%, 68.8%, 81.2%, 81.2%, 62.5%, 81.2%, 81.2%, 62.5%, 87.5%, 62.5%, 75.0%, 56.2%, 75.0%, 62.5%, 56.2%, 68.8%, 62.5%, 56.2%, 93.8%, 62.5%, 62.5%, 56.2%, 81.2%, 68.8%, 56.2%, 43.8%, 75.0%, 75.0%, 68.8%, 81.2%, 75.0%, 68.8%, 68.8%, 43.8%, 62.5%, 50.0%, 50.0%, 62.5%, 62.5%, 50.0%, 68.8%, 37.5%, 50.0%, 37.5%, 68.8%, 68.8%, 56.2%, 12.5%, 75.0%, 50.0%, 50.0%, 37.5%, 68.8%, 25.0%, 81.2%, 43.8%, 56.2%, 62.5%, 37.5%, 43.8%, 43.8%, 37.5%, 43.8%, 31.2%, 43.8%, 50.0%, 25%, 43.8%, 37.5%, 37.5%, 31.2%, 25.0%, 25.0%, 56.2%, 31.2%, 43.8%, 56.2%, 50.0%, 37.5%, 31.2%, 31.2%, 37.5%, 50.0%, 12.5%, 31.2%, 56.2%, 18.8%, 43.8%, 18.8%, 37.5%, 31.2%, 37.5%, 50.0%, 50.0%, 12.5%, 31.2%, 31.2%, 31.2%, 31.2%, 50.0%, 37.5%, 18.8%, 37.5%, 50.0%, 43.8%, 18.8%, 43.8%, 31.2%, 18.8%, 50.0%, 25.0%, 31.2%, 31.2%, 18.8%, 43.8%, 6.2%, 25.0%, 12.5%, 31.2%, 12.5%, 18.8%, 37.5%, 6.2%, 31.2%, 6.2%, 18.8%, 6.2%, 18.8%, 6.2%, 12.5%, 18.8%, 6.2%, 12.5%, 6.2%, 50.0%, 68.8%, 31.2%, and 25.0%, respectively, in the validation cohort. As seen from Comparative Example mentioned later, the existing markers CEA and CA19-9 had sensitivity of 12.5% (when the abnormal value of CEA was defined as 5 ng/ml or higher) and 12.5% (when the abnormal value of CA19-9 was defined as 37 U/ml or higher), respectively, in the validation cohort, demonstrating that all of the polynucleotides consisting of the nucleotide sequences represented by SEQ ID NOs: 1 to 169 can discriminate, each alone, stomach cancer in the validation cohort with sensitivity beyond CEA and CA19-9.

For example, 4 polynucleotides consisting of the nucleotide sequences represented by SEQ ID NOs: 3, 5, 21, and 28 were able to correctly determine stomach cancer as to 9 stage IA stomach cancer samples contained in the validation cohort. Thus, these polynucleotides can detect even early stomach cancer and contribute to the early diagnosis of stomach cancer.

### Example 2

### <Method for Evaluating Stomach Cancer Discriminant Performance by Combination of Multiple Gene Markers Using Samples in the Validation Cohort>

In this Example, a method for evaluating stomach cancer discriminant performance by a combination of the gene markers selected in Example 1 was studied. Specifically, Fisher's discriminant analysis was conducted as to 14,190 combinations of two expression level measurement values comprising at least one or more of the expression level measurement values of the newly found polynucleotides consisting of the nucleotide sequences represented by SEQ ID NOs: 1 to 165 among any of the polynucleotides consisting of the nucleotide sequences represented by SEQ ID NOs: 1 to 169 selected in Example 1, to construct a discriminant for determining the presence or absence of stomach cancer. Next, accuracy, sensitivity, and specificity in the validation cohort were calculated using the discriminant thus prepared, and the discriminant performance of the selected polynucleotides was validated using the independent samples.

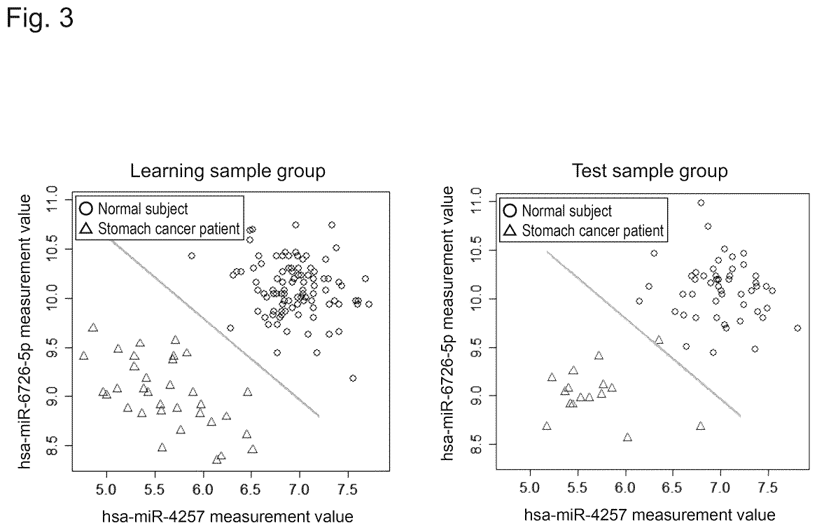

For example, the expression level measurement values of the nucleotide sequences represented by SEQ ID NO: 1 and SEQ ID NO: 2 were compared between the healthy subjects (100 persons) and the stomach cancer patients (34 persons) in the training cohort. As a result, a scatter diagram that significantly separated the expression level measurement values of the stomach cancer patient group from those of the healthy subject group was obtained (see the left diagram of FIG. 3). These results were also reproducible in the healthy subjects (50 persons) and the stomach cancer patients (16 persons) in the validation cohort (see the right diagram of FIG. 3). Likewise, a scatter diagram that significantly separated the gene expression level measurement values of the stomach cancer patient group from those of the healthy subject group was also obtained as to the other combinations of two expression level measurement values comprising at least one or more of the expression level measurement values of the newly found polynucleotides consisting of the nucleotide sequences represented by SEQ ID NOs: 1 to 165 among the polynucleotides consisting of the nucleotide sequences represented by SEQ ID NOs: 1 to 169. These results were able to be validated in the validation cohort. For example, as for these nucleotide sequences represented by SEQ ID NO: 1 and SEQ ID NO: 2, the number of samples that were correctly or incorrectly identified in the detection of stomach cancer was calculated using the function (0=0.83x+y−14.78) that was set in the training cohort and discriminated between the two groups. As a result, 15 true positives, 50 true negatives, 0 false positives, and 1 false negative were obtained. From these values, 98.5% accuracy, 93.8% sensitivity, and 100% specificity were obtained as the detection performance. In this way, the detection performance was calculated as to all of the combinations of two expression level measurement values comprising at least one or more of the expression level measurement values of any of the newly found polynucleotides consisting of the nucleotide sequences represented by SEQ ID NOs: 1 to 165 among the polynucleotides consisting of the nucleotide sequences represented by SEQ ID NOs: 1 to 169. Among them, 168 combinations comprising the expression level measurement value of the polynucleotide consisting of the nucleotide sequence represented by SEQ ID NO: 1 and the detection performance thereof were described in Table 6 as an example. For example, a combination of the expression level measurement values of the polynucleotides consisting of the nucleotide sequences represented by SEQ ID NOs: 1 and 14 exhibited sensitivity of 100% in the validation cohort. Also, all of combinations of two polynucleotides consisting of nucleotide sequences represented by SEQ ID NO: 1 and any of SEQ ID NOs: 2, 4, 14, 17, 22, 24, 27, 32, 39, 43, 46, 48, 53, 65, 66, 67, 78, 89, 91, 98, 99, 113, 116, 122, 129, 141, 144, 148, 150, 154, and 156 exhibited specificity of 100%. 14,159 combinations of the expression level measurement values of polynucleotides having sensitivity beyond the existing marker CEA or CA19-9 (both 12.5% in Table 5) were obtained in the validation cohort. All of the nucleotide sequences 1 to 165 described in Table 2 obtained in Example 1 were employed at least once in these combinations. These results demonstrated that the combined use of two of the polynucleotides consisting of the nucleotide sequences represented by SEQ ID NOs: 1 to 165 can also discriminate stomach cancer with excellent performance beyond the existing marker. Thus, the combinations of two expression level measurement values of the polynucleotides consisting of the nucleotide sequences also produced excellent stomach cancer detection sensitivity.

Markers for the detection of stomach cancer with better sensitivity are obtained by further combining 3, 4, 5, 6, 7, 8, 9, 10 or more of the expression level measurement values of the polynucleotides consisting of the nucleotide sequences represented by SEQ ID NOs: 1 to 169. For example, the newly found polynucleotides consisting of the nucleotide sequences represented by SEQ ID NOs: 1 to 165 among the polynucleotides consisting of the nucleotide sequences represented by SEQ ID NOs: 1 to 169 selected in Example 1 were measured to obtain their expression levels between the healthy subject group and the stomach cancer group in the validation cohort. All of the polynucleotides were ranked in the descending order of their P values based on the Student's t-test which indicates statistical significance of difference between groups (i.e., one having the lowest P value was ranked in the first place), and stomach cancer detection sensitivity was evaluated using combinations of one or more polynucleotides to which the polynucleotides were added one by one from the top to the bottom according to the rank. In short, the order in which the polynucleotides were combined in this evaluation is in reverse in terms of SEQ ID NOs from SEQ ID NO: 165 to SEQ ID NOs: 164, 163, . . . shown in Table 2. As a result, the sensitivity in the validation cohort was 6.2% for 1 polynucleotide (SEQ ID NO: 165), 62.5% for 2 polynucleotides (SEQ ID NOs: 165 and 164), 68.8% for 4 polynucleotides (SEQ ID NOs: 162 to 165), 75.0% for 8 polynucleotides (SEQ ID NOs: 158 to 165), 87.5% for 13 polynucleotides (SEQ ID NOs: 153 to 165), 93.8% for 15 polynucleotides (SEQ ID NOs: 151 to 165), 100% for 23 polynucleotides (SEQ ID NOs: 143 to 165), 100% for 50 polynucleotides (SEQ ID NOs: 116 to 165), 100% for 80 polynucleotides (SEQ ID NOs: 86 to 165), 100% for 100 polynucleotides (SEQ ID NOs: 66 to 165), 100% for 150 polynucleotides (SEQ ID NOs: 16 to 165), and 100% for 165 polynucleotides (SEQ ID NOs: 1 to 165).

These results demonstrated that a combination of multiple polynucleotides can produce higher stomach cancer discriminant performance than that of each polynucleotide alone or a combination of a fewer number of polynucleotides. In this context, the combinations of multiple polynucleotides are not limited to the combinations of the polynucleotides added in the order of statistically significant difference as described above, and any combination of multiple polynucleotides can be used in the detection of stomach cancer.

From these results, it can be concluded that all of the polynucleotides consisting of the nucleotide sequences represented by SEQ ID NOs: 1 to 169 serve as excellent markers for the detection of stomach cancer.

TABLE 2

| | | | Expression level in |
|---|---|---|---|
| | | P value after | stomach cancer |
| | | Bonferroni | patient relative to |
| SEQ ID NO: | Gene name | correction | healthy subject |
| 1 | hsa-miR-4257 | 1.77.E−35 | − |
| 2 | hsa-miR-6726-5p | 1.21.E−34 | − |
| 3 | hsa-miR-1343-3p | 2.35.E−27 | − |
| 4 | hsa-miR-1247-3p | 1.41.E−25 | + |
| 5 | hsa-miR-6787-5p | 9.96.E−25 | − |
| 6 | hsa-miR-6875-5p | 3.18.E−23 | + |
| 7 | hsa-miR-1225-3p | 4.17.E−23 | + |
| 8 | hsa-miR-8063 | 1.39.E−22 | − |
| 9 | hsa-miR-6781-5p | 4.80.E−22 | + |
| 10 | hsa-miR-4746-3p | 7.08.E−22 | + |
| 11 | hsa-miR-1908-5p | 1.66.E−21 | + |
| 12 | hsa-miR-6756-5p | 2.78.E−21 | − |
| 13 | hsa-miR-204-3p | 3.60.E−21 | − |
| 14 | hsa-miR-4651 | 3.74.E−21 | − |
| 15 | hsa-miR-6757-5p | 5.50.E−21 | − |
| 16 | hsa-miR-6825-5p | 7.04.E−20 | + |
| 17 | hsa-miR-7108-5p | 8.87.E−20 | + |
| 18 | hsa-miR-4792 | 1.50.E−19 | + |
| 19 | hsa-miR-7641 | 2.77.E−19 | − |
| 20 | hsa-miR-3188 | 4.51.E−19 | + |
| 21 | hsa-miR-3131 | 1.03.E−18 | − |
| 22 | hsa-miR-6780b-5p | 1.44.E−18 | + |
| 23 | hsa-miR-8069 | 2.56.E−18 | + |
| 24 | hsa-miR-6840-3p | 3.01.E−18 | − |
| 25 | hsa-miR-8072 | 4.25.E−18 | + |
| 26 | hsa-miR-1233-5p | 2.25.E−17 | − |
| 27 | hsa-miR-6887-5p | 4.74.E−17 | − |
| 28 | hsa-miR-1231 | 5.08.E−17 | + |
| 29 | hsa-miR-5572 | 1.08.E−16 | + |
| 30 | hsa-miR-6738-5p | 1.16.E−16 | − |
| 31 | hsa-miR-6784-5p | 1.68.E−16 | + |
| 32 | hsa-miR-6791-5p | 3.16.E−16 | + |
| 33 | hsa-miR-6749-5p | 3.69.E−16 | − |
| 34 | hsa-miR-6741-5p | 5.38.E−16 | − |
| 35 | hsa-miR-128-1-5p | 1.67.E−15 | + |
| 36 | hsa-miR-4419b | 2.16.E−15 | − |
| 37 | hsa-miR-6746-5p | 2.49.E−15 | − |
| 38 | hsa-miR-3184-5p | 2.56.E−15 | + |
| 39 | hsa-miR-3679-5p | 2.88.E−15 | + |
| 40 | hsa-miR-7110-5p | 3.95.E−15 | + |
| 41 | hsa-miR-4516 | 4.43.E−15 | − |
| 42 | hsa-miR-6717-5p | 4.77.E−15 | − |
| 43 | hsa-miR-6826-5p | 4.94.E−15 | − |
| 44 | hsa-miR-4433b-3p | 5.34.E−15 | + |
| 45 | hsa-miR-3679-3p | 2.55.E−14 | + |
| 46 | hsa-miR-3135b | 3.35.E−14 | − |
| 47 | hsa-miR-3622a-5p | 4.36.E−14 | − |
| 48 | hsa-miR-711 | 5.86.E−14 | + |
| 49 | hsa-miR-4467 | 7.26.E−14 | + |
| 50 | hsa-miR-6857-5p | 2.73.E−13 | + |
| 51 | hsa-miR-6515-3p | 3.28.E−13 | + |
| 52 | hsa-miR-1225-5p | 4.67.E−13 | + |
| 53 | hsa-miR-187-5p | 5.39.E−13 | − |
| 54 | hsa-miR-3185 | 6.80.E−13 | + |
| 55 | hsa-miR-642b-3p | 8.60.E−13 | − |
| 56 | hsa-miR-1249 | 1.16.E−12 | + |
| 57 | hsa-miR-744-5p | 2.15.E−12 | + |
| 58 | hsa-miR-4442 | 3.26.E−12 | − |
| 59 | hsa-miR-1228-3p | 4.54.E−12 | + |
| 60 | hsa-miR-939-5p | 7.77.E−12 | + |
| 61 | hsa-miR-6845-5p | 9.25.E−12 | + |
| 62 | hsa-miR-887-3p | 1.35.E−11 | + |
| 63 | hsa-miR-7845-5p | 1.81.E−11 | + |
| 64 | hsa-miR-6729-5p | 2.80.E−11 | + |
| 65 | hsa-miR-4632-5p | 6.45.E−11 | + |
| 66 | hsa-miR-615-5p | 7.56.E−11 | − |
| 67 | hsa-miR-6724-5p | 8.75.E−11 | + |
| 68 | hsa-miR-4728-5p | 1.05.E−10 | − |
| 69 | hsa-miR-6732-5p | 1.23.E−10 | + |
| 70 | hsa-miR-6816-5p | 1.35.E−10 | + |
| 71 | hsa-miR-4695-5p | 4.88.E−10 | + |
| 72 | hsa-miR-6088 | 5.46.E−10 | − |
| 73 | hsa-miR-7975 | 5.48.E−10 | − |
| 74 | hsa-miR-3197 | 5.56.E−10 | + |
| 75 | hsa-miR-6125 | 6.01.E−10 | + |
| 76 | hsa-miR-4433-3p | 6.04.E−10 | + |
| 77 | hsa-miR-6727-5p | 8.92.E−10 | − |
| 78 | hsa-miR-4706 | 1.09.E−09 | − |
| 79 | hsa-miR-7847-3p | 1.25.E−09 | − |
| 80 | hsa-miR-6805-3p | 1.57.E−09 | + |
| 81 | hsa-miR-6766-3p | 1.95.E−09 | + |
| 82 | hsa-miR-1913 | 2.12.E−09 | + |
| 83 | hsa-miR-4649-5p | 2.42.E−09 | − |
| 84 | hsa-miR-602 | 2.50.E−09 | + |
| 85 | hsa-miR-3663-3p | 2.83.E−09 | − |
| 86 | hsa-miR-6893-5p | 3.40.E−09 | − |
| 87 | hsa-miR-6861-5p | 3.53.E−09 | − |
| 88 | hsa-miR-4449 | 4.40.E−09 | + |
| 89 | hsa-miR-6842-5p | 4.48.E−09 | + |
| 90 | hsa-miR-4454 | 4.77.E−09 | − |
| 91 | hsa-miR-5195-3p | 6.01.E−09 | − |
| 92 | hsa-miR-663b | 9.12.E−09 | − |
| 93 | hsa-miR-6765-5p | 2.06.E−08 | + |
| 94 | hsa-miR-4513 | 2.61.E−08 | − |
| 95 | hsa-miR-614 | 4.92.E−08 | − |
| 96 | hsa-miR-6785-5p | 5.85.E−08 | − |
| 97 | hsa-miR-6777-5p | 6.02.E−08 | − |
| 98 | hsa-miR-940 | 8.08.E−08 | + |
| 99 | hsa-miR-4741 | 9.53.E−08 | + |
| 100 | hsa-miR-6870-5p | 1.07.E−07 | + |
| 101 | hsa-miR-6131 | 1.21.E−07 | − |
| 102 | hsa-miR-150-3p | 1.31.E−07 | − |
| 103 | hsa-miR-4707-5p | 1.70.E−07 | + |
| 104 | hsa-miR-1915-3p | 2.00.E−07 | + |
| 105 | hsa-miR-3937 | 2.17.E−07 | + |
| 106 | hsa-miR-937-5p | 2.85.E−07 | − |
| 107 | hsa-miR-4443 | 3.12.E−07 | + |
| 108 | hsa-miR-1914-3p | 3.23.E−07 | − |
| 109 | hsa-miR-3620-5p | 3.97.E−07 | + |
| 110 | hsa-miR-1268b | 5.51.E−07 | + |
| 111 | hsa-miR-1227-5p | 8.69.E−07 | + |
| 112 | hsa-miR-6880-5p | 9.59.E−07 | + |
| 113 | hsa-miR-4417 | 1.28.E−06 | + |
| 114 | hsa-miR-6802-5p | 1.30.E−06 | − |
| 115 | hsa-miR-6769a-5p | 1.32.E−06 | − |
| 116 | hsa-miR-663a | 1.42.E−06 | + |
| 117 | hsa-miR-6721-5p | 1.73.E−06 | + |
| 118 | hsa-miR-4532 | 2.01.E−06 | − |
| 119 | hsa-miR-7977 | 2.27.E−06 | − |
| 120 | hsa-miR-92b-5p | 2.37.E−06 | + |
| 121 | hsa-miR-371a-5p | 2.37.E−06 | − |
| 122 | hsa-miR-6126 | 2.47.E−06 | + |
| 123 | hsa-miR-4734 | 2.53.E−06 | + |
| 124 | hsa-miR-4665-3p | 2.71.E−06 | + |
| 125 | hsa-miR-423-5p | 4.04.E−06 | − |
| 126 | hsa-miR-1469 | 8.08.E−06 | + |
| 127 | hsa-miR-4675 | 8.36.E−06 | − |
| 128 | hsa-miR-1915-5p | 8.49.E−06 | − |
| 129 | hsa-miR-6716-5p | 9.56.E−06 | + |
| 130 | hsa-miR-718 | 1.59.E−05 | + |
| 131 | hsa-miR-4281 | 1.59.E−05 | − |
| 132 | hsa-miR-6820-5p | 1.88.E−05 | − |
| 133 | hsa-miR-6795-5p | 3.14.E−05 | − |
| 134 | hsa-miR-6779-5p | 3.55.E−05 | − |
| 135 | hsa-miR-7109-5p | 4.02.E−05 | − |
| 136 | hsa-miR-6798-5p | 4.28.E−05 | + |
| 137 | hsa-miR-4648 | 6.38.E−05 | + |
| 138 | hsa-miR-8059 | 7.15.E−05 | − |
| 139 | hsa-miR-6765-3p | 8.47.E−05 | − |
| 140 | hsa-miR-6132 | 1.28.E−04 | + |
| 141 | hsa-miR-4492 | 1.51.E−04 | + |
| 142 | hsa-miR-7107-5p | 1.64.E−04 | − |
| 143 | hsa-miR-3195 | 1 73.E−04 | + |
| 144 | hsa-miR-3180 | 2.82.E−04 | + |
| 145 | hsa-miR-296-3p | 2.89.E−04 | − |
| 146 | hsa-miR-564 | 4.75.E−04 | − |
| 147 | hsa-miR-1268a | 5.55.E−04 | + |
| 148 | hsa-miR-6848-5p | 6.07.E−04 | + |
| 149 | hsa-miR-762 | 8.99.E−04 | + |
| 150 | hsa-miR-2861 | 1.57.E−03 | − |
| 151 | hsa-miR-1203 | 1.91.E−03 | + |
| 152 | hsa-miR-1260b | 2.01.E−03 | − |
| 153 | hsa-miR-4476 | 2.45.E−03 | − |
| 154 | hsa-miR-6885-5p | 2.83.E−03 | − |
| 155 | hsa-miR-6769b-5p | 2.84.E−03 | − |
| 156 | hsa-miR-23b-3p | 2.87.E−03 | − |
| 157 | hsa-miR-1343-5p | 3.95.E−03 | + |
| 158 | hsa-miR-3621 | 4.31.E−03 | − |
| 159 | hsa-miR-4688 | 4.77.E−03 | − |
| 160 | hsa-miR-4286 | 4.90.E−03 | − |
| 161 | hsa-miR-4640-5p | 6.06.E−03 | + |
| 162 | hsa-miR-4739 | 6.13.E−03 | + |
| 163 | hsa-miR-1260a | 7.24.E−03 | − |
| 164 | hsa-miR-4276 | 8.00.E−03 | + |
| 165 | hsa-miR-7106-5p | 9.50.E−03 | − |
| 166 | hsa-miR-128-2-5p | 1.79.E−09 | − |
| 167 | lisa-miR-125a-3p | 1.81.E−09 | − |
| 168 | hsa-miR-92a-2-5p | 2.01.E−05 | + |
| 169 | hsa-miR-486-3p | 2.60.E−03 | − |

TABLE 3

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 1 | 95.5 | 85.3 | 99.0 | 95.5 | 87.5 | 98.0 |
| 2 | 94.8 | 88.2 | 97.0 | 97.0 | 93.8 | 98.0 |
| 3 | 97.0 | 91.2 | 99.0 | 97.0 | 93.8 | 98.0 |
| 4 | 93.3 | 82.4 | 97.0 | 90.9 | 81.2 | 94.0 |
| 5 | 93.3 | 79.4 | 98.0 | 97.0 | 93.8 | 98.0 |
| 6 | 94.0 | 91.2 | 95.0 | 87.9 | 87.5 | 88.0 |
| 7 | 92.5 | 82.4 | 96.0 | 97.0 | 87.5 | 100.0 |
| 8 | 90.3 | 88.2 | 91.0 | 90.9 | 81.2 | 94.0 |
| 9 | 94.8 | 85.3 | 98.0 | 86.4 | 68.8 | 92.0 |
| 10 | 91.0 | 76.5 | 96.0 | 95.5 | 87.5 | 98.0 |
| 11 | 90.3 | 82.4 | 93.0 | 89.4 | 75.0 | 94.0 |
| 12 | 90.3 | 73.5 | 96.0 | 87.9 | 81.2 | 90.0 |
| 13 | 91.0 | 73.5 | 97.0 | 81.8 | 87.5 | 80.0 |
| 14 | 91.8 | 79.4 | 96.0 | 92.4 | 75.0 | 98.0 |
| 15 | 90.3 | 76.5 | 95.0 | 95.5 | 81.2 | 100.0 |
| 16 | 88.1 | 82.4 | 90.0 | 92.4 | 93.8 | 92.0 |
| 17 | 88.1 | 73.5 | 93.0 | 83.3 | 68.8 | 88.0 |
| 18 | 92.5 | 88.2 | 94.0 | 93.9 | 81.2 | 98.0 |
| 19 | 88.1 | 73.5 | 93.0 | 83.3 | 56.2 | 92.0 |
| 20 | 91.8 | 85.3 | 94.0 | 90.9 | 68.8 | 98.0 |
| 21 | 91.8 | 76.5 | 97.0 | 95.5 | 87.5 | 98.0 |
| 22 | 88.8 | 70.6 | 95.0 | 89.4 | 56.2 | 100.0 |
| 23 | 88.1 | 67.6 | 95.0 | 87.9 | 62.5 | 96.0 |
| 24 | 87.3 | 64.7 | 95.0 | 87.9 | 62.5 | 96.0 |
| 25 | 88.1 | 61.8 | 97.0 | 83.3 | 62.5 | 90.0 |
| 26 | 90.3 | 76.5 | 95.0 | 89.4 | 75.0 | 94.0 |
| 27 | 91.0 | 67.6 | 99.0 | 89.4 | 56.2 | 100.0 |
| 28 | 90.3 | 79.4 | 94.0 | 90.9 | 87.5 | 92.0 |
| 29 | 85.8 | 79.4 | 88.0 | 90.9 | 93.8 | 90.0 |
| 30 | 89.6 | 76.5 | 94.0 | 86.4 | 62.5 | 94.0 |
| 31 | 85.1 | 73.5 | 89.0 | 87.9 | 87.5 | 88.0 |
| 32 | 85.1 | 67.6 | 91.0 | 89.4 | 62.5 | 98.0 |
| 33 | 87.3 | 61.8 | 96.0 | 90.9 | 68.8 | 98.0 |
| 34 | 90.3 | 70.6 | 97.0 | 89.4 | 81.2 | 92.0 |
| 35 | 89.6 | 82.4 | 92.0 | 84.8 | 81.2 | 86.0 |
| 36 | 90.3 | 73.5 | 96.0 | 89.4 | 62.5 | 98.0 |
| 37 | 90.3 | 70.6 | 97.0 | 92.4 | 81.2 | 96.0 |
| 38 | 87.3 | 82.4 | 89.0 | 90.9 | 81.2 | 94.0 |
| 39 | 90.3 | 76.5 | 95.0 | 90.9 | 62.5 | 100.0 |
| 40 | 87.3 | 76.5 | 91.0 | 89.4 | 87.5 | 90.0 |
| 41 | 90.3 | 61.8 | 100.0 | 90.9 | 62.5 | 100.0 |
| 42 | 90.3 | 61.8 | 100.0 | 93.9 | 75.0 | 100.0 |
| 43 | 90.3 | 67.6 | 98.0 | 89.4 | 56.2 | 100.0 |
| 44 | 87.3 | 73.5 | 92.0 | 81.8 | 75.0 | 84.0 |
| 45 | 90.3 | 82.4 | 93.0 | 83.3 | 62.5 | 90.0 |
| 46 | 90.3 | 70.6 | 97.0 | 87.9 | 56.2 | 98.0 |
| 47 | 85.1 | 47.1 | 98.0 | 90.9 | 68.8 | 98.0 |
| 48 | 86.6 | 64.7 | 94.0 | 89.4 | 62.5 | 98.0 |
| 49 | 83.6 | 73.5 | 87.0 | 86.4 | 56.2 | 96.0 |
| 50 | 90.3 | 79.4 | 94.0 | 95.5 | 93.8 | 96.0 |
| 51 | 84.3 | 61.8 | 92.0 | 77.3 | 62.5 | 82.0 |
| 52 | 87.3 | 64.7 | 95.0 | 84.8 | 62.5 | 92.0 |
| 53 | 84.3 | 52.9 | 95.0 | 87.9 | 56.2 | 98.0 |
| 54 | 85.8 | 67.6 | 92.0 | 90.9 | 81.2 | 94.0 |
| 55 | 87.3 | 64.7 | 95.0 | 90.9 | 68.8 | 98.0 |
| 56 | 86.5 | 67.6 | 92.9 | 80.3 | 56.2 | 88.0 |
| 57 | 83.6 | 52.9 | 94.0 | 84.8 | 43.8 | 98.0 |
| 58 | 85.8 | 70.6 | 91.0 | 87.9 | 75.0 | 92.0 |
| 59 | 84.3 | 55.9 | 94.0 | 86.4 | 75.0 | 90.0 |
| 60 | 82.8 | 73.5 | 86.0 | 83.3 | 68.8 | 88.0 |
| 61 | 85.1 | 52.9 | 96.0 | 87.9 | 81.2 | 90.0 |
| 62 | 82.8 | 61.8 | 90.0 | 84.8 | 75.0 | 88.0 |
| 63 | 85.1 | 58.8 | 94.0 | 86.4 | 68.8 | 92.0 |
| 64 | 79.9 | 50.0 | 90.0 | 81.8 | 68.8 | 86.0 |
| 65 | 88.1 | 61.8 | 97.0 | 84.8 | 43.8 | 98.0 |
| 66 | 83.6 | 41.2 | 98.0 | 89.4 | 62.5 | 98.0 |
| 67 | 82.8 | 55.9 | 92.0 | 78.8 | 50.0 | 88.0 |
| 68 | 78.4 | 44.1 | 90.0 | 81.8 | 50.0 | 92.0 |
| 69 | 82.1 | 61.8 | 89.0 | 80.3 | 62.5 | 86.0 |
| 70 | 82.1 | 58.8 | 90.0 | 84.8 | 62.5 | 92.0 |
| 71 | 79.9 | 47.1 | 91.0 | 83.3 | 50.0 | 94.0 |
| 72 | 79.9 | 50.0 | 90.0 | 86.4 | 68.8 | 92.0 |
| 73 | 80.6 | 41.2 | 94.0 | 78.8 | 37.5 | 92.0 |
| 74 | 85.8 | 61.8 | 94.0 | 83.3 | 50.0 | 94.0 |
| 75 | 81.3 | 44.1 | 94.0 | 81.8 | 37.5 | 96.0 |
| 76 | 81.3 | 61.8 | 88.0 | 83.3 | 68.8 | 88.0 |
| 77 | 86.6 | 67.6 | 93.0 | 90.9 | 68.8 | 98.0 |
| 78 | 85.1 | 58.8 | 94.0 | 84.8 | 56.2 | 94.0 |
| 79 | 85.1 | 44.1 | 99.0 | 78.8 | 12.5 | 100.0 |
| 80 | 79.9 | 50.0 | 90.0 | 89.4 | 75.0 | 94.0 |
| 81 | 82.8 | 47.1 | 95.0 | 80.3 | 50.0 | 90.0 |
| 82 | 82.1 | 55.9 | 91.0 | 78.5 | 50.0 | 87.8 |
| 83 | 84.3 | 50.0 | 96.0 | 81.8 | 37.5 | 96.0 |
| 84 | 82.8 | 52.9 | 93.0 | 87.9 | 68.8 | 94.0 |
| 85 | 82.8 | 47.1 | 95.0 | 78.8 | 25.0 | 96.0 |
| 86 | 85.8 | 52.9 | 97.0 | 92.4 | 81.2 | 96.0 |
| 87 | 84.3 | 50.0 | 96.0 | 81.8 | 43.8 | 94.0 |
| 88 | 80.6 | 38.2 | 95.0 | 86.4 | 56.2 | 96.0 |
| 89 | 81.3 | 41.2 | 95.0 | 87.9 | 62.5 | 96.0 |
| 90 | 81.3 | 47.1 | 93.0 | 78.8 | 37.5 | 92.0 |
| 91 | 82.1 | 47.1 | 94.0 | 81.8 | 43.8 | 94.0 |
| 92 | 83.6 | 47.1 | 96.0 | 86.4 | 43.8 | 100.0 |
| 93 | 82.8 | 47.1 | 95.0 | 80.3 | 37.5 | 94.0 |
| 94 | 79.1 | 29.4 | 96.0 | 83.3 | 43.8 | 96.0 |
| 95 | 76.9 | 38.2 | 90.0 | 78.8 | 31.2 | 94.0 |
| 96 | 81.3 | 44.1 | 94.0 | 83.3 | 43.8 | 96.0 |
| 97 | 79.9 | 38.2 | 94.0 | 83.3 | 50.0 | 94.0 |
| 98 | 80.6 | 44.1 | 93.0 | 78.8 | 25.0 | 96.0 |
| 99 | 82.1 | 50.0 | 93.0 | 80.3 | 43.8 | 92.0 |
| 100 | 81.3 | 38.2 | 96.0 | 84.8 | 37.5 | 100.0 |
| 101 | 85.1 | 50.0 | 97.0 | 83.3 | 37.5 | 98.0 |
| 102 | 80.6 | 29.4 | 98.0 | 77.3 | 31.2 | 92.0 |
| 103 | 76.1 | 32.4 | 91.0 | 75.8 | 25.0 | 92.0 |
| 104 | 84.3 | 50.0 | 96.0 | 80.3 | 25.0 | 98.0 |
| 105 | 79.1 | 38.2 | 93.0 | 84.8 | 56.2 | 94.0 |
| 106 | 82.8 | 44.1 | 96.0 | 81.8 | 31.2 | 98.0 |
| 107 | 82.1 | 44.1 | 95.0 | 83.3 | 43.8 | 96.0 |
| 108 | 81.3 | 47.1 | 93.0 | 80.3 | 56.2 | 88.0 |
| 109 | 79.9 | 38.2 | 94.0 | 81.8 | 50.0 | 92.0 |
| 110 | 74.6 | 32.4 | 89.0 | 72.7 | 37.5 | 84.0 |
| 111 | 81.3 | 44.1 | 94.0 | 75.8 | 31.2 | 90.0 |
| 112 | 82.8 | 47.1 | 95.0 | 80.3 | 31.2 | 96.0 |
| 113 | 81.3 | 35.3 | 97.0 | 81.8 | 37.5 | 96.0 |
| 114 | 86.6 | 52.9 | 98.0 | 86.4 | 50.0 | 98.0 |
| 115 | 79.1 | 38.2 | 93.0 | 75.8 | 12.5 | 96.0 |
| 116 | 81.3 | 41.2 | 95.0 | 78.8 | 31.2 | 94.0 |
| 117 | 76.9 | 38.2 | 90.0 | 80.3 | 56.2 | 88.0 |
| 118 | 81.3 | 38.2 | 96.0 | 75.8 | 18.8 | 94.0 |
| 119 | 78.4 | 35.3 | 93.0 | 81.8 | 43.8 | 94.0 |
| 120 | 78.4 | 38.2 | 92.0 | 78.8 | 18.8 | 98.0 |
| 121 | 79.1 | 35.3 | 94.0 | 75.8 | 37.5 | 88.0 |
| 122 | 78.4 | 38.2 | 92.0 | 81.8 | 31.2 | 98.0 |
| 123 | 78.4 | 32.4 | 94.0 | 80.3 | 37.5 | 94.0 |
| 124 | 87.3 | 52.9 | 99.0 | 86.4 | 50.0 | 98.0 |
| 125 | 76.9 | 32.4 | 92.0 | 75.8 | 50.0 | 84.0 |
| 126 | 78.4 | 29.4 | 95.0 | 71.2 | 12.5 | 90.0 |
| 127 | 80.6 | 41.2 | 94.0 | 83.3 | 31.2 | 100.0 |
| 128 | 79.9 | 32.4 | 96.0 | 78.8 | 31.2 | 94.0 |
| 129 | 77.6 | 26.5 | 95.0 | 77.3 | 31.2 | 92.0 |
| 130 | 76.1 | 26.5 | 93.0 | 75.8 | 31.2 | 90.0 |
| 131 | 78.4 | 35.3 | 93.0 | 84.8 | 50.0 | 96.0 |
| 132 | 80.6 | 29.4 | 98.0 | 77.3 | 37.5 | 90.0 |
| 133 | 79.9 | 23.5 | 99.0 | 80.3 | 18.8 | 100.0 |
| 134 | 75.4 | 32.4 | 90.0 | 83.3 | 37.5 | 98.0 |
| 135 | 73.9 | 23.5 | 91.0 | 80.3 | 50.0 | 90.0 |
| 136 | 78.4 | 44.1 | 90.0 | 74.2 | 43.8 | 84.0 |
| 137 | 73.9 | 20.6 | 92.0 | 80.3 | 18.8 | 100.0 |
| 138 | 79.1 | 29.4 | 96.0 | 81.8 | 43.8 | 94.0 |
| 139 | 82.1 | 41.2 | 96.0 | 80.3 | 31.2 | 96.0 |
| 140 | 79.9 | 29.4 | 97.0 | 78.8 | 18.8 | 98.0 |
| 141 | 79.1 | 32.4 | 95.0 | 81.8 | 50.0 | 92.0 |
| 142 | 75.4 | 29.4 | 91.0 | 75.8 | 25.0 | 92.0 |
| 143 | 78.4 | 38.2 | 92.0 | 77.3 | 31.2 | 92.0 |
| 144 | 79.9 | 32.4 | 96.0 | 81.8 | 31.2 | 98.0 |
| 145 | 81.2 | 27.3 | 99.0 | 77.3 | 18.8 | 96.0 |
| 146 | 81.3 | 35.3 | 97.0 | 84.8 | 43.8 | 98.0 |
| 147 | 73.1 | 14.7 | 93.0 | 71.2 | 6.2 | 92.0 |
| 148 | 77.6 | 23.5 | 96.0 | 78.8 | 25.0 | 96.0 |
| 149 | 77.6 | 23.5 | 96.0 | 72.3 | 12.5 | 91.8 |
| 150 | 77.6 | 23.5 | 96.0 | 80.3 | 31.2 | 96.0 |
| 151 | 74.6 | 14.7 | 95.0 | 78.8 | 12.5 | 100.0 |
| 152 | 79.9 | 32.4 | 96.0 | 75.8 | 18.8 | 94.0 |
| 153 | 76.1 | 20.6 | 95.0 | 78.8 | 37.5 | 92.0 |
| 154 | 81.3 | 32.4 | 98.0 | 75.8 | 6.2 | 98.0 |
| 155 | 76.9 | 20.6 | 96.0 | 81.8 | 31.2 | 98.0 |
| 156 | 79.1 | 23.5 | 98.0 | 72.7 | 6.2 | 94.0 |
| 157 | 82.1 | 35.3 | 98.0 | 80.3 | 18.8 | 100.0 |
| 158 | 74.6 | 11.8 | 96.0 | 74.2 | 6.2 | 96.0 |
| 159 | 77.6 | 20.6 | 97.0 | 78.8 | 18.8 | 98.0 |
| 160 | 75.4 | 20.6 | 94.0 | 71.2 | 6.2 | 92.0 |
| 161 | 78.4 | 26.5 | 96.0 | 78.8 | 12.5 | 100.0 |
| 162 | 79.1 | 20.6 | 99.0 | 78.8 | 18.8 | 98.0 |
| 163 | 76.1 | 20.6 | 95.0 | 72.7 | 6.2 | 94.0 |
| 164 | 76.9 | 14.7 | 98.0 | 78.8 | 12.5 | 100.0 |
| 165 | 74.6 | 8.8 | 97.0 | 75.8 | 6.2 | 98.0 |
| 166 | 85.1 | 52.9 | 96.0 | 87.9 | 50.0 | 100.0 |
| 167 | 85.8 | 50.0 | 98.0 | 89.4 | 68.8 | 96.0 |
| 168 | 80.6 | 41.2 | 94.0 | 77.3 | 31.2 | 92.0 |
| 169 | 80.6 | 32.4 | 97.0 | 81.8 | 25.0 | 100.0 |

TABLE 4

| | Discriminant | Constant |
|---|---|---|
| SEQ ID NO: | coefficient | term |
| 1 | 2.731 | 17.177 |
| 2 | 3.324 | 31.765 |
| 3 | 1.836 | 12.851 |
| 4 | 4.010 | 25.699 |
| 5 | 3.767 | 31.391 |
| 6 | 3.829 | 35.096 |
| 7 | 3.144 | 17.990 |
| 8 | 2.726 | 21.913 |
| 9 | 5.443 | 57.639 |
| 10 | 2.686 | 18.095 |
| 11 | 4.398 | 51.318 |
| 12 | 5.286 | 43.176 |
| 13 | 1.921 | 24.270 |
| 14 | 5.407 | 58.548 |
| 15 | 2.820 | 19.731 |
| 16 | 2.197 | 14.682 |
| 17 | 4.707 | 43.642 |
| 18 | 2.022 | 13.892 |
| 19 | 1.268 | 8.665 |
| 20 | 3.417 | 21.034 |
| 21 | 2.266 | 15.207 |
| 22 | 3.039 | 27.590 |
| 23 | 7.728 | 99.955 |
| 24 | 3.052 | 26.321 |
| 25 | 5.366 | 66.791 |
| 26 | 2.810 | 30.883 |
| 27 | 2.291 | 13.933 |
| 28 | 3.580 | 23.815 |
| 29 | 2.466 | 16.690 |
| 30 | 3.715 | 25.964 |
| 31 | 3.866 | 49.046 |
| 32 | 4.847 | 44.998 |
| 33 | 4.518 | 44.908 |
| 34 | 4.174 | 28.253 |
| 35 | 2.781 | 21.080 |
| 36 | 2.163 | 12.587 |
| 37 | 2.399 | 14.923 |
| 38 | 2.387 | 19.533 |
| 39 | 2.662 | 18.538 |
| 40 | 1.844 | 14.656 |
| 41 | 4.162 | 54.280 |
| 42 | 1.861 | 10.860 |
| 43 | 1.882 | 10.852 |
| 44 | 3.955 | 32.182 |
| 45 | 3.509 | 21.353 |
| 46 | 2.764 | 21.183 |
| 47 | 2.237 | 12.508 |
| 48 | 3.474 | 29.057 |
| 49 | 2.348 | 23.412 |
| 50 | 1.601 | 8.585 |
| 51 | 4.385 | 29.783 |
| 52 | 3.501 | 25.951 |
| 53 | 2.121 | 20.821 |
| 54 | 2.398 | 17.081 |
| 55 | 2.333 | 21.669 |
| 56 | 3.979 | 23.944 |
| 57 | 2.618 | 18.423 |
| 58 | 3.487 | 32.829 |
| 59 | 4.222 | 26.720 |
| 60 | 2.479 | 18.929 |
| 61 | 3.944 | 38.152 |
| 62 | 2.371 | 17.392 |
| 63 | 2.987 | 20.097 |
| 64 | 9.232 | 116.333 |
| 65 | 4.246 | 34.038 |
| 66 | 1.900 | 12.014 |
| 67 | 4.891 | 49.041 |
| 68 | 5.062 | 35.194 |
| 69 | 3.378 | 28.973 |
| 70 | 4.587 | 46.523 |
| 71 | 4.446 | 33.529 |
| 72 | 3.367 | 33.945 |
| 73 | 2.155 | 21.186 |
| 74 | 2.768 | 26.384 |
| 75 | 5.220 | 62.722 |
| 76 | 3.883 | 28.652 |
| 77 | 5.643 | 71.747 |
| 78 | 3.610 | 27.579 |
| 79 | 2.457 | 15.182 |
| 80 | 2.520 | 19.029 |
| 81 | 3.853 | 22.961 |
| 82 | 3.525 | 21.894 |
| 83 | 2.531 | 25.858 |
| 84 | 3.041 | 19.506 |
| 85 | 3.868 | 46.680 |
| 86 | 2.117 | 17.685 |
| 87 | 3.724 | 26.711 |
| 88 | 3.680 | 23.968 |
| 89 | 3.374 | 20.135 |
| 90 | 2.196 | 25.309 |
| 91 | 2.976 | 20.156 |
| 92 | 2.933 | 25.402 |
| 93 | 5.009 | 53.145 |
| 94 | 2.567 | 14.765 |
| 95 | 1.729 | 11.402 |
| 96 | 2.393 | 21.401 |
| 97 | 3.112 | 20.031 |
| 98 | 3.065 | 19.720 |
| 99 | 3.850 | 38.303 |
| 100 | 3.191 | 23.796 |
| 101 | 1.739 | 18.155 |
| 102 | 1.790 | 11.695 |
| 103 | 4.223 | 31.086 |
| 104 | 3.902 | 43.384 |
| 105 | 4.394 | 38.067 |
| 106 | 3.808 | 31.650 |
| 107 | 2.442 | 15.680 |
| 108 | 4.742 | 35.456 |
| 109 | 4.065 | 32.357 |
| 110 | 3.132 | 31.233 |
| 111 | 6.253 | 59.917 |
| 112 | 2.144 | 16.593 |
| 113 | 5.077 | 41.640 |
| 114 | 4.331 | 36.232 |
| 115 | 4.104 | 26.007 |
| 116 | 4.365 | 44.632 |
| 117 | 4.092 | 30.958 |
| 118 | 3.410 | 40.413 |
| 119 | 2.277 | 22.244 |
| 120 | 3.385 | 27.099 |
| 121 | 3.662 | 26.864 |
| 122 | 3.020 | 32.940 |
| 123 | 5.127 | 61.295 |
| 124 | 2.499 | 14.725 |
| 125 | 2.372 | 17.057 |
| 126 | 5.371 | 54.883 |
| 127 | 2.882 | 21.683 |
| 128 | 1.355 | 8.339 |
| 129 | 3.793 | 24.904 |
| 130 | 3.673 | 25.051 |
| 131 | 3.824 | 44.211 |
| 132 | 2.858 | 20.620 |
| 133 | 2.687 | 15.927 |
| 134 | 6.294 | 44.652 |
| 135 | 5.392 | 39.920 |
| 136 | 2.883 | 30.122 |
| 137 | 1.419 | 8.435 |
| 138 | 3.372 | 25.593 |
| 139 | 1.616 | 14.087 |
| 140 | 3.505 | 27.638 |
| 141 | 5.430 | 57.153 |
| 142 | 4.737 | 36.945 |
| 143 | 4.079 | 33.703 |
| 144 | 4.615 | 40.322 |
| 145 | 1.899 | 11.130 |
| 146 | 1.461 | 8.484 |
| 147 | 3.248 | 36.484 |
| 148 | 4.537 | 33.621 |
| 149 | 6.451 | 87.375 |
| 150 | 5.814 | 72.020 |
| 151 | 2.391 | 14.618 |
| 152 | 2.345 | 19.966 |
| 153 | 1.746 | 12.413 |
| 154 | 2.794 | 30.977 |
| 155 | 3.878 | 24.272 |
| 156 | 1.014 | 5.894 |
| 157 | 3.451 | 35.923 |
| 158 | 4.810 | 57.343 |
| 159 | 3.755 | 26.714 |
| 160 | 2.474 | 18.364 |
| 161 | 4.014 | 31.043 |
| 162 | 3.561 | 40.868 |
| 163 | 2.408 | 16.644 |
| 164 | 1.795 | 10.022 |
| 165 | 2.135 | 12.545 |
| 166 | 2.652 | 28.430 |
| 167 | 1.220 | 7.446 |
| 168 | 2.017 | 19.036 |
| 169 | 2.835 | 22.505 |

TABLE 5-1
Training cohort

| Sample name | Cancer stage | CEA | CA19-9 |
|---|---|---|---|
| SC03 | IA | 2.9(−) | 77.4(+) |
| SC04 | IA | 2.9(−) | 0.1(−) |
| SC05 | IA | 2.9(−) | 21.8(−) |
| SC06 | IA | 1.7(−) | 41.9(+) |
| SC07 | IB | 1.5(−) | 25.1(−) |
| SC09 | IA | 2.3(−) | 17.5(−) |
| SC10 | IIB | 1.2(−) | 10.0(−) |
| SC12 | IA | 3.3(−) | 8.5(−) |
| SC13 | IA | 3.4(−) | 8.2(−) |
| SC15 | IA | 3.7(−) | 6.3(−) |
| SC17 | IIB | 2.8(−) | 4.3(−) |
| SC18 | IB | 6.9(+) | 20.2(−) |
| SC19 | IA | 3.1(−) | 5.0(−) |
| SC20 | IIIC | 3.3(−) | 20.1(−) |
| SC23 | IB | 2.5(−) | 0.1(−) |
| SC24 | IA | 3.1(−) | 43.2(−) |
| SC25 | IIIA | 2.6(−) | 16.4(−) |
| SC26 | IA | 0.9(−) | 7.3(−) |
| SC27 | IA | 2.0(−) | 9.2(−) |
| SC29 | IIA | 1.3(−) | 35.6(−) |
| SC30 | IA | 2.8(−) | 0.1(−) |
| SC31 | IA | 2.4(−) | 14.0(−) |
| SC32 | IA | 4.0(−) | 10.5(−) |
| SC34 | IA | 2.4(−) | 17.0(−) |
| SC36 | IIIC | 1.5(−) | 14.1(−) |
| SC38 | IA | 4.8(−) | 47.6(+) |
| SC40 | IIA | 1.7(−) | 29.4(−) |
| SC41 | IA | 0.3(−) | 10.9(−) |
| SC42 | IIIA | 2.2(−) | 12.2(−) |
| SC45 | IIIC | 0.8(−) | 6.5(−) |
| SC47 | IB | 1.3(−) | 26.3(−) |
| SC48 | IIIA | 1.9(−) | 6.3(−) |
| SC49 | IA | 2.9(−) | 41.1(+) |
| SC50 | IB | 1.4(−) | 11.4(−) |

TABLE 5-2
Validation cohort

| Sample name | Cancer stage | CEA | CA19/9 |
|---|---|---|---|
| SC01 | IA | 3.7(−) | 0.1(−) |
| SC02 | IA | 4.9(−) | 65.2(+) |
| SC08 | IA | 1.1(−) | 9.9(−) |
| SC11 | IA | 1.8(−) | 9.4(−) |
| SC14 | IB | 2.0(−) | 26.1(−) |
| SC16 | IA | 3.1(−) | 9.5(−) |
| SC21 | IIA | 0.7(−) | 9.1(−) |
| SC22 | IA | 1.4(−) | 6.0(−) |
| SC28 | IA | 3.3(−) | 6.6(−) |
| SC33 | IIIA | 5.6(+) | 14.7(−) |
| SC35 | IA | 3.7(−) | 7.8(−) |
| SC37 | IIB | 4.2(−) | 0.1(−) |
| SC39 | IIC | 17.5(+) | 7.0(−) |
| SC43 | IIA | 4.6(−) | 10.1(−) |
| SC44 | IA | 1.8(−) | 5.8(−) |
| SC46 | IB | 2.7(−) | 37.1(+) |

For CEA, 5 ng/mL or lower was indicated as “−”, and for A19-9, 37 U/mL or lower was indicated as “−”, while values exceeding these were indicated as “+”.

TABLE 6

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 1_2 | 100.0 | 100.0 | 100.0 | 98.5 | 93.8 | 100.0 |
| 1_3 | 97.8 | 94.1 | 99.0 | 95.5 | 87.5 | 98.0 |
| 1_4 | 98.5 | 94.1 | 100.0 | 98.5 | 93.8 | 100.0 |
| 1_5 | 96.3 | 88.2 | 99.0 | 95.5 | 87.5 | 98.0 |
| 1_6 | 97.0 | 91.2 | 99.0 | 93.9 | 87.5 | 96.0 |
| 1_7 | 96.3 | 91.2 | 98.0 | 95.5 | 87.5 | 98.0 |
| 1_8 | 95.5 | 88.2 | 98.0 | 95.5 | 87.5 | 98.0 |
| 1_9 | 97.0 | 91.2 | 99.0 | 95.5 | 87.5 | 98.0 |
| 1_10 | 97.0 | 91.2 | 99.0 | 95.5 | 87.5 | 98.0 |
| 1_11 | 96.3 | 88.2 | 99.0 | 95.5 | 87.5 | 98.0 |
| 1_12 | 96.3 | 88.2 | 99.0 | 93.9 | 87.5 | 96.0 |
| 1_13 | 100.0 | 100.0 | 100.0 | 93.9 | 93.8 | 94.0 |
| 1_14 | 97.8 | 94.1 | 99.0 | 100.0 | 100.0 | 100.0 |
| 1_15 | 95.5 | 85.3 | 99.0 | 95.5 | 87.5 | 98.0 |
| 1_16 | 96.3 | 88.2 | 99.0 | 95.5 | 87.5 | 98.0 |
| 1_17 | 96.3 | 91.2 | 98.0 | 95.5 | 81.2 | 100.0 |
| 1_18 | 96.3 | 88.2 | 99.0 | 95.5 | 87.5 | 98.0 |
| 1_19 | 100.0 | 100.0 | 100.0 | 95.5 | 87.5 | 98.0 |
| 1_20 | 96.3 | 88.2 | 99.0 | 95.5 | 87.5 | 98.0 |
| 1_21 | 95.5 | 85.3 | 99.0 | 95.5 | 87.5 | 98.0 |
| 1_22 | 97.8 | 91.2 | 100.0 | 95.5 | 81.2 | 100.0 |
| 1_23 | 99.3 | 97.1 | 100.0 | 95.5 | 87.5 | 98.0 |
| 1_24 | 96.3 | 88.2 | 99.0 | 97.0 | 87.5 | 100.0 |
| 1_25 | 99.3 | 97.1 | 100.0 | 95.5 | 87.5 | 98.0 |
| 1_26 | 96.3 | 88.2 | 99.0 | 93.9 | 87.5 | 96.0 |
| 1_27 | 96.3 | 88.2 | 99.0 | 97.0 | 87.5 | 100.0 |
| 1_28 | 96.3 | 88.2 | 99.0 | 97.0 | 93.8 | 98.0 |
| 1_29 | 95.5 | 88.2 | 98.0 | 95.5 | 87.5 | 98.0 |
| 1_30 | 95.5 | 88.2 | 98.0 | 95.5 | 87.5 | 98.0 |
| 1_31 | 96.3 | 88.2 | 99.0 | 93.9 | 87.5 | 96.0 |
| 1_32 | 96.3 | 88.2 | 99.0 | 97.0 | 87.5 | 100.0 |
| 1_33 | 96.3 | 88.2 | 99.0 | 95.5 | 87.5 | 98.0 |
| 1_34 | 96.3 | 88.2 | 99.0 | 95.5 | 87.5 | 98.0 |
| 1_35 | 96.3 | 88.2 | 99.0 | 95.5 | 87.5 | 98.0 |
| 1_36 | 96.3 | 88.2 | 99.0 | 95.5 | 87.5 | 98.0 |
| 1_37 | 96.3 | 88.2 | 99.0 | 95.5 | 87.5 | 98.0 |
| 1_38 | 96.3 | 88.2 | 99.0 | 95.5 | 87.5 | 98.0 |
| 1_39 | 96.3 | 88.2 | 99.0 | 97.0 | 87.5 | 100.0 |
| 1_40 | 95.5 | 88.2 | 98.0 | 95.5 | 87.5 | 98.0 |
| 1_41 | 95.5 | 85.3 | 99.0 | 95.5 | 87.5 | 98.0 |
| 1_42 | 95.5 | 85.3 | 99.0 | 95.5 | 87.5 | 98.0 |
| 1_43 | 95.5 | 85.3 | 99.0 | 97.0 | 87.5 | 100.0 |
| 1_44 | 95.5 | 88.2 | 98.0 | 93.9 | 87.5 | 96.0 |
| 1_45 | 97.8 | 94.1 | 99.0 | 95.5 | 87.5 | 98.0 |
| 1_46 | 97.0 | 91.2 | 99.0 | 97.0 | 87.5 | 100.0 |
| 1_47 | 97.0 | 94.1 | 98.0 | 97.0 | 93.8 | 98.0 |
| 1_48 | 96.3 | 88.2 | 99.0 | 97.0 | 87.5 | 100.0 |
| 1_49 | 97.8 | 91.2 | 100.0 | 93.9 | 87.5 | 96.0 |
| 1_50 | 95.5 | 85.3 | 99.0 | 95.5 | 87.5 | 98.0 |
| 1_51 | 98.5 | 97.1 | 99.0 | 95.5 | 87.5 | 98.0 |
| 1_52 | 95.5 | 85.3 | 99.0 | 95.5 | 87.5 | 98.0 |
| 1_53 | 95.5 | 85.3 | 99.0 | 97.0 | 87.5 | 100.0 |
| 1_54 | 95.5 | 85.3 | 99.0 | 95.5 | 87.5 | 98.0 |
| 1_55 | 96.3 | 88.2 | 99.0 | 95.5 | 87.5 | 98.0 |
| 1_56 | 97.7 | 94.1 | 99.0 | 95.5 | 87.5 | 98.0 |
| 1_57 | 97.8 | 91.2 | 100.0 | 97.0 | 93.8 | 98.0 |
| 1_58 | 95.5 | 88.2 | 98.0 | 93.9 | 87.5 | 96.0 |
| 1_59 | 97.0 | 94.1 | 98.0 | 93.9 | 87.5 | 96.0 |
| 1_60 | 94.8 | 85.3 | 98.0 | 95.5 | 87.5 | 98.0 |
| 1_61 | 95.5 | 85.3 | 99.0 | 97.0 | 93.8 | 98.0 |
| 1_62 | 96.3 | 88.2 | 99.0 | 95.5 | 87.5 | 98.0 |
| 1_63 | 96.3 | 88.2 | 99.0 | 95.5 | 87.5 | 98.0 |
| 1_64 | 96.3 | 88.2 | 99.0 | 95.5 | 87.5 | 98.0 |
| 1_65 | 96.3 | 88.2 | 99.0 | 97.0 | 87.5 | 100.0 |
| 1_66 | 97.0 | 91.2 | 99.0 | 97.0 | 87.5 | 100.0 |
| 1_67 | 95.5 | 88.2 | 98.0 | 97.0 | 87.5 | 100.0 |
| 1_68 | 95.5 | 85.3 | 99.0 | 95.5 | 87.5 | 98.0 |
| 1_69 | 95.5 | 88.2 | 98.0 | 95.5 | 87.5 | 98.0 |
| 1_70 | 96.3 | 88.2 | 99.0 | 95.5 | 87.5 | 98.0 |
| 1_71 | 96.3 | 88.2 | 99.0 | 95.5 | 87.5 | 98.0 |
| 1_72 | 95.5 | 85.3 | 99.0 | 95.5 | 87.5 | 98.0 |
| 1_73 | 95.5 | 85.3 | 99.0 | 95.5 | 87.5 | 98.0 |
| 1_74 | 95.5 | 85.3 | 99.0 | 95.5 | 87.5 | 98.0 |
| 1_75 | 98.5 | 94.1 | 100.0 | 95.5 | 87.5 | 98.0 |
| 1_76 | 95.5 | 85.3 | 99.0 | 93.9 | 87.5 | 96.0 |
| 1_77 | 95.5 | 85.3 | 99.0 | 95.5 | 87.5 | 98.0 |
| 1_78 | 96.3 | 88.2 | 99.0 | 98.5 | 93.8 | 100.0 |
| 1_79 | 97.0 | 91.2 | 99.0 | 95.5 | 87.5 | 98.0 |
| 1_80 | 95.5 | 88.2 | 98.0 | 95.5 | 87.5 | 98.0 |
| 1_81 | 97.0 | 91.2 | 99.0 | 95.5 | 87.5 | 98.0 |
| 1_82 | 95.5 | 85.3 | 99.0 | 95.4 | 87.5 | 98.0 |
| 1_83 | 96.3 | 88.2 | 99.0 | 95.5 | 87.5 | 98.0 |
| 1_84 | 94.8 | 82.4 | 99.0 | 95.5 | 87.5 | 98.0 |
| 1_85 | 96.3 | 88.2 | 99.0 | 95.5 | 87.5 | 98.0 |
| 1_86 | 99.3 | 97.1 | 100.0 | 95.5 | 93.8 | 96.0 |
| 1_87 | 95.5 | 85.3 | 99.0 | 95.5 | 87.5 | 98.0 |
| 1_88 | 96.3 | 88.2 | 99.0 | 95.5 | 87.5 | 98.0 |
| 1_89 | 96.3 | 88.2 | 99.0 | 97.0 | 87.5 | 100.0 |
| 1_90 | 95.5 | 85.3 | 99.0 | 95.5 | 87.5 | 98.0 |
| 1_91 | 96.3 | 91.2 | 98.0 | 97.0 | 87.5 | 100.0 |
| 1_92 | 96.3 | 88.2 | 99.0 | 95.5 | 87.5 | 98.0 |
| 1_93 | 96.3 | 88.2 | 99.0 | 95.5 | 87.5 | 98.0 |
| 1_94 | 96.3 | 88.2 | 99.0 | 95.5 | 87.5 | 98.0 |
| 1_95 | 96.3 | 88.2 | 99.0 | 95.5 | 87.5 | 98.0 |
| 1_96 | 95.5 | 85.3 | 99.0 | 95.5 | 87.5 | 98.0 |
| 1_97 | 97.0 | 91.2 | 99.0 | 95.5 | 87.5 | 98.0 |
| 1_98 | 95.5 | 91.2 | 97.0 | 97.0 | 87.5 | 100.0 |
| 1_99 | 95.5 | 85.3 | 99.0 | 97.0 | 87.5 | 100.0 |
| 1_100 | 96.3 | 88.2 | 99.0 | 95.5 | 87.5 | 98.0 |
| 1_101 | 96.3 | 88.2 | 99.0 | 95.5 | 87.5 | 98.0 |
| 1_102 | 99.3 | 97.1 | 100.0 | 97.0 | 93.8 | 98.0 |
| 1_103 | 95.5 | 85.3 | 99.0 | 95.5 | 87.5 | 98.0 |
| 1_104 | 95.5 | 85.3 | 99.0 | 93.9 | 87.5 | 96.0 |
| 1_105 | 95.5 | 85.3 | 99.0 | 95.5 | 87.5 | 98.0 |
| 1_106 | 96.3 | 88.2 | 99.0 | 95.5 | 87.5 | 98.0 |
| 1_107 | 96.3 | 88.2 | 99.0 | 95.5 | 87.5 | 98.0 |
| 1_108 | 97.0 | 91.2 | 99.0 | 93.9 | 87.5 | 96.0 |
| 1_109 | 95.5 | 85.3 | 99.0 | 95.5 | 87.5 | 98.0 |
| 1_110 | 96.3 | 88.2 | 99.0 | 95.5 | 87.5 | 98.0 |
| 1_111 | 96.3 | 88.2 | 99.0 | 95.5 | 87.5 | 98.0 |
| 1_112 | 95.5 | 85.3 | 99.0 | 95.5 | 87.5 | 98.0 |
| 1_113 | 97.0 | 91.2 | 99.0 | 98.5 | 93.8 | 100.0 |
| 1_114 | 95.5 | 85.3 | 99.0 | 95.5 | 87.5 | 98.0 |
| 1_115 | 95.5 | 85.3 | 99.0 | 95.5 | 87.5 | 98.0 |
| 1_116 | 96.3 | 91.2 | 98.0 | 98.5 | 93.8 | 100.0 |
| 1_117 | 96.3 | 88.2 | 99.0 | 95.5 | 87.5 | 98.0 |
| 1_118 | 96.3 | 88.2 | 99.0 | 95.5 | 87.5 | 98.0 |
| 1_119 | 95.5 | 85.3 | 99.0 | 95.5 | 87.5 | 98.0 |
| 1_120 | 97.0 | 91.2 | 99.0 | 95.5 | 87.5 | 98.0 |
| 1_121 | 96.3 | 88.2 | 99.0 | 95.5 | 87.5 | 98.0 |
| 1_122 | 96.3 | 88.2 | 99.0 | 97.0 | 87.5 | 100.0 |
| 1_123 | 97.8 | 91.2 | 100.0 | 97.0 | 93.8 | 98.0 |
| 1_124 | 95.5 | 85.3 | 99.0 | 95.5 | 87.5 | 98.0 |
| 1_125 | 98.5 | 94.1 | 100.0 | 97.0 | 93.8 | 98.0 |
| 1_126 | 97.0 | 91.2 | 99.0 | 97.0 | 93.8 | 98.0 |
| 1_127 | 96.3 | 88.2 | 99.0 | 95.5 | 87.5 | 98.0 |
| 1_128 | 96.3 | 88.2 | 99.0 | 95.5 | 87.5 | 98.0 |
| 1_129 | 97.0 | 91.2 | 99.0 | 97.0 | 87.5 | 100.0 |
| 1_130 | 96.3 | 88.2 | 99.0 | 95.5 | 87.5 | 98.0 |
| 1_131 | 96.3 | 88.2 | 99.0 | 95.5 | 87.5 | 98.0 |
| 1_132 | 97.0 | 91.2 | 99.0 | 95.5 | 87.5 | 98.0 |
| 1_133 | 96.3 | 88.2 | 99.0 | 95.5 | 87.5 | 98.0 |
| 1_134 | 96.3 | 88.2 | 99.0 | 95.5 | 87.5 | 98.0 |
| 1_135 | 96.3 | 88.2 | 99.0 | 97.0 | 93.8 | 98.0 |
| 1_136 | 95.5 | 85.3 | 99.0 | 95.5 | 87.5 | 98.0 |
| 1_137 | 95.5 | 85.3 | 99.0 | 97.0 | 93.8 | 98.0 |
| 1_138 | 96.3 | 88.2 | 99.0 | 95.5 | 87.5 | 98.0 |
| 1_139 | 95.5 | 85.3 | 99.0 | 95.5 | 87.5 | 98.0 |
| 1_140 | 97.0 | 91.2 | 99.0 | 95.5 | 87.5 | 98.0 |
| 1_141 | 95.5 | 85.3 | 99.0 | 98.5 | 93.8 | 100.0 |
| 1_142 | 97.8 | 91.2 | 100.0 | 93.9 | 87.5 | 96.0 |
| 1_143 | 95.5 | 85.3 | 99.0 | 95.5 | 87.5 | 98.0 |
| 1_144 | 96.3 | 88.2 | 99.0 | 97.0 | 87.5 | 100.0 |
| 1_145 | 96.2 | 87.9 | 99.0 | 95.5 | 87.5 | 98.0 |
| 1_146 | 99.3 | 97.1 | 100.0 | 97.0 | 93.8 | 98.0 |
| 1_147 | 96.3 | 88.2 | 99.0 | 95.5 | 87.5 | 98.0 |
| 1_148 | 96.3 | 88.2 | 99.0 | 98.5 | 93.8 | 100.0 |
| 1_149 | 95.5 | 85.3 | 99.0 | 95.4 | 87.5 | 98.0 |
| 1_150 | 97.0 | 91.2 | 99.0 | 97.0 | 87.5 | 100.0 |
| 1_151 | 95.5 | 88.2 | 98.0 | 95.5 | 87.5 | 98.0 |
| 1_152 | 95.5 | 85.3 | 99.0 | 95.5 | 87.5 | 98.0 |
| 1_153 | 99.3 | 97.1 | 100.0 | 97.0 | 93.8 | 98.0 |
| 1_154 | 96.3 | 88.2 | 99.0 | 97.0 | 87.5 | 100.0 |
| 1_155 | 96.3 | 88.2 | 99.0 | 95.5 | 87.5 | 98.0 |
| 1_156 | 96.3 | 88.2 | 99.0 | 97.0 | 87.5 | 100.0 |
| 1_157 | 96.3 | 88.2 | 99.0 | 97.0 | 93.8 | 98.0 |
| 1_158 | 95.5 | 85.3 | 99.0 | 95.5 | 87.5 | 98.0 |
| 1_159 | 95.5 | 85.3 | 99.0 | 95.5 | 87.5 | 98.0 |
| 1_160 | 95.5 | 85.3 | 99.0 | 95.5 | 87.5 | 98.0 |
| 1_161 | 95.5 | 85.3 | 99.0 | 95.5 | 87.5 | 98.0 |
| 1_162 | 96.3 | 88.2 | 99.0 | 95.5 | 87.5 | 98.0 |
| 1_163 | 95.5 | 85.3 | 99.0 | 95.5 | 87.5 | 98.0 |
| 1_164 | 96.3 | 88.2 | 99.0 | 95.5 | 87.5 | 98.0 |
| 1_165 | 97.8 | 94.1 | 99.0 | 95.5 | 87.5 | 98.0 |
| 1_166 | 96.3 | 88.2 | 99.0 | 95.5 | 87.5 | 98.0 |
| 1_167 | 98.5 | 97.1 | 99.0 | 97.0 | 93.8 | 98.0 |
| 1_168 | 97.0 | 91.2 | 99.0 | 95.5 | 87.5 | 98.0 |
| 1_169 | 96.3 | 88.2 | 99.0 | 95.5 | 87.5 | 98.0 |

### Example 3

### <Selection of Gene Markers Using all Samples and Method for Evaluating Stomach Cancer Discriminant Performance of Acquired Gene Markers>

In this Example, the samples of the training cohort and the validation cohort used in Examples 1 and 2 described above were integrated, and selection of a gene marker and evaluation of its stomach cancer discriminant performance were conducted using all of the samples.

Specifically, the expression levels in the sera of the 50 stomach cancer patients and the 150 healthy subject miRNA s obtained in the preceding Reference Examples were normalized by quantile normalization. In order to acquire diagnosis markers with higher reliability, only genes having a gene expression level of 2⁶ or higher in 50% or more of the samples in either of the stomach cancer patient group or the healthy subject group were selected in the gene marker selection. In order to further acquire statistical significance for discriminating a stomach cancer patient group from a healthy subject group, the P value obtained by two-tailed t-test assuming equal variance as to each gene expression level was corrected by the Bonferroni method, and genes that satisfied p<0.01 were selected as gene markers for use in explanatory variables of a discriminant and described in Table 7. In this way, hsa-miR-3196, hsa-miR-211-3p, hsa-miR-4271, hsa-miR-6851-5p, hsa-miR-149-3p, hsa-miR-4667-5p, hsa-miR-135a-3p, hsa-miR-4486, hsa-miR-4697-5p, hsa-miR-4725-3p, hsa-miR-6510-5p, hsa-miR-5001-5p, hsa-miR-4673, hsa-miR-4466, hsa-miR-23a-3p, hsa-miR-3656, hsa-miR-6782-5p, hsa-miR-4689, hsa-miR-451a, hsa-miR-4446-3p, hsa-miR-3180-3p, hsa-miR-642a-3p, hsa-miR-6889-5p, hsa-miR-3178, hsa-miR-4665-5p, hsa-miR-6722-3p, hsa-miR-30c-1-3p, hsa-miR-4507, hsa-miR-3141 and hsa-miR-1199-5p genes, and the nucleotide sequences of SEQ ID NOs: 170 to 199 related thereto were found in addition to the genes described in Table 2. As with the nucleotide sequences shown in SEQ ID NOs: 1 to 169, the results obtained about the polynucleotides shown in SEQ ID NOs: 170 to 199 also showed that the measurement values were significantly lower (−) or higher (+) in the stomach cancer patient group than in the healthy subject group (Table 7). These results were able to be validated in the validation cohort. Thus, the presence or absence of stomach cancer in the newly obtained samples can be determined by the methods described in Examples 1 and 2 by using, alone or in combination, the gene expression level measurement values described in Table 7.

TABLE 7

| | | | Expression level in |
|---|---|---|---|
| | | P value after | stomach cancer |
| | | Bonferroni | patient relative to |
| SEQ ID NO: | Gene name | correction | healthy subject |
| 1 | hsa-miR-4257 | 1.17.E−53 | − |
| 2 | hsa-miR-6726-5p | 1.13.E−52 | − |
| 3 | hsa-niiR-1343-3p | 1.41.E−44 | − |
| 4 | hsa-miR-1247-3p | 5.94.E−35 | + |
| 5 | hsa-miR-6787-5p | 2.22.E−39 | − |
| 6 | hsa-miR-6875-5p | 1.92.E−30 | + |
| 7 | hsa-miR-1225-3p | 6.99.E−36 | + |
| 8 | hsa-miR-8063 | 7.15.E−31 | − |
| 9 | hsa-miR-6781-5p | 4.27.E−31 | + |
| 10 | hsa-miR-4746-3p | 1.93.E−35 | + |
| 11 | hsa-miR-1908-5p | 1.34.E−32 | + |
| 12 | hsa-miR-6756-5p | 2.25.E−28 | − |
| 13 | hsa-miR-204-3p | 5.11.E−30 | − |
| 14 | hsa-miR-4651 | 2.11.E−33 | − |
| 15 | hsa-miR-6757-5p | 2.11.E−34 | − |
| 16 | hsa-miR-6825-5p | 1.20.E−31 | + |
| 17 | hsa-miR-7108-5p | 3.88.E−25 | + |
| 18 | hsa-miR-4792 | 5.31.E−29 | + |
| 19 | hsa-miR-7641 | 1.72.E−27 | − |
| 20 | hsa-miR-3188 | 3.58.E−30 | + |
| 21 | hsa-miR-3131 | 3.98.E−33 | − |
| 22 | hsa-miR-6780b-5p | 4.88.E−28 | + |
| 23 | hsa-miR-8069 | 7.94.E−21 | + |
| 24 | hsa-miR-6840-3p | 4.43.E−23 | − |
| 25 | hsa-miR-8072 | 1.55.E−23 | + |
| 26 | hsa-miR-1233-5p | 3.51.E−26 | − |
| 27 | hsa-miR-6887-5p | 1.34.E−24 | − |
| 28 | hsa-miR-1231 | 9.31.E−26 | + |
| 29 | hsa-miR-5572 | 3.97.E−25 | + |
| 30 | hsa-miR-6738-5p | 2.02.E−21 | − |
| 31 | hsa-miR-6784-5p | 1.03.E−23 | + |
| 32 | hsa-miR-6791-5p | 2.63.E−22 | + |
| 33 | hsa-miR-6749-5p | 6.36.E−23 | − |
| 34 | hsa-miR-6741-5p | 6.07.E−23 | − |
| 35 | hsa-miR-128-1-5p | 3.13.E−20 | + |
| 36 | hsa-miR-4419b | 9.02.E−24 | − |
| 37 | hsa-miR-6746-5p | 1.60.E−25 | − |
| 38 | hsa-miR-3184-5p | 1.38.E−23 | + |
| 39 | hsa-miR-3679-5p | 6.33.E−26 | + |
| 40 | hsa-miR-7110-5p | 3.06.E−24 | + |
| 41 | hsa-miR-4516 | 1.26.E−23 | − |
| 42 | hsa-miR-6717-5p | 6.77.E−26 | − |
| 43 | hsa-miR-6826-5p | 8.66.E−25 | − |
| 44 | hsa-miR-4433b-3p | 5.71.E−19 | + |
| 45 | hsa-miR-3679-3p | 2.22.E−19 | + |
| 46 | hsa-miR-3135b | 7.59.E−15 | − |
| 47 | hsa-miR-3622a-5p | 4.66.E−24 | − |
| 48 | hsa-miR-711 | 9.88.E−22 | + |
| 49 | hsa-miR-4467 | 3.85.E−21 | + |
| 50 | hsa-miR-6857-5p | 1.03.E−19 | + |
| 51 | hsa-miR-6515-3p | 5.53.E−16 | + |
| 52 | hsa-miR-1225-5p | 2.33.E−19 | + |
| 53 | hsa-miR-187-5p | 1.31.E−20 | − |
| 54 | hsa-miR-3185 | 1.30.E−19 | + |
| 55 | hsa-miR-642b-3p | 2.56.E−18 | − |
| 56 | hsa-miR-1249 | 5.41.E−19 | + |
| 57 | hsa-miR-744-5p | 9.41.E−20 | + |
| 58 | hsa-miR-4442 | 1.75.E−17 | − |
| 59 | hsa-miR-1228-3p | 1.10.E−19 | + |
| 60 | hsa-miR-939-5p | 1.54.E−16 | + |
| 61 | hsa-miR-6845-5p | 5.15.E−20 | + |
| 62 | hsa-miR-887-3p | 2.86.E−15 | + |
| 63 | hsa-miR-7845-5p | 3.21.E−16 | + |
| 64 | hsa-miR-6729-5p | 6.04.E−16 | + |
| 65 | hsa-miR-4632-5p | 7.17.E−16 | + |
| 66 | hsa-miR-615-5p | 3.13.E−17 | − |
| 67 | hsa-miR-6724-5p | 6.37.E−15 | + |
| 68 | hsa-miR-4728-5p | 1.26.E−16 | − |
| 69 | hsa-miR-6732-5p | 5.05.E−14 | + |
| 70 | hsa-miR-6816-5p | 8.52.E−17 | + |
| 71 | hsa-miR-4695-5p | 2.40.E−14 | + |
| 72 | hsa-miR-6088 | 7.13.E−16 | − |
| 73 | hsa-miR-7975 | 1.51.E−14 | − |
| 74 | hsa-miR-3197 | 5.56.E−16 | + |
| 75 | hsa-miR-6125 | 2.29.E−15 | + |
| 76 | hsa-miR-4433-3p | 1.66.E−13 | + |
| 77 | hsa-miR-6727-5p | 1.77.E−15 | − |
| 78 | hsa-miR-4706 | 2.89.E−15 | − |
| 79 | hsa-miR-7847-3p | 1.35.E−14 | − |
| 80 | hsa-miR-6805-3p | 5.87.E−17 | + |
| 81 | hsa-miR-6766-3p | 1.02.E−14 | + |
| 82 | hsa-miR-1913 | 6.53.E−14 | + |
| 83 | hsa-miR-4649-5p | 1.20.E−13 | − |
| 84 | hsa-miR-602 | 3.43.E−17 | + |
| 85 | hsa-miR-3663-3p | 2.10.E−13 | − |
| 86 | hsa-miR-6893-5p | 3.43.E−17 | − |
| 87 | hsa-miR-6861-5p | 4.41.E−14 | − |
| 88 | hsa-miR-4449 | 2.00.E−16 | + |
| 89 | hsa-miR-6842-5p | 1.49.E−15 | + |
| 90 | hsa-miR-4454 | 1.57.E−13 | − |
| 91 | hsa-miR-5195-3p | 6.87.E−14 | − |
| 92 | hsa-miR-663b | 1.51.E−12 | − |
| 93 | hsa-miR-6765-5p | 5.17.E−11 | + |
| 94 | hsa-miR-4513 | 3.77.E−14 | − |
| 95 | hsa-miR-614 | 1.11.E−11 | − |
| 96 | hsa-miR-6785-5p | 6.54.E−12 | − |
| 97 | hsa-miR-6777-5p | 2.92.E−14 | − |
| 98 | hsa-miR-940 | 1.38.E−13 | + |
| 99 | hsa-miR-4741 | 2.04.E−12 | + |
| 100 | hsa-miR-6870-5p | 4.12.E−14 | + |
| 101 | hsa-miR-6131 | 1.02.E−12 | − |
| 102 | hsa-miR-150-3p | 1.47.E−10 | − |
| 103 | hsa-miR-4707-5p | 8.76.E−12 | + |
| 104 | hsa-miR-1915-3p | 4.55.E−13 | + |
| 105 | hsa-miR-3937 | 6.01.E−12 | + |
| 106 | hsa-miR-937-5p | 3.22.E−11 | − |
| 107 | hsa-miR-4443 | 3.16.E−10 | + |
| 108 | hsa-miR-1914-3p | 7.61.E−11 | − |
| 109 | hsa-miR-3620-5p | 7.63 E−11 | + |
| 110 | hsa-miR-1268b | 2.01.E−09 | + |
| 111 | hsa-miR-1227-5p | 3.14.E−10 | + |
| 112 | hsa-miR-6880-5p | 3.80.E−09 | + |
| 113 | hsa-miR-4417 | 7.19.E−10 | + |
| 114 | hsa-miR-6802-5p | 4.37.E−11 | − |
| 115 | hsa-miR-6769a-5p | 3.34.E−09 | − |
| 116 | hsa-miR-663a | 7.98.E−11 | + |
| 117 | hsa-miR-6721-5p | 1.38.E−09 | + |
| 118 | hsa-miR-4532 | 9.58.E−08 | − |
| 119 | hsa-miR-7977 | 9.99.E−11 | − |
| 120 | hsa-miR-92b-5p | 1.77.E−08 | + |
| 121 | hsa-miR-371a-5p | 8.63.E−09 | − |
| 122 | hsa-miR-6126 | 1.93.E−10 | + |
| 123 | hsa-miR-4734 | 3.27.E−09 | + |
| 124 | hsa-miR-4665-3p | 6.99.E−14 | + |
| 125 | hsa-miR-423-5p | 1.58.E−08 | − |
| 126 | hsa-miR-1469 | 8.71.E−07 | + |
| 127 | hsa-miR-4675 | 2.67.E−10 | − |
| 128 | hsa-miR-1915-5p | 1.06.E−08 | − |
| 129 | hsa-miR-6716-5p | 7.56.E−09 | + |
| 130 | hsa-miR-718 | 1.99.E−09 | + |
| 131 | hsa-miR-4281 | 9.46.E−11 | − |
| 132 | hsa-miR-6820-5p | 1.42.E−08 | − |
| 133 | hsa-miR-6795-5p | 4.38.E−10 | − |
| 134 | hsa-miR-6779-5p | 2.99.E−08 | − |
| 135 | hsa-miR-7109-5p | 7.06.E−08 | − |
| 136 | hsa-miR-6798-5p | 7.93.E−07 | + |
| 137 | hsa-miR-4648 | 2.21.E−09 | + |
| 138 | hsa-miR-8059 | 1.44.E−08 | − |
| 139 | hsa-miR-6765-3p | 6.59.E−08 | − |
| 140 | hsa-miR-6132 | 3.82.E−06 | + |
| 141 | hsa-miR-4492 | 1.34.E−08 | + |
| 142 | hsa-miR-7107-5p | 1.84.E−06 | − |
| 143 | hsa-miR-3195 | 6.91.E−08 | + |
| 144 | hsa-miR-3180 | 1.11.E−07 | + |
| 145 | hsa-miR-296-3p | 2.56.E−06 | − |
| 146 | hsa-miR-564 | 1.32.E−07 | − |
| 147 | hsa-miR-1268a | 1.25.E−04 | + |
| 148 | hsa-miR-6848-5p | 2.82.E−06 | + |
| 149 | hsa-miR-762 | 5.66.E−04 | + |
| 150 | hsa-miR-2861 | 1.45.E−06 | − |
| 151 | hsa-miR-1203 | 7.90.E−07 | + |
| 152 | hsa-miR-1260b | 2.26.E−04 | − |
| 153 | hsa-miR-4476 | 5.95.E−06 | − |
| 154 | hsa-miR-6885-5p | 5.73.E−05 | − |
| 155 | hsa-miR-6769b-5p | 1.91.E−07 | − |
| 156 | hsa-miR-23b-3p | 1.38.E−05 | − |
| 157 | hsa-miR-1343-5p | 7.73.E−06 | + |
| 158 | hsa-miR-3621 | 3.64.E−05 | − |
| 159 | hsa-miR-4688 | 1.47.E−05 | − |
| 160 | hsa-miR-4286 | 3.79.E−03 | − |
| 161 | hsa-miR-4640-5p | 1.79.E−05 | + |
| 162 | hsa-miR-4739 | 2.45.E−05 | + |
| 163 | hsa-miR-1260a | 7.35.E−04 | − |
| 164 | hsa-miR-4276 | 3.45.E−07 | + |
| 165 | hsa-miR-7106-5p | 4.60.E−04 | − |
| 166 | hsa-miR-128-2-5p | 1.05.E−13 | − |
| 167 | lisa-miR-125a-3p | 2.30.E−15 | − |
| 168 | hsa-miR-92a-2-5p | 5.42.E−09 | + |
| 169 | hsa-miR-486-3p | 2.00.E−05 | − |
| 170 | hsa-miR-3196 | 2.06.E−06 | + |
| 171 | hsa-miR-211-3p | 2.03.E−05 | − |
| 172 | hsa-miR-4271 | 2.31.E−05 | − |
| 173 | hsa-miR-6851-5p | 2.68.E−05 | + |
| 174 | hsa-miR-149-3p | 2.75.E−05 | − |
| 175 | hsa-miR-4667-5p | 4.05.E−05 | + |
| 176 | hsa-miR-135a-3p | 4.28.E−05 | + |
| 177 | hsa-miR-4486 | 6.68.E−05 | + |
| 178 | hsa-miR-4697-5p | 7.18.E−05 | − |
| 179 | hsa-miR-4725-3p | 8.16.E−05 | + |
| 180 | hsa-miR-6510-5p | 8.18.E−05 | + |
| 181 | hsa-miR-5001-5p | 1.92.E−04 | − |
| 182 | hsa-miR-4673 | 2.32.E−04 | + |
| 183 | hsa-miR-4466 | 3.06.E−04 | − |
| 184 | hsa-miR-23a-3p | 5.28.E−04 | − |
| 185 | hsa-miR-3656 | 5.41.E−04 | + |
| 186 | hsa-miR-6782-5p | 7.05.E−04 | + |
| 187 | hsa-miR-4689 | 1.01.E−03 | − |
| 188 | hsa-miR-451a | 1.22.E−03 | − |
| 189 | hsa-miR-4446-3p | 1.51.E−03 | − |
| 190 | hsa-miR-3180-3p | 1.64.E−03 | + |
| 191 | hsa-miR-642a-3p | 1.80.E−03 | − |
| 192 | hsa-miR-6889-5p | 1.91.E−03 | + |
| 193 | hsa-miR-3178 | 2.08.E−03 | + |
| 194 | hsa-miR-4665-5p | 2.84.E−03 | − |
| 195 | hsa-miR-6722-3p | 3.22.E−03 | + |
| 196 | hsa-miR-30c-1-3p | 4.13.E−03 | + |
| 197 | hsa-miR-4507 | 6.12.E−03 | + |
| 198 | hsa-miR-3141 | 6.13.E−03 | + |
| 199 | hsa-miR-1199-5p | 7.28.E−03 | − |

### Example 4

### <Method for Evaluating Stomach Cancer-Specific Discriminant Performance by Combination of Multiple Gene Markers Using Samples of Validation Cohort>

In this Example, gene markers for diagnosis were selected by comparing gene expression levels of miRNAs in sera of stomach cancer patients with that of a control group consisting of healthy subjects, pancreatic cancer patients, bile duct cancer patients, colorectal cancer patients, liver cancer patients, and benign pancreaticobiliary disease patients in the same way as the method described in Example 1 using the gene markers selected in Example 1 with respect to the training cohort as the sample group described in Reference Example 2. The polynucleotides consisting of the nucleotide sequences represented by SEQ ID NOs: 635 to 642 thus selected were further combined therewith to study a method for evaluating stomach cancer-specific discriminant performance.

Specifically, first, the miRNA expression levels in the training cohort and the validation cohort obtained in Reference Example 2 mentioned above were combined and normalized by quantile normalization. Next, Fisher's discriminant analysis was conducted as to combinations of 1 to 6 expression level measurement values comprising at least one or more of the expression level measurement values of the polynucleotides consisting of the nucleotide sequences represented by SEQ ID NOs: 1 to 165 and 635 to 642, to construct a discriminant for determining the presence or absence of stomach cancer. Next, accuracy, sensitivity, and specificity in the validation cohort were calculated using the discriminant thus prepared, with the stomach cancer patient group as a positive sample group and, on the other hand, the healthy subject group, the pancreatic cancer patient group, the bile duct cancer patient group, the colorectal cancer patient group, the liver cancer patient group, and the benign pancreaticobiliary disease patient group as a negative sample groups. The discriminant performance of the selected polynucleotides was validated using the independent samples.

Most of polynucleotides consisting of the nucleotide sequences represented by any of these SEQ ID NOs (SEQ ID NOs: 1 to 165 and 635 to 642 corresponding to the miRNA markers of Table 1) or complementary sequences thereof mentioned above were able to provide relatively high accuracy, sensitivity, and specificity in the determination of the presence or absence of stomach cancer, and furthermore, were able to specifically discriminate stomach cancer from the other cancers. For example, among the combinations of multiple polynucleotides selected from the group consisting of polynucleotides consisting of the nucleotide sequences represented by SEQ ID NOs: 9, 13, 21, 27, 34, 36, 66, 75, 95, 98, 108, 130, 135, 143, 155, 183, 185, 187, 191, 193, 194, 635, 636, 637, 638, 639, 640, 641 and 642 or complementary sequences thereof (the cancer type-specific polynucleotide group 1) as polynucleotides capable of specifically binding to target markers, combinations comprising at least one or more polynucleotides selected from the group consisting of polynucleotides consisting of the nucleotide sequences represented by SEQ ID NOs: 21, 34, 36, 98, and 155 or complementary sequences thereof (the cancer type-specific polynucleotide group 2) included in the cancer type-specific polynucleotide group 1 were able to specifically discriminate stomach cancer from the other cancers with high accuracy.

The number of the aforementioned polynucleotides with cancer type specificity in the combination can be 2, 3, 4, 5, 6, 7, 8, 9, 10 or more for the combination. The combinations of 6 or more of these polynucleotides were able to exhibit discriminant accuracy of 80% or higher.

Specifically, the discriminant accuracy of the measurement using the polynucleotide consisting of the nucleotide sequence represented by SEQ ID NO: 21 or a complementary sequence thereof is shown in Table 8-1. The measurement using the polynucleotide consisting of the nucleotide sequence represented by SEQ ID NO: 21 or a complementary sequence thereof exhibited the highest accuracy of 79.8% in the training cohort and accuracy of 83.8% in the validation cohort. Also, for example, the measurement using the combinations of two polynucleotides comprising at least one polynucleotide consisting of the nucleotide sequence represented by SEQ ID NO: 21 or a complementary sequence thereof exhibited the highest accuracy of 82.4% in the training cohort and accuracy of 80.9% in the validation cohort. Furthermore, for example, the measurement using the combinations of three polynucleotides comprising at least one polynucleotide consisting of the nucleotide sequence represented by SEQ ID NO: 21 or a complementary sequence thereof exhibited the highest accuracy of 84.1% in the training cohort and accuracy of 83.8% in the validation cohort. Furthermore, for example, the measurement using the combinations of four polynucleotides comprising at least one polynucleotide consisting of the nucleotide sequence represented by SEQ ID NO: 21 or a complementary sequence thereof exhibited the highest accuracy of 85.9% in the training cohort and accuracy of 82.7% in the validation cohort. Furthermore, for example, the measurement using the combinations of five polynucleotides comprising at least one polynucleotide consisting of the nucleotide sequence represented by SEQ ID NO: 21 or a complementary sequence thereof exhibited the highest accuracy of 87.9% in the training cohort and accuracy of 88.4% in the validation cohort. Furthermore, for example, the measurement using the combinations of six polynucleotides comprising at least one polynucleotide consisting of the nucleotide sequence represented by SEQ ID NO: 21 or a complementary sequence thereof exhibited the highest accuracy of 87.0% in the training cohort and accuracy of 87.3% in the validation cohort.

Specifically, the discriminant accuracy of the measurement using the polynucleotide consisting of the nucleotide sequence represented by SEQ ID NO: 34 or a complementary sequence thereof is shown in Table 8-2. The measurement using the polynucleotide consisting of the nucleotide sequence represented by SEQ ID NO: 34 or a complementary sequence thereof exhibited the highest accuracy of 62.8% in the training cohort and accuracy of 60.7% in the validation cohort. Also, for example, the measurement using the combinations of two polynucleotides comprising at least one polynucleotide consisting of the nucleotide sequence represented by SEQ ID NO: 34 or a complementary sequence thereof exhibited the highest accuracy of 81.0% in the training cohort and accuracy of 82.1% in the validation cohort. Furthermore, for example, the measurement using the combinations of three polynucleotides comprising at least one polynucleotide consisting of the nucleotide sequence represented by SEQ ID NO: 34 or a complementary sequence thereof exhibited the highest accuracy of 84.4% in the training cohort and accuracy of 82.1% in the validation cohort. Furthermore, for example, the measurement using the combinations of four polynucleotides comprising at least one polynucleotide consisting of the nucleotide sequence represented by SEQ ID NO: 34 or a complementary sequence thereof exhibited the highest accuracy of 87.0% in the training cohort and accuracy of 88.4% in the validation cohort. Furthermore, for example, the measurement using the combinations of five polynucleotides comprising at least one polynucleotide consisting of the nucleotide sequence represented by SEQ ID NO: 34 or a complementary sequence thereof exhibited the highest accuracy of 88.2% in the training cohort and accuracy of 87.9% in the validation cohort. Furthermore, for example, the measurement using the combinations of six polynucleotides comprising at least one polynucleotide consisting of the nucleotide sequence represented by SEQ ID NO: 34 or a complementary sequence thereof exhibited the highest accuracy of 87.3% in the training cohort and accuracy of 88.4% in the validation cohort.

Specifically, the discriminant accuracy of the measurement using the polynucleotide consisting of the nucleotide sequence represented by SEQ ID NO: 36 or a complementary sequence thereof is shown in Table 8-3. The measurement using the polynucleotide consisting of the nucleotide sequence represented by SEQ ID NO: 36 or a complementary sequence thereof exhibited the highest accuracy of 78.7% in the training cohort and accuracy of 78.6% in the validation cohort. Also, for example, the measurement using the combinations of two polynucleotides comprising at least one polynucleotide consisting of the nucleotide sequence represented by SEQ ID NO: 36 or a complementary sequence thereof exhibited the highest accuracy of 82.7% in the training cohort and accuracy of 82.7% in the validation cohort. Furthermore, for example, the measurement using the combinations of three polynucleotides comprising at least one polynucleotide consisting of the nucleotide sequence represented by SEQ ID NO: 36 or a complementary sequence thereof exhibited the highest accuracy of 85.0% in the training cohort and accuracy of 86.7% in the validation cohort. Furthermore, for example, the measurement using the combinations of four polynucleotides comprising at least one polynucleotide consisting of the nucleotide sequence represented by SEQ ID NO: 36 or a complementary sequence thereof exhibited the highest accuracy of 87.0% in the training cohort and accuracy of 85.5% in the validation cohort. Furthermore, for example, the measurement using the combinations of five polynucleotides comprising at least one polynucleotide consisting of the nucleotide sequence represented by SEQ ID NO: 36 or a complementary sequence thereof exhibited the highest accuracy of 87.9% in the training cohort and accuracy of 86.1% in the validation cohort. Furthermore, for example, the measurement using the combinations of six polynucleotides comprising at least one polynucleotide consisting of the nucleotide sequence represented by SEQ ID NO: 36 or a complementary sequence thereof exhibited the highest accuracy of 87.3% in the training cohort and accuracy of 87.9% in the validation cohort.

Specifically, the discriminant accuracy of the measurement using the polynucleotide consisting of the nucleotide sequence represented by SEQ ID NO: 98 or a complementary sequence thereof is shown in Table 8-4. The measurement using the polynucleotide consisting of the nucleotide sequence represented by SEQ ID NO: 98 or a complementary sequence thereof exhibited the highest accuracy of 70.9% in the training cohort and accuracy of 70.5% in the validation cohort. Also, for example, the measurement using the combinations of two polynucleotides comprising at least one polynucleotide consisting of the nucleotide sequence represented by SEQ ID NO: 98 or a complementary sequence thereof exhibited the highest accuracy of 82.4% in the training cohort and accuracy of 82.1% in the validation cohort. Furthermore, for example, the measurement using the combinations of three polynucleotides comprising at least one polynucleotide consisting of the nucleotide sequence represented by SEQ ID NO: 98 or a complementary sequence thereof exhibited the highest accuracy of 84.1% in the training cohort and accuracy of 85.5% in the validation cohort. Furthermore, for example, the measurement using the combinations of four polynucleotides comprising at least one polynucleotide consisting of the nucleotide sequence represented by SEQ ID NO: 98 or a complementary sequence thereof exhibited the highest accuracy of 86.7% in the training cohort and accuracy of 89.6% in the validation cohort. Furthermore, for example, the measurement using the combinations of five polynucleotides comprising at least one polynucleotide consisting of the nucleotide sequence represented by SEQ ID NO: 98 or a complementary sequence thereof exhibited the highest accuracy of 88.2% in the training cohort and accuracy of 87.9% in the validation cohort. Furthermore, for example, the measurement using the combinations of six polynucleotides comprising at least one polynucleotide consisting of the nucleotide sequence represented by SEQ ID NO: 98 or a complementary sequence thereof exhibited the highest accuracy of 87.9% in the training cohort and accuracy of 88.4% in the validation cohort.

Specifically, the discriminant accuracy of the measurement using the polynucleotide consisting of the nucleotide sequence represented by SEQ ID NO: 155 or a complementary sequence thereof is shown in Table 8-5. The measurement using the polynucleotide consisting of the nucleotide sequence represented by SEQ ID NO: 155 or a complementary sequence thereof exhibited the highest accuracy of 67.1% in the training cohort and accuracy of 69.9% in the validation cohort. Also, for example, the measurement using the combinations of two polynucleotides comprising at least one polynucleotide consisting of the nucleotide sequence represented by SEQ ID NO: 155 or a complementary sequence thereof exhibited the highest accuracy of 81.6% in the training cohort and accuracy of 75.7% in the validation cohort. Furthermore, for example, the measurement using the combinations of three polynucleotides comprising at least one polynucleotide consisting of the nucleotide sequence represented by SEQ ID NO: 155 or a complementary sequence thereof exhibited the highest accuracy of 84.4% in the training cohort and accuracy of 85.0% in the validation cohort. Furthermore, for example, the measurement using the combinations of four polynucleotides comprising at least one polynucleotide consisting of the nucleotide sequence represented by SEQ ID NO: 155 or a complementary sequence thereof exhibited the highest accuracy of 87.0% in the training cohort and accuracy of 89.0% in the validation cohort. Furthermore, for example, the measurement using the combinations of five polynucleotides comprising at least one polynucleotide consisting of the nucleotide sequence represented by SEQ ID NO: 155 or a complementary sequence thereof exhibited the highest accuracy of 88.2% in the training cohort and accuracy of 87.3% in the validation cohort. Furthermore, for example, the measurement using the combinations of six polynucleotides comprising at least one polynucleotide consisting of the nucleotide sequence represented by SEQ ID NO: 155 or a complementary sequence thereof exhibited the highest accuracy of 88.2% in the training cohort and accuracy of 89.6% in the validation cohort.

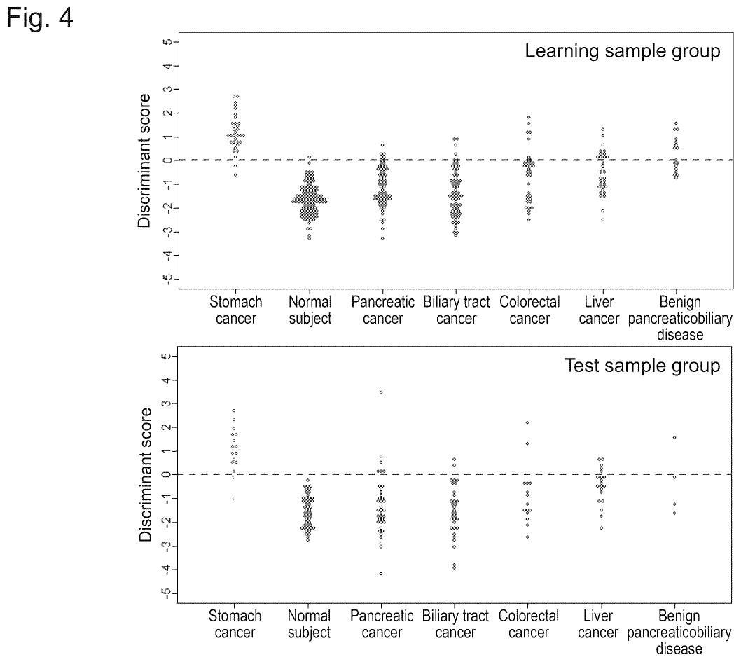

The expression level measurement values of the nucleotide sequences represented by SEQ ID NOs: 9, 13, 143, 155, 194, and 639 were compared among 34 stomach cancer patients, 102 healthy subjects, 63 pancreatic cancer patients, 65 bile duct cancer patients, 35 colorectal cancer patients, 32 liver cancer patients, and 17 benign pancreaticobiliary disease patients in the training cohort. As a result, a scatter diagram that significantly separated the discriminant score of the stomach cancer patient group from the discriminant scores of the other groups was obtained in the training cohort (see the upper diagram of FIG. 4). These results were also reproducible for the validation cohort (see the lower diagram of FIG. 4).

TABLE 8-1

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 21 | 79.8 | 82.4 | 79.6 | 83.8 | 81.2 | 84.1 |
| 9_21 | 82.4 | 97.1 | 80.8 | 80.9 | 81.2 | 80.9 |
| 9_21_34 | 84.1 | 91.2 | 83.4 | 83.8 | 75 | 84.7 |
| 9_21_34_36 | 85.9 | 91.2 | 85.3 | 82.7 | 68.8 | 84.1 |
| 9_21_34_36_98 | 87.9 | 97.1 | 86.9 | 88.4 | 81.2 | 89.2 |
| 9_21_36_98_130_637 | 83.6 | 100 | 81.8 | 85 | 87.5 | 84.7 |
| 9_21_34_36_98_637 | 87 | 94.1 | 86.3 | 87.3 | 87.5 | 87.3 |
| 9_21_34_36_98_155 | 86.7 | 97.1 | 85.6 | 89 | 81.2 | 89.8 |
| 21_36_75_98_155_635 | 83 | 97.1 | 81.5 | 87.9 | 87.5 | 87.9 |
| 9_21_36_98_108_155 | 86.7 | 100 | 85.3 | 86.7 | 81.2 | 87.3 |

TABLE 8-2

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 34 | 62.8 | 67.6 | 62.3 | 60.7 | 81.2 | 58.6 |
| 21_34 | 81 | 82.4 | 80.8 | 82.1 | 81.2 | 82.2 |
| 9_34_36 | 84.4 | 94.1 | 83.4 | 82.1 | 68.8 | 83.4 |
| 9_34_36_98 | 87 | 97.1 | 85.9 | 88.4 | 87.5 | 88.5 |
| 9_34_36_98_635 | 88.2 | 97.1 | 87.2 | 87.9 | 87.5 | 87.9 |
| 34_36_143_155_187_635 | 86.2 | 94.1 | 85.3 | 86.1 | 87.5 | 86 |
| 9_34_36_66_98_187 | 87.3 | 97.1 | 86.3 | 88.4 | 81.2 | 89.2 |
| 9_34_36_98_187_637 | 86.5 | 94.1 | 85.6 | 87.3 | 87.5 | 87.3 |
| 9_34_36_98_185_637 | 86.7 | 97.1 | 85.6 | 86.7 | 87.5 | 86.6 |
| 9_34_36_98_637_639 | 86.5 | 97.1 | 85.3 | 87.9 | 87.5 | 87.9 |

TABLE 8-3

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 36 | 78.7 | 82.4 | 78.3 | 78.6 | 81.2 | 78.3 |
| 9_36 | 82.7 | 97.1 | 81.2 | 82.7 | 75 | 83.4 |
| 9_36_98 | 85 | 100 | 83.4 | 86.7 | 87.5 | 86.6 |
| 9_36_75_98 | 87 | 100 | 85.6 | 85.5 | 81.2 | 86 |
| 9_13_36_108_194 | 87.9 | 94.3 | 87.2 | 86.1 | 75 | 87.3 |
| 9_36_98_108_638_639 | 85.6 | 94.1 | 84.7 | 88.4 | 87.5 | 88.5 |
| 36_98_155_194_635_642 | 85.3 | 100 | 83.7 | 86.1 | 81.2 | 86.6 |
| 9_34_36_75_98_637 | 87.3 | 97.1 | 86.3 | 87.9 | 87.5 | 87.9 |
| 21_36_98_155_185_635 | 83.9 | 97.1 | 82.4 | 89 | 87.5 | 89.2 |
| 9_36_98_108_355_635 | 85.9 | 97.1 | 84.7 | 87.3 | 81.2 | 87.9 |

TABLE 8-4

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 98 | 70.9 | 64.7 | 71.6 | 70.5 | 62.5 | 71.3 |
| 36_98 | 82.4 | 88.2 | 81.8 | 82.1 | 75 | 82.8 |
| 9_98_639 | 84.1 | 91.2 | 83.4 | 85.5 | 81.2 | 86 |
| 9_36_98_194 | 86.7 | 97.1 | 85.6 | 89.6 | 81.2 | 90.4 |
| 9_98_130_135_639 | 88.2 | 97.1 | 87.2 | 87.9 | 100 | 86.6 |
| 9_36_98_130_194_637 | 87.3 | 100 | 85.9 | 89.6 | 87.5 | 89.8 |
| 21_36_98_108_155_635 | 85.6 | 94.1 | 84.7 | 89.6 | 87.5 | 89.8 |
| 9_36_98_108_155_639 | 87.9 | 97.1 | 86.9 | 88.4 | 87.5 | 88.5 |
| 9_36_98_155_187_639 | 87 | 97.1 | 85.9 | 88.4 | 93.8 | 87.9 |
| 9_36_98_155_187_637 | 85.9 | 100 | 84.3 | 85.5 | 81.2 | 86 |

TABLE 8-5

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 155 | 67.1 | 58.8 | 68.1 | 69.9 | 75 | 69.4 |
| 9_155 | 81.6 | 94.1 | 80.2 | 75.7 | 75 | 75.8 |
| 9_155_639 | 84.4 | 97.1 | 83.1 | 85 | 87.5 | 84.7 |
| 9_130_155639 | 87 | 91.2 | 86.6 | 89 | 100 | 87.9 |
| 9_34_130_155_639 | 88.2 | 91.2 | 87.9 | 87.3 | 93.8 | 86.6 |
| 9_36_75_98_155_635 | 85.6 | 100 | 84 | 86.1 | 81.2 | 86.6 |
| 36_98_130_155_185_635 | 85.9 | 94.1 | 85 | 86.1 | 87.5 | 86 |
| 9_13_143_155_194_639 | 88.2 | 94.1 | 87.5 | 89.6 | 87.5 | 89.8 |
| 9_13_34_36_98_155 | 87 | 97.1 | 85.9 | 89 | 81.2 | 89.8 |
| 36_98_108_155_193_635 | 85.3 | 94.1 | 84.3 | 86.7 | 81.2 | 87.3 |

### Comparative Example 1

### <Stomach Cancer Discriminant Performance of Existing Tumor Markers in Blood>

The concentrations of the existing tumor markers CEA and CA19-9 in blood were measured in the training cohort and the validation cohort obtained in the preceding Reference Examples. When the concentrations of these tumor markers in blood are higher than the reference values described in Kim, H. J. et al., Acta Oncologica, 2009, No. 48, p. 385 to 390 (CEA: 5 ng/mL, CA19-9: 37 U/mL), subjects are usually suspected of having cancer. Thus, whether or not the concentrations of CEA and CA19-9 in blood exceeded their reference values was confirmed for each sample, and the results were assessed for the ability of these tumor markers to detect cancer in stomach cancer patients. The sensitivity of each existing marker in the training cohort and the validation cohort was calculated. The results are shown in Table 5. The sensitivity of CEA and CA19-9 was as low as 2.9% in the training cohort, and was as low as 12.5% and 12.5%, respectively, in the validation cohort, demonstrating that neither of the markers are useful in the detection of stomach cancer (Table 5).

On the other hand, as shown above in Tables 3 and 6 of Examples 1 and 2, it can be concluded that in all of the polynucleotides consisting of the nucleotide sequences represented by SEQ ID NOs: 1 to 169 combinations of 1 or 2 polynucleotides exhibiting sensitivity beyond the existing stomach cancer markers are present, and thus such polynucleotides serve as excellent diagnosis markers.

As shown in these Examples and Comparative Example, the kit, etc., and the method of the present invention can detect stomach cancer with higher sensitivity than the existing tumor markers and therefore permit early detection and treatment of stomach cancer. As a result, improvement in survival rate resulting from reduction in the risk of recurrence, and a therapeutic option of stomach-conserving therapy can also be provided.

### INDUSTRIAL APPLICABILITY

According to the present invention, stomach cancer can be effectively detected by a simple and inexpensive method. This enables early detection, diagnosis and treatment of stomach cancer. The method of the present invention can detect stomach cancer with limited invasiveness using the blood of a patient and therefore allows stomach cancer to be detected conveniently and rapidly.

All publications, patents, and patent applications cited herein are incorporated herein by reference in their entirety.

1. A method for detecting stomach cancer in a human subject, comprising:
measuring an expression level of hsa-miR-8063 in a blood, serum or plasma sample from the subject;
comparing the measured expression level of hsa-miR-8063 to a control expression level for a healthy subject;
detecting a decreased level of hsa-miR-8063 in the sample from the subject as compared to the control expression level from the sample from the healthy subject;
wherein the decreased level of hsa-miR-8063 indicates that the subject has stomach cancer; and
wherein the method further comprises treating the subject for the stomach cancer or performing a diagnostic procedure on the subject with the stomach cancer;
wherein the treating comprises surgery, radiotherapy, chemotherapy or a combination thereof; and
wherein the diagnostic procedure comprises gastric X-ray examination, gastroscopy, or imaging of the stomach.

2. The method according to claim 1, comprising performing the diagnostic procedure on the subject.

3. The method according to claim 1, wherein the expression level of hsa-miR-8063 in the sample is measured by using a device or kit comprising a nucleic acid(s) that specifically binds to hsa-miR-8063.

4. The method according to claim 3, wherein the device or kit further comprises a nucleic acid(s) capable of specifically binding to one or more polynucleotides selected from the group consisting of the following other stomach cancer markers:
miR-6726-5p, miR-1343-3p, miR-1247-3p, miR-6787-5p, miR-6875-5p, miR-1225-3p, miR-6781-5p, miR-4746-3p, miR-1908-5p, miR-6756-5p, miR-204-3p, miR-4651, miR-6757-5p, miR-6825-5p, miR-7108-5p, miR-4792, miR-7641, miR-3188, miR-3131, miR-6780b-5p, miR-8069, miR-6840-3p, miR-8072, miR-1233-5p, miR-6887-5p, miR-1231, miR-5572, miR-6738-5p, miR-6784-5p, miR-6791-5p, miR-6749-5p, miR-6741-5p, miR-128-1-5p, miR-4419b, miR-6746-5p, miR-3184-5p, miR-3679-5p, miR-7110-5p, miR-4516, miR-6717-5p, miR-6826-5p, miR-4433b-3p, miR-3679-3p, miR-3135b, miR-3622a-5p, miR-711, miR-4467, miR-6857-5p, miR-6515-3p, miR-1225-5p, miR-187-5p, miR-3185, miR-642b-3p, miR-1249, miR-744-5p, miR-4442, miR-1228-3p, miR-939-5p, miR-6845-5p, miR-887-3p, miR-7845-5p, miR-6729-5p, miR-4632-5p, miR-615-5p, miR-6724-5p, miR-4728-5p, miR-6732-5p, miR-6816-5p, miR-4695-5p, miR-7975, miR-3197, miR-6125, miR-4433-3p, miR-6727-5p, miR-4706, miR-7847-3p, miR-6805-3p, miR-6766-3p, miR-1913, miR-4649-5p, miR-602, miR-3663-3p, miR-6893-5p, miR-6861-5p, miR-4449, miR-6842-5p, miR-4454, miR-5195-3p, miR-663b, miR-6765-5p, miR-4513, miR-614, miR-6785-5p, miR-6777-5p, miR-940, miR-4741, miR-6870-5p, miR-6131, miR-150-3p, miR-4707-5p, miR-1915-3p, miR-3937, miR-937-5p, miR-4443, miR-1914-3p, miR-3620-5p, miR-1268b, miR-1227-5p, miR-6880-5p, miR-4417, miR-6802-5p, miR-6769a-5p, miR-663a, miR-6721-5p, miR-4532, miR-7977, miR-92b-5p, miR-371a-5p, miR-6126, miR-4734, miR-4665-3p, miR-423-5p, miR-1469, miR-4675, miR-6716-5p, miR-4281, miR-6820-5p, miR-6795-5p, miR-6779-5p, miR-7109-5p, miR-6798-5p, miR-4648, miR-8059, miR-6765-3p, miR-6132, miR-4492, miR-7107-5p, miR-3195, miR-3180, miR-296-3p, miR-564, miR-1268a, miR-6848-5p, miR-762, miR-2861, miR-1203, miR-1260b, miR-4476, miR-6885-5p, miR-6769b-5p, miR-23b-3p, miR-1343-5p, miR-3621, miR-4688, miR-4286, miR-4640-5p, miR-4739, miR-1260a, miR-4276, miR-7106-5p, miR-6794-5p, miR-6774-5p, miR-4707-3p, miR-4534, miR-4294, miR-6850-5p, miR-6089 and miR-671-5p; and/or
miR-128-2-5p, miR-125a-3p, miR-92a-2-5p, miR-486-3p, miR-3196, miR-211-3p, miR-4271, miR-6851-5p, miR-149-3p, miR-4667-5p, miR-135a-3p, miR-4486, miR-4697-5p, miR-4725-3p, miR-6510-5p, miR-5001-5p, miR-4673, miR-4466, miR-23a-3p, miR-3656, miR-6782-5p, miR-4689, miR-451a, miR-4446-3p, miR-3180-3p, miR-642a-3p, miR-6889-5p, miR-3178, miR-4665-5p, miR-6722-3p, miR-30c-1-3p, miR-4507, miR-3141 and miR-1199-5p.


SEQUENCE LISTING (657 sequences)

SEQ ID NO: 1 - RNA, 18 nt, Homo sapiens
  source 1..18: mol_type = transcribed RNA
ccagaggtggggactgag

SEQ ID NO: 2 - RNA, 21 nt, Homo sapiens
  source 1..21: mol_type = transcribed RNA
cgggagctggggtctgcaggt

SEQ ID NO: 3 - RNA, 22 nt, Homo sapiens
  source 1..22: mol_type = transcribed RNA
ctcctggggcccgcactctcgc

SEQ ID NO: 4 - RNA, 24 nt, Homo sapiens
  source 1..24: mol_type = transcribed RNA
ccccgggaacgtcgagactggagc

SEQ ID NO: 5 - RNA, 22 nt, Homo sapiens
  source 1..22: mol_type = transcribed RNA
tggcgggggtagagctggctgc

SEQ ID NO: 6 - RNA, 21 nt, Homo sapiens
  source 1..21: mol_type = transcribed RNA
tgagggacccaggacaggaga

SEQ ID NO: 7 - RNA, 22 nt, Homo sapiens
  source 1..22: mol_type = transcribed RNA
tgagcccctgtgccgcccccag

SEQ ID NO: 8 - RNA, 22 nt, Homo sapiens
  source 1..22: mol_type = transcribed RNA
tcaaaatcaggagtcggggctt

SEQ ID NO: 9 - RNA, 21 nt, Homo sapiens
  source 1..21: mol_type = transcribed RNA
cgggccggaggtcaagggcgt

SEQ ID NO: 10 - RNA, 21 nt, Homo sapiens
  source 1..21: mol_type = transcribed RNA
agcggtgctcctgcgggccga

SEQ ID NO: 11 - RNA, 21 nt, Homo sapiens
  source 1..21: mol_type = transcribed RNA
cggcggggacggcgattggtc

SEQ ID NO: 12 - RNA, 23 nt, Homo sapiens
  source 1..23: mol_type = transcribed RNA
agggtggggctggaggtggggct

SEQ ID NO: 13 - RNA, 21 nt, Homo sapiens
  source 1..21: mol_type = transcribed RNA
gctgggaaggcaaagggacgt

SEQ ID NO: 14 - RNA, 20 nt, Homo sapiens
  source 1..20: mol_type = transcribed RNA
cggggtgggtgaggtcgggc

SEQ ID NO: 15 - RNA, 22 nt, Homo sapiens
  source 1..22: mol_type = transcribed RNA
tagggatgggaggccaggatga

SEQ ID NO: 16 - RNA, 22 nt, Homo sapiens
  source 1..22: mol_type = transcribed RNA
tggggaggtgtggagtcagcat

SEQ ID NO: 17 - RNA, 21 nt, Homo sapiens
  source 1..21: mol_type = transcribed RNA
gtgtggccggcaggcgggtgg

SEQ ID NO: 18 - RNA, 18 nt, Homo sapiens
  source 1..18: mol_type = transcribed RNA
cggtgagcgctcgctggc

SEQ ID NO: 19 - RNA, 19 nt, Homo sapiens
  source 1..19: mol_type = transcribed RNA
ttgatctcggaagctaagc

SEQ ID NO: 20 - RNA, 23 nt, Homo sapiens
  source 1..23: mol_type = transcribed RNA
agaggctttgtgcggatacgggg

SEQ ID NO: 21 - RNA, 23 nt, Homo sapiens
  source 1..23: mol_type = transcribed RNA
tcgaggactggtggaagggcctt

SEQ ID NO: 22 - RNA, 23 nt, Homo sapiens
  source 1..23: mol_type = transcribed RNA
tggggaaggcttggcagggaaga

SEQ ID NO: 23 - RNA, 23 nt, Homo sapiens
  source 1..23: mol_type = transcribed RNA
ggatggttgggggcggtcggcgt

SEQ ID NO: 24 - RNA, 22 nt, Homo sapiens
  source 1..22: mol_type = transcribed RNA
gcccaggactttgtgcggggtg

SEQ ID NO: 25 - RNA, 20 nt, Homo sapiens
  source 1..20: mol_type = transcribed RNA
ggcggcggggaggtaggcag

SEQ ID NO: 26 - RNA, 22 nt, Homo sapiens
  source 1..22: mol_type = transcribed RNA
agtgggaggccagggcacggca

SEQ ID NO: 27 - RNA, 23 nt, Homo sapiens
  source 1..23: mol_type = transcribed RNA
tggggggacagatggagaggaca

SEQ ID NO: 28 - RNA, 20 nt, Homo sapiens
  source 1..20: mol_type = transcribed RNA
gtgtctgggcggacagctgc

SEQ ID NO: 29 - RNA, 21 nt, Homo sapiens
  source 1..21: mol_type = transcribed RNA
gttggggtgcaggggtctgct

SEQ ID NO: 30 - RNA, 23 nt, Homo sapiens
  source 1..23: mol_type = transcribed RNA
cgaggggtagaagagcacagggg

SEQ ID NO: 31 - RNA, 20 nt, Homo sapiens
  source 1..20: mol_type = transcribed RNA
gccggggctttgggtgaggg

SEQ ID NO: 32 - RNA, 22 nt, Homo sapiens
  source 1..22: mol_type = transcribed RNA
cccctggggctgggcaggcgga

SEQ ID NO: 33 - RNA, 22 nt, Homo sapiens
  source 1..22: mol_type = transcribed RNA
tcgggcctggggttgggggagc

SEQ ID NO: 34 - RNA, 22 nt, Homo sapiens
  source 1..22: mol_type = transcribed RNA
gtgggtgctggtgggagccgtg

SEQ ID NO: 35 - RNA, 23 nt, Homo sapiens
  source 1..23: mol_type = transcribed RNA
cggggccgtagcactgtctgaga

SEQ ID NO: 36 - RNA, 18 nt, Homo sapiens
  source 1..18: mol_type = transcribed RNA
gaggctgaaggaagatgg

SEQ ID NO: 37 - RNA, 22 nt, Homo sapiens
  source 1..22: mol_type = transcribed RNA
ccgggagaaggaggtggcctgg

SEQ ID NO: 38 - RNA, 24 nt, Homo sapiens
  source 1..24: mol_type = transcribed RNA
tgaggggcctcagaccgagctttt

SEQ ID NO: 39 - RNA, 23 nt, Homo sapiens
  source 1..23: mol_type = transcribed RNA
tgaggatatggcagggaagggga

SEQ ID NO: 40 - RNA, 21 nt, Homo sapiens
  source 1..21: mol_type = transcribed RNA
tgggggtgtggggagagagag

SEQ ID NO: 41 - RNA, 17 nt, Homo sapiens
  source 1..17: mol_type = transcribed RNA
gggagaagggtcggggc

SEQ ID NO: 42 - RNA, 22 nt, Homo sapiens
  source 1..22: mol_type = transcribed RNA
aggcgatgtggggatgtagaga

SEQ ID NO: 43 - RNA, 23 nt, Homo sapiens
  source 1..23: mol_type = transcribed RNA
tcaataggaaagaggtgggacct

SEQ ID NO: 44 - RNA, 21 nt, Homo sapiens
  source 1..21: mol_type = transcribed RNA
caggagtggggggtgggacgt

SEQ ID NO: 45 - RNA, 22 nt, Homo sapiens
  source 1..22: mol_type = transcribed RNA
cttccccccagtaatcttcatc

SEQ ID NO: 46 - RNA, 22 nt, Homo sapiens
  source 1..22: mol_type = transcribed RNA
ggctggagcgagtgcagtggtg

SEQ ID NO: 47 - RNA, 22 nt, Homo sapiens
  source 1..22: mol_type = transcribed RNA
caggcacgggagctcaggtgag

SEQ ID NO: 48 - RNA, 22 nt, Homo sapiens
  source 1..22: mol_type = transcribed RNA
gggacccagggagagacgtaag

SEQ ID NO: 49 - RNA, 22 nt, Homo sapiens
  source 1..22: mol_type = transcribed RNA
tggcggcggtagttatgggctt

SEQ ID NO: 50 - RNA, 22 nt, Homo sapiens
  source 1..22: mol_type = transcribed RNA
ttggggattgggtcaggccagt

SEQ ID NO: 51 - RNA, 20 nt, Homo sapiens
  source 1..20: mol_type = transcribed RNA
tctcttcatctaccccccag

SEQ ID NO: 52 - RNA, 22 nt, Homo sapiens
  source 1..22: mol_type = transcribed RNA
gtgggtacggcccagtgggggg

SEQ ID NO: 53 - RNA, 22 nt, Homo sapiens
  source 1..22: mol_type = transcribed RNA
ggctacaacacaggacccgggc

SEQ ID NO: 54 - RNA, 23 nt, Homo sapiens
  source 1..23: mol_type = transcribed RNA
agaagaaggcggtcggtctgcgg

SEQ ID NO: 55 - RNA, 22 nt, Homo sapiens
  source 1..22: mol_type = transcribed RNA
agacacatttggagagggaccc

SEQ ID NO: 56 - RNA, 22 nt, Homo sapiens
  source 1..22: mol_type = transcribed RNA
acgcccttcccccccttcttca

SEQ ID NO: 57 - RNA, 22 nt, Homo sapiens
  source 1..22: mol_type = transcribed RNA
tgcggggctagggctaacagca

SEQ ID NO: 58 - RNA, 17 nt, Homo sapiens
  source 1..17: mol_type = transcribed RNA
gccggacaagagggagg

SEQ ID NO: 59 - RNA, 20 nt, Homo sapiens
  source 1..20: mol_type = transcribed RNA
tcacacctgcctcgcccccc

SEQ ID NO: 60 - RNA, 24 nt, Homo sapiens
  source 1..24: mol_type = transcribed RNA
tggggagctgaggctctgggggtg

SEQ ID NO: 61 - RNA, 19 nt, Homo sapiens
  source 1..19: mol_type = transcribed RNA
cggggccagagcagagagc

SEQ ID NO: 62 - RNA, 22 nt, Homo sapiens
  source 1..22: mol_type = transcribed RNA
gtgaacgggcgccatcccgagg

SEQ ID NO: 63 - RNA, 21 nt, Homo sapiens
  source 1..21: mol_type = transcribed RNA
aagggacagggagggtcgtgg

SEQ ID NO: 64 - RNA, 22 nt, Homo sapiens
  source 1..22: mol_type = transcribed RNA
tgggcgagggcggctgagcggc

SEQ ID NO: 65 - RNA, 23 nt, Homo sapiens
  source 1..23: mol_type = transcribed RNA
gagggcagcgtgggtgtggcgga

SEQ ID NO: 66 - RNA, 22 nt, Homo sapiens
  source 1..22: mol_type = transcribed RNA
gggggtccccggtgctcggatc

SEQ ID NO: 67 - RNA, 23 nt, Homo sapiens
  source 1..23: mol_type = transcribed RNA
ctgggcccgcggcgggcgtgggg

SEQ ID NO: 68 - RNA, 23 nt, Homo sapiens
  source 1..23: mol_type = transcribed RNA
tgggaggggagaggcagcaagca

SEQ ID NO: 69 - RNA, 20 nt, Homo sapiens
  source 1..20: mol_type = transcribed RNA
tagggggtggcaggctggcc

SEQ ID NO: 70 - RNA, 21 nt, Homo sapiens
  source 1..21: mol_type = transcribed RNA
tggggcggggcaggtccctgc

SEQ ID NO: 71 - RNA, 22 nt, Homo sapiens
  source 1..22: mol_type = transcribed RNA
caggaggcagtgggcgagcagg

SEQ ID NO: 72 - RNA, 20 nt, Homo sapiens
  source 1..20: mol_type = transcribed RNA
agagatgaagcgggggggcg

SEQ ID NO: 73 - RNA, 18 nt, Homo sapiens
  source 1..18: mol_type = transcribed RNA
atcctagtcacggcacca

SEQ ID NO: 74 - RNA, 23 nt, Homo sapiens
  source 1..23: mol_type = transcribed RNA
ggaggcgcaggctcggaaaggcg

SEQ ID NO: 75 - RNA, 20 nt, Homo sapiens
  source 1..20: mol_type = transcribed RNA
gcggaaggcggagcggcgga

SEQ ID NO: 76 - RNA, 21 nt, Homo sapiens
  source 1..21: mol_type = transcribed RNA
acaggagtgggggtgggacat

SEQ ID NO: 77 - RNA, 23 nt, Homo sapiens
  source 1..23: mol_type = transcribed RNA
ctcggggcaggcggctgggagcg

SEQ ID NO: 78 - RNA, 25 nt, Homo sapiens
  source 1..25: mol_type = transcribed RNA
agcggggaggaagtgggcgctgctt

SEQ ID NO: 79 - RNA, 21 nt, Homo sapiens
  source 1..21: mol_type = transcribed RNA
cgtggaggacgaggaggaggc

SEQ ID NO: 80 - RNA, 23 nt, Homo sapiens
  source 1..23: mol_type = transcribed RNA
ttgctctgctcccccgcccccag

SEQ ID NO: 81 - RNA, 22 nt, Homo sapiens
  source 1..22: mol_type = transcribed RNA
tgattgtcttcccccaccctca

SEQ ID NO: 82 - RNA, 22 nt, Homo sapiens
  source 1..22: mol_type = transcribed RNA
tctgccccctccgctgctgcca

SEQ ID NO: 83 - RNA, 24 nt, Homo sapiens
  source 1..24: mol_type = transcribed RNA
tgggcgaggggtgggctctcagag

SEQ ID NO: 84 - RNA, 23 nt, Homo sapiens
  source 1..23: mol_type = transcribed RNA
gacacgggcgacagctgcggccc

SEQ ID NO: 85 - RNA, 23 nt, Homo sapiens
  source 1..23: mol_type = transcribed RNA
tgagcaccacacaggccgggcgc

SEQ ID NO: 86 - RNA, 21 nt, Homo sapiens
  source 1..21: mol_type = transcribed RNA
caggcaggtgtagggtggagc

SEQ ID NO: 87 - RNA, 22 nt, Homo sapiens
  source 1..22: mol_type = transcribed RNA
actgggtaggtggggctccagg

SEQ ID NO: 88 - RNA, 22 nt, Homo sapiens
  source 1..22: mol_type = transcribed RNA
cgtcccggggctgcgcgaggca

SEQ ID NO: 89 - RNA, 22 nt, Homo sapiens
  source 1..22: mol_type = transcribed RNA
tgggggtggtctctagccaagg

SEQ ID NO: 90 - RNA, 20 nt, Homo sapiens
  source 1..20: mol_type = transcribed RNA
ggatccgagtcacggcacca

SEQ ID NO: 91 - RNA, 21 nt, Homo sapiens
  source 1..21: mol_type = transcribed RNA
atccagttctctgagggggct

SEQ ID NO: 92 - RNA, 22 nt, Homo sapiens
  source 1..22: mol_type = transcribed RNA
ggtggcccggccgtgcctgagg

SEQ ID NO: 93 - RNA, 25 nt, Homo sapiens
  source 1..25: mol_type = transcribed RNA
gtgaggcggggccaggagggtgtgt

SEQ ID NO: 94 - RNA, 22 nt, Homo sapiens
  source 1..22: mol_type = transcribed RNA
agactgacggctggaggcccat

SEQ ID NO: 95 - RNA, 23 nt, Homo sapiens
  source 1..23: mol_type = transcribed RNA
gaacgcctgttcttgccaggtgg

SEQ ID NO: 96 - RNA, 22 nt, Homo sapiens
  source 1..22: mol_type = transcribed RNA
tgggagggcgtggatgatggtg

SEQ ID NO: 97 - RNA, 23 nt, Homo sapiens
  source 1..23: mol_type = transcribed RNA
acggggagtcaggcagtggtgga

SEQ ID NO: 98 - RNA, 21 nt, Homo sapiens
  source 1..21: mol_type = transcribed RNA
aaggcagggcccccgctcccc

SEQ ID NO: 99 - RNA, 23 nt, Homo sapiens
  source 1..23: mol_type = transcribed RNA
cgggctgtccggaggggtcggct

SEQ ID NO: 100 - RNA, 19 nt, Homo sapiens
  source 1..19: mol_type = transcribed RNA
tgggggagatgggggttga

SEQ ID NO: 101 - RNA, 19 nt, Homo sapiens
  source 1..19: mol_type = transcribed RNA
ggctggtcagatgggagtg

SEQ ID NO: 102 - RNA, 22 nt, Homo sapiens
  source 1..22: mol_type = transcribed RNA
ctggtacaggcctgggggacag

SEQ ID NO: 103 - RNA, 23 nt, Homo sapiens
  source 1..23: mol_type = transcribed RNA
gccccggcgcgggcgggttctgg

SEQ ID NO: 104 - RNA, 20 nt, Homo sapiens
  source 1..20: mol_type = transcribed RNA
ccccagggcgacgcggcggg

SEQ ID NO: 105 - RNA, 23 nt, Homo sapiens
  source 1..23: mol_type = transcribed RNA
acaggcggctgtagcaatggggg

SEQ ID NO: 106 - RNA, 20 nt, Homo sapiens
  source 1..20: mol_type = transcribed RNA
gtgagtcagggtggggctgg

SEQ ID NO: 107 - RNA, 17 nt, Homo sapiens
  source 1..17: mol_type = transcribed RNA
ttggaggcgtgggtttt

SEQ ID NO: 108 - RNA, 22 nt, Homo sapiens
  source 1..22: mol_type = transcribed RNA
ggaggggtcccgcactgggagg

SEQ ID NO: 109 - RNA, 22 nt, Homo sapiens
  source 1..22: mol_type = transcribed RNA
gtgggctgggctgggctgggcc

SEQ ID NO: 110 - RNA, 20 nt, Homo sapiens
  source 1..20: mol_type = transcribed RNA
cgggcgtggtggtgggggtg

SEQ ID NO: 111 - RNA, 17 nt, Homo sapiens
  source 1..17: mol_type = transcribed RNA
gtggggccaggcggtgg

SEQ ID NO: 112 - RNA, 22 nt, Homo sapiens
  source 1..22: mol_type = transcribed RNA
tggtggaggaagagggcagctc

SEQ ID NO: 113 - RNA, 18 nt, Homo sapiens
  source 1..18: mol_type = transcribed RNA
ggtgggcttcccggaggg

SEQ ID NO: 114 - RNA, 20 nt, Homo sapiens
  source 1..20: mol_type = transcribed RNA
ctaggtggggggcttgaagc

SEQ ID NO: 115 - RNA, 21 nt, Homo sapiens
  source 1..21: mol_type = transcribed RNA
aggtgggtatggaggagccct

SEQ ID NO: 116 - RNA, 22 nt, Homo sapiens
  source 1..22: mol_type = transcribed RNA
aggcggggcgccgcgggaccgc

SEQ ID NO: 117 - RNA, 23 nt, Homo sapiens
  source 1..23: mol_type = transcribed RNA
tgggcaggggcttattgtaggag

SEQ ID NO: 118 - RNA, 17 nt, Homo sapiens
  source 1..17: mol_type = transcribed RNA
ccccggggagcccggcg

SEQ ID NO: 119 - RNA, 18 nt, Homo sapiens
  source 1..18: mol_type = transcribed RNA
ttcccagccaacgcacca

SEQ ID NO: 120 - RNA, 22 nt, Homo sapiens
  source 1..22: mol_type = transcribed RNA
agggacgggacgcggtgcagtg

SEQ ID NO: 121 - RNA, 20 nt, Homo sapiens
  source 1..20: mol_type = transcribed RNA
actcaaactgtgggggcact

SEQ ID NO: 122 - RNA, 18 nt, Homo sapiens
  source 1..18: mol_type = transcribed RNA
gtgaaggcccggcggaga

SEQ ID NO: 123 - RNA, 22 nt, Homo sapiens
  source 1..22: mol_type = transcribed RNA
gctgcgggctgcggtcagggcg

SEQ ID NO: 124 - RNA, 26 nt, Homo sapiens
  source 1..26: mol_type = transcribed RNA
ctcggccgcggcgcgtagcccccgcc

SEQ ID NO: 125 - RNA, 23 nt, Homo sapiens
  source 1..23: mol_type = transcribed RNA
tgaggggcagagagcgagacttt

SEQ ID NO: 126 - RNA, 22 nt, Homo sapiens
  source 1..22: mol_type = transcribed RNA
ctcggcgcggggcgcgggctcc

SEQ ID NO: 127 - RNA, 22 nt, Homo sapiens
  source 1..22: mol_type = transcribed RNA
ggggctgtgattgaccagcagg

SEQ ID NO: 128 - RNA, 22 nt, Homo sapiens
  source 1..22: mol_type = transcribed RNA
accttgccttgctgcccgggcc

SEQ ID NO: 129 - RNA, 20 nt, Homo sapiens
  source 1..20: mol_type = transcribed RNA
tgggaatgggggtaagggcc

SEQ ID NO: 130 - RNA, 21 nt, Homo sapiens
  source 1..21: mol_type = transcribed RNA
cttccgccccgccgggcgtcg

SEQ ID NO: 131 - RNA, 18 nt, Homo sapiens
  source 1..18: mol_type = transcribed RNA
gggtcccggggagggggg

SEQ ID NO: 132 - RNA, 19 nt, Homo sapiens
  source 1..19: mol_type = transcribed RNA
tgcggcagagctggggtca

SEQ ID NO: 133 - RNA, 24 nt, Homo sapiens
  source 1..24: mol_type = transcribed RNA
tggggggacaggatgagaggctgt

SEQ ID NO: 134 - RNA, 21 nt, Homo sapiens
  source 1..21: mol_type = transcribed RNA
ctgggaggggctgggtttggc

SEQ ID NO: 135 - RNA, 21 nt, Homo sapiens
  source 1..21: mol_type = transcribed RNA
ctggggggaggagaccctgct

SEQ ID NO: 136 - RNA, 23 nt, Homo sapiens
  source 1..23: mol_type = transcribed RNA
ccagggggatgggcgagcttggg

SEQ ID NO: 137 - RNA, 20 nt, Homo sapiens
  source 1..20: mol_type = transcribed RNA
tgtgggactgcaaatgggag

SEQ ID NO: 138 - RNA, 22 nt, Homo sapiens
  source 1..22: mol_type = transcribed RNA
ggggaactgtagatgaaaaggc

SEQ ID NO: 139 - RNA, 21 nt, Homo sapiens
  source 1..21: mol_type = transcribed RNA
tcacctggctggcccgcccag

SEQ ID NO: 140 - RNA, 19 nt, Homo sapiens
  source 1..19: mol_type = transcribed RNA
agcagggctggggattgca

SEQ ID NO: 141 - RNA, 17 nt, Homo sapiens
  source 1..17: mol_type = transcribed RNA
ggggctgggcgcgcgcc

SEQ ID NO: 142 - RNA, 22 nt, Homo sapiens
  source 1..22: mol_type = transcribed RNA
tcggcctggggaggaggaaggg

SEQ ID NO: 143 - RNA, 17 nt, Homo sapiens
  source 1..17: mol_type = transcribed RNA
cgcgccgggcccgggtt

SEQ ID NO: 144 - RNA, 19 nt, Homo sapiens
  source 1..19: mol_type = transcribed RNA
tggggcggagcttccggag

SEQ ID NO: 145 - RNA, 22 nt, Homo sapiens
  source 1..22: mol_type = transcribed RNA
gagggttgggtggaggctctcc

SEQ ID NO: 146 - RNA, 19 nt, Homo sapiens
  source 1..19: mol_type = transcribed RNA
aggcacggtgtcagcaggc

SEQ ID NO: 147 - RNA, 18 nt, Homo sapiens
  source 1..18: mol_type = transcribed RNA
cgggcgtggtggtggggg

SEQ ID NO: 148 - RNA, 23 nt, Homo sapiens
  source 1..23: mol_type = transcribed RNA
tgggggctgggatgggccatggt

SEQ ID NO: 149 - RNA, 22 nt, Homo sapiens
  source 1..22: mol_type = transcribed RNA
ggggctggggccggggccgagc

SEQ ID NO: 150 - RNA, 19 nt, Homo sapiens
  source 1..19: mol_type = transcribed RNA
ggggcctggcggtgggcgg

SEQ ID NO: 151 - RNA, 21 nt, Homo sapiens
  source 1..21: mol_type = transcribed RNA
cccggagccaggatgcagctc

SEQ ID NO: 152 - RNA, 19 nt, Homo sapiens
  source 1..19: mol_type = transcribed RNA
atcccaccactgccaccat

SEQ ID NO: 153 - RNA, 22 nt, Homo sapiens
  source 1..22: mol_type = transcribed RNA
caggaaggatttagggacaggc

SEQ ID NO: 154 - RNA, 25 nt, Homo sapiens
  source 1..25: mol_type = transcribed RNA
aggggggcactgcgcaagcaaagcc

SEQ ID NO: 155 - RNA, 23 nt, Homo sapiens
  source 1..23: mol_type = transcribed RNA
tggtgggtggggaggagaagtgc

SEQ ID NO: 156 - RNA, 21 nt, Homo sapiens
  source 1..21: mol_type = transcribed RNA
atcacattgccagggattacc

SEQ ID NO: 157 - RNA, 22 nt, Homo sapiens
  source 1..22: mol_type = transcribed RNA
tggggagcggcccccgggtggg

SEQ ID NO: 158 - RNA, 20 nt, Homo sapiens
  source 1..20: mol_type = transcribed RNA
cgcgggtcggggtctgcagg

SEQ ID NO: 159 - RNA, 22 nt, Homo sapiens
  source 1..22: mol_type = transcribed RNA
taggggcagcagaggacctggg

SEQ ID NO: 160 - RNA, 17 nt, Homo sapiens
  source 1..17: mol_type = transcribed RNA
accccactcctggtacc

SEQ ID NO: 161 - RNA, 23 nt, Homo sapiens
  source 1..23: mol_type = transcribed RNA
tgggccagggagcagctggtggg

SEQ ID NO: 162 - RNA, 25 nt, Homo sapiens
  source 1..25: mol_type = transcribed RNA
aagggaggaggagcggaggggccct

SEQ ID NO: 163 - RNA, 18 nt, Homo sapiens
  source 1..18: mol_type = transcribed RNA
atcccacctctgccacca

SEQ ID NO: 164 - RNA, 17 nt, Homo sapiens
  source 1..17: mol_type = transcribed RNA
ctcagtgactcatgtgc

SEQ ID NO: 165 - RNA, 20 nt, Homo sapiens
  source 1..20: mol_type = transcribed RNA
tgggaggaggggatcttggg

SEQ ID NO: 166 - RNA, 23 nt, Homo sapiens
  source 1..23: mol_type = transcribed RNA
gggggccgatacactgtacgaga

SEQ ID NO: 167 - RNA, 22 nt, Homo sapiens
  source 1..22: mol_type = transcribed RNA
acaggtgaggttcttgggagcc

SEQ ID NO: 168 - RNA, 22 nt, Homo sapiens
  source 1..22: mol_type = transcribed RNA
gggtggggatttgttgcattac

SEQ ID NO: 169 - RNA, 21 nt, Homo sapiens
  source 1..21: mol_type = transcribed RNA
cggggcagctcagtacaggat

SEQ ID NO: 170 - RNA, 18 nt, Homo sapiens
  source 1..18: mol_type = transcribed RNA
cggggcggcaggggcctc

SEQ ID NO: 171 - RNA, 21 nt, Homo sapiens
  source 1..21: mol_type = transcribed RNA
gcagggacagcaaaggggtgc

SEQ ID NO: 172 - RNA, 19 nt, Homo sapiens
  source 1..19: mol_type = transcribed RNA
gggggaagaaaaggtgggg

SEQ ID NO: 173 - RNA, 23 nt, Homo sapiens
  source 1..23: mol_type = transcribed RNA
aggaggtggtactaggggccagc

SEQ ID NO: 174 - RNA, 21 nt, Homo sapiens
  source 1..21: mol_type = transcribed RNA
agggagggacgggggctgtgc

SEQ ID NO: 175 - RNA, 22 nt, Homo sapiens
  source 1..22: mol_type = transcribed RNA
actggggagcagaaggagaacc

SEQ ID NO: 176 - RNA, 22 nt, Homo sapiens
  source 1..22: mol_type = transcribed RNA
tatagggattggagccgtggcg

SEQ ID NO: 177 - RNA, 17 nt, Homo sapiens
  source 1..17: mol_type = transcribed RNA
gctgggcgaggctggca

SEQ ID NO: 178 - RNA, 22 nt, Homo sapiens
  source 1..22: mol_type = transcribed RNA
agggggcgcagtcactgacgtg

SEQ ID NO: 179 - RNA, 22 nt, Homo sapiens
  source 1..22: mol_type = transcribed RNA
tggggaaggcgtcagtgtcggg

SEQ ID NO: 180 - RNA, 22 nt, Homo sapiens
  source 1..22: mol_type = transcribed RNA
cagcaggggagagagaggagtc

SEQ ID NO: 181 - RNA, 24 nt, Homo sapiens
  source 1..24: mol_type = transcribed RNA
agggctggactcagcggcggagct

SEQ ID NO: 182 - RNA, 22 nt, Homo sapiens
  source 1..22: mol_type = transcribed RNA
tccaggcaggagccggactgga

SEQ ID NO: 183 - RNA, 18 nt, Homo sapiens
  source 1..18: mol_type = transcribed RNA
gggtgcgggccggcgggg

SEQ ID NO: 184 - RNA, 21 nt, Homo sapiens
  source 1..21: mol_type = transcribed RNA
atcacattgccagggatttcc

SEQ ID NO: 185 - RNA, 17 nt, Homo sapiens
  source 1..17: mol_type = transcribed RNA
ggcgggtgcgggggtgg

SEQ ID NO: 186 - RNA, 25 nt, Homo sapiens
  source 1..25: mol_type = transcribed RNA
taggggtgggggaattcaggggtgt

SEQ ID NO: 187 - RNA, 22 nt, Homo sapiens
  source 1..22: mol_type = transcribed RNA
ttgaggagacatggtgggggcc

SEQ ID NO: 188 - RNA, 22 nt, Homo sapiens
  source 1..22: mol_type = transcribed RNA
aaaccgttaccattactgagtt

SEQ ID NO: 189 - RNA, 22 nt, Homo sapiens
  source 1..22: mol_type = transcribed RNA
cagggctggcagtgacatgggt

SEQ ID NO: 190 - RNA, 22 nt, Homo sapiens
  source 1..22: mol_type = transcribed RNA
tggggcggagcttccggaggcc

SEQ ID NO: 191 - RNA, 22 nt, Homo sapiens
  source 1..22: mol_type = transcribed RNA
agacacatttggagagggaacc

SEQ ID NO: 192 - RNA, 23 nt, Homo sapiens
  source 1..23: mol_type = transcribed RNA
tcggggagtctggggtccggaat

SEQ ID NO: 193 - RNA, 17 nt, Homo sapiens
  source 1..17: mol_type = transcribed RNA
ggggcgcggccggatcg

SEQ ID NO: 194 - RNA, 23 nt, Homo sapiens
  source 1..23: mol_type = transcribed RNA
ctgggggacgcgtgagcgcgagc

SEQ ID NO: 195 - RNA, 22 nt, Homo sapiens
  source 1..22: mol_type = transcribed RNA
tgcaggggtcgggtgggccagg

SEQ ID NO: 196 - RNA, 22 nt, Homo sapiens
  source 1..22: mol_type = transcribed RNA
ctgggagagggttgtttactcc

SEQ ID NO: 197 - RNA, 20 nt, Homo sapiens
  source 1..20: mol_type = transcribed RNA
ctgggttgggctgggctggg

SEQ ID NO: 198 - RNA, 19 nt, Homo sapiens
  source 1..19: mol_type = transcribed RNA
gagggcgggtggaggagga

SEQ ID NO: 199 - RNA, 20 nt, Homo sapiens
  source 1..20: mol_type = transcribed RNA
cctgagcccgggccgcgcag

SEQ ID NO: 200 - RNA, 86 nt, Homo sapiens
  source 1..86: mol_type = transcribed RNA
ggcttagaaacagtccctaggtaggatttggggaggagctaagaagcccctacagggcccagaggtggggactgagccttagttgg

SEQ ID NO: 201 - RNA, 61 nt, Homo sapiens
  source 1..61: mol_type = transcribed RNA
gggggcgggagctggggtctgcaggttcgcactgatgcctgctcgccctgtctcccgctag

SEQ ID NO: 202 - RNA, 84 nt, Homo sapiens
  source 1..84: mol_type = transcribed RNA
gctggcgtcggtgctggggagcggcccccgggtgggcctctgctctggcccctcctggggcccgcactctcgctctgggcccgc

SEQ ID NO: 203 - RNA, 136 nt, Homo sapiens
  source 1..136: mol_type = transcribed RNA
ccgcttgcctcgcccagcgcagccccggccgctgggcgcacccgtcccgttcgtccccggacgttgctctctaccccgggaacgtcgagactggagcgcccgaactgagccaccttcgcggaccccgagagcggcg

SEQ ID NO: 204 - RNA, 61 nt, Homo sapiens
  source 1..61: mol_type = transcribed RNA
tcggctggcgggggtagagctggctgcaggcccggcccctctcagctgctgccctctccag

SEQ ID NO: 205 - RNA, 72 nt, Homo sapiens
  source 1..72: mol_type = transcribed RNA
gagtctgagggacccaggacaggagaaggcctatggtgatttgcattcttcctgccctggctccatcctcag

SEQ ID NO: 206 - RNA, 90 nt, Homo sapiens
  source 1..90: mol_type = transcribed RNA
gtgggtacggcccagtgggggggagagggacacgccctgggctctgcccagggtgcagccggactgactgagcccctgtgccgcccccag

SEQ ID NO: 207 - RNA, 81 nt, Homo sapiens
  source 1..81: mol_type = transcribed RNA
tagaggcagtttcaacagatgtgtagacttttgatatgagaaattggtttcaaaatcaggagtcggggctttactgctttt

SEQ ID NO: 208 - RNA, 64 nt, Homo sapiens
  source 1..64: mol_type = transcribed RNA
aaccccgggccggaggtcaagggcgtcgcttctccctaatgttgcctcttttccacggcctcag

SEQ ID NO: 209 - RNA, 71 nt, Homo sapiens
  source 1..71: mol_type = transcribed RNA
gtgtctgtgccggtcccaggagaacctgcagaggcatcgggtcagcggtgctcctgcgggccgacactcac

SEQ ID NO: 210 - RNA, 80 nt, Homo sapiens
  source 1..80: mol_type = transcribed RNA
cgggaatgccgcggcggggacggcgattggtccgtatgtgtggtgccaccggccgccggctccgccccggcccccgcccc

SEQ ID NO: 211 - RNA, 63 nt, Homo sapiens
  source 1..63: mol_type = transcribed RNA
accctagggtggggctggaggtggggctgaggctgagtcttcctccccttcctccctgcccag

SEQ ID NO: 212 - RNA, 110 nt, Homo sapiens
  source 1..110: mol_type = transcribed RNA
ggctacagtctttcttcatgtgactcgtggacttccctttgtcatcctatgcctgagaatatatgaaggaggctgggaaggcaaagggacgttcaattgtcatcactggc

SEQ ID NO: 213 - RNA, 73 nt, Homo sapiens
  source 1..73: mol_type = transcribed RNA
cggcgacggcggggtgggtgaggtcgggccccaagactcggggtttgccgggcgcctcagttcaccgcggccg

SEQ ID NO: 214 - RNA, 69 nt, Homo sapiens
  source 1..69: mol_type = transcribed RNA
gggcttagggatgggaggccaggatgaagattaatccctaatccccaacactggccttgctatccccag

SEQ ID NO: 215 - RNA, 66 nt, Homo sapiens
  source 1..66: mol_type = transcribed RNA
gggcatggggaggtgtggagtcagcatggggctaggaggccccgcgctgacccgccttctccgcag

SEQ ID NO: 216 - RNA, 87 nt, Homo sapiens
  source 1..87: mol_type = transcribed RNA
gtgtggccggcaggcgggtgggcgggggcggccggtgggaaccccgccccgccccgcgcccgcactcacccgcccgtctccccacag

SEQ ID NO: 217 - RNA, 74 nt, Homo sapiens
  source 1..74: mol_type = transcribed RNA
gcagcccggtgagcgctcgctggcctggcagtgcgtcggaagaacagggcgggtggggccgcgcacatctctgc

SEQ ID NO: 218 - RNA, 61 nt, Homo sapiens
  source 1..61: mol_type = transcribed RNA
tctcgtttgatctcggaagctaagcagggttgggcctggttagtacttggatgggaaactt

SEQ ID NO: 219 - RNA, 53 nt, Homo sapiens
  source 1..53: mol_type = transcribed RNA
gtttgatctcggaagctaagcagggtcgggcctggttagtacttggatgggag

SEQ ID NO: 220 - RNA, 85 nt, Homo sapiens
  source 1..85: mol_type = transcribed RNA
ggcgcctcctgctctgctgtgccgccagggcctcccctagcgcgccttctggagaggctttgtgcggatacggggctggaggcct

SEQ ID NO: 221 - RNA, 63 nt, Homo sapiens
  source 1..63: mol_type = transcribed RNA
gagtcgaggactggtggaagggcctttcccctcagaccaaggccctggccccagcttcttctc

SEQ ID NO: 222 - RNA, 79 nt, Homo sapiens
  source 1..79: mol_type = transcribed RNA
cagcctggggaaggcttggcagggaagacacatgagcagtgcctccacttcacgcctctcccttgtctcctttccctag

SEQ ID NO: 223 - RNA, 86 nt, Homo sapiens
  source 1..86: mol_type = transcribed RNA
cgcctgagcgtgcagcaggacatcttcctgacctggtaataattaggtgagaaggatggttgggggcggtcggcgtaactcaggga

SEQ ID NO: 224 - RNA, 71 nt, Homo sapiens
  source 1..71: mol_type = transcribed RNA
tgaccacccccgggcaaagacctgcagatcccctgttagagacgggcccaggactttgtgcggggtgccca

SEQ ID NO: 225 - RNA, 80 nt, Homo sapiens
  source 1..80: mol_type = transcribed RNA
gcgtcaagatggcggcggggaggtaggcagagcaggacgccgctgctgccgccgccaccgccgcctccgctccagtcgcc

SEQ ID NO: 226 - RNA, 82 nt, Homo sapiens
  source 1..82: mol_type = transcribed RNA
gtgagtgggaggccagggcacggcagggggagctgcagggctatgggaggggccccagcgtctgagccctgtcctcccgcag

SEQ ID NO: 227 - RNA, 82 nt, Homo sapiens; identical to SEQ ID NO: 226
  source 1..82: mol_type = transcribed RNA

SEQ ID NO: 228 - RNA, 65 nt, Homo sapiens
  source 1..65: mol_type = transcribed RNA
gagaatggggggacagatggagaggacacaggctggcactgaggtcccctccactttcctcctag

SEQ ID NO: 229 - RNA, 92 nt, Homo sapiens
  source 1..92: mol_type = transcribed RNA
gtcagtgtctgggcggacagctgcaggaaagggaagaccaaggcttgctgtctgtccagtctgccaccctaccctgtctgttcttgccacag

SEQ ID NO: 230 - RNA, 137 nt, Homo sapiens
  source 1..137: mol_type = transcribed RNA
agccagacaagagggtcatggggagtcactgtcaacccagagcaggcactgcccctgcgaccagcctggggcatcggttggggtgcaggggtctgctggtgatgctttccatctctttgctttgtcctgattgtagc

SEQ ID NO: 231 - RNA, 64 nt, Homo sapiens
  source 1..64: mol_type = transcribed RNA
gaaggcgaggggtagaagagcacaggggttctgataaacccttctgcctgcattctactcccag

SEQ ID NO: 232 - RNA, 67 nt, Homo sapiens
  source 1..67: mol_type = transcribed RNA
tacaggccggggctttgggtgagggacccccggagtctgtcacggtctcaccccaactctgccccag

SEQ ID NO: 233 - RNA, 67 nt, Homo sapiens
  source 1..67: mol_type = transcribed RNA
ccagacccctggggctgggcaggcggaaagaggtctgaactgcctctgcctccttggtctccggcag

SEQ ID NO: 234 - RNA, 69 nt, Homo sapiens
  source 1..69: mol_type = transcribed RNA
ggccctcgggcctggggttgggggagctctgtcctgtctcactcattgctcctcccctgcctggcccag

SEQ ID NO: 235 - RNA, 63 nt, Homo sapiens
  source 1..63: mol_type = transcribed RNA
aatgggtgggtgctggtgggagccgtgccctggccactcattcggctctctccctcaccctag

SEQ ID NO: 236 - RNA, 82 nt, Homo sapiens
  source 1..82: mol_type = transcribed RNA
tgagctgttggattcggggccgtagcactgtctgagaggtttacatttctcacagtgaaccggtctctttttcagctgcttc

SEQ ID NO: 237 - RNA, 68 nt, Homo sapiens
  source 1..68: mol_type = transcribed RNA
ctcaggctcagtggtgcatgcttatagtcccagccactctggaggctgaaggaagatggcttgagcct

SEQ ID NO: 238 - RNA, 63 nt, Homo sapiens
  source 1..63: mol_type = transcribed RNA
cttgcccgggagaaggaggtggcctggagagctgctgtctccagccgccgcctgtctccacag

SEQ ID NO: 239 - RNA, 75 nt, Homo sapiens
  source 1..75: mol_type = transcribed RNA
aagcaagactgaggggcctcagaccgagcttttggaaaatagaaaagtctcgctctctgcccctcagcctaactt

SEQ ID NO: 240 - RNA, 68 nt, Homo sapiens
  source 1..68: mol_type = transcribed RNA
cgtggtgaggatatggcagggaaggggagtttccctctattcccttccccccagtaatcttcatcatg

SEQ ID NO: 241 - RNA, 86 nt, Homo sapiens
  source 1..86: mol_type = transcribed RNA
ggggctgggggtgtggggagagagagtgcacagccagctcagggattaaagctctttctctctctctctctcccacttccctgcag

SEQ ID NO: 242 - RNA, 86 nt, Homo sapiens
  source 1..86: mol_type = transcribed RNA
agggagaagggtcggggcagggagggcagggcaggctctggggtggggggtctgtgagtcagccacggctctgcccacgtctcccc

SEQ ID NO: 243 - RNA, 73 nt, Homo sapiens
  source 1..73: mol_type = transcribed RNA
ctggtgtttgaggcgatgtggggatgtagagacaacttcccagtctcatttcctcatcctgccaggccaccat

SEQ ID NO: 244 - RNA, 98 nt, Homo sapiens
  source 1..98: mol_type = transcribed RNA
cttggtcaataggaaagaggtgggacctcctggcttttcctctgcagcatggctcggacctagtgcaatgtttaagctcccctctctttcctgttcag

SEQ ID NO: 245 - RNA, 102 nt, Homo sapiens
  source 1..102: mol_type = transcribed RNA
tgtgttccctatcctccttatgtcccacccccactcctgtttgaatatttcaccagaaacaggagtggggggtgggacgtaaggaggatgggggaaagaaca

SEQ ID NO: 246 - RNA, 68 nt, Homo sapiens
  source 1..68: mol_type = transcribed RNA
tgcccaggctggagcgagtgcagtggtgcagtcagtcctagctcactgcagcctcgaactcctgggct

SEQ ID NO: 247 - RNA, 83 nt, Homo sapiens
  source 1..83: mol_type = transcribed RNA
aatagagggtgcacaggcacgggagctcaggtgaggcagggagctgagctcacctgacctcccatgcctgtgcaccctctatt

SEQ ID NO: 248 - RNA, 76 nt, Homo sapiens
  source 1..76: mol_type = transcribed RNA
actgactttgagtctctcctcagggtgctgcaggcaaagctggggacccagggagagacgtaagtgaggggagatg

SEQ ID NO: 249 - RNA, 63 nt, Homo sapiens
  source 1..63: mol_type = transcribed RNA
tggtggcggcggtagttatgggcttctctttctcaccagcagcccctgggccgccgcctccct

SEQ ID NO: 250 - RNA, 93 nt, Homo sapiens
  source 1..93: mol_type = transcribed RNA
gcttgttggggattgggtcaggccagtgttcaagggcccctcctctagtactccctgtttgtgttctgccactgactgagcttctccccacag

SEQ ID NO: 251 - RNA, 57 nt, Homo sapiens
  source 1..57: mol_type = transcribed RNA
cattggagggtgtggaagacatctgggccaactctgatctcttcatctaccccccag

SEQ ID NO: 252 - RNA, 109 nt, Homo sapiens
  source 1..109: mol_type = transcribed RNA
ggtcgggctcaccatgacacagtgtgagacctcgggctacaacacaggacccgggcgctgctctgacccctcgtgtcttgtgttgcagccggagggacgcaggtccgca

SEQ ID NO: 253 - RNA, 68 nt, Homo sapiens
  source 1..68: mol_type = transcribed RNA
gaatggaagaagaaggcggtcggtctgcgggagccaggccgcagagccatccgccttctgtccatgtc

SEQ ID NO: 254 - RNA, 77 nt, Homo sapiens
  source 1..77: mol_type = transcribed RNA
gagttgggaggttccctctccaaatgtgtcttgatcccccaccccaagacacatttggagagggaccctcccaactc

SEQ ID NO: 255 - RNA, 66 nt, Homo sapiens
  source 1..66: mol_type = transcribed RNA
gggaggagggaggagatgggccaagttccctctggctggaacgcccttcccccccttcttcacctg

SEQ ID NO: 256 - RNA, 98 nt, Homo sapiens
  source 1..98: mol_type = transcribed RNA
ttgggcaaggtgcggggctagggctaacagcagtcttactgaaggtttcctggaaaccacgcacatgctgttgccactaacctcaaccttactcggtc

SEQ ID NO: 257 - RNA, 67 nt, Homo sapiens
  source 1..67: mol_type = transcribed RNA
gcgccctccctctctccccggtgtgcaaatgtgtgtgtgcggtgttatgccggacaagagggaggtg

SEQ ID NO: 258 - RNA, 73 nt, Homo sapiens
  source 1..73: mol_type = transcribed RNA
gtgggcgggggcaggtgtgtggtgggtggtggcctgcggtgagcagggccctcacacctgcctcgccccccag

SEQ ID NO: 259 - RNA, 82 nt, Homo sapiens
  source 1..82: mol_type = transcribed RNA
tgtgggcagggccctggggagctgaggctctgggggtggccggggctgaccctgggcctctgctccccagtgtctgaccgcg

SEQ ID NO: 260 - RNA, 61 nt, Homo sapiens
  source 1..61: mol_type = transcribed RNA
aactgcggggccagagcagagagcccttgcacaccaccagcctctcctccctgtgccccag

SEQ ID NO: 261 - RNA, 79 nt, Homo sapiens
  source 1..79: mol_type = transcribed RNA
gtgcagatccttgggagccctgttagactctggattttacacttggagtgaacgggcgccatcccgaggctttgcacag

SEQ ID NO: 262 - RNA, 99 nt, Homo sapiens
  source 1..99: mol_type = transcribed RNA
gcaagggacagggagggtcgtggcgacactcgcgccagctcccgggacggctgggctcgggctggtcgccgacctccgaccctccactagatgcctggc

SEQ ID NO: 263 - RNA, 65 nt, Homo sapiens
  source 1..65: mol_type = transcribed RNA
gagggtgggcgagggcggctgagcggctccatcccccggcctgctcatccccctcgccctctcag

SEQ ID NO: 264 - RNA, 61 nt, Homo sapiens
  source 1..61: mol_type = transcribed RNA
gagggcagcgtgggtgtggcggaggcaggcgtgaccgtttgccgccctctcgctgctctag

SEQ ID NO: 265 - RNA, 96 nt, Homo sapiens
  source 1..96: mol_type = transcribed RNA
ctcgggaggggcgggaggggggtccccggtgctcggatctcgagggtgcttattgttcggtccgagcctgggtctccctcttccccccaacccccc

SEQ ID NO: 266 - RNA, 92 nt, Homo sapiens
  source 1..92: mol_type = transcribed RNA
cgctgcgcttctgggcccgcggcgggcgtggggctgcccgggccggtcgaccagcgcgccgtagctcccgaggcccgagccgcgacccgcgg

SEQ ID NO: 267 - RNA, 67 nt, Homo sapiens
  source 1..67: mol_type = transcribed RNA
gtgggaggggagaggcagcaagcacacagggcctgggactagcatgctgacctccctcctgccccag

SEQ ID NO: 268 - RNA, 60 nt, Homo sapiens
  source 1..60: mol_type = transcribed RNA
aggcctagggggtggcaggctggccatcagtgtgggctaaccctgtcctctccctcccag

SEQ ID NO: 269 - RNA, 66 nt, Homo sapiens
  source 1..66: mol_type = transcribed RNA
ccgagtggggcggggcaggtccctgcagggactgtgacactgaaggacctgcaccttcgcccacag

SEQ ID NO: 270 - RNA, 74 nt, Homo sapiens
  source 1..74: mol_type = transcribed RNA
cctgcaggaggcagtgggcgagcaggcggggcagcccaatgccatgggcctgatctcaccgctgcctccttccc

SEQ ID NO: 271 - RNA, 51 nt, Homo sapiens
  source 1..51: mol_type = transcribed RNA
agagatgaagcgggggggcggggtcttgctctattgcctacgctgatctca

SEQ ID NO: 272 - RNA, 68 nt, Homo sapiens
  source 1..68: mol_type = transcribed RNA
gtgcaaagagcaggaggacaggggatttatctcccaagggaggtcccctgatcctagtcacggcacca

SEQ ID NO: 273 - RNA, 73 nt, Homo sapiens
  source 1..73: mol_type = transcribed RNA
ggcgaggggaggcgcaggctcggaaaggcgcgcgaggctccaggctccttcccgatccaccgctctcctcgct

SEQ ID NO: 274 - RNA, 96 nt, Homo sapiens
  source 1..96: mol_type = transcribed RNA
gctctggggcgtgccgccgccgtcgctgccacctcccctaccgctagtggaagaagatggcggaaggcggagcggcggatctggacacccagcggt

SEQ ID NO: 275 - RNA, 81 nt, Homo sapiens
  source 1..81: mol_type = transcribed RNA
catcctccttacgtcccaccccccactcctgtttctggtgaaatattcaaacaggagtgggggtgggacataaggaggata

SEQ ID NO: 276 - RNA, 65 nt, Homo sapiens
  source 1..65: mol_type = transcribed RNA
gggtgctcggggcaggcggctgggagcggccctcacattgatggctcctgccacctcctccgcag

SEQ ID NO: 277 - RNA, 82 nt, Homo sapiens
  source 1..82: mol_type = transcribed RNA
gctacggggagcggggaggaagtgggcgctgcttctgcgttatctggaaggagcagcccactcctgtcctgggctctgtggt

SEQ ID NO: 278 - RNA, 103 nt, Homo sapiens
  source 1..103: mol_type = transcribed RNA
gtgtcggctgtggcgtgactgtccctctgtgtcccccactaggcccactgctcagtggagcgtggaggacgaggaggaggccgtccacgagcaatgccagcat

SEQ ID NO: 279 - RNA, 62 nt, Homo sapiens
  source 1..62: mol_type = transcribed RNA
tggcctagggggcggcttgtggagtgtatgggctgagccttgctctgctcccccgcccccag

SEQ ID NO: 280 - RNA, 72 nt, Homo sapiens
  source 1..72: mol_type = transcribed RNA
atgagcgggtgggagcagatcttattgagagttccttctcctgctcctgattgtcttcccccaccctcacag

SEQ ID NO: 281 - RNA, 80 nt, Homo sapiens
  source 1..80: mol_type = transcribed RNA
acctctacctcccggcagaggaggctgcagaggctggctttccaaaactctgccccctccgctgctgccaagtggctggt

SEQ ID NO: 282 - RNA, 64 nt, Homo sapiens
  source 1..64: mol_type = transcribed RNA
tctgggcgaggggtgggctctcagaggggctggcagtactgctctgaggcctgcctctccccag

SEQ ID NO: 283 - RNA, 98 nt, Homo sapiens
  source 1..98: mol_type = transcribed RNA
ttctcacccccgcctgacacgggcgacagctgcggcccgctgtgttcactcgggccgagtgcgtctcctgtcaggcaagggagagcagagcccccctg

SEQ ID NO: 284 - RNA, 97 nt, Homo sapiens
  source 1..97: mol_type = transcribed RNA
cccgggaccttggtccaggcgctggtctgcgtggtgctcgggtggataagtctgatctgagcaccacacaggccgggcgccgggaccaagggggctc

SEQ ID NO: 285 - RNA, 69 nt, Homo sapiens
  source 1..69: mol_type = transcribed RNA
ccgggcaggcaggtgtagggtggagcccactgtggctcctgactcagccctgctgccttcacctgccag

SEQ ID NO: 286 - RNA, 64 nt, Homo sapiens
  source 1..64: mol_type = transcribed RNA
gaggcactgggtaggtggggctccagggctcctgacacctggacctctcctccccaggcccaca

SEQ ID NO: 287 - RNA, 66 nt, Homo sapiens
  source 1..66: mol_type = transcribed RNA
agcagccctcggcggcccggggggcgggcggcggtgcccgtcccggggctgcgcgaggcacaggcg

SEQ ID NO: 288 - RNA, 65 nt, Homo sapiens
  source 1..65: mol_type = transcribed RNA
agccctgggggtggtctctagccaaggctctggggtctcacccttggctggtctctgctccgcag

SEQ ID NO: 289 - RNA, 55 nt, Homo sapiens
  source 1..55: mol_type = transcribed RNA
ccggatccgagtcacggcaccaaatttcatgcgtgtccgtgtgaagagaccacca

SEQ ID NO: 290 - RNA, 115 nt, Homo sapiens
  source 1..115: mol_type = transcribed RNA
gagcaaaaaccagagaacaacatgggagcgttcctaacccctaaggcaactggatgggagacctgacccatccagttctctgagggggctcttgtgtgttctacaaggttgttca

SEQ ID NO: 291 - RNA, 115 nt, Homo sapiens
  source 1..115: mol_type = transcribed RNA
ggtgccgagggccgtccggcatcctaggcgggtcgctgcggtacctccctcctgtctgtggcggtgggatcccgtggccgtgttttcctggtggcccggccgtgcctgaggtttc

SEQ ID NO: 292 - RNA, 87 nt, Homo sapiens
  source 1..87: mol_type = transcribed RNA
gtgaggcggggccaggagggtgtgtggcgtgggtgctgcggggccgtcagggtgcctgcgggacgctcacctggctggcccgcccag

SEQ ID NO: 293 - RNA, 86 nt, Homo sapiens
  source 1..86: mol_type = transcribed RNA
attctaggtggggagactgacggctggaggcccataagctgtctaaaacttcggcccccagatttctggtctccccacttcagaac

SEQ ID NO: 294 - RNA, 90 nt, Homo sapiens
  source 1..90: mol_type = transcribed RNA
tctaagaaacgcagtggtctctgaagcctgcaggggcaggccagccctgcactgaacgcctgttcttgccaggtggcagaaggttgctgc

SEQ ID NO: 295 - RNA, 81 nt, Homo sapiens
  source 1..81: mol_type = transcribed RNA
ctccctgggagggcgtggatgatggtgggagaggagccccactgtggaagtctgacccccacatcgccccaccttccccag

SEQ ID NO: 296 - RNA, 66 nt, Homo sapiens
  source 1..66: mol_type = transcribed RNA
tcaagacggggagtcaggcagtggtggagatggagagccctgagcctccactctcctggcccccag

SEQ ID NO: 297 - RNA, 94 nt, Homo sapiens
  source 1..94: mol_type = transcribed RNA
gtgaggtgtgggcccggccccaggagcggggcctgggcagccccgtgtgttgaggaaggaaggcagggcccccgctccccgggcctgaccccac

SEQ ID NO: 298 - RNA, 90 nt, Homo sapiens
  source 1..90: mol_type = transcribed RNA
cgggcggggcgggtccggccgcctccgagcccggccggcagcccccggccttaaagcgcgggctgtccggaggggtcggctttcccaccg

SEQ ID NO: 299 - RNA, 60 nt, Homo sapiens
  source 1..60: mol_type = transcribed RNA
caaggtgggggagatgggggttgaacttcatttctcatgctcatccccatctcctttcag

SEQ ID NO: 300 - RNA, 109 nt, Homo sapiens
  source 1..109: mol_type = transcribed RNA
tcccgcattccctctgctttggtcaggtggtgccctccttccatgggtagagccagagatggtgggttctggctggtcagatgggagtggacagagacccggggtcctc

SEQ ID NO: 301 - RNA, 84 nt, Homo sapiens
  source 1..84: mol_type = transcribed RNA
ctccccatggccctgtctcccaacccttgtaccagtgctgggctcagaccctggtacaggcctgggggacagggacctggggac

SEQ ID NO: 302 - RNA, 80 nt, Homo sapiens
  source 1..80: mol_type = transcribed RNA
ggttccggagccccggcgcgggcgggttctggggtgtagacgctgctggccagcccgccccagccgaggttctcggcacc

SEQ ID NO: 303 - RNA, 80 nt, Homo sapiens
  source 1..80: mol_type = transcribed RNA
tgagaggccgcaccttgccttgctgcccgggccgtgcacccgtgggccccagggcgacgcggcgggggcggccctagcga

SEQ ID NO: 304 - RNA, 106 nt, Homo sapiens
  source 1..106: mol_type = transcribed RNA
agaagaatgcccaaccagccctcagttgctacagttccctgttgtttcagctcgacaacaacaggcggctgtagcaatggggggctggatgggcatctcaatgtgc

SEQ ID NO: 305 - RNA, 86 nt, Homo sapiens
  source 1..86: mol_type = transcribed RNA
agcactgcccccggtgagtcagggtggggctggccccctgcttcgtgcccatccgcgctctgactctctgcccacctgcaggagct

SEQ ID NO: 306 - RNA, 53 nt, Homo sapiens
  source 1..53: mol_type = transcribed RNA
ggtgggggttggaggcgtgggttttagaacctatccctttctagccctgagca

SEQ ID NO: 307 - RNA, 80 nt, Homo sapiens
  source 1..80: mol_type = transcribed RNA
cgtgtgagcccgccctgtgcccggcccacttctgcttcctcttagcgcaggaggggtcccgcactgggaggggccctcac

SEQ ID NO: 308 - RNA, 79 nt, Homo sapiens
  source 1..79: mol_type = transcribed RNA
gtgaggtgggggccagcagggagtgggctgggctgggctgggccaaggtacaaggcctcaccctgcatcccgcacccag

SEQ ID NO: 309 - RNA, 50 nt, Homo sapiens
  source 1..50: mol_type = transcribed RNA
acccgggcgtggtggtgggggtgggtgcctgtaattccagctagttggga

SEQ ID NO: 310 - RNA, 88 nt, Homo sapiens
  source 1..88: mol_type = transcribed RNA
gtggggccaggcggtggtgggcactgctggggtgggcacagcagccatgcagagcgggcatttgaccccgtgccacccttttccccag

SEQ ID NO: 311 - RNA, 62 nt, Homo sapiens
  source 1..62: mol_type = transcribed RNA
gagggtggtggaggaagagggcagctcccatgactgcctgaccgccttctctcctcccccag

SEQ ID NO: 312 - RNA, 73 nt, Homo sapiens
  source 1..73: mol_type = transcribed RNA
gaaaacaaccaggtgggcttcccggagggcggaacacccagccccagcatccagggctcacctaccacgtttg

SEQ ID NO: 313 - RNA, 65 nt, Homo sapiens
  source 1..65: mol_type = transcribed RNA
gagggctaggtggggggcttgaagccccgagatgcctcacgtcttcacccctctcacctaagcag

SEQ ID NO: 314 - RNA, 73 nt, Homo sapiens
  source 1..73: mol_type = transcribed RNA
aggccaggtgggtatggaggagccctcatatggcagttggcgagggcccagtgagcccctctctgctctccag

SEQ ID NO: 315 - RNA, 93 nt, Homo sapiens
  source 1..93: mol_type = transcribed RNA
ccttccggcgtcccaggcggggcgccgcgggaccgccctcgtgtctgtggcggtgggatcccgcggccgtgttttcctggtggcccggccatg

SEQ ID NO: 316 - RNA, 87 nt, Homo sapiens
  source 1..87: mol_type = transcribed RNA
ccctcatctctgggcaggggcttattgtaggagtctctgaagagagctgtggactgacctgctttaacccttccccaggttcccatt

SEQ ID NO: 317 - RNA, 51 nt, Homo sapiens
  source 1..51: mol_type = transcribed RNA
acagaccccggggagcccggcggtgaagctcctggtatcctgggtgtctga

SEQ ID NO: 318 - RNA, 49 nt, Homo sapiens
  source 1..49: mol_type = transcribed RNA
ttcccagccaacgcaccaaaaatgatatgggtctgttgtctggagaaac

SEQ ID NO: 319 - RNA, 96 nt, Homo sapiens
  source 1..96: mol_type = transcribed RNA
cgggccccgggcgggcgggagggacgggacgcggtgcagtgttgttttttcccccgccaatattgcactcgtcccggcctccggcccccccggccc

SEQ ID NO: 320 - RNA, 67 nt, Homo sapiens
  source 1..67: mol_type = transcribed RNA
gtggcactcaaactgtgggggcactttctgctctctggtgaaagtgccgccatcttttgagtgttac

SEQ ID NO: 321 - RNA, 89 nt, Homo sapiens
  source 1..89: mol_type = transcribed RNA
agcctgtgggaaagagaagagcagggcagggtgaaggcccggcggagacactctgcccaccccacaccctgcctatgggccacacagct

SEQ ID NO: 322 - RNA, 70 nt, Homo sapiens
  source 1..70: mol_type = transcribed RNA
ctcgggcccgaccgcgccggcccgcacctcccggcccggagctgcgggctgcggtcagggcgatcccggg

SEQ ID NO: 323 - RNA, 79 nt, Homo sapiens
  source 1..79: mol_type = transcribed RNA
ctcgaggtgctgggggacgcgtgagcgcgagccgcttcctcacggctcggccgcggcgcgtagcccccgccacatcggg

SEQ ID NO: 324 - RNA, 94 nt, Homo sapiens
  source 1..94: mol_type = transcribed RNA
ataaaggaagttaggctgaggggcagagagcgagacttttctattttccaaaagctcggtctgaggcccctcagtcttgcttcctaacccgcgc

SEQ ID NO: 325 - RNA, 47 nt, Homo sapiens
  source 1..47: mol_type = transcribed RNA
ctcggcgcggggcgcgggctccgggttggggcgagccaacgccgggg

SEQ ID NO: 326 - RNA, 77 nt, Homo sapiens
  source 1..77: mol_type = transcribed RNA
catgagaaatcctgctggtcaaccatagccctggtcagactctccggggctgtgattgaccagcaggacttctcatg

SEQ ID NO: 327 - RNA, 80 nt, Homo sapiens
  source 1..80: mol_type = transcribed RNA
gagaggccaagaccttgggaatgggggtaagggccttctgagcccaggtccgaactctccattcctctgcagagcgctct

SEQ ID NO: 328 - RNA, 70 nt, Homo sapiens
  source 1..70: mol_type = transcribed RNA
ggccgcggcgcgcaagatggcggcgggcccgggcaccgccccttccgccccgccgggcgtcgcacgaggc

SEQ ID NO: 329 - RNA, 62 nt, Homo sapiens
  source 1..62: mol_type = transcribed RNA
gctgggggtcccccgacagtgtggagctggggccgggtcccggggaggggggttctgggcag

SEQ ID NO: 330 - RNA, 62 nt, Homo sapiens
  source 1..62: mol_type = transcribed RNA
ccttctgcggcagagctggggtcaccagccctcatgtacttgtgacttctcccctgccacag

SEQ ID NO: 331 - RNA, 68 nt, Homo sapiens
  source 1..68: mol_type = transcribed RNA
agggttggggggacaggatgagaggctgtcttcattccctcttgaccacccctcgtttcttcccccag

SEQ ID NO: 332 - RNA, 64 nt, Homo sapiens
  source 1..64: mol_type = transcribed RNA
gagctctgggaggggctgggtttggcaggacagtttccaagccctgtctcctcccatcttccag

SEQ ID NO: 333 - RNA, 65 nt, Homo sapiens
  source 1..65: mol_type = transcribed RNA
gtctcctggggggaggagaccctgctctccctggcagcaagcctctcctgcccttccagattagc

SEQ ID NO: 334 - RNA, 67 nt, Homo sapiens
  source 1..67: mol_type = transcribed RNA
ggcagccagggggatgggcgagcttgggcccattcctttccttaccctaccccccatccccctgtag

SEQ ID NO: 335 - RNA, 72 nt, Homo sapiens
  source 1..72: mol_type = transcribed RNA
tgtgggactgcaaatgggagctcagcacctgcctgccacccacgcagaccagcccctgctctgttcccacag

SEQ ID NO: 336 - RNA, 81 nt, Homo sapiens
  source 1..81: mol_type = transcribed RNA
tacaggtgcaggggaactgtagatgaaaaggcttggcacttgagggaaagcctcagttcattctcattttgctcacctgtt

SEQ ID NO: 337 - RNA, 109 nt, Homo sapiens
  source 1..109: mol_type = transcribed RNA
tgctattgtcttactgctacagcagggctggggattgcagtatccgctgttgctgctgctcccagtcctgcccctgctgctacctagtccagcctcaccgcatcccaga

SEQ ID NO: 338 - RNA, 80 nt, Homo sapiens
  source 1..80: mol_type = transcribed RNA
ctgcagcgtgcttctccaggccccgcgcgcggacagacacacggacaagtcccgccaggggctgggcgcgcgccagccgg

SEQ ID NO: 339 - RNA, 80 nt, Homo sapiens
  source 1..80: mol_type = transcribed RNA
tgccgtcggcctggggaggaggaagggcaagtccaaaggtatacagttggtctgttcattctctctttttggcctacaag

SEQ ID NO: 340 - RNA, 84 nt, Homo sapiens
  source 1..84: mol_type = transcribed RNA
ccgcagccgccgcgccgggcccgggttggccgctgacccccgcggggcccccggcggccggggcgggggcgggggctgccccgg

SEQ ID NO: 341 - RNA, 153 nt, Homo sapiens
  source 1..153: mol_type = transcribed RNA
gctccgccccacgtcgcatgcgccccgggaacgcgtggggcggagcttccggaggccccgctctgctgccgaccctgtggagcggagggtgaagcctccggatgccagtccctcatcgctggcctggtcgcgctgtggcgaagggggcggagc

SEQ ID NO: 342 - RNA, 153 nt, Homo sapiens
  source 1..153: mol_type = transcribed RNA
gctccgccccacgtcgcatgcgccccgggaacgcgtggggcggagcttccggaggccccgccctgctgccgaccctgtggagcggagggtgaagcctccggatgccagtccctcatcgctggcccggtcgcgctgtggcgaagggggcggagc

SEQ ID NO: 343 - RNA, 80 nt, Homo sapiens
  source 1..80: mol_type = transcribed RNA
aggacccttccagagggccccccctcaatcctgttgtgcctaattcagagggttgggtggaggctctcctgaagggctct

SEQ ID NO: 344 - RNA, 94 nt, Homo sapiens
  source 1..94: mol_type = transcribed RNA
cgggcagcgggtgccaggcacggtgtcagcaggcaacatggccgagaggccggggcctccgggcggcgccgtgtccgcgaccgcgtaccctgac

SEQ ID NO: 345 - RNA, 52 nt, Homo sapiens
  source 1..52: mol_type = transcribed RNA
tagccgggcgtggtggtgggggcctgtggtcccagctactttggaggctgag

SEQ ID NO: 346 - RNA, 70 nt, Homo sapiens
  source 1..70: mol_type = transcribed RNA
gtccctgggggctgggatgggccatggtgtgctctgatccccctgtggtctcttggcccccaggaactcc

SEQ ID NO: 347 - RNA, 83 nt, Homo sapiens
  source 1..83: mol_type = transcribed RNA
ggcccggctccgggtctcggcccgtacagtccggccggccatgctggcggggctggggccggggccgagcccgcggcggggcc

SEQ ID NO: 348 - RNA, 90 nt, Homo sapiens
  source 1..90: mol_type = transcribed RNA
ggcgcctctgcagctccggctccccctggcctctcgggaactacaagtcccagggggcctggcggtgggcggcgggcggaagaggcgggg

SEQ ID NO: 349 - RNA, 85 nt, Homo sapiens
  source 1..85: mol_type = transcribed RNA
tcctccccggagccaggatgcagctcaagccacagcagggtgtttagcgctcttcagtggctccagattgtggcgctggtgcagg

SEQ ID NO: 350 - RNA, 89 nt, Homo sapiens
  source 1..89: mol_type = transcribed RNA
tctccgtttatcccaccactgccaccattattgctactgttcagcaggtgctgctggtggtgatggtgatagtctggtgggggcggtgg

SEQ ID NO: 351 - RNA, 70 nt, Homo sapiens
  source 1..70: mol_type = transcribed RNA
aaaagcctgtccctaagtccctcccagccttccagagttggtgccaggaaggatttagggacaggctttg

SEQ ID NO: 352 - RNA, 66 nt, Homo sapiens
  source 1..66: mol_type = transcribed RNA
cctggaggggggcactgcgcaagcaaagccagggaccctgagaggctttgcttcctgctcccctag

SEQ ID NO: 353 - RNA, 62 nt, Homo sapiens
  source 1..62: mol_type = transcribed RNA
cttcctggtgggtggggaggagaagtgccgtcctcatgagcccctctctgtcccacccatag

SEQ ID NO: 354 - RNA, 97 nt, Homo sapiens
  source 1..97: mol_type = transcribed RNA
ctcaggtgctctggctgcttgggttcctggcatgctgatttgtgacttaagattaaaatcacattgccagggattaccacgcaaccacgaccttggc

SEQ ID NO: 355 - RNA, 85 nt, Homo sapiens
  source 1..85: mol_type = transcribed RNA
gtgagctgctggggacgcgggtcggggtctgcagggcggtgcggcagccgccacctgacgccgcgcctttgtctgtgtcccacag

SEQ ID NO: 356 - RNA, 83 nt, Homo sapiens
  source 1..83: mol_type = transcribed RNA
gtctactcccagggtgccaagctgtttcgtgttccctccctaggggatcccaggtaggggcagcagaggacctgggcctggac

SEQ ID NO: 357 - RNA, 93 nt, Homo sapiens
  source 1..93: mol_type = transcribed RNA
tacttatggcaccccactcctggtaccatagtcataagttaggagatgttagagctgtgagtaccatgacttaagtgtggtggcttaaacatg

SEQ ID NO: 358 - RNA, 90 nt, Homo sapiens
  source 1..90: mol_type = transcribed RNA
ctgtgggctgggccagggagcagctggtgggtgggaagtaagatctgacctggactccatcccacccaccccctgtttcctggcccacag

SEQ ID NO: 359 - RNA, 74 nt, Homo sapiens
  source 1..74: mol_type = transcribed RNA
gggaggaagaagggaggaggagcggaggggcccttgtcttcccagagcctctcccttcctcccctccccctccc

SEQ ID NO: 360 - RNA, 73 nt, Homo sapiens
  source 1..73: mol_type = transcribed RNA
acctttccagctcatcccacctctgccaccaaaacactcatcgcggggtcagagggagtgccaaaaaaggtaa

SEQ ID NO: 361 - RNA, 70 nt, Homo sapiens
  source 1..70: mol_type = transcribed RNA
cacagtctgactcagtgactcatgtgctggcagtggccacgtaaatagagctactgtgtctgaaagcaat

SEQ ID NO: 362 - RNA, 65 nt, Homo sapiens
  source 1..65: mol_type = transcribed RNA
gcttctgggaggaggggatcttgggagtgatcccaacagctgagctccctgaatccctgtcccag

SEQ ID NO: 363 - RNA, 84 nt, Homo sapiens
  source 1..84: mol_type = transcribed RNA
tgtgcagtgggaaggggggccgatacactgtacgagagtgagtagcaggtctcacagtgaaccggtctctttccctactgtgtc

SEQ ID NO: 364 - RNA, 86 nt, Homo sapiens
  source 1..86: mol_type = transcribed RNA
tgccagtctctaggtccctgagaccctttaacctgtgaggacatccagggtcacaggtgaggttcttgggagcctggcgtctggcc

SEQ ID NO: 365 - RNA, 75 nt, Homo sapiens
  source 1..75: mol_type = transcribed RNA
tcatccctgggtggggatttgttgcattacttgtgttctatataaagtattgcacttgtcccggcctgtggaaga

SEQ ID NO: 366 - RNA, 68 nt, Homo sapiens
  source 1..68: mol_type = transcribed RNA
gcatcctgtactgagctgccccgaggcccttcatgctgcccagctcggggcagctcagtacaggatac

SEQ ID NO: 367 - RNA, 64 nt, Homo sapiens
  source 1..64: mol_type = transcribed RNA
tcctgtactgagctgccccgagctgggcagcatgaagggcctcggggcagctcagtacaggatg

SEQ ID NO: 368 - RNA, 64 nt, Homo sapiens
  source 1..64: mol_type = transcribed RNA
gggtgggggcggggcggcaggggcctcccccagtgccaggccccattctgcttctctcccagct

SEQ ID NO: 369 - RNA, 110 nt, Homo sapiens
  source 1..110: mol_type = transcribed RNA
tcacctggccatgtgacttgtgggcttccctttgtcatccttcgcctagggctctgagcagggcagggacagcaaaggggtgctcagttgtcacttcccacagcacggag

SEQ ID NO: 370 - RNA, 67 nt, Homo sapiens
  source 1..67: mol_type = transcribed RNA
aaatctctctccatatctttcctgcagcccccaggtgggggggaagaaaaggtggggaattagattc

SEQ ID NO: 371 - RNA, 67 nt, Homo sapiens
  source 1..67: mol_type = transcribed RNA
cagggaggaggtggtactaggggccagcaacctgattacccctctttggccctttgtacccctccag

SEQ ID NO: 372 - RNA, 89 nt, Homo sapiens
  source 1..89: mol_type = transcribed RNA
gccggcgcccgagctctggctccgtgtcttcactcccgtgcttgtccgaggagggagggagggacgggggctgtgctggggcagctgga

SEQ ID NO: 373 - RNA, 66 nt, Homo sapiens
  source 1..66: mol_type = transcribed RNA
tgactggggagcagaaggagaacccaagaaaagctgacttggaggtccctccttctgtccccacag

SEQ ID NO: 374 - RNA, 90 nt, Homo sapiens
  source 1..90: mol_type = transcribed RNA
aggcctcgctgttctctatggctttttattcctatgtgattctactgctcactcatatagggattggagccgtggcgcacggcggggaca

SEQ ID NO: 375 - RNA, 63 nt, Homo sapiens
  source 1..63: mol_type = transcribed RNA
gcatgctgggcgaggctggcatctagcacaggcggtagatgcttgctcttgccattgcaatga

SEQ ID NO: 376 - RNA, 78 nt, Homo sapiens
  source 1..78: mol_type = transcribed RNA
gggcccagaagggggcgcagtcactgacgtgaagggaccacatcccgcttcatgtcagtgactcctgccccttggtct

SEQ ID NO: 377 - RNA, 90 nt, Homo sapiens
  source 1..90: mol_type = transcribed RNA
gtgtctctctggagaccctgcagccttcccacccaccagggagctttccatgggctgtggggaaggcgtcagtgtcgggtgagggaacac

SEQ ID NO: 378 - RNA, 54 nt, Homo sapiens
  source 1..54: mol_type = transcribed RNA
agcagcaggggagagagaggagtcctctagacaccgactctgtctcctgcagat

SEQ ID NO: 379 - RNA, 100 nt, Homo sapiens
  source 1..100: mol_type = transcribed RNA
agctcagggcggctgcgcagagggctggactcagcggcggagctggctgctggcctcagttctgcctctgtccaggtccttgtgacccgcccgctctcct

SEQ ID NO: 380 - RNA, 59 nt, Homo sapiens
  source 1..59: mol_type = transcribed RNA
gtccaggcaggagccggactggacctcagggaagaggctgacccggcccctcttgcggc

SEQ ID NO: 381 - RNA, 54 nt, Homo sapiens
  source 1..54: mol_type = transcribed RNA
acgcgggtgcgggccggcggggtagaagccacccggcccggcccggcccggcga

SEQ ID NO: 382 - RNA, 73 nt, Homo sapiens
  source 1..73: mol_type = transcribed RNA
ggccggctggggttcctggggatgggatttgcttcctgtcacaaatcacattgccagggatttccaaccgacc

SEQ ID NO: 383 - RNA, 69 nt, Homo sapiens
  source 1..69: mol_type = transcribed RNA
ctttcggccagcgggacggcatccgaggtgggctaggctcgggcccgtggcgggtgcgggggtgggagg

SEQ ID NO: 384 - RNA, 69 nt, Homo sapiens
  source 1..69: mol_type = transcribed RNA
tggggtaggggtgggggaattcaggggtgtcgaactcatggctgccacctttgtgtccccatcctgcag

SEQ ID NO: 385 - RNA, 70 nt, Homo sapiens
  source 1..70: mol_type = transcribed RNA
ggtttctccttgaggagacatggtgggggccggtcaggcagcccatgccatgtgtcctcatggagaggcc

SEQ ID NO: 386 - RNA, 72 nt, Homo sapiens
  source 1..72: mol_type = transcribed RNA
cttgggaatggcaaggaaaccgttaccattactgagtttagtaatggtaatggttctcttgctatacccaga

SEQ ID NO: 387 - RNA, 67 nt, Homo sapiens
  source 1..67: mol_type = transcribed RNA
ctggtccatttccctgccattcccttggcttcaatttactcccagggctggcagtgacatgggtcaa

SEQ ID NO: 388 - RNA, 94 nt, Homo sapiens
  source 1..94: mol_type = transcribed RNA
cagtgcgacgggcggagcttccagacgctccgccccacgtcgcatgcgccccgggaaagcgtggggcggagcttccggaggccccgccctgctg

SEQ ID NO: 389 - RNA, 88 nt, Homo sapiens
  source 1..88: mol_type = transcribed RNA
gcgacgggcggagcttccagacgctccgccccacgtcgcatgcgccccgggaaagcgtggggcggagcttccggaggccccgccctgc

SEQ ID NO: 390 - RNA, 94 nt, Homo sapiens; identical to SEQ ID NO: 388
  source 1..94: mol_type = transcribed RNA

SEQ ID NO: 391 - RNA, 94 nt, Homo sapiens; identical to SEQ ID NO: 388
  source 1..94: mol_type = transcribed RNA

SEQ ID NO: 392 - RNA, 59 nt, Homo sapiens
  source 1..59: mol_type = transcribed RNA
ctgtgtcggggagtctggggtccggaattctccagagcctctgtgcccctacttcccag

SEQ ID NO: 393 - RNA, 84 nt, Homo sapiens
  source 1..84: mol_type = transcribed RNA
gaggctgggcggggcgcggccggatcggtcgagagcgtcctggctgatgacggtctcccgtgcccacgccccaaacgcagtctc

SEQ ID NO: 394 - RNA, 78 nt, Homo sapiens
  source 1..78: mol_type = transcribed RNA
ggcctcaggcaggcgcacccgaccacatgcatggctggtggcggcgtgcaggggtcgggtgggccaggctgtggggcg

SEQ ID NO: 395 - RNA, 89 nt, Homo sapiens
  source 1..89: mol_type = transcribed RNA
accatgctgtagtgtgtgtaaacatcctacactctcagctgtgagctcaaggtggctgggagagggttgtttactccttctgccatgga

SEQ ID NO: 396 - RNA, 52 nt, Homo sapiens
  source 1..52: mol_type = transcribed RNA
tctgggctgagccgagctgggttaagccgagctgggttgggctgggctgggt

SEQ ID NO: 397 - RNA, 61 nt, Homo sapiens
  source 1..61: mol_type = transcribed RNA
tcacccggtgagggcgggtggaggaggagggtccccaccatcagccttcactgggacggga

SEQ ID NO: 398 - RNA, 119 nt, Homo sapiens
  source 1..119: mol_type = transcribed RNA
agcctgcgccggagccggggcctgagcccgggccgcgcaggccgtgaactcgtcgagctgcgcgtgcggccggtgctcaacctgccgggtcctggccccgcgctcccgcgcgccctgga

SEQ ID NO: 399 - RNA, 23 nt, Homo sapiens
  source 1..23: mol_type = transcribed RNA
ctcctggggcccgcactctcgct

SEQ ID NO: 400 - RNA, 18 nt, Homo sapiens
  source 1..18: mol_type = transcribed RNA
ctcctggggcccgcactc

SEQ ID NO: 401 - RNA, 22 nt, Homo sapiens
  source 1..22: mol_type = transcribed RNA
ccgggaacgtcgagactggagc

SEQ ID NO: 402 - RNA, 15 nt, Homo sapiens
  source 1..15: mol_type = transcribed RNA
cgggaacgtcgagac

SEQ ID NO: 403 - RNA, 22 nt, Homo sapiens
  source 1..22: mol_type = transcribed RNA
cgcggcggggacggcgattggt

SEQ ID NO: 404 - RNA, 17 nt, Homo sapiens
  source 1..17: mol_type = transcribed RNA
cggcggggacggcgatt

SEQ ID NO: 405 - RNA, 24 nt, Homo sapiens
  source 1..24: mol_type = transcribed RNA
gaggctgggaaggcaaagggacgt

SEQ ID NO: 406 - RNA, 15 nt, Homo sapiens
  source 1..15: mol_type = transcribed RNA
gaaggaggctgggaa

SEQ ID NO: 407 - RNA, 23 nt, Homo sapiens
  source 1..23: mol_type = transcribed RNA
ggtgggtgaggtcgggccccaag

SEQ ID NO: 408 - RNA, 20 nt, Homo sapiens
  source 1..20: mol_type = transcribed RNA
cggggtgggtgaggtcgggc

SEQ ID NO: 409 - RNA, 17 nt, Homo sapiens
  source 1..17: mol_type = transcribed RNA
ggtgagcgctcgctggc

SEQ ID NO: 410 - RNA, 15 nt, Homo sapiens
  source 1..15: mol_type = transcribed RNA
cggtgagcgctcgct

SEQ ID NO: 411 - RNA, 26 nt, Homo sapiens
  source 1..26: mol_type = transcribed RNA
ccttctggagaggctttgtgcggata

SEQ ID NO: 412 - RNA, 15 nt, Homo sapiens
  source 1..15: mol_type = transcribed RNA
ccttctggagaggct

SEQ ID NO: 413 - RNA, 24 nt, Homo sapiens
  source 1..24: mol_type = transcribed RNA
tcgaggactggtggaagggccttt

SEQ ID NO: 414 - RNA, 16 nt, Homo sapiens
  source 1..16: mol_type = transcribed RNA
tcgaggactggtggaa

SEQ ID NO: 415 - RNA, 19 nt, Homo sapiens
  source 1..19: mol_type = transcribed RNA
agtgggaggccagggcacg

SEQ ID NO: 416 - RNA, 15 nt, Homo sapiens
  source 1..15: mol_type = transcribed RNA
agggggagctgcagg

SEQ ID NO: 417 - RNA, 16 nt, Homo sapiens
  source 1..16: mol_type = transcribed RNA
tgctggtgatgctttc

SEQ ID NO: 418 - RNA, 16 nt, Homo sapiens
  source 1..16: mol_type = transcribed RNA
tgctggtgatgctttc

SEQ ID NO: 419 - RNA, 23 nt, Homo sapiens
  source 1..23: mol_type = transcribed RNA
cggggccgtagcactgtctgaga

SEQ ID NO: 420 - RNA, 20 nt, Homo sapiens
  source 1..20: mol_type = transcribed RNA
cggggccgtagcactgtctg

SEQ ID NO: 421 - RNA, 18 nt, Homo sapiens
  source 1..18: mol_type = transcribed RNA
gaggctgaaggaagatgg

SEQ ID NO: 422 - RNA, 15 nt, Homo sapiens
  source 1..15: mol_type = transcribed RNA
gaggctgaaggaaga

SEQ ID NO: 423 - RNA, 23 nt, Homo sapiens
  source 1..23: mol_type = transcribed RNA
tgaggatatggcagggaagggga

SEQ ID NO: 424 - RNA, 19 nt, Homo sapiens
  source 1..19: mol_type = transcribed RNA
tgaggatatggcagggaag

SEQ ID NO: 425 - RNA, 23 nt, Homo sapiens
  source 1..23: mol_type = transcribed RNA
agggtcggggcagggagggcagg

SEQ ID NO: 426 - RNA, 15 nt, Homo sapiens
  source 1..15: mol_type = transcribed RNA
gggagaagggtcggg

SEQ ID NO: 427 - RNA, 21 nt, Homo sapiens
  source 1..21: mol_type = transcribed RNA
gaggcgatgtggggatgtaga

SEQ ID NO: 428 - RNA, 20 nt, Homo sapiens
  source 1..20: mol_type = transcribed RNA
cccagtctcatttcctcatc

SEQ ID NO: 429 - RNA, 21 nt, Homo sapiens
  source 1..21: mol_type = transcribed RNA
cttccccccagtaatcttcat

SEQ ID NO: 430 - RNA, 21 nt, Homo sapiens
  source 1..21: mol_type = transcribed RNA
cttccccccagtaatcttcat

SEQ ID NO: 431 - RNA, 21 nt, Homo sapiens
  source 1..21: mol_type = transcribed RNA
cccaggctggagcgagtgcag

SEQ ID NO: 432 - RNA, 15 nt, Homo sapiens
  source 1..15: mol_type = transcribed RNA
agctcactgcagcct

SEQ ID NO: 433 - RNA, 22 nt, Homo sapiens
  source 1..22: mol_type = transcribed RNA
caggcacgggagctcaggtgag

SEQ ID NO: 434 - RNA, 17 nt, Homo sapiens
  source 1..17: mol_type = transcribed RNA
caggcacgggagctcag

SEQ ID NO: 435 - RNA, 16 nt, Homo sapiens
  source 1..16: mol_type = transcribed RNA
ggacccagggagagac

SEQ ID NO: 436 - RNA, 16 nt, Homo sapiens
  source 1..16: mol_type = transcribed RNA
ggacccagggagagac

SEQ ID NO: 437 - RNA, 25 nt, Homo sapiens
  source 1..25: mol_type = transcribed RNA
tggcggcggtagttatgggcttctc

SEQ ID NO: 438 - RNA, 25 nt, Homo sapiens
  source 1..25: mol_type = transcribed RNA
tggcggcggtagttatgggcttctc

SEQ ID NO: 439 - RNA, 21 nt, Homo sapiens
  source 1..21: mol_type = transcribed RNA
caactctgatctcttcatcta

SEQ ID NO: 440 - RNA, 20 nt, Homo sapiens
  source 1..20: mol_type = transcribed RNA
tctcttcatctaccccccag

SEQ ID NO: 441 - RNA, 23 nt, Homo sapiens
  source 1..23: mol_type = transcribed RNA
ggctacaacacaggacccgggcg

SEQ ID NO: 442 - RNA, 21 nt, Homo sapiens
  source 1..21: mol_type = transcribed RNA
ggctacaacacaggacccggg

SEQ ID NO: 443 - RNA, 23 nt, Homo sapiens
  source 1..23: mol_type = transcribed RNA
agaagaaggcggtcggtctgcgg

SEQ ID NO: 444 - RNA, 21 nt, Homo sapiens
  source 1..21: mol_type = transcribed RNA
aagaaggcggtcggtctgcgg

SEQ ID NO: 445 - RNA, 20 nt, Homo sapiens
  source 1..20: mol_type = transcribed RNA
aagacacatttggagaggga

SEQ ID NO: 446 - RNA, 16 nt, Homo sapiens
  source 1..16: mol_type = transcribed RNA
agacacatttggagag

SEQ ID NO: 447 - RNA, 26 nt, Homo sapiens
  source 1..26: mol_type = transcribed RNA
aggagggaggagatgggccaagttcc

SEQ ID NO: 448 - RNA, 15 nt, Homo sapiens
  source 1..15: mol_type = transcribed RNA
gggaggagggaggag

SEQ ID NO: 449 - RNA, 25 nt, Homo sapiens
  source 1..25: mol_type = transcribed RNA
tgcggggctagggctaacagcagtc

SEQ ID NO: 450 - RNA, 15 nt, Homo sapiens
  source 1..15: mol_type = transcribed RNA
tgcggggctagggct

SEQ ID NO: 451 - RNA, 20 nt, Homo sapiens
  source 1..20: mol_type = transcribed RNA
ctccccggtgtgcaaatgtg

SEQ ID NO: 452 - RNA, 15 nt, Homo sapiens
  source 1..15: mol_type = transcribed RNA
gtgtgcggtgttatg

SEQ ID NO: 453 - RNA, 22 nt, Homo sapiens
  source 1..22: mol_type = transcribed RNA
cctcacacctgcctcgcccccc

SEQ ID NO: 454 - RNA, 15 nt, Homo sapiens
  source 1..15: mol_type = transcribed RNA
tcacacctgcctcgc

SEQ ID NO: 455 - RNA, 24 nt, Homo sapiens
  source 1..24: mol_type = transcribed RNA
tggggagctgaggctctgggggtg

SEQ ID NO: 456 - RNA, 15 nt, Homo sapiens
  source 1..15: mol_type = transcribed RNA
ggccctggggagctg

SEQ ID NO: 457 - RNA, 27 nt, Homo sapiens
  source 1..27: mol_type = transcribed RNA
gtgaacgggcgccatcccgaggctttg

SEQ ID NO: 458 - RNA, 16 nt, Homo sapiens
  source 1..16: mol_type = transcribed RNA
gtgaacgggcgccatc

SEQ ID NO: 459 - RNA, 21 nt, Homo sapiens
  source 1..21: mol_type = transcribed RNA
gagggcagcgtgggtgtggcg

SEQ ID NO: 460 - RNA, 21 nt, Homo sapiens
  source 1..21: mol_type = transcribed RNA
gagggcagcgtgggtgtggcg

SEQ ID NO: 461 - RNA, 23 nt, Homo sapiens
  source 1..23: mol_type = transcribed RNA
gggggtccccggtgctcggatct

SEQ ID NO: 462 - RNA, 16 nt, Homo sapiens
  source 1..16: mol_type = transcribed RNA
tcgggaggggcgggag

SEQ ID NO: 463 - RNA, 25 nt, Homo sapiens
  source 1..25: mol_type = transcribed RNA
ttctgggcccgcggcgggcgtgggg

SEQ ID NO: 464 - RNA, 15 nt, Homo sapiens
  source 1..15: mol_type = transcribed RNA
cgcggcgggcgtggg

SEQ ID NO: 465 - RNA, 22 nt, Homo sapiens
  source 1..22: mol_type = transcribed RNA
tgggaggggagaggcagcaagc

SEQ ID NO: 466 - RNA, 22 nt, Homo sapiens
  source 1..22: mol_type = transcribed RNA
tgggaggggagaggcagcaagc

SEQ ID NO: 467 - RNA, 21 nt, Homo sapiens
  source 1..21: mol_type = transcribed RNA
aggaggcagtgggcgagcagg

SEQ ID NO: 468 - RNA, 21 nt, Homo sapiens
  source 1..21: mol_type = transcribed RNA
aggaggcagtgggcgagcagg

SEQ ID NO: 469 - RNA, 15 nt, Homo sapiens
  source 1..15: mol_type = transcribed RNA
tgaagcgggggggcg

SEQ ID NO: 470 - RNA, 15 nt, Homo sapiens
  source 1..15: mol_type = transcribed RNA
tgaagcgggggggcg

SEQ ID NO: 471 - RNA, 17 nt, Homo sapiens
  source 1..17: mol_type = transcribed RNA
tcctagtcacggcacca

SEQ ID NO: 472 - RNA, 17 nt, Homo sapiens
  source 1..17: mol_type = transcribed RNA
tcctagtcacggcacca

SEQ ID NO: 473 - RNA, 23 nt, Homo sapiens
  source 1..23: mol_type = transcribed RNA
ggaggcgcaggctcggaaaggcg

SEQ ID NO: 474 - RNA, 15 nt, Homo sapiens
  source 1..15: mol_type = transcribed RNA
gcaggctcggaaagg

SEQ ID NO: 475 - RNA, 23 nt, Homo sapiens
  source 1..23: mol_type = transcribed RNA
ctagtggaagaagatggcggaag

SEQ ID NO: 476 - RNA, 15 nt, Homo sapiens
  source 1..15: mol_type = transcribed RNA
tagtggaagaagatg

SEQ ID NO: 477 - RNA, 23 nt, Homo sapiens
  source 1..23: mol_type = transcribed RNA
acaggagtgggggtgggacataa

SEQ ID NO: 478 - RNA, 20 nt, Homo sapiens
  source 1..20: mol_type = transcribed RNA
acaggagtgggggtgggaca

SEQ ID NO: 479 - RNA, 25 nt, Homo sapiens
  source 1..25: mol_type = transcribed RNA
agcggggaggaagtgggcgctgctt

SEQ ID NO: 480 - RNA, 21 nt, Homo sapiens
  source 1..21: mol_type = transcribed RNA
agcggggaggaagtgggcgct

SEQ ID NO: 481 - RNA, 22 nt, Homo sapiens
  source 1..22: mol_type = transcribed RNA
ccggcagaggaggctgcagagg

SEQ ID NO: 482 - RNA, 19 nt, Homo sapiens
  source 1..19: mol_type = transcribed RNA
ccggcagaggaggctgcag

SEQ ID NO: 483 - RNA, 15 nt, Homo sapiens
  source 1..15: mol_type = transcribed RNA
tctgggcgaggggtg

SEQ ID NO: 484 - RNA, 15 nt, Homo sapiens
  source 1..15: mol_type = transcribed RNA
tctgggcgaggggtg

SEQ ID NO: 485 - RNA, 26 nt, Homo sapiens
  source 1..26: mol_type = transcribed RNA
gtcccggggctgcgcgaggcacaggc

SEQ ID NO: 486 - RNA, 15 nt, Homo sapiens
  source 1..15: mol_type = transcribed RNA
ggcccggggggcggg

SEQ ID NO: 487 - RNA, 21 nt, Homo sapiens
  source 1..21: mol_type = transcribed RNA
cggatccgagtcacggcacca

SEQ ID NO: 488 - RNA, 15 nt, Homo sapiens
  source 1..15: mol_type = transcribed RNA
ggatccgagtcacgg

SEQ ID NO: 489 - RNA, 21 nt, Homo sapiens
  source 1..21: mol_type = transcribed RNA
atccagttctctgagggggct

SEQ ID NO: 490 - RNA, 21 nt, Homo sapiens
  source 1..21: mol_type = transcribed RNA
atccagttctctgagggggct

SEQ ID NO: 491 - RNA, 23 nt, Homo sapiens
  source 1..23: mol_type = transcribed RNA
ggcccggccgtgcctgaggtttc

SEQ ID NO: 492 - RNA, 15 nt, Homo sapiens
  source 1..15: mol_type = transcribed RNA
ggcggtgggatcccg

SEQ ID NO: 493 - RNA, 18 nt, Homo sapiens
  source 1..18: mol_type = transcribed RNA
tctaggtggggagactga

SEQ ID NO: 494 - RNA, 16 nt, Homo sapiens
  source 1..16: mol_type = transcribed RNA
gtggggagactgacgg

SEQ ID NO: 495 - RNA, 17 nt, Homo sapiens
  source 1..17: mol_type = transcribed RNA
tgcaggggcaggccagc

SEQ ID NO: 496 - RNA, 17 nt, Homo sapiens
  source 1..17: mol_type = transcribed RNA
tgcaggggcaggccagc

SEQ ID NO: 497 - RNA, 25 nt, Homo sapiens
  source 1..25: mol_type = transcribed RNA
aaggcagggcccccgctccccgggc

SEQ ID NO: 498 - RNA, 15 nt, Homo sapiens
  source 1..15: mol_type = transcribed RNA
gtgtgttgaggaagg

SEQ ID NO: 499 - RNA, 26 nt, Homo sapiens
  source 1..26: mol_type = transcribed RNA
gcgggctgtccggaggggtcggcttt

SEQ ID NO: 500 - RNA, 16 nt, Homo sapiens
  source 1..16: mol_type = transcribed RNA
gctgtccggaggggtc

SEQ ID NO: 501 - RNA, 20 nt, Homo sapiens
  source 1..20: mol_type = transcribed RNA
ggctggtcagatgggagtgg

SEQ ID NO: 502 - RNA, 20 nt, Homo sapiens
  source 1..20: mol_type = transcribed RNA
ggctggtcagatgggagtgg

SEQ ID NO: 503 - RNA, 24 nt, Homo sapiens
  source 1..24: mol_type = transcribed RNA
ctggtacaggcctgggggacaggg

SEQ ID NO: 504 - RNA, 18 nt, Homo sapiens
  source 1..18: mol_type = transcribed RNA
ctggtacaggcctggggg

SEQ ID NO: 505 - RNA, 23 nt, Homo sapiens
  source 1..23: mol_type = transcribed RNA
gccccggcgcgggcgggttctgg

SEQ ID NO: 506 - RNA, 16 nt, Homo sapiens
  source 1..16: mol_type = transcribed RNA
ggagccccggcgcggg

SEQ ID NO: 507 - RNA, 20 nt, Homo sapiens
  source 1..20: mol_type = transcribed RNA
ccccagggcgacgcggcggg

SEQ ID NO: 508 - RNA, 15 nt, Homo sapiens
  source 1..15: mol_type = transcribed RNA
cgcggcgggggcggc

SEQ ID NO: 509 - RNA, 21 nt, Homo sapiens
  source 1..21: mol_type = transcribed RNA
gtgagtcagggtggggctggc

SEQ ID NO: 510 - RNA, 21 nt, Homo sapiens
  source 1..21: mol_type = transcribed RNA
gtgagtcagggtggggctggc

SEQ ID NO: 511 - RNA, 21 nt, Homo sapiens
  source 1..21: mol_type = transcribed RNA
gttggaggcgtgggttttaga

SEQ ID NO: 512 - RNA, 15 nt, Homo sapiens
  source 1..15: mol_type = transcribed RNA
gttggaggcgtgggt

SEQ ID NO: 513 - RNA, 23 nt, Homo sapiens
  source 1..23: mol_type = transcribed RNA
aggaggggtcccgcactgggagg

SEQ ID NO: 514 - RNA, 15 nt, Homo sapiens
  source 1..15: mol_type = transcribed RNA
tgggaggggccctca

SEQ ID NO: 515 - RNA, 23 nt, Homo sapiens
  source 1..23: mol_type = transcribed RNA
gtgggctgggctgggctgggcca

SEQ ID NO: 516 - RNA, 15 nt, Homo sapiens
  source 1..15: mol_type = transcribed RNA
gggctgggctgggct

SEQ ID NO: 517 - RNA, 24 nt, Homo sapiens
  source 1..24: mol_type = transcribed RNA
cgggcgtggtggtgggggtgggtg

SEQ ID NO: 518 - RNA, 15 nt, Homo sapiens
  source 1..15: mol_type = transcribed RNA
cgggcgtggtggtgg

SEQ ID NO: 519 - RNA, 18 nt, Homo sapiens
  source 1..18: mol_type = transcribed RNA
ggtgggcttcccggaggg

SEQ ID NO: 520 - RNA, 15 nt, Homo sapiens
  source 1..15: mol_type = transcribed RNA
ggtgggcttcccgga

SEQ ID NO: 521 - RNA, 26 nt, Homo sapiens
  source 1..26: mol_type = transcribed RNA
cggtgggatcccgcggccgtgttttc

SEQ ID NO: 522 - RNA, 15 nt, Homo sapiens
  source 1..15: mol_type = transcribed RNA
ggggcgccgcgggac

SEQ ID NO: 523 - RNA, 25 nt, Homo sapiens
  source 1..25: mol_type = transcribed RNA
tgggcaggggcttattgtaggagtc

SEQ ID NO: 524 - RNA, 19 nt, Homo sapiens
  source 1..19: mol_type = transcribed RNA
tgggcaggggcttattgta

SEQ ID NO: 525 - RNA, 20 nt, Homo sapiens
  source 1..20: mol_type = transcribed RNA
ccccggggagcccggcggtg

SEQ ID NO: 526 - RNA, 15 nt, Homo sapiens
  source 1..15: mol_type = transcribed RNA
accccggggagcccg

SEQ ID NO: 527 - RNA, 26 nt, Homo sapiens
  source 1..26: mol_type = transcribed RNA
agggacgggacgcggtgcagtgttgt

SEQ ID NO: 528 - RNA, 15 nt, Homo sapiens
  source 1..15: mol_type = transcribed RNA
ggcgggcgggaggga

SEQ ID NO: 529 - RNA, 22 nt, Homo sapiens
  source 1..22: mol_type = transcribed RNA
actcaaactgtgggggcacttt

SEQ ID NO: 530 - RNA, 19 nt, Homo sapiens
  source 1..19: mol_type = transcribed RNA
actcaaactgtgggggcac

SEQ ID NO: 531 - RNA, 16 nt, Homo sapiens
  source 1..16: mol_type = transcribed RNA
gtgaaggcccggcgga

SEQ ID NO: 532 - RNA, 15 nt, Homo sapiens
  source 1..15: mol_type = transcribed RNA
gtgaaggcccggcgg

SEQ ID NO: 533 - RNA, 24 nt, Homo sapiens
  source 1..24: mol_type = transcribed RNA
gctgcgggctgcggtcagggcgat

SEQ ID NO: 534 - RNA, 20 nt, Homo sapiens
  source 1..20: mol_type = transcribed RNA
gctgcgggctgcggtcaggg

SEQ ID NO: 535 - RNA, 30 nt, Homo sapiens
  source 1..30: mol_type = transcribed RNA
tgaggggcagagagcgagacttttctattt

SEQ ID NO: 536 - RNA, 15 nt, Homo sapiens
  source 1..15: mol_type = transcribed RNA
tgaggggcagagagc

SEQ ID NO: 537 - RNA, 23 nt, Homo sapiens
  source 1..23: mol_type = transcribed RNA
caccttgccttgctgcccgggcc

SEQ ID NO: 538 - RNA, 22 nt, Homo sapiens
  source 1..22: mol_type = transcribed RNA
caccttgccttgctgcccgggc

SEQ ID NO: 539 - RNA, 21 nt, Homo sapiens
  source 1..21: mol_type = transcribed RNA
tgggaatgggggtaagggcct

SEQ ID NO: 540 - RNA, 15 nt, Homo sapiens
  source 1..15: mol_type = transcribed RNA
cttctgagcccaggt

SEQ ID NO: 541 - RNA, 15 nt, Homo sapiens
  source 1..15: mol_type = transcribed RNA
ggcggcgggcccggg

SEQ ID NO: 542 - RNA, 15 nt, Homo sapiens
  source 1..15: mol_type = transcribed RNA
ggcggcgggcccggg

SEQ ID NO: 543 - RNA, 16 nt, Homo sapiens
  source 1..16: mol_type = transcribed RNA
ctgggggtcccccgac

SEQ ID NO: 544 - RNA, 15 nt, Homo sapiens
  source 1..15: mol_type = transcribed RNA
gtgtggagctggggc

SEQ ID NO: 545 - RNA, 22 nt, Homo sapiens
  source 1..22: mol_type = transcribed RNA
tgtgggactgcaaatgggagct

SEQ ID NO: 546 - RNA, 22 nt, Homo sapiens
  source 1..22: mol_type = transcribed RNA
tgtgggactgcaaatgggagct

SEQ ID NO: 547 - RNA, 23 nt, Homo sapiens
  source 1..23: mol_type = transcribed RNA
acagcagggctggggattgcagt

SEQ ID NO: 548 - RNA, 19 nt, Homo sapiens
  source 1..19: mol_type = transcribed RNA
tgctgctcccagtcctgcc

SEQ ID NO: 549 - RNA, 17 nt, Homo sapiens
  source 1..17: mol_type = transcribed RNA
aggggctgggcgcgcgc

SEQ ID NO: 550 - RNA, 15 nt, Homo sapiens
  source 1..15: mol_type = transcribed RNA
caggggctgggcgcg

SEQ ID NO: 551 - RNA, 17 nt, Homo sapiens
  source 1..17: mol_type = transcribed RNA
ggggcgggggcgggggc

SEQ ID NO: 552 - RNA, 15 nt, Homo sapiens
  source 1..15: mol_type = transcribed RNA
cgcgccgggcccggg

SEQ ID NO: 553 - RNA, 23 nt, Homo sapiens
  source 1..23: mol_type = transcribed RNA
tggggcggagcttccggaggccc

SEQ ID NO: 554 - RNA, 16 nt, Homo sapiens
  source 1..16: mol_type = transcribed RNA
atcgctggcctggtcg

SEQ ID NO: 555 - RNA, 22 nt, Homo sapiens
  source 1..22: mol_type = transcribed RNA
gagggttgggtggaggctctcc

SEQ ID NO: 556 - RNA, 15 nt, Homo sapiens
  source 1..15: mol_type = transcribed RNA
gagggttgggtggag

SEQ ID NO: 557 - RNA, 18 nt, Homo sapiens
  source 1..18: mol_type = transcribed RNA
ctccgggcggcgccgtgt

SEQ ID NO: 558 - RNA, 18 nt, Homo sapiens
  source 1..18: mol_type = transcribed RNA
ctccgggcggcgccgtgt

SEQ ID NO: 559 - RNA, 21 nt, Homo sapiens
  source 1..21: mol_type = transcribed RNA
gccgggcgtggtggtgggggc

SEQ ID NO: 560 - RNA, 15 nt, Homo sapiens
  source 1..15: mol_type = transcribed RNA
tagccgggcgtggtg

SEQ ID NO: 561 - RNA, 16 nt, Homo sapiens
  source 1..16: mol_type = transcribed RNA
ggcggtgggcggcggg

SEQ ID NO: 562 - RNA, 15 nt, Homo sapiens
  source 1..15: mol_type = transcribed RNA
ggcctctcgggaact

SEQ ID NO: 563 - RNA, 20 nt, Homo sapiens
  source 1..20: mol_type = transcribed RNA
atcccaccactgccaccatt

SEQ ID NO: 564 - RNA, 15 nt, Homo sapiens
  source 1..15: mol_type = transcribed RNA
atcccaccactgcca

SEQ ID NO: 565 - RNA, 25 nt, Homo sapiens
  source 1..25: mol_type = transcribed RNA
caggaaggatttagggacaggcttt

SEQ ID NO: 566 - RNA, 19 nt, Homo sapiens
  source 1..19: mol_type = transcribed RNA
caggaaggatttagggaca

SEQ ID NO: 567 - RNA, 26 nt, Homo sapiens
  source 1..26: mol_type = transcribed RNA
aaaatcacattgccagggattaccac

SEQ ID NO: 568 - RNA, 15 nt, Homo sapiens
  source 1..15: mol_type = transcribed RNA
aatcacattgccagg

SEQ ID NO: 569 - RNA, 23 nt, Homo sapiens
  source 1..23: mol_type = transcribed RNA
taggggcagcagaggacctgggc

SEQ ID NO: 570 - RNA, 20 nt, Homo sapiens
  source 1..20: mol_type = transcribed RNA
taggggcagcagaggacctg

SEQ ID NO: 571 - RNA, 22 nt, Homo sapiens
  source 1..22: mol_type = transcribed RNA
accccactcctggtaccatagt

SEQ ID NO: 572 - RNA, 15 nt, Homo sapiens
  source 1..15: mol_type = transcribed RNA
accccactcctggta

SEQ ID NO: 573 - RNA, 25 nt, Homo sapiens
  source 1..25: mol_type = transcribed RNA
ctgggccagggagcagctggtgggt

SEQ ID NO: 574 - RNA, 20 nt, Homo sapiens
  source 1..20: mol_type = transcribed RNA
tgggccagggagcagctggt

SEQ ID NO: 575 - RNA, 23 nt, Homo sapiens
  source 1..23: mol_type = transcribed RNA
aagggaggaggagcggaggggcc

SEQ ID NO: 576 - RNA, 15 nt, Homo sapiens
  source 1..15: mol_type = transcribed RNA
gggaggaggagcgga

SEQ ID NO: 577 - RNA, 20 nt, Homo sapiens
  source 1..20: mol_type = transcribed RNA
atcccacctctgccaccaaa

SEQ ID NO: 578 - RNA, 15 nt, Homo sapiens
  source 1..15: mol_type = transcribed RNA
atcccacctctgcca

SEQ ID NO: 579 - RNA, 23 nt, Homo sapiens
  source 1..23: mol_type = transcribed RNA
gggggccgatacactgtacgaga

SEQ ID NO: 580 - RNA, 20 nt, Homo sapiens
  source 1..20: mol_type = transcribed RNA
gggggccgatacactgtacg

SEQ ID NO: 581 - RNA, 23 nt, Homo sapiens
  source 1..23: mol_type = transcribed RNA
cacaggtgaggttcttgggagcc

SEQ ID NO: 582 - RNA, 15 nt, Homo sapiens
  source 1..15: mol_type = transcribed RNA
acaggtgaggttctt

SEQ ID NO: 583 - RNA, 25 nt, Homo sapiens
  source 1..25: mol_type = transcribed RNA
gggtggggatttgttgcattacttg

SEQ ID NO: 584 - RNA, 20 nt, Homo sapiens
  source 1..20: mol_type = transcribed RNA
gggtggggatttgttgcatt

SEQ ID NO: 585 - RNA, 23 nt, Homo sapiens
  source 1..23: mol_type = transcribed RNA
cggggcagctcagtacaggatac

SEQ ID NO: 586 - RNA, 15 nt, Homo sapiens
  source 1..15: mol_type = transcribed RNA
agctcagtacaggat

SEQ ID NO: 587 - RNA, 17 nt, Homo sapiens
  source 1..17: mol_type = transcribed RNA
gcggggcggcaggggcc

SEQ ID NO: 588 - RNA, 15 nt, Homo sapiens
  source 1..15: mol_type = transcribed RNA
gggggcggggcggca

SEQ ID NO: 589 - RNA, 22 nt, Homo sapiens
  source 1..22: mol_type = transcribed RNA
ggcagggacagcaaaggggtgc

SEQ ID NO: 590 - RNA, 18 nt, Homo sapiens
  source 1..18: mol_type = transcribed RNA
gcagggacagcaaagggg

SEQ ID NO: 591 - RNA, 16 nt, Homo sapiens
  source 1..16: mol_type = transcribed RNA
tgggggggaagaaaag

SEQ ID NO: 592 - RNA, 16 nt, Homo sapiens
  source 1..16: mol_type = transcribed RNA
tgggggggaagaaaag

SEQ ID NO: 593 - RNA, 23 nt, Homo sapiens
  source 1..23: mol_type = transcribed RNA
gagggagggacgggggctgtgct

SEQ ID NO: 594 - RNA, 15 nt, Homo sapiens
  source 1..15: mol_type = transcribed RNA
gaggagggagggagg

SEQ ID NO: 595 - RNA, 24 nt, Homo sapiens
  source 1..24: mol_type = transcribed RNA
tgactggggagcagaaggagaacc

SEQ ID NO: 596 - RNA, 15 nt, Homo sapiens
  source 1..15: mol_type = transcribed RNA
gactggggagcagaa

SEQ ID NO: 597 - RNA, 22 nt, Homo sapiens
  source 1..22: mol_type = transcribed RNA
atatagggattggagccgtggc

SEQ ID NO: 598 - RNA, 20 nt, Homo sapiens
  source 1..20: mol_type = transcribed RNA
atatagggattggagccgtg

SEQ ID NO: 599 - RNA, 19 nt, Homo sapiens
  source 1..19: mol_type = transcribed RNA
gctgggcgaggctggcatc

SEQ ID NO: 600 - RNA, 17 nt, Homo sapiens
  source 1..17: mol_type = transcribed RNA
gctgggcgaggctggca

SEQ ID NO: 601 - RNA, 23 nt, Homo sapiens
  source 1..23: mol_type = transcribed RNA
tggggaaggcgtcagtgtcgggt

SEQ ID NO: 602 - RNA, 16 nt, Homo sapiens
  source 1..16: mol_type = transcribed RNA
tggggaaggcgtcagt

SEQ ID NO: 603 - RNA, 21 nt, Homo sapiens
  source 1..21: mol_type = transcribed RNA
cagcaggggagagagaggagt

SEQ ID NO: 604 - RNA, 20 nt, Homo sapiens
  source 1..20: mol_type = transcribed RNA
cagcaggggagagagaggag

SEQ ID NO: 605 - RNA, 26 nt, Homo sapiens
  source 1..26: mol_type = transcribed RNA
agggctggactcagcggcggagctgg

SEQ ID NO: 606 - RNA, 17 nt, Homo sapiens
  source 1..17: mol_type = transcribed RNA
gcggcggagctggctgc

SEQ ID NO: 607 - RNA, 24 nt, Homo sapiens
  source 1..24: mol_type = transcribed RNA
caggcaggagccggactggacctc

SEQ ID NO: 608 - RNA, 21 nt, Homo sapiens
  source 1..21: mol_type = transcribed RNA
tccaggcaggagccggactgg

SEQ ID NO: 609 - RNA, 19 nt, Homo sapiens
  source 1..19: mol_type = transcribed RNA
gggtgcgggccggcggggt

SEQ ID NO: 610 - RNA, 15 nt, Homo sapiens
  source 1..15: mol_type = transcribed RNA
tgcgggccggcgggg

SEQ ID NO: 611 - RNA, 27 nt, Homo sapiens
  source 1..27: mol_type = transcribed RNA
atcacattgccagggatttccaaccga

SEQ ID NO: 612 - RNA, 15 nt, Homo sapiens
  source 1..15: mol_type = transcribed RNA
aatcacattgccagg

SEQ ID NO: 613 - RNA, 19 nt, Homo sapiens
  source 1..19: mol_type = transcribed RNA
tggcgggtgcgggggtggg

SEQ ID NO: 614 - RNA, 15 nt, Homo sapiens
  source 1..15: mol_type = transcribed RNA
tggcgggtgcggggg

SEQ ID NO: 615 - RNA, 21 nt, Homo sapiens
  source 1..21: mol_type = transcribed RNA
ttgaggagacatggtgggggc

SEQ ID NO: 616 - RNA, 15 nt, Homo sapiens
  source 1..15: mol_type = transcribed RNA
ttgaggagacatggt

SEQ ID NO: 617 - RNA, 27 nt, Homo sapiens
  source 1..27: mol_type = transcribed RNA
aaaccgttaccattactgagtttagta

SEQ ID NO: 618 - RNA, 15 nt, Homo sapiens
  source 1..15: mol_type = transcribed RNA
gaaaccgttaccatt

SEQ ID NO: 619 - RNA, 23 nt, Homo sapiens
  source 1..23: mol_type = transcribed RNA
ccagggctggcagtgacatgggt

SEQ ID NO: 620 - RNA, 19 nt, Homo sapiens
  source 1..19: mol_type = transcribed RNA
cagggctggcagtgacatg

SEQ ID NO: 621 - RNA, 23 nt, Homo sapiens
  source 1..23: mol_type = transcribed RNA
tggggcggagcttccggaggccc

SEQ ID NO: 622 - RNA, 15 nt, Homo sapiens
  source 1..15: mol_type = transcribed RNA
gccccgggaaagcgt

SEQ ID NO: 623 - RNA, 24 nt, Homo sapiens
  source 1..24: mol_type = transcribed RNA
agacacatttggagagggaacctc

SEQ ID NO: 624 - RNA, 16 nt, Homo sapiens
  source 1..16: mol_type = transcribed RNA
agacacatttggagag

SEQ ID NO: 625 - RNA, 24 nt, Homo sapiens
  source 1..24: mol_type = transcribed RNA
gatcggtcgagagcgtcctggctg

SEQ ID NO: 626 - RNA, 15 nt, Homo sapiens
  source 1..15: mol_type = transcribed RNA
gctgggcggggcgcg

SEQ ID NO: 627 - RNA, 23 nt, Homo sapiens
  source 1..23: mol_type = transcribed RNA
ctgggggacgcgtgagcgcgagc

SEQ ID NO: 628 - RNA, 21 nt, Homo sapiens
  source 1..21: mol_type = transcribed RNA
ctgggggacgcgtgagcgcga

SEQ ID NO: 629 - RNA, 27 nt, Homo sapiens
  source 1..27: mol_type = transcribed RNA
caaggtggctgggagagggttgtttac

SEQ ID NO: 630 - RNA, 15 nt, Homo sapiens
  source 1..15: mol_type = transcribed RNA
gtgagctcaaggtgg

SEQ ID NO: 631 - RNA, 29 nt, Homo sapiens
  source 1..29: mol_type = transcribed RNA
gctgggttaagccgagctgggttgggctg

SEQ ID NO: 632 - RNA, 19 nt, Homo sapiens
  source 1..19: mol_type = transcribed RNA
ctgggttgggctgggctgg

SEQ ID NO: 633 - RNA, 19 nt, Homo sapiens
  source 1..19: mol_type = transcribed RNA
gagggcgggtggaggagga

SEQ ID NO: 634 - RNA, 15 nt, Homo sapiens
  source 1..15: mol_type = transcribed RNA
gcgggtggaggagga

SEQ ID NO: 635 - RNA, 20 nt, Homo sapiens
  source 1..20: mol_type = transcribed RNA
cagggggactgggggtgagc

SEQ ID NO: 636 - RNA, 25 nt, Homo sapiens
  source 1..25: mol_type = transcribed RNA
acttgggcaggagggaccctgtatg

SEQ ID NO: 637 - RNA, 22 nt, Homo sapiens
  source 1..22: mol_type = transcribed RNA
agcccgccccagccgaggttct

SEQ ID NO: 638 - RNA, 17 nt, Homo sapiens
  source 1..17: mol_type = transcribed RNA
ggatggaggaggggtct

SEQ ID NO: 639 - RNA, 17 nt, Homo sapiens
  source 1..17: mol_type = transcribed RNA
gggagtctacagcaggg

SEQ ID NO: 640 - RNA, 22 nt, Homo sapiens
  source 1..22: mol_type = transcribed RNA
gtgcggaacgctggccggggcg

SEQ ID NO: 641 - RNA, 24 nt, Homo sapiens
  source 1..24: mol_type = transcribed RNA
ggaggccggggtggggcggggcgg

SEQ ID NO: 642 - RNA, 23 nt, Homo sapiens
  source 1..23: mol_type = transcribed RNA
aggaagccctggaggggctggag

SEQ ID NO: 643 - RNA, 68 nt, Homo sapiens
  source 1..68: mol_type = transcribed RNA
gggcgcagggggactgggggtgagcaggcccagaacccagctcgtgctcactctcagtccctccctag

SEQ ID NO: 644 - RNA, 70 nt, Homo sapiens
  source 1..70: mol_type = transcribed RNA
tgtgcacttgggcaggagggaccctgtatgtctccccgcagcaccgtcatcgtgtccctcttgtccacag

SEQ ID NO: 645 - RNA, 80 nt, Homo sapiens; identical to SEQ ID NO: 302
  source 1..80: mol_type = transcribed RNA

SEQ ID NO: 646 - RNA, 60 nt, Homo sapiens
  source 1..60: mol_type = transcribed RNA
tgtgaatgacccccttccagagccaaaatcaccagggatggaggaggggtcttgggtact

SEQ ID NO: 647 - RNA, 76 nt, Homo sapiens
  source 1..76: mol_type = transcribed RNA
ccgatgcctcgggagtctacagcagggccatgtctgtgagggcccaagggtgcatgtgtctcccaggtttcggtgc

SEQ ID NO: 648 - RNA, 61 nt, Homo sapiens
  source 1..61: mol_type = transcribed RNA
gtgcggaacgctggccggggcgggaggggaagggacgcccggccggaacgccgcactcacg

SEQ ID NO: 649 - RNA, 64 nt, Homo sapiens
  source 1..64: mol_type = transcribed RNA
ccccgggcccggcgttccctccccttccgtgcgccagtggaggccggggtggggcggggcgggg

SEQ ID NO: 650 - RNA, 64 nt, Homo sapiens; identical to SEQ ID NO: 649
  source 1..64: mol_type = transcribed RNA

SEQ ID NO: 651 - RNA, 118 nt, Homo sapiens
  source 1..118: mol_type = transcribed RNA
gcaggtgaactggcaggccaggaagaggaggaagccctggaggggctggaggtgatggatgttttcctccggttctcagggctccacctctttcgggccgtagagccagggctggtgc

SEQ ID NO: 652 - RNA, 23 nt, Homo sapiens
  source 1..23: mol_type = transcribed RNA
cagcccgccccagccgaggttct

SEQ ID NO: 653 - RNA, 17 nt, Homo sapiens
  source 1..17: mol_type = transcribed RNA
agcccgccccagccgag

SEQ ID NO: 654 - RNA, 16 nt, Homo sapiens
  source 1..16: mol_type = transcribed RNA
cgggcccggcgttccc

SEQ ID NO: 655 - RNA, 15 nt, Homo sapiens
  source 1..15: mol_type = transcribed RNA
ccgggcccggcgttc

SEQ ID NO: 656 - RNA, 25 nt, Homo sapiens
  source 1..25: mol_type = transcribed RNA
aggaagccctggaggggctggaggt

SEQ ID NO: 657 - RNA, 15 nt, Homo sapiens
  source 1..15: mol_type = transcribed RNA
aggaagaggaggaag